SEQUENCE LISTING (12 sequences)

SEQ ID NO: 1 - DNA, 6240 bp, Homo sapiens
agatgcgatccagcggctctgggggcggcagcggtggtagcagctggtacctcccgccgcctctgttcggagggtcgcggggcaccgaggtgctttccggccgccctctggtcggccacccaaagccgcgggcgctgatgatgggtgaggagggggcggcaagatttcgggcgcccctgccctgaacgccctcagctgctgccgccggggccgctccagtgcctgcgaactctgaggagccgaggcgccggtgagagcaaggacgctgcaaacttgcgcagcgcgggggctgggattcacgcccagaagttcagcaggcagacagtccgaagccttcccgcagcggagagatagcttgagggtgcgcaagacggcagcctccgccctcggttcccgcccagaccgggcagaagagcttggaggagccaaaaggaacgcaaaaggcggccaggacagcgtgcagcagctgggagccgccgttctcagccttaaaagttgcagagattggaggctgccccgagaggggacagaccccagctccgactgcggggggcaggagaggacggtacccaactgccacctcccttcaaccatagtagttcctctgtaccgagcgcagcgagctacagacgggggcgcggcactcggcgcggagagcgggaggctcaaggtcccagccagtgagcccagtgtgcttgagtgtctctggactcgcccctgagcttccaggtctgtttcatttagactcctgctcgcctccgtgcagttgggggaaagcaagagacttgcgcgcacgcacagtcctctggagatcaggtggaaggagccgctgggtaccaaggactgttcagagcctcttcccatctcggggagagcgaagggtgaggctgggcccggagagcagtgtaaacggcctcctccggcgggatgggagccatcgggctcctgtggctcctgccgctgctgctttccacggcagctgtgggctccgggatggggaccggccagcgcgcgggctccccagctgcggggccgccgctgcagccccgggagccactcagctactcgcgcctgcagaggaagagtctggcagttgacttcgtggtgccctcgctcttccgtgtctacgcccgggacctactgctgccaccatcctcctcggagctgaaggctggcaggcccgaggcccgcggctcgctagctctggactgcgccccgctgctcaggttgctggggccggcgccgggggtctcctggaccgccggttcaccagccccggcagaggcccggacgctgtccagggtgctgaagggcggctccgtgcgcaagctccggcgtgccaagcagttggtgctggagctgggcgaggaggcgatcttggagggttgcgtcgggccccccggggaggcggctgtggggctgctccagttcaatctcagcgagctgttcagttggtggattcgccaaggcgaagggcgactgaggatccgcctgatgcccgagaagaaggcgtcggaagtgggcagagagggaaggctgtccgcggcaattcgcgcctcccagccccgccttctcttccagatcttcgggactggtcatagctccttggaatcaccaacaaacatgccttctccttctcctgattattttacatggaatctcacctggataatgaaagactccttccctttcctgtctcatcgcagccgatatggtctggagtgcagctttgacttcccctgtgagctggagtattcccctccactgcatgacctcaggaaccagagctggtcctggcgccgcatcccctccgaggaggcctcccagatggacttgctggatgggcctggggcagagcgttctaaggagatgcccagaggctcctttctccttctcaacacctcagctgactccaagcacaccatcctgagtccgtggatgaggagcagcagtgagcactgcacactggccgtctcggtgcacaggcacctgcagccctctggaaggtacattgcccagctgctgccccacaacgaggctgcaagagagatcctcctgatgcccactccagggaagcatggttggacagtgctccagggaagaatcgggcgtccagacaacccatttcgagtggccctggaatacatctccagtggaaaccgcagcttgtctgcagtggacttctttgccctgaagaactgcagtgaaggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa

SEQ ID NO: 2 - DNA, 6829 bp, Homo sapiens
gcagactgtgctggagctggtgctgaaaaagggggtttgcagaggctgccctggggctggtgctgaaagaagagcccacagctgacttcatggtgctacaataacctcagaatctacttttcactctcaggagaacccacatgtctaatatttagacatgatggcaaactgggcggaagcaagacctctcctcattcttattgttttattagggcaatttgtctcaataaaagcccaggaagaagacgaggatgaaggatatggtgaagaaatagcctgcactcagaatggccagatgtacttaaacagggacatttggaaacctgccccttgtcagatctgtgtctgtgacaatggagccattctctgtgacaagatagaatgccaggatgtgctggactgtgccgaccctgtaacgccccctggggaatgctgtcctgtctgttcacaaacacctggaggtggcaataccaattttggtagaggaagaaagggacaaaagggagaaccaggattagtgcctgttgtaacaggcatacgtggtcgtccaggaccggcaggacctccaggatcacagggaccaagaggagagcgagggccaaaaggaagacctggccctcgtggacctcagggaattgatggagaaccaggtgttcctggtcaacctggtgctccaggacctcctggacatccgtcccacccaggacccgatggcttgagcaggccgttttcagctcaaatggctgggttggatgaaaaatctggacttgggagtcaagtaggactaatgcctggctctgtgggtcctgttggcccaaggggaccacagggtttacaaggacagcaaggtggtgcaggacctacaggacctcctggtgaacctggtgatcctggaccaatgggtccgattggttcacgtggaccagagggccctcctggtaaacctggggaagatggtgaacctggcagaaatggaaatcctggtgaagtgggatttgcaggatctccgggagctcgtggatttcctggggctcctggtcttccaggtctgaagggtcaccgaggacacaaaggtcttgaaggccctaaaggtgaagttggagcacctggttccaagggtgaagctggccccactggtccaatgggtgccatgggtcctctgggtccgaggggaatgccaggagagagagggagacttgggccacagggtgctcctggacaacgaggtgcacatggtatgcctggaaaacctggaccaatgggtcctcttgggataccaggctcttctggttttccaggaaatcctggaatgaagggagaagcaggtcctacaggggcgcgaggccctgaaggtcctcaggggcagagaggtgaaactgggcccccaggtccagttggctctccaggtcttcctggtgcaataggaactgatggtactcctggtgccaaaggcccaacgggctctccaggtacctctggtcctcctggctcagcagggcctcctggatctccaggacctcagggtagcactggtcctcagggaattcgaggccaaccgggtgatccaggagttccaggtttcaaaggagaagctggcccaaaaggggaaccagggccacatggtattcagggtccgataggcccacccggtgaagaaggcaaaagaggtcccagaggtgacccaggaacagttggtcctccagggccagtgggagaaaggggtgctcctggcaatcgtggttttccaggctctgatggtttacctgggccaaagggtgctcaaggagaacggggtcctgtaggttcttcaggacccaaaggaagccagggggatccaggacgtccaggggaacctgggcttccaggtgctcggggtttgacaggaaatcctggtgttcaaggtcctgaaggaaaacttggacctttgggtgcgccaggggaagatggccgtccaggtcctccaggctccataggaatcagagggcagcccgggagcatgggccttccaggccccaaaggtagcagtggtgaccctgggaaacctggagaagcaggaaatgctggagttcctgggcagaggggagctcctggaaaagatggtgaagttggtccttctggtcctgtgggcccgccgggtctagctggtgaaagaggagaacaaggacctccaggccccacaggttttcaggggcttcctggtcctccagggcctcctggagaaggtggaaaaccaggtgatcaaggtgttcctggagatcccggagcagttggcccgttaggacctagaggagaacgaggaaatcctggggaaagaggagaacctgggataactggactccctggtgagaagggaatggctggaggacatggtcctgatggcccaaaaggcagtccaggtccatctgggacccctggagatacaggcccaccaggtcttcaaggtatgccgggagaaagaggaattgcaggaactcctggccccaagggtgacagaggtggcataggagaaaaaggtgctgaaggcacagctggaaatgatggtgcaagaggtcttccaggtcctttgggccctccaggtccggcaggtcctactggagaaaagggtgaacctggtcctcgaggtttagttggccctcctggctcccggggcaatcctggttctcgaggtgaaaatgggccaactggagctgttggttttgccggaccccagggtcctgacggacagcctggagtaaaaggtgaacctggagagccaggacagaagggagatgctggttctcctggaccacaaggtttagcaggatcccctggccctcatggtcctaatggtgttcctggactaaaaggtggtcgaggaacccaaggtccgcctggtgctacaggatttcctggttctgcgggcagagttggacctccaggccctgctggagctccaggacctgcgggacccctaggggaacccgggaaggagggacctccaggtcttcgtggggaccctggctctcatgggcgtgtgggagatcgaggaccagctggcccccctggtggcccaggagacaaaggggacccaggagaagatgggcaacctggtccagatggcccccctggtccagctggaacgaccgggcagagaggaattgttggcatgcctgggcaacgtggagagagaggcatgcccggcctaccaggcccagcgggaacaccaggaaaagtaggaccaactggtgcaacaggagataaaggtccacctggacctgtggggcccccaggctccaatggtcctgtaggggaacctggaccagaaggtccagctggcaatgatggtaccccaggacgggatggtgctgttggagaacgtggtgatcgtggagaccctgggcctgcaggtctgccaggctctcagggtgcccctggaactcctggccctgtgggtgctccaggagatgcaggacaaagaggagatccgggttctcggggtcctataggaccacctggtcgagctgggaaacgtggattacctggaccccaaggacctcgtggtgacaaaggtgatcatggagaccgaggtgacagaggtcagaagggccacagaggctttactggtcttcagggtcttcctggccctcctggtccaaatggtgaacaaggaagtgctggaatccctggaccatttggcccaagaggtcctccaggcccagttggtccttcaggtaaagaaggaaaccctgggccacttgggccaattggacctccaggtgtacgaggcagtgtaggagaagcaggacctgagggccctcctggtgagcctggcccacctggccctccgggtccccctggccaccttacagctgctcttggggatatcatggggcactatgatgaaagcatgccagatccacttcctgagtttactgaagatcaggcggctcctgatgacaaaaacaaaacggacccaggggttcatgctaccctgaagtcactcagtagtcagattgaaaccatgcgcagccccgatggctcgaaaaagcacccagcccgcacgtgtgatgacctaaagctttgccattccgcaaagcagagtggtgaatactggattgatcctaaccaaggatctgttgaagatgcaatcaaagtttactgcaacatggaaacaggagaaacatgtatttcagcaaacccatccagtgtaccacgtaaaacctggtgggccagtaaatctcctgacaataaacctgtttggtatggtcttgatatgaacagagggtctcagttcgcttatggagaccaccaatcacctaatacagccattactcagatgacttttttgcgccttttatcaaaagaagcctcccagaacatcacttacatctgtaaaaacagtgtaggatacatggacgatcaagctaagaacctcaaaaaagctgtggttctcaaaggggcaaatgacttagatatcaaagcagagggaaatattagattccggtatatcgttcttcaagacacttgctctaagcggaatggaaatgtgggcaagactgtctttgaatatagaacacagaatgtggcacgcttgcccatcatagatcttgctcctgtggatgttggcggcacagaccaggaattcggcgttgaaattgggccagtttgttttgtgtaaagtaagccaagacacatcgacaatgagcaccaccatcaatgaccaccgccattcacaagaactttgactgtttgaagttgatcctgagactcttgaagtaatggctgatcctgcatcagcattgtatatatggtcttaagtgcctggcctccttatccttcagaatatttattttacttacaatcctcaagttttaattgattttaaatatttttcaatacaacagtttaggtttaagatgaccaatgacaatgaccacctttgcagaaagtaaactgattgaataaataaatctccgttttcttcaatttatttcagtgtaatgaaaaagttgcttagtatttatgaggaaattcttcttcctggcaggtagcttaaagagtggggtatatagagccacaacacatgtttattttgcttggctgcagttgaaaaatagaaattagtgcccttttgtgacctctcattccaagattgtcaattaaaaatgagtttaaaatgtttaacttgtgatcgagacctacatgcatgtcttgatattgtgtaactataatagagactctttaaggagaatcttaaaaaaaaaaaaacgtttctcactgtcttaaatagaatttttaaatagtatatattcagtggcattttggagaacaaagtgaatttacttcgacttcttaaatttttgtaaaagactataagtttagacatctttctcattcaaatttaaagatatctttctcctcttgatcaatctatcaatattgatagaagtcacactagtatataccatttaatacatttacactttcttatttaagaagatattgaatgcaaaataattgacatatagaactttacaaacatatgtccaaggactctaaattgagactcttccacatgtacaatctcatcatcctgaagcctataatgaagaaaaagatctagaaactgagttgtggagctgactctaatcaaatgtgatgattggaattagaccatttggcctttgaactttcataggaaaaatgacccaacatttcttagcatgagctacctcatctctagaagctgggatggacttactattcttgtttatattttagatactgaaaggtgctatgcttctgttattattccaagactggagataggcagggctaaaaaggtattattatttttcctttaatgatggtgctaaaattcttcctataaaattccttaaaaataaagatggtttaatcactaccattgtgaaaacataactgttagacttcccgtttctgaaagaaagagcatcgttccaatgcttgttcactgttcctctgtcatactgtatctggaatgctttgtaatacttgcatgcttcttagaccagaacatgtaggtccccttgtgtctcaatactttttttttcttaattgcatttgttggctctattttaatttttttcttttaaaataaacagctgggaccatcccaaaagacaagccatgcatacaactttggtcatgtatctctgcaaagcatcaaattaaatgcacgcttttgtcatgtcagtggtttttgttttgtgaaattcctttgaccatattagatctatttcatttccaatagtgaaaaggagatgtggtggtatactttgtttgccatttgtttaaaagatacaacggataccttctatcatgtatgtactggcttataaatgaaaatctatctacaacattacccacaaaggcaacatgacaccaattatcactgcctctgcccttaaaaatgtcagagtagtattattgataaaaagggcaagcaatagatttttcatgactgaataaactgtaataataaaacatatgtctcaaagtgtatcacatatgaatttagcctaattgttttcagtttcattctcaatatttagtttacaacatcattttcccctaaactggttatattttgacctgtatatcttaaatttgagtatttatatgcctaaatacatgtgtgagttttgtttgacttccaagtccaaactataagattatataagttcatatagatgaatcagaaatatgtggtaatactattaagtcacaaacactaacaatttccaactatagaaataacagttcttatttggattttgggaatgctaccaataaaagcctgcccagacca

SEQ ID NO: 3 - DNA, 5490 bp, Homo sapiens
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtggagaatactgggttgaccctaaccaaggatgcaaattggatgctatcaaggtattctgtaatatggaaactggggaaacatgcataagtgccaatcctttgaatgttccacggaaacactggtggacagattctagtgctgagaagaaacacgtttggtttggagagtccatggatggtggttttcagtttagctacggcaatcctgaacttcctgaagatgtccttgatgtgcatctggcattccttcgacttctctccagccgagcttcccagaacatcacatatcactgcaaaaatagcattgcatacatggatcaggccagtggaaatgtaaagaaggccctgaagctgatggggtcaaatgaaggtgaattcaaggctgaaggaaatagcaaattcacctacacagttctggaggatggttgcacgaaacacactggggaatggagcaaaacagtctttgaatatcgaacacgcaaggctgtgagactacctattgtagatattgcaccctatgacattggtggtcctgatcaagaatttggtgtggacgttggccctgtttgctttttataaaccaaactctatctgaaatcccaacaaaaaaaatttaactccatatgtgttcctcttgttctaatcttgtcaaccagtgcaagtgaccgacaaaattccagttatttatttccaaaatgtttggaaacagtataatttgacaaagaaaaatgatacttctctttttttgctgttccaccaaatacaattcaaatgctttttgttttatttttttaccaattccaatttcaaaatgtctcaatggtgctataataaataaacttcaacactctttatgataacaacactgtgttatattctttgaatcctagcccatctgcagagcaatgactgtgctcaccagtaaaagataacctttctttctgaaatagtcaaatacgaaattagaaaagccctccctattttaactacctcaactggtcagaaacacagattgtattctatgagtcccagaagatgaaaaaaattttatacgttgataaaacttataaatttcattgattaatctcctggaagattggtttaaaaagaaaagtgtaatgcaagaatttaaagaaatatttttaaagccacaattattttaatattggatatcaactgcttgtaaaggtgctcctcttttttcttgtcattgctggtcaagattactaatatttgggaaggctttaaagacgcatgttatggtgctaatgtactttcacttttaaactctagatcagaattgttgacttgcattcagaacataaatgcacaaaatctgtacatgtctcccatcagaaagattcattggcatgccacaggggattctcctccttcatcctgtaaaggtcaacaataaaaaccaaattatggggctgcttttgtcacactagcatagagaatgtgttgaaatttaactttgtaagcttgtatgtggttgttgatcttttttttccttacagacacccataataaaatatcatattaaaattc

SEQ ID NO: 4 - protein, 1620 aa, Homo sapiens
MGAIGLLWLLPLLLSTAAVGSGMGTGQRAGSPAAGPPLQPREPLSYSRLQRKSLAVDFVVPSLFRVYARDLLLPPSSSELKAGRPEARGSLALDCAPLLRLLGPAPGVSWTAGSPAPAEARTLSRVLKGGSVRKLRRAKQLVLELGEEAILEGCVGPPGEAAVGLLQFNLSELFSWWIRQGEGRLRIRLMPEKKASEVGREGRLSAAIRASQPRLLFQIFGTGHSSLESPTNMPSPSPDYFTWNLTWIMKDSFPFLSHRSRYGLECSFDFPCELEYSPPLHDLRNQSWSWRRIPSEEASQMDLLDGPGAERSKEMPRGSFLLLNTSADSKHTILSPWMRSSSEHCTLAVSVHRHLQPSGRYIAQLLPHNEAAREILLMPTPGKHGWTVLQGRIGRPDNPFRVALEYISSGNRSLSAVDFFALKNCSEGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 5 - protein, 1499 aa, Homo sapiens
MMANWAEARPLLILIVLLGQFVSIKAQEEDEDEGYGEEIACTQNGQMYLNRDIWKPAPCQICVCDNGAILCDKIECQDVLDCADPVTPPGECCPVCSQTPGGGNTNFGRGRKGQKGEPGLVPVVTGIRGRPGPAGPPGSQGPRGERGPKGRPGPRGPQGIDGEPGVPGQPGAPGPPGHPSHPGPDGLSRPFSAQMAGLDEKSGLGSQVGLMPGSVGPVGPRGPQGLQGQQGGAGPTGPPGEPGDPGPMGPIGSRGPEGPPGKPGEDGEPGRNGNPGEVGFAGSPGARGFPGAPGLPGLKGHRGHKGLEGPKGEVGAPGSKGEAGPTGPMGAMGPLGPRGMPGERGRLGPQGAPGQRGAHGMPGKPGPMGPLGIPGSSGFPGNPGMKGEAGPTGARGPEGPQGQRGETGPPGPVGSPGLPGAIGTDGTPGAKGPTGSPGTSGPPGSAGPPGSPGPQGSTGPQGIRGQPGDPGVPGFKGEAGPKGEPGPHGIQGPIGPPGEEGKRGPRGDPGTVGPPGPVGERGAPGNRGFPGSDGLPGPKGAQGERGPVGSSGPKGSQGDPGRPGEPGLPGARGLTGNPGVQGPEGKLGPLGAPGEDGRPGPPGSIGIRGQPGSMGLPGPKGSSGDPGKPGEAGNAGVPGQRGAPGKDGEVGPSGPVGPPGLAGERGEQGPPGPTGFQGLPGPPGPPGEGGKPGDQGVPGDPGAVGPLGPRGERGNPGERGEPGITGLPGEKGMAGGHGPDGPKGSPGPSGTPGDTGPPGLQGMPGERGIAGTPGPKGDRGGIGEKGAEGTAGNDGARGLPGPLGPPGPAGPTGEKGEPGPRGLVGPPGSRGNPGSRGENGPTGAVGFAGPQGPDGQPGVKGEPGEPGQKGDAGSPGPQGLAGSPGPHGPNGVPGLKGGRGTQGPPGATGFPGSAGRVGPPGPAGAPGPAGPLGEPGKEGPPGLRGDPGSHGRVGDRGPAGPPGGPGDKGDPGEDGQPGPDGPPGPAGTTGQRGIVGMPGQRGERGMPGLPGPAGTPGKVGPTGATGDKGPPGPVGPPGSNGPVGEPGPEGPAGNDGTPGRDGAVGERGDRGDPGPAGLPGSQGAPGTPGPVGAPGDAGQRGDPGSRGPIGPPGRAGKRGLPGPQGPRGDKGDHGDRGDRGQKGHRGFTGLQGLPGPPGPNGEQGSAGIPGPFGPRGPPGPVGPSGKEGNPGPLGPIGPPGVRGSVGEAGPEGPPGEPGPPGPPGPPGHLTAALGDIMGHYDESMPDPLPEFTEDQAAPDDKNKTDPGVHATLKSLSSQIETMRSPDGSKKHPARTCDDLKLCHSAKQSGEYWIDPNQGSVEDAIKVYCNMETGETCISANPSSVPRKTWWASKSPDNKPVWYGLDMNRGSQFAYGDHQSPNTAITQMTFLRLLSKEASQNITYICKNSVGYMDDQAKNLKKAVVLKGANDLDIKAEGNIRFRYIVLQDTCSKRNGNVGKTVFEYRTQNVARLPIIDLAPVDVGGTDQEFGVEIGPVCFV

SEQ ID NO: 6 - protein, 1466 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSGEYWVDPNQGCKLDAIKVFCNMETGETCISANPLNVPRKHWWTDSSAEKKHVWFGESMDGGFQFSYGNPELPEDVLDVHLAFLRLLSSRASQNITYHCKNSIAYMDQASGNVKKALKLMGSNEGEFKAEGNSKFTYTVLEDGCTKHTGEWSKTVFEYRTRKAVRLPIVDIAPYDIGGPDQEFGVDVGPVCFL

SEQ ID NO: 7 - DNA, 4285 bp, Homo sapiens
gcagactgtgctggagctggtgctgaaaaagggggtttgcagaggctgccctggggctggtgctgaaagaagagcccacagctgacttcatggtgctacaataacctcagaatctacttttcactctcaggagaacccacatgtctaatatttagacatgatggcaaactgggcggaagcaagacctctcctcattcttattgttttattagggcaatttgtctcaataaaagcccaggaagaagacgaggatggaacatccccaggctccaagatggccctgcagagctccttcacttgttggaatgggacagtcctccagcttgggcaggcctgtgacttccaccaggactgtgcccagggagaagatgagagccagatgtgccggaaactgcctgtgggtttttactgcaactttgaagatggcttctgtggctggacccaaggcacactgtcaccccacactcctcaatggcaggtcaggaccctaaaggatgcccggttccaggaccaccaagaccatgctctattgctcagtaccactgatgtccccgcttctgaaagtgctacagtgaccagtgctacgtttcctgcaccgatcaagagctctccatgtgagctccgaatgtcctggctcattcgtggagtcttgaggggaaacgtgtccttggtgctagtggagaacaaaaccgggaaggagcaaggcaggatggtctggcatgtcgccgcctatgaaggcttgagcctgtggcagtggatggtgttgcctctcctcgatgtgtctgacaggttctggctgcagatggtcgcatggtggggacaaggatccagagccatcgtggcttttgacaatatctccatcagcctggactgctacctcaccattagcggagaggacaagatcctgcagaatacagcacccaaatcaagaaacctgtttgagagaaacccaaacaaggagctgaaacccggggaaaattcaccaagacagacccccatctttgaccctacagttcattggctgttcaccacatgtggggccagcgggccccatggccccacccaggcacagtgcaacaacgcctaccagaactccaacctgagcgtggaggtggggagcgagggccccctgaaaggcatccagatctggaaggtgccagccaccgacacctacagcatctcgggctacggagctgctggcgggaaaggcgggaagaacaccatgatgcggtcccacggcgtgtctgtgctgggcatcttcaacctggagaaggatgacatgctgtacatcctggttgggcagcagggagaggacgcctgccccagtacaaaccagttaatccagaaagtctgcattggagagaacaatgtgatagaagaagaaatccgtgtgaacagaagcgtgcatgagtgggcaggaggcggaggaggagggggtggagccacctacgtatttaagatgaaggatggagtgccggtgcccctgatcattgcagccggaggtggtggcagggcctacggggccaagacagacacgttccacccagagagactggagaataactcctcggttctagggctaaacggcaattccggagccgcaggtggtggaggtggctggaatgataacacttccttgctctgggccggaaaatctttgcaggagggtgccaccggaggacattcctgcccccaggccatgaagaagtgggggtgggagacaagagggggtttcggagggggtggaggggggtgctcctcaggtggaggaggcggaggatatataggcggcaatgcagcctcaaacaatgaccccgaaatggatggggaagatggggtttccttcatcagtccactgggcatcctgtacaccccagctttaaaagtgatggaaggccacggggaagtgaatattaagcattatctaaactgcagtcactgtgaggtagacgaatgtcacatggaccctgaaagccacaaggtcatctgcttctgtgaccacgggacggtgctggctgaggatggcgtctcctgcattgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa

SEQ ID NO: 8 - DNA, 2631 bp, Homo sapiens
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccactgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa

SEQ ID NO: 9 - DNA, 6186 bp, Homo sapiens
ggctgagttttatgacgggcccggtgctgaagggcagggaacaacttgatggtgctactttgaactgcttttcttttctcctttttgcacaaagagtctcatgtctgatatttagacatgatgagctttgtgcaaaaggggagctggctacttctcgctctgcttcatcccactattattttggcacaacaggaagctgttgaaggaggatgttcccatcttggtcagtcctatgcggatagagatgtctggaagccagaaccatgccaaatatgtgtctgtgactcaggatccgttctctgcgatgacataatatgtgacgatcaagaattagactgccccaacccagaaattccatttggagaatgttgtgcagtttgcccacagcctccaactgctcctactcgccctcctaatggtcaaggacctcaaggccccaagggagatccaggccctcctggtattcctgggagaaatggtgaccctggtattccaggacaaccagggtcccctggttctcctggcccccctggaatctgtgaatcatgccctactggtcctcagaactattctccccagtatgattcatatgatgtcaagtctggagtagcagtaggaggactcgcaggctatcctggaccagctggccccccaggccctcccggtccccctggtacatctggtcatcctggttcccctggatctccaggataccaaggaccccctggtgaacctgggcaagctggtccttcaggccctccaggacctcctggtgctataggtccatctggtcctgctggaaaagatggagaatcaggtagacccggacgacctggagagcgaggattgcctggacctccaggtatcaaaggtccagctgggatacctggattccctggtatgaaaggacacagaggcttcgatggacgaaatggagaaaagggtgaaacaggtgctcctggattaaagggtgaaaatggtcttccaggcgaaaatggagctcctggacccatgggtccaagaggggctcctggtgagcgaggacggccaggacttcctggggctgcaggtgctcggggtaatgacggtgctcgaggcagtgatggtcaaccaggccctcctggtcctcctggaactgccggattccctggatcccctggtgctaagggtgaagttggacctgcagggtctcctggttcaaatggtgcccctggacaaagaggagaacctggacctcagggacacgctggtgctcaaggtcctcctggccctcctgggattaatggtagtcctggtggtaaaggcgaaatgggtcccgctggcattcctggagctcctggactgatgggagcccggggtcctccaggaccagccggtgctaatggtgctcctggactgcgaggtggtgcaggtgagcctggtaagaatggtgccaaaggagagcccggaccacgtggtgaacgcggtgaggctggtattccaggtgttccaggagctaaaggcgaagatggcaaggatggatcacctggagaacctggtgcaaatgggcttccaggagctgcaggagaaaggggtgcccctgggttccgaggacctgctggaccaaatggcatcccaggagaaaagggtcctgctggagagcgtggtgctccaggccctgcagggcccagaggagctgctggagaacctggcagagatggcgtccctggaggtccaggaatgaggggcatgcccggaagtccaggaggaccaggaagtgatgggaaaccagggcctcccggaagtcaaggagaaagtggtcgaccaggtcctcctgggccatctggtccccgaggtcagcctggtgtcatgggcttccccggtcctaaaggaaatgatggtgctcctggtaagaatggagaacgaggtggccctggaggacctggccctcagggtcctcctggaaagaatggtgaaactggacctcagggacccccagggcctactgggcctggtggtgacaaaggagacacaggaccccctggtccacaaggattacaaggcttgcctggtacaggtggtcctccaggagaaaatggaaaacctggggaaccaggtccaaagggtgatgccggtgcacctggagctccaggaggcaagggtgatgctggtgcccctggtgaacgtggacctcctggattggcaggggccccaggacttagaggtggagctggtccccctggtcccgaaggaggaaagggtgctgctggtcctcctgggccacctggtgctgctggtactcctggtctgcaaggaatgcctggagaaagaggaggtcttggaagtcctggtccaaagggtgacaagggtgaaccaggcggtccaggtgctgatggtgtcccagggaaagatggcccaaggggtcctactggtcctattggtcctcctggcccagctggccagcctggagataagggtgaaggtggtgcccccggacttccaggtatagctggacctcgtggtagccctggtgagagaggtgaaactggccctccaggacctgctggtttccctggtgctcctggacagaatggtgaacctggtggtaaaggagaaagaggggctccgggtgagaaaggtgaaggaggccctcctggagttgcaggaccccctggaggttctggacctgctggtcctcctggtccccaaggtgtcaaaggtgaacgtggcagtcctggtggacctggtgctgctggcttccctggtgctcgtggtcttcctggtcctcctggtagtaatggtaacccaggacccccaggtcccagcggttctccaggcaaggatgggcccccaggtcctgcgggtaacactggtgctcctggcagccctggagtgtctggaccaaaaggtgatgctggccaaccaggagagaagggatcgcctggtgcccagggcccaccaggagctccaggcccacttgggattgctgggatcactggagcacggggtcttgcaggaccaccaggcatgccaggtcctaggggaagccctggccctcagggtgtcaagggtgaaagtgggaaaccaggagctaacggtctcagtggagaacgtggtccccctggaccccagggtcttcctggtctggctggtacagctggtgaacctggaagagatggaaaccctggatcagatggtcttccaggccgagatggatctcctggtggcaagggtgatcgtggtgaaaatggctctcctggtgcccctggcgctcctggtcatccaggcccacctggtcctgtcggtccagctggaaagagtggtgacagaggagaaagtggccctgctggccctgctggtgctcccggtcctgctggttcccgaggtgctcctggtcctcaaggcccacgtggtgacaaaggtgaaacaggtgaacgtggagctgctggcatcaaaggacatcgaggattccctggtaatccaggtgccccaggttctccaggccctgctggtcagcagggtgcaatcggcagtccaggacctgcaggccccagaggacctgttggacccagtggacctcctggcaaagatggaaccagtggacatccaggtcccattggaccaccagggcctcgaggtaacagaggtgaaagaggatctgagggctccccaggccacccagggcaaccaggccctcctggacctcctggtgcccctggtccttgctgtggtggtgttggagccgctgccattgctgggattggaggtgaaaaagctggcggttttgccccgtattatggagatgaaccaatggatttcaaaatcaacaccgatgagattatgacttcactcaagtctgttaatggacaaatagaaagcctcattagtcctgatggttctcgtaaaaaccccgctagaaactgcagagacctgaaattctgccatcctgaactcaagagtgtgtcacccaccccggagccacacctgccactctcgctgatcctctctgtggtgacctctgccctcgtggccgccctggtcctggctttctccggcatcatgattgtgtaccgccggaagcaccaggagctgcaagccatgcagatggagctgcagagccctgagtacaagctgagcaagctccgcacctcgaccatcatgaccgactacaaccccaactactgctttgctggcaagacctcctccatcagtgacctgaaggaggtgccgcggaaaaacatcaccctcattcggggtctgggccatggcgcctttggggaggtgtatgaaggccaggtgtccggaatgcccaacgacccaagccccctgcaagtggctgtgaagacgctgcctgaagtgtgctctgaacaggacgaactggatttcctcatggaagccctgatcatcagcaaattcaaccaccagaacattgttcgctgcattggggtgagcctgcaatccctgccccggttcatcctgctggagctcatggcggggggagacctcaagtccttcctccgagagacccgccctcgcccgagccagccctcctccctggccatgctggaccttctgcacgtggctcgggacattgcctgtggctgtcagtatttggaggaaaaccacttcatccaccgagacattgctgccagaaactgcctcttgacctgtccaggccctggaagagtggccaagattggagacttcgggatggcccgagacatctacagggcgagctactatagaaagggaggctgtgccatgctgccagttaagtggatgcccccagaggccttcatggaaggaatattcacttctaaaacagacacatggtcctttggagtgctgctatgggaaatcttttctcttggatatatgccataccccagcaaaagcaaccaggaagttctggagtttgtcaccagtggaggccggatggacccacccaagaactgccctgggcctgtataccggataatgactcagtgctggcaacatcagcctgaagacaggcccaactttgccatcattttggagaggattgaatactgcacccaggacccggatgtaatcaacaccgctttgccgatagaatatggtccacttgtggaagaggaagagaaagtgcctgtgaggcccaaggaccctgagggggttcctcctctcctggtctctcaacaggcaaaacgggaggaggagcgcagcccagctgccccaccacctctgcctaccacctcctctggcaaggctgcaaagaaacccacagctgcagagatctctgttcgagtccctagagggccggccgtggaagggggacacgtgaatatggcattctctcagtccaaccctccttcggagttgcacaaggtccacggatccagaaacaagcccaccagcttgtggaacccaacgtacggctcctggtttacagagaaacccaccaaaaagaataatcctatagcaaagaaggagccacacgacaggggtaacctggggctggagggaagctgtactgtcccacctaacgttgcaactgggagacttccgggggcctcactgctcctagagccctcttcgctgactgccaatatgaaggaggtacctctgttcaggctacgtcacttcccttgtgggaatgtcaattacggctaccagcaacagggcttgcccttagaagccgctactgcccctggagctggtcattacgaggataccattctgaaaagcaagaatagcatgaaccagcctgggccctgagctcggtcgcacactcacttctcttccttgggatccctaagaccgtggaggagagagaggcaatggctccttcacaaaccagagaccaaatgtcacgttttgttttgtgccaacctattttgaagtaccaccaaaaaagctgtattttgaaaatgctttagaaaggttttgagcatgggttcatcctattctttcgaaagaagaaaatatcataaaaatgagtgataaatacaaggcccagatgtggttgcataaggtttttatgcatgtttgttgtatacttccttatgcttctttcaaattgtgtgtgctctgcttcaatgtagtcagaattagctgcttctatgtttcatagttggggtcatagatgtttccttgccttgttgatgtggacatgagccatttgaggggagagggaacggaaataaaggagttatttgtaatgactaa

SEQ ID NO: 10 - protein, 1225 aa, Homo sapiens
MMANWAEARPLLILIVLLGQFVSIKAQEEDEDGTSPGSKMALQSSFTCWNGTVLQLGQACDFHQDCAQGEDESQMCRKLPVGFYCNFEDGFCGWTQGTLSPHTPQWQVRTLKDARFQDHQDHALLLSTTDVPASESATVTSATFPAPIKSSPCELRMSWLIRGVLRGNVSLVLVENKTGKEQGRMVWHVAAYEGLSLWQWMVLPLLDVSDRFWLQMVAWWGQGSRAIVAFDNISISLDCYLTISGEDKILQNTAPKSRNLFERNPNKELKPGENSPRQTPIFDPTVHWLFTTCGASGPHGPTQAQCNNAYQNSNLSVEVGSEGPLKGIQIWKVPATDTYSISGYGAAGGKGGKNTMMRSHGVSVLGIFNLEKDDMLYILVGQQGEDACPSTNQLIQKVCIGENNVIEEEIRVNRSVHEWAGGGGGGGGATYVFKMKDGVPVPLIIAAGGGGRAYGAKTDTFHPERLENNSSVLGLNGNSGAAGGGGGWNDNTSLLWAGKSLQEGATGGHSCPQAMKKWGWETRGGFGGGGGGCSSGGGGGGYIGGNAASNNDPEMDGEDGVSFISPLGILYTPALKVMEGHGEVNIKHYLNCSHCEVDECHMDPESHKVICFCDHGTVLAEDGVSCIVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 11 - protein, 687 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPLSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

SEQ ID NO: 12 - protein, 1872 aa, Homo sapiens
MMSFVQKGSWLLLALLHPTIILAQQEAVEGGCSHLGQSYADRDVWKPEPCQICVCDSGSVLCDDIICDDQELDCPNPEIPFGECCAVCPQPPTAPTRPPNGQGPQGPKGDPGPPGIPGRNGDPGIPGQPGSPGSPGPPGICESCPTGPQNYSPQYDSYDVKSGVAVGGLAGYPGPAGPPGPPGPPGTSGHPGSPGSPGYQGPPGEPGQAGPSGPPGPPGAIGPSGPAGKDGESGRPGRPGERGLPGPPGIKGPAGIPGFPGMKGHRGFDGRNGEKGETGAPGLKGENGLPGENGAPGPMGPRGAPGERGRPGLPGAAGARGNDGARGSDGQPGPPGPPGTAGFPGSPGAKGEVGPAGSPGSNGAPGQRGEPGPQGHAGAQGPPGPPGINGSPGGKGEMGPAGIPGAPGLMGARGPPGPAGANGAPGLRGGAGEPGKNGAKGEPGPRGERGEAGIPGVPGAKGEDGKDGSPGEPGANGLPGAAGERGAPGFRGPAGPNGIPGEKGPAGERGAPGPAGPRGAAGEPGRDGVPGGPGMRGMPGSPGGPGSDGKPGPPGSQGESGRPGPPGPSGPRGQPGVMGFPGPKGNDGAPGKNGERGGPGGPGPQGPPGKNGETGPQGPPGPTGPGGDKGDTGPPGPQGLQGLPGTGGPPGENGKPGEPGPKGDAGAPGAPGGKGDAGAPGERGPPGLAGAPGLRGGAGPPGPEGGKGAAGPPGPPGAAGTPGLQGMPGERGGLGSPGPKGDKGEPGGPGADGVPGKDGPRGPTGPIGPPGPAGQPGDKGEGGAPGLPGIAGPRGSPGERGETGPPGPAGFPGAPGQNGEPGGKGERGAPGEKGEGGPPGVAGPPGGSGPAGPPGPQGVKGERGSPGGPGAAGFPGARGLPGPPGSNGNPGPPGPSGSPGKDGPPGPAGNTGAPGSPGVSGPKGDAGQPGEKGSPGAQGPPGAPGPLGIAGITGARGLAGPPGMPGPRGSPGPQGVKGESGKPGANGLSGERGPPGPQGLPGLAGTAGEPGRDGNPGSDGLPGRDGSPGGKGDRGENGSPGAPGAPGHPGPPGPVGPAGKSGDRGESGPAGPAGAPGPAGSRGAPGPQGPRGDKGETGERGAAGIKGHRGFPGNPGAPGSPGPAGQQGAIGSPGPAGPRGPVGPSGPPGKDGTSGHPGPIGPPGPRGNRGERGSEGSPGHPGQPGPPGPPGAPGPCCGGVGAAAIAGIGGEKAGGFAPYYGDEPMDFKINTDEIMTSLKSVNGQIESLISPDGSRKNPARNCRDLKFCHPELKSVSPTPEPHLPLSLILSVVTSALVAALVLAFSGIMIVYRRKHQELQAMQMELQSPEYKLSKLRTSTIMTDYNPNYCFAGKTSSISDLKEVPRKNITLIRGLGHGAFGEVYEGQVSGMPNDPSPLQVAVKTLPEVCSEQDELDFLMEALIISKFNHQNIVRCIGVSLQSLPRFILLELMAGGDLKSFLRETRPRPSQPSSLAMLDLLHVARDIACGCQYLEENHFIHRDIAARNCLLTCPGPGRVAKIGDFGMARDIYRASYYRKGGCAMLPVKWMPPEAFMEGIFTSKTDTWSFGVLLWEIFSLGYMPYPSKSNQEVLEFVTSGGRMDPPKNCPGPVYRIMTQCWQHQPEDRPNFAIILERIEYCTQDPDVINTALPIEYGPLVEEEEKVPVRPKDPEGVPPLLVSQQAKREEERSPAAPPPLPTTSSGKAAKKPTAAEISVRVPRGPAVEGGHVNMAFSQSNPPSELHKVHGSRNKPTSLWNPTYGSWFTEKPTKKNNPIAKKEPHDRGNLGLEGSCTVPPNVATGRLPGASLLLEPSSLTANMKEVPLFRLRHFPCGNVNYGYQQQGLPLEAATAPGAGHYEDTILKSKNSMNQPGP

(19) United States
(12) Patent Application Publication
(10) Pub. No.: US 2024/0093304 A1
(43) Pub. Date: Mar. 21, 2024

(54) ALK FUSION GENES AND USES THEREOF

(71) Applicant: Foundation Medicine, Inc., Cambridge, MA (US)

(72) Inventors: Rachel ERBACH, Arlington, MA (US); Mark ROSENZWEIG, Norfolk, MA (US); Megan FRIEDRICHSEN, Cambridge, MA (US); Pierre VANDEN BORRE, Hopkinton, NH (US); Russell MADISON, Encinitas, CA (US)

(73) Assignee: Foundation Medicine, Inc., Cambridge, MA (US)

(21) Appl. No.: 18/269,937

(22) PCT Filed: Dec. 29, 2021
(86) PCT No.: PCT/US2021/065561

§ 371 Date: Jun. 27, 2023

Related U.S. Application Data
(60) Provisional application No. 63/243,002, filed on Sep. 10, 2021.
(60) Provisional application No. 63/132,085, filed on Dec. 30, 2020.

(51) Int. Cl. C12Q 1/6886; G01N 33/574

(52) U.S. Cl. CPC C12Q 1/6886; G01N 33/57484; C12Q 2600/106; C12Q 2600/156; G01N 2333/78; G01N 2800/52

(57) ABSTRACT

The present disclosure provides COL5A2-ALK fusion nucleic acid molecules and polypeptides, and COL3A1-ALK fusion nucleic acid molecules and polypeptides, as well as methods, kits and reagents for detecting such fusion nucleic acid molecules and polypeptides. The disclosure also provides methods for evaluating, identifying, assessing, and/or treating an individual having a cancer.

### CROSS-REFERENCE TO RELATED APPLICATIONS

This application claims the benefit of U.S. Provisional Application No. 63/243,002, filed Sep. 10, 2021, and U.S. Provisional Application No. 63/132,085, filed Dec. 30, 2020, the contents of each of which are hereby incorporated by reference in their entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 197102003340SEQLIST.TXT, date recorded: Dec. 27, 2021, size: 107,652 bytes).

### FIELD OF THE INVENTION

The present disclosure relates to ALK fusion nucleic acid molecules and polypeptides, methods of detecting ALK fusion nucleic acid molecules and polypeptides, as well as methods of diagnosis, assessment, and treatment of diseases such as cancer.

### BACKGROUND OF THE INVENTION

Cancer represents the phenotypic end-point of multiple genetic lesions that endow cells with a full range of biological properties required for tumorigenesis. Indeed, a hallmark genomic feature of many cancers is the presence of numerous complex chromosome structural aberrations, including translocations, intra-chromosomal inversions, point mutations, deletions, gene copy number changes, gene expression level changes, gene fusions, and germline mutations, among others.

The anaplastic lymphoma receptor tyrosine kinase (ALK) gene is a known oncogene that has been associated with cancerous phenotypes, including inflammatory myofibroblastic tumors, neuroblastoma, lung cancer, non-Hodgkin's lymphoma, and anaplastic large cell lymphoma, among others. Chromosomal rearrangements involving the ALK gene have been found in certain cancers. For example, a chromosomal rearrangement that generates a fusion gene resulting in the juxtaposition of the N-terminal region of nucleophosmin (NPM) with the kinase domain of ALK is known to be associated with non-Hodgkin's lymphoma (Morris, SW (1994) Science 263: 1281-1284).

Accordingly, there is a need in the art for identifying novel genetic lesions associated with cancer, such as genetic lesions involving ALK. Such genetic lesions can be an effective approach to develop compositions, methods and assays for evaluating and treating cancer patients.

All references cited herein, including patents, patent applications and publications, are hereby incorporated by reference in their entirety. To the extent that any reference incorporated by reference conflicts with the instant disclosure, the instant disclosure shall control.

### BRIEF SUMMARY OF THE INVENTION

In one aspect, provided herein is a method of treating or delaying progression of cancer, comprising administering to an individual an effective amount of a treatment comprising an anti-cancer therapy, wherein the cancer comprises a collagen alpha-2(V) chain (COL5A2)-anaplastic lymphoma kinase (ALK) fusion nucleic acid molecule or polypeptide or a collagen alpha-1(III) chain (COL3A1)-ALK fusion nucleic acid molecule or polypeptide.

In another aspect, provided herein is a method of treating or delaying progression of cancer, comprising, responsive to knowledge of a COL5A2-ALK fusion nucleic acid molecule or polypeptide or a COL3A1-ALK fusion nucleic acid molecule or polypeptide in a sample from an individual, administering to the individual an effective amount of a treatment comprising an anti-cancer therapy.

In another aspect, provided herein is a method of identifying one or more treatment options for an individual having cancer, the method comprising: (a) acquiring knowledge of a COL5A2-ALK fusion nucleic acid molecule or polypeptide or a COL3A1-ALK fusion nucleic acid molecule or polypeptide in a sample from the individual; and (b) generating a report comprising one or more treatment options identified for the individual based at least in part on said knowledge, wherein the one or more treatment options comprise an anti-cancer therapy.

In another aspect, provided herein is a method of selecting treatment for an individual having cancer, comprising acquiring knowledge of a COL5A2-ALK fusion nucleic acid molecule or polypeptide or a COL3A1-ALK fusion nucleic acid molecule or polypeptide in a sample from an individual having cancer, wherein responsive to the acquisition of said knowledge: (i) the individual is classified as a candidate to receive a treatment comprising an anti-cancer therapy; and/or (ii) the individual is identified as likely to respond to a treatment comprising an anti-cancer therapy.

In another aspect, provided herein is a method of treating or delaying progression of cancer, comprising: (a) acquiring knowledge of a COL5A2-ALK fusion nucleic acid molecule or polypeptide or a COL3A1-ALK fusion nucleic acid molecule or polypeptide in a sample from an individual; and (b) responsive to said knowledge, administering to the individual an effective amount of a treatment comprising an anti-cancer therapy.

In another aspect, provided herein is a method of predicting survival of an individual having cancer treated with an anti-cancer therapy, comprising acquiring knowledge of a COL5A2-ALK fusion nucleic acid molecule or polypeptide or a COL3A1-ALK fusion nucleic acid molecule or polypeptide in a sample from the individual, wherein responsive to the acquisition of said knowledge, the individual is predicted to have longer survival after treatment with the anti-cancer therapy, as compared to an individual whose cancer does not exhibit the COL5A2-ALK fusion nucleic acid molecule or polypeptide, or the COL3A1-ALK fusion nucleic acid molecule or polypeptide.

In another aspect, provided herein is a method of screening an individual having cancer, suspected of having cancer, being tested for cancer, being treated for cancer, or being tested for a susceptibility to cancer, comprising acquiring knowledge of a COL5A2-ALK fusion nucleic acid molecule or polypeptide or a COL3A1-ALK fusion nucleic acid molecule or polypeptide in a sample from the individual, wherein responsive to the acquisition of said knowledge, the individual is predicted to have increased risk of cancer recurrence, aggressive cancer, anti-cancer therapy resistance, or poor prognosis, as compared to an individual whose cancer does not exhibit the COL5A2-ALK fusion nucleic acid molecule or polypeptide, or the COL3A1-ALK fusion nucleic acid molecule or polypeptide.

In another aspect, provided herein is a method of monitoring an individual having cancer, comprising acquiring knowledge of a COL5A2-ALK fusion nucleic acid molecule or polypeptide or a COL3A1-ALK fusion nucleic acid molecule or polypeptide in a sample from the individual, wherein responsive to the acquisition of said knowledge, the individual is predicted to have increased risk of cancer recurrence, aggressive cancer, anti-cancer therapy resistance, or poor prognosis, as compared to an individual whose cancer does not exhibit the COL5A2-ALK fusion nucleic acid molecule or polypeptide, or the COL3A1-ALK fusion nucleic acid molecule or polypeptide, optionally wherein the individual is being treated for cancer.

In some embodiments of any of the methods provided herein, the acquiring knowledge comprises detecting the COL5A2-ALK fusion nucleic acid molecule or polypeptide or the COL3A1-ALK fusion nucleic acid molecule or polypeptide in a sample from the individual.

In another aspect, provided herein is a method of identifying an individual having cancer who may benefit from a treatment comprising an anti-cancer therapy, the method comprising detecting a COL5A2-ALK fusion nucleic acid molecule or polypeptide or a COL3A1-ALK fusion nucleic acid molecule or polypeptide in a sample from the individual, wherein the presence of the COL5A2-ALK fusion nucleic acid molecule or polypeptide or the COL3A1-ALK fusion nucleic acid molecule or polypeptide in the sample identifies the individual as one who may benefit from the treatment comprising an anti-cancer therapy.

In another aspect, provided herein is a method of selecting a therapy for an individual having cancer, the method comprising detecting a COL5A2-ALK fusion nucleic acid molecule or polypeptide or a COL3A1-ALK fusion nucleic acid molecule or polypeptide in a sample from the individual, wherein the presence of the COL5A2-ALK fusion nucleic acid molecule or polypeptide, or the COL3A1-ALK fusion nucleic acid molecule or polypeptide in the sample identifies the individual as one who may benefit from a treatment comprising an anti-cancer therapy.

In another aspect, provided herein is a method of identifying one or more treatment options for an individual having cancer, the method comprising: (a) detecting a COL5A2-ALK fusion nucleic acid molecule or polypeptide or a COL3A1-ALK fusion nucleic acid molecule or polypeptide in a sample from the individual; and (b) generating a report comprising one or more treatment options identified for the individual based at least in part on the presence of the COL5A2-ALK fusion nucleic acid molecule or polypeptide or the COL3A1-ALK fusion nucleic acid molecule or polypeptide in the sample, wherein the one or more treatment options comprise an anti-cancer therapy.

In another aspect, provided herein is a method of treating or delaying progression of cancer, comprising: (a) detecting a COL5A2-ALK fusion nucleic acid molecule or polypeptide or a COL3A1-ALK fusion nucleic acid molecule or polypeptide in a sample from an individual; and (b) administering to the individual an effective amount of a treatment comprising an anti-cancer therapy.

In another aspect, provided herein is a method of detecting a COL5A2-ALK fusion nucleic acid molecule or polypeptide or a COL3A1-ALK fusion nucleic acid molecule or polypeptide, the method comprising detecting the COL5A2-ALK fusion nucleic acid molecule or polypeptide, or the COL3A1-ALK fusion nucleic acid molecule or polypeptide in a sample from an individual.

In another aspect, provided herein is a method of assessing a COL5A2-ALK fusion nucleic acid molecule or polypeptide or a COL3A1-ALK fusion nucleic acid molecule or polypeptide, the method comprising: (a) detecting a COL5A2-ALK fusion nucleic acid molecule or polypeptide or a COL3A1-ALK fusion nucleic acid molecule or polypeptide in a sample from an individual; and (b) providing an assessment of the COL5A2-ALK fusion nucleic acid molecule or polypeptide or the COL3A1-ALK fusion nucleic acid molecule or polypeptide.

In some embodiments of any of the methods provided herein, the individual has cancer, is suspected of having cancer, is being tested for cancer, is being treated for cancer, or is being tested for a susceptibility to cancer. In some embodiments, the methods provided herein further comprising selectively enriching for one or more nucleic acids comprising a COL5A2-ALK fusion nucleic acid molecule or a COL3A1-ALK fusion nucleic acid molecule nucleotide sequences to produce an enriched sample. In some embodiments, the cancer is a hematologic malignancy or a solid tumor malignancy. In some embodiments, the cancer is selected from anaplastic large cell lymphoma (ALCL), non-small cell lung cancer (NSCLC), colorectal cancer (CRC), sarcoma, sarcoma not otherwise specified (NOS), inflammatory myofibroblastic tumor (IMT), rhabdomyosarcoma, acute myeloid leukemia, histiocytosis, leiomyosarcoma, ALK-positive large B-cell lymphoma, epithelioid fibrous histiocytoma, a pulmonary carcinoma, a renal cell carcinoma, a thyroid carcinoma, a pancreatic carcinoma, carcinoma of unknown primary, ovarian carcinoma, glioma, mesothelioma, melanoma, or a Spitzoid tumor. In some embodiments, the cancer is a sarcoma. In some embodiments, the cancer is a uterus leiomyosarcoma, soft tissue inflammatory myofibroblastic tumor, or soft tissue sarcoma not otherwise specified (NOS).

In some embodiments of any of the methods provided herein, the cancer is rhabdomyosarcoma, and the rhabdomyosarcoma comprises a COL5A2-ALK fusion nucleic acid molecule or polypeptide. In some embodiments of any of the methods provided herein, the cancer is rhabdomyosarcoma, and an anti-cancer therapy is administered to the individual responsive to acquiring knowledge of a COL5A2-ALK fusion nucleic acid molecule or polypeptide in the sample. In some embodiments of any of the methods provided herein, the cancer is rhabdomyosarcoma, and the acquiring knowledge comprises acquiring knowledge of a COL5A2-ALK fusion nucleic acid molecule or polypeptide in the sample. In some embodiments of any of the methods provided herein, the cancer is rhabdomyosarcoma, and the detecting comprises detecting a COL5A2-ALK fusion nucleic acid molecule or polypeptide in the sample. In some embodiments of any of the methods provided herein, the cancer is rhabdomyosarcoma, and the selectively enriching comprises selectively enriching for one or more nucleic acids comprising COL5A2-ALK fusion nucleic acid molecule nucleotide sequences.

In some embodiments of any of the methods provided herein, the cancer is leiomyosarcoma, inflammatory myofibroblastic tumor (IMT), or sarcoma not otherwise specified (NOS), and the leiomyosarcoma, inflammatory myofibroblastic tumor (IMT), or sarcoma not otherwise specified (NOS) comprises a COL3A1-ALK fusion nucleic acid molecule or polypeptide. In some embodiments of any of the methods provided herein, the cancer is leiomyosarcoma, inflammatory myofibroblastic tumor (IMT), or sarcoma not otherwise specified (NOS), and an anti-cancer therapy is administered to the individual responsive to acquiring knowledge of a COL3A1-ALK fusion nucleic acid molecule or polypeptide in the sample. In some embodiments of any of the methods provided herein, the cancer is leiomyosarcoma, inflammatory myofibroblastic tumor (IMT), or sarcoma not otherwise specified (NOS), and the acquiring knowledge comprises acquiring knowledge of a COL3A1-ALK fusion nucleic acid molecule or polypeptide in the sample. In some embodiments of any of the methods provided herein, the cancer is leiomyosarcoma, inflammatory myofibroblastic tumor (IMT), or sarcoma not otherwise specified (NOS), and the detecting comprises detecting a COL3A1-ALK fusion nucleic acid molecule or polypeptide in the sample. In some embodiments of any of the methods provided herein, the cancer is leiomyosarcoma, inflammatory myofibroblastic tumor (IMT), or sarcoma not otherwise specified (NOS), and the selectively enriching comprises selectively enriching for one or more nucleic acids comprising COL3A1-ALK fusion nucleic acid molecule nucleotide sequences. In some embodiments of any of the methods provided herein, the cancer is a uterus leiomyosarcoma, soft tissue inflammatory myofibroblastic tumor, or soft tissue sarcoma not otherwise specified (NOS), and the acquiring knowledge comprises acquiring knowledge of a COL3A1-ALK fusion nucleic acid molecule or polypeptide in the sample.

In some embodiments of any of the methods provided herein, the anti-cancer therapy comprises a small molecule inhibitor, an antibody, a cellular therapy, or a nucleic acid. In some embodiments, the anti-cancer therapy comprises an ALK-targeted therapy. In some embodiments, the ALK-targeted therapy is a kinase inhibitor. In some embodiments, the kinase inhibitor is selected from crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). In some embodiments, the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy. In some embodiments, the nucleic acid comprises a double-stranded RNA (dsRNA), a small interfering RNA (siRNA), or a small hairpin RNA (shRNA).

In some embodiments, the anti-cancer therapy comprises a heat shock protein (HSP) inhibitor, a MYC inhibitor, an HDAC inhibitor, an immunotherapy, an ALK neoantigen a vaccine, or a cellular therapy. In some embodiments, the HSP inhibitor is an HSP90 inhibitor. In some embodiments, the HSP90 inhibitor is ganetespib. In some embodiments, the treatment or the one or more treatment options further comprise a second therapeutic agent. In some embodiments, the second therapeutic agent is an immune checkpoint inhibitor, a VEGF inhibitor, an Integrin 03 inhibitor, a statin, an EGFR inhibitor, an mTOR inhibitor, a PI3K inhibitor, a MAPK inhibitor, or a CDK4/6 inhibitor. In some embodiments, the immune checkpoint inhibitor is a PD-1 or a PD-L1 inhibitor. In some embodiments, the PD-1 inhibitor is nivolumab. In some embodiments, the VEGF inhibitor is bevacizumab.

In some embodiments of any of the methods provided herein, the COL5A2-ALK fusion nucleic acid molecule comprises: exon 1 or a portion thereof of COL5A2 fused to intron 5 or a portion thereof of ALK; intron 1 or a portion thereof of COL5A2 fused to intron 5 or a portion thereof of ALK; exon 1 or a portion thereof of COL5A2 fused to exon 6 or a portion thereof of ALK; or intron 1 or a portion thereof of COL5A2 fused to exon 6 or a portion thereof of ALK. In some embodiments, the COL5A2-ALK fusion nucleic acid molecule comprises a nucleotide sequence comprising, in the 5′ to 3′ direction, exon 1 or a portion thereof of COL5A2, and exon 6 or a portion thereof and exons 7-29 of ALK. In some embodiments, the COL5A2-ALK fusion nucleic acid molecule results from a breakpoint in exon 1 or in intron 1 of COL5A2, and in intron 5 or in exon 6 of ALK. In some embodiments, the COL5A2-ALK fusion nucleic acid molecule comprises the nucleotide sequence of SEQ ID NO: 7, or a nucleotide sequence at least 70%, 75%, 80%, 85%, 90%, 91%, 92%, 93%, 94%, 95%, 96%, 97%, 98%, 99%, or 99.5% identical thereto. In some embodiments, the COL5A2-ALK fusion polypeptide comprises: an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK; or an amino acid sequence at least about 85% identical to an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK. In some embodiments, the COL5A2-ALK fusion polypeptide comprises the amino acid sequence of SEQ ID NO: 10, or an amino acid sequence at least 70%, 75%, 80%, 85%, 90%, 91%, 92%, 93%, 94%, 95%, 96%, 97%, 98%, 99%, or 99.5% identical thereto.

In some embodiments of any of the methods provided herein, the COL3A1-ALK fusion nucleic acid molecule comprises: (a) exon 48 or a portion thereof of COL3A1 fused to intron 18 or a portion thereof of ALK; exon 48 or a portion thereof of COL3A1 fused to exon 19 or a portion thereof of ALK; intron 48 or a portion thereof of COL3A1 fused to intron 18 or a portion thereof of ALK; or intron 48 or a portion thereof of COL3A1 fused to exon 19 or a portion thereof of ALK; or (b) exon 2 or a portion thereof of COL3A1 fused to intron 18 or a portion thereof of ALK; exon 2 or a portion thereof of COL3A1 fused to exon 19 or a portion thereof of ALK; intron 2 or a portion thereof of COL3A1 fused to intron 18 or a portion thereof of ALK; or intron 2 or a portion thereof of COL3A1 fused to exon 19 or a portion thereof of ALK. In some embodiments, the COL3A1-ALK fusion nucleic acid molecule comprises a nucleotide sequence comprising, in the 5′ to 3′ direction: (a) exons 1-47 and exon 48 or a portion thereof of COL3A1, and exon 19 or a portion thereof and exons 20-29 of ALK; or (b) exon 1 and exon 2 or a portion thereof of COL3A1, and exon 19 or a portion thereof and exons 20-29 of ALK. In some embodiments, the COL3A1-ALK fusion nucleic acid molecule results from: (a) a breakpoint in exon 2 or intron 2 of COL3A1, and in intron 18 or exon 19 of ALK; or a breakpoint joining Chr2:189849674 with Chr2:29448496; or (b) a breakpoint in exon 48 or intron 48 of COL3A1, and in intron 18 or exon 19 of ALK; a breakpoint joining Chr2:189874528 with Chr2:29448490; or a breakpoint joining Chr2:189874814 with Chr2:29449440. In some embodiments, the chromosome positions correspond to chromosome positions of human genome version hg19. In some embodiments, the COL3A1-ALK fusion nucleic acid molecule comprises the nucleotide sequence of SEQ ID NO: 8 or 9, or a nucleotide sequence at least 70%, 75%, 80%, 85%, 90%, 91%, 92%, 93%, 94%, 95%, 96%, 97%, 98%, 99%, or 99.5% identical thereto. In some embodiments, the COL3A1-ALK fusion polypeptide comprises: (a) an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK; or an amino acid sequence at least about 85% identical to an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK; or (b) an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK; or an amino acid sequence at least about 85% identical to an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK. In some embodiments, the COL3A1-ALK fusion polypeptide comprises the amino acid sequence of SEQ ID NO: 11 or 12, or an amino acid sequence at least 70%, 75%, 80%, 85%, 90%, 91%, 92%, 93%, 94%, 95%, 96%, 97%, 98%, 99%, or 99.5% identical thereto.

In some embodiments of any of the methods provided herein, the COL5A2-ALK fusion polypeptide, or the COL3A1-ALK fusion polypeptide has kinase activity.

In some embodiments of any of the methods provided herein, the sample from the individual comprises fluid, cells, or tissue. In some embodiments, the sample from the individual comprises a tumor biopsy or a circulating tumor cell. In some embodiments, the sample from the individual is a nucleic acid sample. In some embodiments, the nucleic acid sample comprises mRNA, genomic DNA, circulating tumor DNA, cell-free DNA, or cell-free RNA. In some embodiments, the COL5A2-ALK fusion nucleic acid molecule, or the COL3A1-ALK fusion nucleic acid molecule is detected in the sample by one or more methods selected from a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping. In some embodiments, the sample from the individual is a protein sample. In some embodiments, the COL5A2-ALK fusion polypeptide, or the COL3A1-ALK fusion polypeptide is detected in the sample by one or more methods selected from the group consisting of immunoblotting, enzyme linked immunosorbent assay (ELISA), immunohistochemistry, and mass spectrometry.

In another aspect, provided herein is a COL5A2-ALK fusion nucleic acid molecule comprising: exon 1 or a portion thereof of COL5A2 fused to intron 5 or a portion thereof of ALK; intron 1 or a portion thereof of COL5A2 fused to intron 5 or a portion thereof of ALK; exon 1 or a portion thereof of COL5A2 fused to exon 6 or a portion thereof of ALK; or intron 1 or a portion thereof of COL5A2 fused to exon 6 or a portion thereof of ALK.

In another aspect, provided herein is a COL5A2-ALK fusion nucleic acid molecule comprising a nucleotide sequence comprising, in the 5′ to 3′ direction, exon 1 or a portion thereof of COL5A2, and exon 6 or a portion thereof and exons 7-29 of ALK.

In some embodiments, the COL5A2-ALK fusion nucleic acid molecule results from a breakpoint in exon 1 or in intron 1 of COL5A2, and in intron 5 or in exon 6 of ALK. In some embodiments, the COL5A2-ALK fusion nucleic acid molecule comprises the nucleotide sequence of SEQ ID NO: 7, or a nucleotide sequence at least 70%, 75%, 80%, 85%, 90%, 91%, 92%, 93%, 94%, 95%, 96%, 97%, 98%, 99%, or 99.5% identical thereto.

In another aspect, provided herein is a COL5A2-ALK fusion polypeptide comprising: an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK; or an amino acid sequence at least about 85% identical to an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK. In some embodiments, the COL5A2-ALK fusion polypeptide comprises the amino acid sequence of SEQ ID NO: 10, or an amino acid sequence at least 70%, 75%, 80%, 85%, 90%, 91%, 92%, 93%, 94%, 95%, 96%, 97%, 98%, 99%, or 99.5% identical thereto.

In another aspect, provided herein is a COL3A1-ALK fusion nucleic acid molecule comprising: (a) exon 48 or a portion thereof of COL3A1 fused to intron 18 or a portion thereof of ALK; exon 48 or a portion thereof of COL3A1 fused to exon 19 or a portion thereof of ALK; intron 48 or a portion thereof of COL3A1 fused to intron 18 or a portion thereof of ALK; or intron 48 or a portion thereof of COL3A1 fused to exon 19 or a portion thereof of ALK; or (b) exon 2 or a portion thereof of COL3A1 fused to intron 18 or a portion thereof of ALK; exon 2 or a portion thereof of COL3A1 fused to exon 19 or a portion thereof of ALK; intron 2 or a portion thereof of COL3A1 fused to intron 18 or a portion thereof of ALK; or intron 2 or a portion thereof of COL3A1 fused to exon 19 or a portion thereof of ALK.

In another aspect, provided herein is a COL3A1-ALK fusion nucleic acid molecule comprising a nucleotide sequence comprising, in the 5′ to 3′ direction: (a) exons 1-47 and exon 48 or a portion thereof of COL3A1, and exon 19 or a portion thereof and exons 20-29 of ALK; or (b) exon 1 and exon 2 or a portion thereof of COL3A1, and exon 19 or a portion thereof and exons 20-29 of ALK.

In some embodiments, the COL3A1-ALK fusion nucleic acid molecule results from: (a) a breakpoint in exon 2 or intron 2 of COL3A1, and in intron 18 or exon 19 of ALK; or a breakpoint joining Chr2:189849674 with Chr2:29448496; or (b) a breakpoint in exon 48 or intron 48 of COL3A1, and in intron 18 or exon 19 of ALK; a breakpoint joining Chr2:189874528 with Chr2:29448490; or a breakpoint joining Chr2:189874814 with Chr2:29449440. In some embodiments, the chromosome positions correspond to chromosome positions of human genome version hg19. In some embodiments, the COL3A1-ALK fusion nucleic acid molecule comprises the nucleotide sequence of SEQ ID NO: 8 or 9, or a nucleotide sequence at least 70%, 75%, 80%, 85%, 90%, 91%, 92%, 93%, 94%, 95%, 96%, 97%, 98%, 99%, or 99.5% identical thereto.

In another aspect, provided herein is a COL3A1-ALK fusion polypeptide comprising: (a) an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK; or an amino acid sequence at least about 85% identical to an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK; or (b) an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK; or an amino acid sequence at least about 85% identical to an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK. In some embodiments, the COL3A1-ALK fusion polypeptide comprises the amino acid sequence of SEQ ID NO: 11 or 12, or an amino acid sequence at least 70%, 75%, 80%, 85%, 90%, 91%, 92%, 93%, 94%, 95%, 96%, 97%, 98%, 99%, or 99.5% identical thereto.

In another aspect, provided herein is an anti-cancer therapy for use in a method of treating or delaying progression of cancer, wherein the method comprises administering the anti-cancer therapy to an individual, wherein a COL5A2-ALK fusion nucleic acid molecule or polypeptide or a COL3A1-ALK fusion nucleic acid molecule or polypeptide has been detected in a sample obtained from the individual.

In another aspect, provided herein is an anti-cancer therapy for use in the manufacture of a medicament for treating or delaying progression of cancer, wherein the medicament is to be administered to an individual, wherein a COL5A2-ALK fusion nucleic acid molecule or polypeptide or a COL3A1-ALK fusion nucleic acid molecule or polypeptide has been detected in a sample obtained from the individual.

In another aspect, provided herein is an in vitro use of one or more oligonucleotides for detecting a COL5A2-ALK fusion nucleic acid molecule or a COL3A1-ALK fusion nucleic acid molecule.

In another aspect, provided herein is a kit comprising one or more oligonucleotides for detecting a COL5A2-ALK fusion nucleic acid molecule or a COL3A1-ALK fusion nucleic acid molecule.

In another aspect, provided herein is an in vitro use of a probe or bait for detecting a COL5A2-ALK fusion nucleic acid molecule or a COL3A1-ALK fusion nucleic acid molecule, wherein the probe or bait comprises a capture nucleic acid molecule configured to hybridize to a target nucleic acid molecule comprising a COL5A2-ALK fusion nucleic acid molecule or a COL3A1-ALK fusion nucleic acid molecule nucleotide sequences.

In another aspect, provided herein is a kit comprising a probe or bait for detecting a COL5A2-ALK fusion nucleic acid molecule or a COL3A1-ALK fusion nucleic acid molecule.

In another aspect, provided herein is an antibody or antibody fragment that specifically binds to a COL5A2-ALK fusion polypeptide or a COL3A1-ALK fusion polypeptide.

In another aspect, provided herein is a kit comprising an antibody or antibody fragment that specifically binds to a COL5A2-ALK fusion polypeptide or a COL3A1-ALK fusion polypeptide for detecting the COL5A2-ALK fusion polypeptide or the COL3A1-ALK fusion polypeptide.

In another aspect, provided herein is a vector comprising a COL5A2-ALK fusion nucleic acid molecule or a COL3A1-ALK fusion nucleic acid molecule, or a fragment thereof.

In another aspect, provided herein is a host cell comprising a vector that comprises a COL5A2-ALK fusion nucleic acid molecule or a COL3A1-ALK fusion nucleic acid molecule, or a fragment thereof.

It is to be understood that one, some, or all of the properties of the various embodiments described herein may be combined to form other embodiments of the present invention. These and other aspects of the invention will become apparent to one of skill in the art. These and other embodiments of the invention are further described by the detailed description that follows.

### BRIEF DESCRIPTION OF THE DRAWINGS

FIG. 1 shows that analyzing DNA and RNA detects most gene fusions and rearrangements in sarcoma. Total cases are indicated by bar label.

FIG. 2 shows that diverse gene fusions and rearrangements were seen across a wide range of sarcomas.

FIG. 3 shows that analysis of RNA detects ALK fusions with distinct breakpoints not covered by DNA baiting that covers canonical non-small cell lung cancer (NSCLC) breakpoints.

FIGS. 4A & 4B show that, of 41 NTRK1/3 gene fusions detected in DNA, 88% were confirmed by analyzing RNA (5 in DNA only, 36 in DNA and RNA; FIG. 4A). An additional 39 fusions were detected in RNA only (FIG. 4B); 100% were outside of DNA-baited region (NTRK1 intron 7, 8, and 9; NTRK3 no intron baiting).

### DETAILED DESCRIPTION OF THE INVENTION

The present disclosure is based, at least in part, on the discovery of anaplastic lymphoma kinase (ALK) gene fusions in certain cancers. In certain fusions, the ALK gene is fused to a gene encoding a collagen polypeptide. In certain fusions, the ALK gene is fused to a collagen alpha-2(V) chain (COL5A2) gene. In certain fusions, the ALK gene is fused to a collagen alpha-1(III) chain (COL3A1) gene.

The present disclosure also describes the results of comprehensive genomic profiling of DNA and RNA from more than 9,900 sarcoma tissue specimens. These analyses identified diverse rearrangements leading to fusion genes involving ALK, NTRK1, and NTRK3, such as COL3A1-ALK fusion genes. Importantly, analysis of RNA through hybrid capture-based sequencing led to the identification of fusion genes that were not detected by hybrid capture of DNA using baits corresponding to canonical non-small cell lung cancer (NSCLC) breakpoints, thereby increasing the sensitivity for atypical fusions with non-canonical breakpoints. Accordingly, without wishing to be bound by theory, it is thought that the presence of a kinase fusion described herein in a sample, e.g., a liquid biopsy sample comprising ctDNA and/or a tissue sample such as a tumor biopsy, from individuals having cancer may identify cancer patients who are likely to respond to treatment with an anti-cancer therapy such as a targeted anti-cancer therapy, e.g., as described herein.

In some aspects, provided herein are ALK gene fusion nucleic acid molecules and ALK gene fusion polypeptides. In some aspects, provided herein are COL5A2-ALK fusion nucleic acid molecules or COL5A2-ALK fusion polypeptides. In some aspects, provide herein are COL3A1-ALK fusion nucleic acid molecules or COL3A1-ALK fusion polypeptides.

In other aspects, provided herein are methods of treating or delaying progression of cancer. In other aspects, provided herein are methods of identifying one or more treatment options for an individual having cancer. In other aspects, provided herein are methods of selecting treatment for an individual having cancer. In other aspects, provided herein are methods of predicting survival of an individual having cancer treated with an anti-cancer therapy. In other aspects, provided herein are methods of screening an individual having cancer, suspected of having cancer, being tested for cancer, being treated for cancer, or being tested for a susceptibility to cancer. In other aspects, provided herein are methods of monitoring an individual having cancer. In other aspects, provided herein are methods of identifying an individual having cancer who may benefit from a treatment comprising an anti-cancer therapy. In other aspects, provided herein are methods of selecting a therapy for an individual having cancer. In some embodiments, the cancer comprises a COL5A2-ALK fusion nucleic acid molecule or polypeptide or a COL3A1-ALK fusion nucleic acid molecule or polypeptide described herein. In some embodiments, the methods comprise acquiring knowledge of a COL5A2-ALK fusion nucleic acid molecule or polypeptide or a COL3A1-ALK fusion nucleic acid molecule or polypeptide in a sample from an individual. In some embodiments, the methods comprise detecting a COL5A2-ALK fusion nucleic acid molecule or polypeptide or a COL3A1-ALK fusion nucleic acid molecule or polypeptide in a sample from an individual. In some embodiments, the methods comprise generating a report comprising one or more treatment options for the individual. In some embodiments, the methods comprise administering to an individual having cancer an effective amount of a treatment comprising an anti-cancer therapy. In some embodiments, the cancer is a hematologic malignancy or a solid tumor malignancy. In some embodiments, the cancer is a cancer provided herein. In some embodiments, the cancer is anaplastic large cell lymphoma (ALCL), non-small cell lung cancer (NSCLC), colorectal cancer (CRC), sarcoma, sarcoma not otherwise specified (NOS), inflammatory myofibroblastic tumor (IMT), rhabdomyosarcoma, acute myeloid leukemia, histiocytosis, leiomyosarcoma, ALK-positive large B-cell lymphoma, epithelioid fibrous histiocytoma, a pulmonary carcinoma, a renal cell carcinoma, a thyroid carcinoma, a pancreatic carcinoma, carcinoma of unknown primary, ovarian carcinoma, glioma, mesothelioma, melanoma, or a Spitzoid tumor. In some embodiments, the cancer comprises a COL5A2-ALK fusion nucleic acid molecule or polypeptide or a COL3A1-ALK fusion nucleic acid molecule or polypeptide described herein. In some embodiments, the cancer is rhabdomyosarcoma and comprises a COL5A2-ALK fusion nucleic acid molecule or polypeptide described herein. In some embodiments, the cancer is leiomyosarcoma, inflammatory myofibroblastic tumor (IMT), or sarcoma not otherwise specified (NOS) and comprises a COL3A1-ALK fusion nucleic acid molecule or polypeptide described herein. In some embodiments, the anti-cancer therapy is an anti-cancer therapy described herein, such as a small molecule inhibitor, an antibody, a cellular therapy, or a nucleic acid. In some embodiments, the anti-cancer therapy is an ALK-targeted therapy, such as a kinase inhibitor.

In other aspects, provided herein are methods of detecting a COL5A2-ALK fusion nucleic acid molecule or polypeptide or a COL3A1-ALK fusion nucleic acid molecule or polypeptide. In other aspects, provided herein are methods of assessing a COL5A2-ALK fusion nucleic acid molecule or polypeptide or a COL3A1-ALK fusion nucleic acid molecule or polypeptide. In some embodiments, the methods comprise detecting a COL5A2-ALK fusion nucleic acid molecule or polypeptide or a COL3A1-ALK fusion nucleic acid molecule or polypeptide in a sample from an individual. In some embodiments, the methods further comprise providing an assessment of the COL5A2-ALK fusion nucleic acid molecule or polypeptide or the COL3A1-ALK fusion nucleic acid molecule or polypeptide. In some embodiments, the individual has cancer, is suspected of having cancer, is being tested for cancer, is being treated for cancer, or is being tested for a susceptibility to cancer. In some embodiments, the cancer is a hematologic malignancy or a solid tumor malignancy. In some embodiments, the cancer is a cancer provided herein. In some embodiments, the cancer is anaplastic large cell lymphoma (ALCL), non-small cell lung cancer (NSCLC), colorectal cancer (CRC), sarcoma, sarcoma not otherwise specified (NOS), inflammatory myofibroblastic tumor (IMT), rhabdomyosarcoma, acute myeloid leukemia, histiocytosis, leiomyosarcoma, ALK-positive large B-cell lymphoma, epithelioid fibrous histiocytoma, a pulmonary carcinoma, a renal cell carcinoma, a thyroid carcinoma, a pancreatic carcinoma, carcinoma of unknown primary, ovarian carcinoma, glioma, mesothelioma, melanoma, or a Spitzoid tumor. In some embodiments, the cancer comprises a COL5A2-ALK fusion nucleic acid molecule or polypeptide or a COL3A1-ALK fusion nucleic acid molecule or polypeptide described herein. In some embodiments, the cancer is rhabdomyosarcoma and comprises a COL5A2-ALK fusion nucleic acid molecule or polypeptide described herein. In some embodiments, the cancer is leiomyosarcoma, inflammatory myofibroblastic tumor (IMT), or sarcoma not otherwise specified (NOS) and comprises a COL3A1-ALK fusion nucleic acid molecule or polypeptide described herein. In some embodiments, the anti-cancer therapy is an anti-cancer therapy described herein, such as a small molecule inhibitor, an antibody, a cellular therapy, or a nucleic acid. In some embodiments, the anti-cancer therapy is an ALK-targeted therapy, such as a kinase inhibitor.

In some embodiments, the individual is a human.

In other aspects, provided herein are anti-cancer therapies for use in a method of treating or delaying progression of cancer, such as a cancer comprising a COL5A2-ALK fusion nucleic acid molecule or polypeptide or a COL3A1-ALK fusion nucleic acid molecule or polypeptide.

In other aspects, provided herein are anti-cancer therapies for use in the manufacture of a medicament for treating or delaying progression of cancer, such as a cancer comprising a COL5A2-ALK fusion nucleic acid molecule or polypeptide or a COL3A1-ALK fusion nucleic acid molecule or polypeptide.

In other aspects, provided herein are in vitro uses of one or more oligonucleotides, probes, baits or antibodies for detecting or isolating a COL5A2-ALK fusion nucleic acid molecule or polypeptide or a COL3A1-ALK fusion nucleic acid molecule or polypeptide described herein.

In other aspects, provided herein are kits comprising one or more oligonucleotides, probes, baits or antibodies for detecting or isolating a COL5A2-ALK fusion nucleic acid molecule or polypeptide or a COL3A1-ALK fusion nucleic acid molecule or polypeptide described herein.

In other aspects, provided herein are vectors and host cells comprising a COL5A2-ALK fusion nucleic acid molecule or polypeptide or a COL3A1-ALK fusion nucleic acid molecule or polypeptide described herein.

### Definitions

Before describing the invention in detail, it is to be understood that this invention is not limited to particular compositions or biological systems. It is also to be understood that the terminology used herein is for the purpose of describing particular embodiments only, and is not intended to be limiting.

As used in this specification and the appended claims, the singular forms “a”, “an” and “the” include plural referents unless the content clearly dictates otherwise. Thus, for example, reference to “a molecule” optionally includes a combination of two or more such molecules, and the like.

The term “or” is used herein to mean, and is used interchangeably with, the term “and/or”, unless context clearly indicates otherwise.

The terms “about” or “approximately” as used herein refer to the usual error range for the respective value readily known to the skilled person in this technical field, for example, an acceptable degree of error or deviation for the quantity measured given the nature or precision of the measurements. Reference to “about” or “approximately” a value or parameter herein includes (and describes) embodiments that are directed to that value or parameter per se.

The terms “fusion” or “fusion molecule” are used generically herein, and include any fusion molecule (e.g., a gene (e.g., in genomic DNA), a gene product (e.g., cDNA, mRNA, polypeptide, or protein), and variants thereof) that includes a fragment of a first gene or gene product and a fragment of a second gene or gene product described herein. A fusion molecule includes a “breakpoint” or “fusion junction,” which is the transition (i.e., direct fusion) point between the first gene or gene product, or fragment thereof, and the second gene or gene product, or fragment thereof.

The term “isolated” in the context of a nucleic acid molecule or a polypeptide refers to a nucleic acid molecule or polypeptide being separated from other nucleic acid molecules or polypeptides that are present in the natural source of the nucleic acid molecule or polypeptide. In some certain embodiments, the isolated nucleic acid molecule or polypeptide is free of or substantially free of other cellular material or culture medium when produced by recombinant techniques, or free of or substantially free of chemical precursors or other chemicals when chemically synthesized.

As used herein, the term “configured to hybridize to” indicates that a nucleic acid molecule has a nucleotide sequence with sufficient length and sequence complementarity to the nucleotide sequence of a target nucleic acid to allow the nucleic acid molecule to hybridize to the target nucleic acid, e.g., with a Tm of at least 65° C. in an aqueous solution of 1×SCC (150 mM sodium chloride and 15 mM trisodium citrate) and 0.1% SDS. Other hybridization conditions may be used when hybridizing a nucleic acid molecule to a target nucleic acid molecule, for example in the context of a described method.

“Percent (%) sequence identity” with respect to a reference polypeptide or polynucleotide sequence is defined as the percentage of amino acid residues or nucleotides in a sequence that are identical to the amino acid residues or nucleotides in the reference polypeptide or polynucleotide sequence, after aligning the sequences and introducing gaps, if necessary, to achieve the maximum percent sequence identity, and not considering any conservative substitutions as part of the sequence identity.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the individual or subject is a human. In some embodiments, the individual is human patient, e.g., a human patient having a cancer described herein, and/or a fusion nucleic acid molecule or polypeptide described herein.

An “effective amount” or a “therapeutically effective amount” of an agent, e.g., an anti-cancer agent, or a pharmaceutical formulation, refers to an amount effective, at dosages and for periods of time necessary, to achieve the desired therapeutic or prophylactic result, e.g., in the treatment or management of a cancer, for example, delaying or minimizing one or more symptoms associated with the cancer. In some embodiments, an effective amount or a therapeutically effective amount of an agent refers to an amount of the agent at dosages and for periods of time necessary, alone or in combination with other therapeutic agents, which provides a therapeutic or prophylactic benefit in the treatment or management of a disease such as a cancer. In some embodiments, an effective amount or a therapeutically effective amount of an agent enhances the therapeutic or prophylactic efficacy of another therapeutic agent or another therapeutic modality.

As used herein, “treatment” (and grammatical variations thereof such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, delaying progression of disease, alleviation of symptoms, diminishment of any direct or indirect pathological consequences of the disease, decreasing the rate of disease progression, amelioration or palliation of the disease state, and remission or improved prognosis. In some embodiments, the terms “treatment,” “treat,” or “treating” include preventing a disease, such as cancer, e.g., before an individual begins to suffer from a cancer or from re-growth or recurrence of the cancer. In some embodiments, the terms “treatment,” “treat,” or “treating” include inhibiting or reducing the severity of a disease such as a cancer.

“Likely to” or “increased likelihood,” as used herein, refer to an increased probability that an event, item, object, thing or person will occur. Thus, in one example, an individual that is likely to respond to treatment with an anti-cancer therapy, e.g., an anti-cancer therapy provided herein, alone or in combination, has an increased probability of responding to treatment with the anti-cancer therapy alone or in combination, relative to a reference individual or group of individuals. “Unlikely to” refers to a decreased probability that an event, item, object, thing or person will occur relative to a reference individual or group of individuals. Thus, an individual that is unlikely to respond to treatment with an anti-cancer therapy, e.g., an anti-cancer therapy provided herein, alone or in combination, has a decreased probability of responding to treatment with the anti-cancer therapy, alone or in combination, relative to a reference individual or group of individuals.

“Sample,” as used herein, refers to a biological sample obtained or derived from a source of interest, as described herein.

### ALK Fusions of the Disclosure

In some aspects, provided herein are anaplastic lymphoma kinase (ALK) gene fusions, including ALK fusion nucleic acid molecules and ALK fusion polypeptide molecules.

The ALK gene encodes a receptor tyrosine kinase comprising an extracellular ligand-binding domain, a transmembrane domain, and an intracellular tyrosine kinase domain. ALK plays important roles in cellular communication, and in the development and function of the nervous system. For example, ALK is known to activate multiple signal transduction pathways, including the MAPK-ERK, PI3K-AKT, PLCy, CRKL-C3G, and JAK-STAT pathways. An exemplary ALK nucleotide sequence is available as Transcript ID NM_004304 (e.g., available at the website

www[dot]ncbi[dot]nlm[dot]nih[dot]gov/nuccore/NM_004304.4), and provided herein in SEQ ID NO: 1.

(SEQ ID NO: 1)
AGATGCGATCCAGCGGCTCTGGGGGCGGCAGCGGTGGTAGCAGCTGGTACCTCCCGCCGCCT
CTGTTCGGAGGGTCGCGGGGCACCGAGGTGCTTTCCGGCCGCCCTCTGGTCGGCCACCCAAA
GCCGCGGGCGCTGATGATGGGTGAGGAGGGGGCGGCAAGATTTCGGGCGCCCCTGCCCTGAA
CGCCCTCAGCTGCTGCCGCCGGGGCCGCTCCAGTGCCTGCGAACTCTGAGGAGCCGAGGCGC
CGGTGAGAGCAAGGACGCTGCAAACTTGCGCAGCGCGGGGGCTGGGATTCACGCCCAGAAGT
TCAGCAGGCAGACAGTCCGAAGCCTTCCCGCAGCGGAGAGATAGCTTGAGGGTGCGCAAGAC
GGCAGCCTCCGCCCTCGGTTCCCGCCCAGACCGGGCAGAAGAGCTTGGAGGAGCCAAAAGGA
ACGCAAAAGGCGGCCAGGACAGCGTGCAGCAGCTGGGAGCCGCCGTTCTCAGCCTTAAAAGT
TGCAGAGATTGGAGGCTGCCCCGAGAGGGGACAGACCCCAGCTCCGACTGCGGGGGGCAGGA
GAGGACGGTACCCAACTGCCACCTCCCTTCAACCATAGTAGTTCCTCTGTACCGAGCGCAGC
GAGCTACAGACGGGGGCGCGGCACTCGGCGCGGAGAGCGGGAGGCTCAAGGTCCCAGCCAGT
GAGCCCAGTGTGCTTGAGTGTCTCTGGACTCGCCCCTGAGCTTCCAGGTCTGTTTCATTTAG
ACTCCTGCTCGCCTCCGTGCAGTTGGGGGAAAGCAAGAGACTTGCGCGCACGCACAGTCCTC
TGGAGATCAGGTGGAAGGAGCCGCTGGGTACCAAGGACTGTTCAGAGCCTCTTCCCATCTCG
GGGAGAGCGAAGGGTGAGGCTGGGCCCGGAGAGCAGTGTAAACGGCCTCCTCCGGCGGGATG
GGAGCCATCGGGCTCCTGTGGCTCCTGCCGCTGCTGCTTTCCACGGCAGCTGTGGGCTCCGG
GATGGGGACCGGCCAGCGCGCGGGCTCCCCAGCTGCGGGGCCGCCGCTGCAGCCCCGGGAGC
CACTCAGCTACTCGCGCCTGCAGAGGAAGAGTCTGGCAGTTGACTTCGTGGTGCCCTCGCTC
TTCCGTGTCTACGCCCGGGACCTACTGCTGCCACCATCCTCCTCGGAGCTGAAGGCTGGCAG
GCCCGAGGCCCGCGGCTCGCTAGCTCTGGACTGCGCCCCGCTGCTCAGGTTGCTGGGGCCGG
CGCCGGGGGTCTCCTGGACCGCCGGTTCACCAGCCCCGGCAGAGGCCCGGACGCTGTCCAGG
GTGCTGAAGGGCGGCTCCGTGCGCAAGCTCCGGCGTGCCAAGCAGTTGGTGCTGGAGCTGGG
CGAGGAGGCGATCTTGGAGGGTTGCGTCGGGCCCCCCGGGGAGGCGGCTGTGGGGCTGCTCC
AGTTCAATCTCAGCGAGCTGTTCAGTTGGTGGATTCGCCAAGGCGAAGGGCGACTGAGGATC
CGCCTGATGCCCGAGAAGAAGGCGTCGGAAGTGGGCAGAGAGGGAAGGCTGTCCGCGGCAAT
TCGCGCCTCCCAGCCCCGCCTTCTCTTCCAGATCTTCGGGACTGGTCATAGCTCCTTGGAAT
CACCAACAAACATGCCTTCTCCTTCTCCTGATTATTTTACATGGAATCTCACCTGGATAATG
AAAGACTCCTTCCCTTTCCTGTCTCATCGCAGCCGATATGGTCTGGAGTGCAGCTTTGACTT
CCCCTGTGAGCTGGAGTATTCCCCTCCACTGCATGACCTCAGGAACCAGAGCTGGTCCTGGC
GCCGCATCCCCTCCGAGGAGGCCTCCCAGATGGACTTGCTGGATGGGCCTGGGGCAGAGCGT
TCTAAGGAGATGCCCAGAGGCTCCTTTCTCCTTCTCAACACCTCAGCTGACTCCAAGCACAC
CATCCTGAGTCCGTGGATGAGGAGCAGCAGTGAGCACTGCACACTGGCCGTCTCGGTGCACA
GGCACCTGCAGCCCTCTGGAAGGTACATTGCCCAGCTGCTGCCCCACAACGAGGCTGCAAGA
GAGATCCTCCTGATGCCCACTCCAGGGAAGCATGGTTGGACAGTGCTCCAGGGAAGAATCGG
GCGTCCAGACAACCCATTTCGAGTGGCCCTGGAATACATCTCCAGTGGAAACCGCAGCTTGT
CTGCAGTGGACTTCTTTGCCCTGAAGAACTGCAGTGAAGGAACATCCCCAGGCTCCAAGATG
GCCCTGCAGAGCTCCTTCACTTGTTGGAATGGGACAGTCCTCCAGCTTGGGCAGGCCTGTGA
CTTCCACCAGGACTGTGCCCAGGGAGAAGATGAGAGCCAGATGTGCCGGAAACTGCCTGTGG
GTTTTTACTGCAACTTTGAAGATGGCTTCTGTGGCTGGACCCAAGGCACACTGTCACCCCAC
ACTCCTCAATGGCAGGTCAGGACCCTAAAGGATGCCCGGTTCCAGGACCACCAAGACCATGC
TCTATTGCTCAGTACCACTGATGTCCCCGCTTCTGAAAGTGCTACAGTGACCAGTGCTACGT
TTCCTGCACCGATCAAGAGCTCTCCATGTGAGCTCCGAATGTCCTGGCTCATTCGTGGAGTC
TTGAGGGGAAACGTGTCCTTGGTGCTAGTGGAGAACAAAACCGGGAAGGAGCAAGGCAGGAT
GGTCTGGCATGTCGCCGCCTATGAAGGCTTGAGCCTGTGGCAGTGGATGGTGTTGCCTCTCC
TCGATGTGTCTGACAGGTTCTGGCTGCAGATGGTCGCATGGTGGGGACAAGGATCCAGAGCC
ATCGTGGCTTTTGACAATATCTCCATCAGCCTGGACTGCTACCTCACCATTAGCGGAGAGGA
CAAGATCCTGCAGAATACAGCACCCAAATCAAGAAACCTGTTTGAGAGAAACCCAAACAAGG
AGCTGAAACCCGGGGAAAATTCACCAAGACAGACCCCCATCTTTGACCCTACAGTTCATTGG
CTGTTCACCACATGTGGGGCCAGCGGGCCCCATGGCCCCACCCAGGCACAGTGCAACAACGC
CTACCAGAACTCCAACCTGAGCGTGGAGGTGGGGAGCGAGGGCCCCCTGAAAGGCATCCAGA
TCTGGAAGGTGCCAGCCACCGACACCTACAGCATCTCGGGCTACGGAGCTGCTGGCGGGAAA
GGCGGGAAGAACACCATGATGCGGTCCCACGGCGTGTCTGTGCTGGGCATCTTCAACCTGGA
GAAGGATGACATGCTGTACATCCTGGTTGGGCAGCAGGGAGAGGACGCCTGCCCCAGTACAA
ACCAGTTAATCCAGAAAGTCTGCATTGGAGAGAACAATGTGATAGAAGAAGAAATCCGTGTG
AACAGAAGCGTGCATGAGTGGGCAGGAGGCGGAGGAGGAGGGGGTGGAGCCACCTACGTATT
TAAGATGAAGGATGGAGTGCCGGTGCCCCTGATCATTGCAGCCGGAGGTGGTGGCAGGGCCT
ACGGGGCCAAGACAGACACGTTCCACCCAGAGAGACTGGAGAATAACTCCTCGGTTCTAGGG
CTAAACGGCAATTCCGGAGCCGCAGGTGGTGGAGGTGGCTGGAATGATAACACTTCCTTGCT
CTGGGCCGGAAAATCTTTGCAGGAGGGTGCCACCGGAGGACATTCCTGCCCCCAGGCCATGA
AGAAGTGGGGGTGGGAGACAAGAGGGGGTTTCGGAGGGGGTGGAGGGGGGTGCTCCTCAGGT
GGAGGAGGCGGAGGATATATAGGCGGCAATGCAGCCTCAAACAATGACCCCGAAATGGATGG
GGAAGATGGGGTTTCCTTCATCAGTCCACTGGGCATCCTGTACACCCCAGCTTTAAAAGTGA
TGGAAGGCCACGGGGAAGTGAATATTAAGCATTATCTAAACTGCAGTCACTGTGAGGTAGAC
GAATGTCACATGGACCCTGAAAGCCACAAGGTCATCTGCTTCTGTGACCACGGGACGGTGCT
GGCTGAGGATGGCGTCTCCTGCATTGTGTCACCCACCCCGGAGCCACACCTGCCACTCTCGC
TGATCCTCTCTGTGGTGACCTCTGCCCTCGTGGCCGCCCTGGTCCTGGCTTTCTCCGGCATC
ATGATTGTGTACCGCCGGAAGCACCAGGAGCTGCAAGCCATGCAGATGGAGCTGCAGAGCCC
TGAGTACAAGCTGAGCAAGCTCCGCACCTCGACCATCATGACCGACTACAACCCCAACTACT
GCTTTGCTGGCAAGACCTCCTCCATCAGTGACCTGAAGGAGGTGCCGCGGAAAAACATCACC
CTCATTCGGGGTCTGGGCCATGGCGCCTTTGGGGAGGTGTATGAAGGCCAGGTGTCCGGAAT
GCCCAACGACCCAAGCCCCCTGCAAGTGGCTGTGAAGACGCTGCCTGAAGTGTGCTCTGAAC
AGGACGAACTGGATTTCCTCATGGAAGCCCTGATCATCAGCAAATTCAACCACCAGAACATT
GTTCGCTGCATTGGGGTGAGCCTGCAATCCCTGCCCCGGTTCATCCTGCTGGAGCTCATGGC
GGGGGGAGACCTCAAGTCCTTCCTCCGAGAGACCCGCCCTCGCCCGAGCCAGCCCTCCTCCC
TGGCCATGCTGGACCTTCTGCACGTGGCTCGGGACATTGCCTGTGGCTGTCAGTATTTGGAG
GAAAACCACTTCATCCACCGAGACATTGCTGCCAGAAACTGCCTCTTGACCTGTCCAGGCCC
TGGAAGAGTGGCCAAGATTGGAGACTTCGGGATGGCCCGAGACATCTACAGGGCGAGCTACT
ATAGAAAGGGAGGCTGTGCCATGCTGCCAGTTAAGTGGATGCCCCCAGAGGCCTTCATGGAA
GGAATATTCACTTCTAAAACAGACACATGGTCCTTTGGAGTGCTGCTATGGGAAATCTTTTC
TCTTGGATATATGCCATACCCCAGCAAAAGCAACCAGGAAGTTCTGGAGTTTGTCACCAGTG
GAGGCCGGATGGACCCACCCAAGAACTGCCCTGGGCCTGTATACCGGATAATGACTCAGTGC
TGGCAACATCAGCCTGAAGACAGGCCCAACTTTGCCATCATTTTGGAGAGGATTGAATACTG
CACCCAGGACCCGGATGTAATCAACACCGCTTTGCCGATAGAATATGGTCCACTTGTGGAAG
AGGAAGAGAAAGTGCCTGTGAGGCCCAAGGACCCTGAGGGGGTTCCTCCTCTCCTGGTCTCT
CAACAGGCAAAACGGGAGGAGGAGCGCAGCCCAGCTGCCCCACCACCTCTGCCTACCACCTC
CTCTGGCAAGGCTGCAAAGAAACCCACAGCTGCAGAGATCTCTGTTCGAGTCCCTAGAGGGC
CGGCCGTGGAAGGGGGACACGTGAATATGGCATTCTCTCAGTCCAACCCTCCTTCGGAGTTG
CACAAGGTCCACGGATCCAGAAACAAGCCCACCAGCTTGTGGAACCCAACGTACGGCTCCTG
GTTTACAGAGAAACCCACCAAAAAGAATAATCCTATAGCAAAGAAGGAGCCACACGACAGGG
GTAACCTGGGGCTGGAGGGAAGCTGTACTGTCCCACCTAACGTTGCAACTGGGAGACTTCCG
GGGGCCTCACTGCTCCTAGAGCCCTCTTCGCTGACTGCCAATATGAAGGAGGTACCTCTGTT
CAGGCTACGTCACTTCCCTTGTGGGAATGTCAATTACGGCTACCAGCAACAGGGCTTGCCCT
TAGAAGCCGCTACTGCCCCTGGAGCTGGTCATTACGAGGATACCATTCTGAAAAGCAAGAAT
AGCATGAACCAGCCTGGGCCCTGAGCTCGGTCGCACACTCACTTCTCTTCCTTGGGATCCCT
AAGACCGTGGAGGAGAGAGAGGCAATGGCTCCTTCACAAACCAGAGACCAAATGTCACGTTT
TGTTTTGTGCCAACCTATTTTGAAGTACCACCAAAAAAGCTGTATTTTGAAAATGCTTTAGA
AAGGTTTTGAGCATGGGTTCATCCTATTCTTTCGAAAGAAGAAAATATCATAAAAATGAGTG
ATAAATACAAGGCCCAGATGTGGTTGCATAAGGTTTTTATGCATGTTTGTTGTATACTTCCT
TATGCTTCTTTCAAATTGTGTGTGCTCTGCTTCAATGTAGTCAGAATTAGCTGCTTCTATGT
TTCATAGTTGGGGTCATAGATGTTTCCTTGCCTTGTTGATGTGGACATGAGCCATTTGAGGG
GAGAGGGAACGGAAATAAAGGAGTTATTTGTAATGACTAA

An exemplary ALK polypeptide amino acid sequence is provided herein in SEQ ID NO: 4.

(SEQ ID NO: 4)
MGAIGLLWLLPLLLSTAAVGSGMGTGQRAGSPAAGPPLQPREPLSYSRLQRKSLAVDFVVPS
LFRVYARDLLLPPSSSELKAGRPEARGSLALDCAPLLRLLGPAPGVSWTAGSPAPAEARTLS
RVLKGGSVRKLRRAKQLVLELGEEAILEGCVGPPGEAAVGLLQFNLSELFSWWIRQGEGRLR
IRLMPEKKASEVGREGRLSAAIRASQPRLLFQIFGTGHSSLESPTNMPSPSPDYFTWNLTWI
MKDSFPFLSHRSRYGLECSFDFPCELEYSPPLHDLRNQSWSWRRIPSEEASQMDLLDGPGAE
RSKEMPRGSFLLLNTSADSKHTILSPWMRSSSEHCTLAVSVHRHLQPSGRYIAQLLPHNEAA
REILLMPTPGKHGWTVLQGRIGRPDNPERVALEYISSGNRSLSAVDFFALKNCSEGTSPGSK
MALQSSFTCWNGTVLQLGQACDFHQDCAQGEDESQMCRKLPVGFYCNFEDGFCGWTQGTLSP
HTPQWQVRTLKDARFQDHQDHALLLSTTDVPASESATVISATFPAPIKSSPCELRMSWLIRG
VLRGNVSLVLVENKTGKEQGRMVWHVAAYEGLSLWQWMVLPLLDVSDREWLQMVAWWGQGSR
AIVAFDNISISLDCYLTISGEDKILQNTAPKSRNLFERNPNKELKPGENSPRQTPIFDPTVH
WLFTTCGASGPHGPTQAQCNNAYQNSNLSVEVGSEGPLKGIQIWKVPATDTYSISGYGAAGG
KGGKNTMMRSHGVSVLGIFNLEKDDMLYILVGQQGEDACPSTNQLIQKVCIGENNVIEEEIR
VNRSVHEWAGGGGGGGGATYVFKMKDGVPVPLIIAAGGGGRAYGAKTDTFHPERLENNSSVL
GLNGNSGAAGGGGGWNDNTSLLWAGKSLQEGATGGHSCPQAMKKWGWETRGGFGGGGGGCSS
GGGGGGYIGGNAASNNDPEMDGEDGVSFISPLGILYTPALKVMEGHGEVNIKHYLNCSHCEV
DECHMDPESHKVICFCDHGTVLAEDGVSCIVSPTPEPHLPLSLILSVVTSALVAALVLAFSG
IMIVYRRKHQELQAMQMELQSPEYKLSKLRTSTIMTDYNPNYCFAGKTSSISDLKEVPRKNI
TLIRGLGHGAFGEVYEGQVSGMPNDPSPLQVAVKTLPEVCSEQDELDELMEALIISKENHQN
IVRCIGVSLQSLPRFILLELMAGGDLKSFLRETRPRPSQPSSLAMLDLLHVARDIACGCQYL
EENHFIHRDIAARNCLLTCPGPGRVAKIGDFGMARDIYRASYYRKGGCAMLPVKWMPPEAFM
EGIFTSKTDTWSFGVLLWEIFSLGYMPYPSKSNQEVLEFVTSGGRMDPPKNCPGPVYRIMTQ
CWQHQPEDRPNFAIILERIEYCTQDPDVINTALPIEYGPLVEEEEKVPVRPKDPEGVPPLLV
SQQAKREEERSPAAPPPLPTTSSGKAAKKPTAAEISVRVPRGPAVEGGHVNMAFSQSNPPSE
LHKVHGSRNKPTSLWNPTYGSWFTEKPTKKNNPIAKKEPHDRGNLGLEGSCTVPPNVATGRL
PGASLLLEPSSLTANMKEVPLFRLRHFPCGNVNYGYQQQGLPLEAATAPGAGHYEDTILKSK
NSMNQPGP

ALK Fusion Nucleic Acid Molecules

In some aspects, provided herein are ALK gene fusions, wherein the ALK gene or a portion thereof is fused to a gene, or a portion thereof, encoding a collagen polypeptide. In certain fusions provided herein, the ALK gene or a portion thereof is fused to a collagen alpha-2(V) chain (COL5A2) gene or a portion thereof. In certain fusions provided herein, the ALK gene is fused to a collagen alpha-1(III) chain (COL3A1) gene or a portion thereof.

The COL5A2 gene encodes the collagen alpha-2(V) chain protein, which is an alpha chain for one of the low abundance fibrillar collagens. Type V collagen is involved in regulation of assembly of heterotypic fibers composed of both type I and type V collagen. Mutations in the COL5A2 gene have been associated with Ehlers-Danlos syndrome. An exemplary COL5A2 nucleotide sequence is available as Transcript ID NM_000393 (e.g., available at the website www[dot]ncbi[dot]nlm[dot]nih[dot]gov/nuccore/NM_000393), and provided herein in SEQ ID NO: 2.

(SEQ ID NO: 2)
GCAGACTGTGCTGGAGCTGGTGCTGAAAAAGGGGGTTTGCAGAGGCTGCCCTGGGGCTGGTG
CTGAAAGAAGAGCCCACAGCTGACTTCATGGTGCTACAATAACCTCAGAATCTACTTTTCAC
TCTCAGGAGAACCCACATGTCTAATATTTAGACATGATGGCAAACTGGGCGGAAGCAAGACC
TCTCCTCATTCTTATTGTTTTATTAGGGCAATTTGTCTCAATAAAAGCCCAGGAAGAAGACG
AGGATGAAGGATATGGTGAAGAAATAGCCTGCACTCAGAATGGCCAGATGTACTTAAACAGG
GACATTTGGAAACCTGCCCCTTGTCAGATCTGTGTCTGTGACAATGGAGCCATTCTCTGTGA
CAAGATAGAATGCCAGGATGTGCTGGACTGTGCCGACCCTGTAACGCCCCCTGGGGAATGCT
GTCCTGTCTGTTCACAAACACCTGGAGGTGGCAATACCAATTTTGGTAGAGGAAGAAAGGGA
CAAAAGGGAGAACCAGGATTAGTGCCTGTTGTAACAGGCATACGTGGTCGTCCAGGACCGGC
AGGACCTCCAGGATCACAGGGACCAAGAGGAGAGCGAGGGCCAAAAGGAAGACCTGGCCCTC
GTGGACCTCAGGGAATTGATGGAGAACCAGGTGTTCCTGGTCAACCTGGTGCTCCAGGACCT
CCTGGACATCCGTCCCACCCAGGACCCGATGGCTTGAGCAGGCCGTTTTCAGCTCAAATGGC
TGGGTTGGATGAAAAATCTGGACTTGGGAGTCAAGTAGGACTAATGCCTGGCTCTGTGGGTC
CTGTTGGCCCAAGGGGACCACAGGGTTTACAAGGACAGCAAGGTGGTGCAGGACCTACAGGA
CCTCCTGGTGAACCTGGTGATCCTGGACCAATGGGTCCGATTGGTTCACGTGGACCAGAGGG
CCCTCCTGGTAAACCTGGGGAAGATGGTGAACCTGGCAGAAATGGAAATCCTGGTGAAGTGG
GATTTGCAGGATCTCCGGGAGCTCGTGGATTTCCTGGGGCTCCTGGTCTTCCAGGTCTGAAG
GGTCACCGAGGACACAAAGGTCTTGAAGGCCCTAAAGGTGAAGTTGGAGCACCTGGTTCCAA
GGGTGAAGCTGGCCCCACTGGTCCAATGGGTGCCATGGGTCCTCTGGGTCCGAGGGGAATGC
CAGGAGAGAGAGGGAGACTTGGGCCACAGGGTGCTCCTGGACAACGAGGTGCACATGGTATG
CCTGGAAAACCTGGACCAATGGGTCCTCTTGGGATACCAGGCTCTTCTGGTTTTCCAGGAAA
TCCTGGAATGAAGGGAGAAGCAGGTCCTACAGGGGCGCGAGGCCCTGAAGGTCCTCAGGGGC
AGAGAGGTGAAACTGGGCCCCCAGGTCCAGTTGGCTCTCCAGGTCTTCCTGGTGCAATAGGA
ACTGATGGTACTCCTGGTGCCAAAGGCCCAACGGGCTCTCCAGGTACCTCTGGTCCTCCTGG
CTCAGCAGGGCCTCCTGGATCTCCAGGACCTCAGGGTAGCACTGGTCCTCAGGGAATTCGAG
GCCAACCGGGTGATCCAGGAGTTCCAGGTTTCAAAGGAGAAGCTGGCCCAAAAGGGGAACCA
GGGCCACATGGTATTCAGGGTCCGATAGGCCCACCCGGTGAAGAAGGCAAAAGAGGTCCCAG
AGGTGACCCAGGAACAGTTGGTCCTCCAGGGCCAGTGGGAGAAAGGGGTGCTCCTGGCAATC
GTGGTTTTCCAGGCTCTGATGGTTTACCTGGGCCAAAGGGTGCTCAAGGAGAACGGGGTCCT
GTAGGTTCTTCAGGACCCAAAGGAAGCCAGGGGGATCCAGGACGTCCAGGGGAACCTGGGCT
TCCAGGTGCTCGGGGTTTGACAGGAAATCCTGGTGTTCAAGGTCCTGAAGGAAAACTTGGAC
CTTTGGGTGCGCCAGGGGAAGATGGCCGTCCAGGTCCTCCAGGCTCCATAGGAATCAGAGGG
CAGCCCGGGAGCATGGGCCTTCCAGGCCCCAAAGGTAGCAGTGGTGACCCTGGGAAACCTGG
AGAAGCAGGAAATGCTGGAGTTCCTGGGCAGAGGGGAGCTCCTGGAAAAGATGGTGAAGTTG
GTCCTTCTGGTCCTGTGGGCCCGCCGGGTCTAGCTGGTGAAAGAGGAGAACAAGGACCTCCA
GGCCCCACAGGTTTTCAGGGGCTTCCTGGTCCTCCAGGGCCTCCTGGAGAAGGTGGAAAACC
AGGTGATCAAGGTGTTCCTGGAGATCCCGGAGCAGTTGGCCCGTTAGGACCTAGAGGAGAAC
GAGGAAATCCTGGGGAAAGAGGAGAACCTGGGATAACTGGACTCCCTGGTGAGAAGGGAATG
GCTGGAGGACATGGTCCTGATGGCCCAAAAGGCAGTCCAGGTCCATCTGGGACCCCTGGAGA
TACAGGCCCACCAGGTCTTCAAGGTATGCCGGGAGAAAGAGGAATTGCAGGAACTCCTGGCC
CCAAGGGTGACAGAGGTGGCATAGGAGAAAAAGGTGCTGAAGGCACAGCTGGAAATGATGGT
GCAAGAGGTCTTCCAGGTCCTTTGGGCCCTCCAGGTCCGGCAGGTCCTACTGGAGAAAAGGG
TGAACCTGGTCCTCGAGGTTTAGTTGGCCCTCCTGGCTCCCGGGGCAATCCTGGTTCTCGAG
GTGAAAATGGGCCAACTGGAGCTGTTGGTTTTGCCGGACCCCAGGGTCCTGACGGACAGCCT
GGAGTAAAAGGTGAACCTGGAGAGCCAGGACAGAAGGGAGATGCTGGTTCTCCTGGACCACA
AGGTTTAGCAGGATCCCCTGGCCCTCATGGTCCTAATGGTGTTCCTGGACTAAAAGGTGGTC
GAGGAACCCAAGGTCCGCCTGGTGCTACAGGATTTCCTGGTTCTGCGGGCAGAGTTGGACCT
CCAGGCCCTGCTGGAGCTCCAGGACCTGCGGGACCCCTAGGGGAACCCGGGAAGGAGGGACC
TCCAGGTCTTCGTGGGGACCCTGGCTCTCATGGGCGTGTGGGAGATCGAGGACCAGCTGGCC
CCCCTGGTGGCCCAGGAGACAAAGGGGACCCAGGAGAAGATGGGCAACCTGGTCCAGATGGC
CCCCCTGGTCCAGCTGGAACGACCGGGCAGAGAGGAATTGTTGGCATGCCTGGGCAACGTGG
AGAGAGAGGCATGCCCGGCCTACCAGGCCCAGCGGGAACACCAGGAAAAGTAGGACCAACTG
GTGCAACAGGAGATAAAGGTCCACCTGGACCTGTGGGGCCCCCAGGCTCCAATGGTCCTGTA
GGGGAACCTGGACCAGAAGGTCCAGCTGGCAATGATGGTACCCCAGGACGGGATGGTGCTGT
TGGAGAACGTGGTGATCGTGGAGACCCTGGGCCTGCAGGTCTGCCAGGCTCTCAGGGTGCCC
CTGGAACTCCTGGCCCTGTGGGTGCTCCAGGAGATGCAGGACAAAGAGGAGATCCGGGTTCT
CGGGGTCCTATAGGACCACCTGGTCGAGCTGGGAAACGTGGATTACCTGGACCCCAAGGACC
TCGTGGTGACAAAGGTGATCATGGAGACCGAGGTGACAGAGGTCAGAAGGGCCACAGAGGCT
TTACTGGTCTTCAGGGTCTTCCTGGCCCTCCTGGTCCAAATGGTGAACAAGGAAGTGCTGGA
ATCCCTGGACCATTTGGCCCAAGAGGTCCTCCAGGCCCAGTTGGTCCTTCAGGTAAAGAAGG
AAACCCTGGGCCACTTGGGCCAATTGGACCTCCAGGTGTACGAGGCAGTGTAGGAGAAGCAG
GACCTGAGGGCCCTCCTGGTGAGCCTGGCCCACCTGGCCCTCCGGGTCCCCCTGGCCACCTT
ACAGCTGCTCTTGGGGATATCATGGGGCACTATGATGAAAGCATGCCAGATCCACTTCCTGA
GTTTACTGAAGATCAGGCGGCTCCTGATGACAAAAACAAAACGGACCCAGGGGTTCATGCTA
CCCTGAAGTCACTCAGTAGTCAGATTGAAACCATGCGCAGCCCCGATGGCTCGAAAAAGCAC
CCAGCCCGCACGTGTGATGACCTAAAGCTTTGCCATTCCGCAAAGCAGAGTGGTGAATACTG
GATTGATCCTAACCAAGGATCTGTTGAAGATGCAATCAAAGTTTACTGCAACATGGAAACAG
GAGAAACATGTATTTCAGCAAACCCATCCAGTGTACCACGTAAAACCTGGTGGGCCAGTAAA
TCTCCTGACAATAAACCTGTTTGGTATGGTCTTGATATGAACAGAGGGTCTCAGTTCGCTTA
TGGAGACCACCAATCACCTAATACAGCCATTACTCAGATGACTTTTTTGCGCCTTTTATCAA
AAGAAGCCTCCCAGAACATCACTTACATCTGTAAAAACAGTGTAGGATACATGGACGATCAA
GCTAAGAACCTCAAAAAAGCTGTGGTTCTCAAAGGGGCAAATGACTTAGATATCAAAGCAGA
GGGAAATATTAGATTCCGGTATATCGTTCTTCAAGACACTTGCTCTAAGCGGAATGGAAATG
TGGGCAAGACTGTCTTTGAATATAGAACACAGAATGTGGCACGCTTGCCCATCATAGATCTT
GCTCCTGTGGATGTTGGCGGCACAGACCAGGAATTCGGCGTTGAAATTGGGCCAGTTTGTTT
TGTGTAAAGTAAGCCAAGACACATCGACAATGAGCACCACCATCAATGACCACCGCCATTCA
CAAGAACTTTGACTGTTTGAAGTTGATCCTGAGACTCTTGAAGTAATGGCTGATCCTGCATC
AGCATTGTATATATGGTCTTAAGTGCCTGGCCTCCTTATCCTTCAGAATATTTATTTTACTT
ACAATCCTCAAGTTTTAATTGATTTTAAATATTTTTCAATACAACAGTTTAGGTTTAAGATG
ACCAATGACAATGACCACCTTTGCAGAAAGTAAACTGATTGAATAAATAAATCTCCGTTTTC
TTCAATTTATTTCAGTGTAATGAAAAAGTTGCTTAGTATTTATGAGGAAATTCTTCTTCCTG
GCAGGTAGCTTAAAGAGTGGGGTATATAGAGCCACAACACATGTTTATTTTGCTTGGCTGCA
GTTGAAAAATAGAAATTAGTGCCCTTTTGTGACCTCTCATTCCAAGATTGTCAATTAAAAAT
GAGTTTAAAATGTTTAACTTGTGATCGAGACCTACATGCATGTCTTGATATTGTGTAACTAT
AATAGAGACTCTTTAAGGAGAATCTTAAAAAAAAAAAAACGTTTCTCACTGTCTTAAATAGA
ATTTTTAAATAGTATATATTCAGTGGCATTTTGGAGAACAAAGTGAATTTACTTCGACTTCT
TAAATTTTTGTAAAAGACTATAAGTTTAGACATCTTTCTCATTCAAATTTAAAGATATCTTT
CTCCTCTTGATCAATCTATCAATATTGATAGAAGTCACACTAGTATATACCATTTAATACAT
TTACACTTTCTTATTTAAGAAGATATTGAATGCAAAATAATTGACATATAGAACTTTACAAA
CATATGTCCAAGGACTCTAAATTGAGACTCTTCCACATGTACAATCTCATCATCCTGAAGCC
TATAATGAAGAAAAAGATCTAGAAACTGAGTTGTGGAGCTGACTCTAATCAAATGTGATGAT
TGGAATTAGACCATTTGGCCTTTGAACTTTCATAGGAAAAATGACCCAACATTTCTTAGCAT
GAGCTACCTCATCTCTAGAAGCTGGGATGGACTTACTATTCTTGTTTATATTTTAGATACTG
AAAGGTGCTATGCTTCTGTTATTATTCCAAGACTGGAGATAGGCAGGGCTAAAAAGGTATTA
TTATTTTTCCTTTAATGATGGTGCTAAAATTCTTCCTATAAAATTCCTTAAAAATAAAGATG
GTTTAATCACTACCATTGTGAAAACATAACTGTTAGACTTCCCGTTTCTGAAAGAAAGAGCA
TCGTTCCAATGCTTGTTCACTGTTCCTCTGTCATACTGTATCTGGAATGCTTTGTAATACTT
GCATGCTTCTTAGACCAGAACATGTAGGTCCCCTTGTGTCTCAATACTTTTTTTTTCTTAAT
TGCATTTGTTGGCTCTATTTTAATTTTTTTCTTTTAAAATAAACAGCTGGGACCATCCCAAA
AGACAAGCCATGCATACAACTTTGGTCATGTATCTCTGCAAAGCATCAAATTAAATGCACGC
TTTTGTCATGTCAGTGGTTTTTGTTTTGTGAAATTCCTTTGACCATATTAGATCTATTTCAT
TTCCAATAGTGAAAAGGAGATGTGGTGGTATACTTTGTTTGCCATTTGTTTAAAAGATACAA
CGGATACCTTCTATCATGTATGTACTGGCTTATAAATGAAAATCTATCTACAACATTACCCA
CAAAGGCAACATGACACCAATTATCACTGCCTCTGCCCTTAAAAATGTCAGAGTAGTATTAT
TGATAAAAAGGGCAAGCAATAGATTTTTCATGACTGAATAAACTGTAATAATAAAACATATG
TCTCAAAGTGTATCACATATGAATTTAGCCTAATTGTTTTCAGTTTCATTCTCAATATTTAG
TTTACAACATCATTTTCCCCTAAACTGGTTATATTTTGACCTGTATATCTTAAATTTGAGTA
TTTATATGCCTAAATACATGTGTGAGTTTTGTTTGACTTCCAAGTCCAAACTATAAGATTAT
ATAAGTTCATATAGATGAATCAGAAATATGTGGTAATACTATTAAGTCACAAACACTAACAA
TTTCCAACTATAGAAATAACAGTTCTTATTTGGATTTTGGGAATGCTACCAATAAAAGCCTG
CCCAGACCA

An exemplary COL5A2 polypeptide amino acid sequence is provided herein in SEQ ID NO: 5.

(SEQ ID NO: 5)
MMANWAEARPLLILIVLLGQFVSIKAQEEDEDEGYGEEIACTQNGQMYLNRDIWKPAPCQIC
VCDNGAILCDKIECQDVLDCADPVTPPGECCPVCSQTPGGGNTNFGRGRKGQKGEPGLVPVV
TGIRGRPGPAGPPGSQGPRGERGPKGRPGPRGPQGIDGEPGVPGQPGAPGPPGHPSHPGPDG
LSRPFSAQMAGLDEKSGLGSQVGLMPGSVGPVGPRGPQGLQGQQGGAGPTGPPGEPGDPGPM
GPIGSRGPEGPPGKPGEDGEPGRNGNPGEVGFAGSPGARGFPGAPGLPGLKGHRGHKGLEGP
KGEVGAPGSKGEAGPTGPMGAMGPLGPRGMPGERGRLGPQGAPGQRGAHGMPGKPGPMGPLG
IPGSSGFPGNPGMKGEAGPTGARGPEGPQGQRGETGPPGPVGSPGLPGAIGTDGTPGAKGPT
GSPGTSGPPGSAGPPGSPGPQGSTGPQGIRGQPGDPGVPGFKGEAGPKGEPGPHGIQGPIGP
PGEEGKRGPRGDPGTVGPPGPVGERGAPGNRGFPGSDGLPGPKGAQGERGPVGSSGPKGSQG
DPGRPGEPGLPGARGLTGNPGVQGPEGKLGPLGAPGEDGRPGPPGSIGIRGQPGSMGLPGPK
GSSGDPGKPGEAGNAGVPGQRGAPGKDGEVGPSGPVGPPGLAGERGEQGPPGPTGFQGLPGP
PGPPGEGGKPGDQGVPGDPGAVGPLGPRGERGNPGERGEPGITGLPGEKGMAGGHGPDGPKG
SPGPSGTPGDTGPPGLQGMPGERGIAGTPGPKGDRGGIGEKGAEGTAGNDGARGLPGPLGPP
GPAGPTGEKGEPGPRGLVGPPGSRGNPGSRGENGPTGAVGFAGPQGPDGQPGVKGEPGEPGQ
KGDAGSPGPQGLAGSPGPHGPNGVPGLKGGRGTQGPPGATGFPGSAGRVGPPGPAGAPGPAG
PLGEPGKEGPPGLRGDPGSHGRVGDRGPAGPPGGPGDKGDPGEDGQPGPDGPPGPAGTTGQR
GIVGMPGQRGERGMPGLPGPAGTPGKVGPTGATGDKGPPGPVGPPGSNGPVGEPGPEGPAGN
DGTPGRDGAVGERGDRGDPGPAGLPGSQGAPGTPGPVGAPGDAGQRGDPGSRGPIGPPGRAG
KRGLPGPQGPRGDKGDHGDRGDRGQKGHRGFTGLQGLPGPPGPNGEQGSAGIPGPFGPRGPP
GPVGPSGKEGNPGPLGPIGPPGVRGSVGEAGPEGPPGEPGPPGPPGPPGHLTAALGDIMGHY
DESMPDPLPEFTEDQAAPDDKNKTDPGVHATLKSLSSQIETMRSPDGSKKHPARTCDDLKLC
HSAKQSGEYWIDPNQGSVEDAIKVYCNMETGETCISANPSSVPRKTWWASKSPDNKPVWYGL
DMNRGSQFAYGDHQSPNTAITQMTFLRLLSKEASQNITYICKNSVGYMDDQAKNLKKAVVLK
GANDLDIKAEGNIRFRYIVLQDTCSKRNGNVGKTVFEYRTQNVARLPIIDLAPVDVGGTDQE
FGVEIGPVCFV

The COL3A1 gene encodes the collagen type III, alpha-1 chain protein. Type III collagen is a fibrillar collagen with structural roles in hollow organs and many other tissues. Mutations in the COL3A1 gene have been associated with Ehlers-Danlos syndrome. An exemplary COL3A1 nucleotide sequence is available as Transcript ID NM_000090 (e.g., available at the website www[dot]ncbi[dot]nlm[dot]nih[dot]gov/nuccore/NM_000090), and provided herein in SEQ ID NO: 3.

(SEQ ID NO: 3)
GGCTGAGTTTTATGACGGGCCCGGTGCTGAAGGGCAGGGAACAACTTGATGGTGCTACTTTG
AACTGCTTTTCTTTTCTCCTTTTTGCACAAAGAGTCTCATGTCTGATATTTAGACATGATGA
GCTTTGTGCAAAAGGGGAGCTGGCTACTTCTCGCTCTGCTTCATCCCACTATTATTTTGGCA
CAACAGGAAGCTGTTGAAGGAGGATGTTCCCATCTTGGTCAGTCCTATGCGGATAGAGATGT
CTGGAAGCCAGAACCATGCCAAATATGTGTCTGTGACTCAGGATCCGTTCTCTGCGATGACA
TAATATGTGACGATCAAGAATTAGACTGCCCCAACCCAGAAATTCCATTTGGAGAATGTTGT
GCAGTTTGCCCACAGCCTCCAACTGCTCCTACTCGCCCTCCTAATGGTCAAGGACCTCAAGG
CCCCAAGGGAGATCCAGGCCCTCCTGGTATTCCTGGGAGAAATGGTGACCCTGGTATTCCAG
GACAACCAGGGTCCCCTGGTTCTCCTGGCCCCCCTGGAATCTGTGAATCATGCCCTACTGGT
CCTCAGAACTATTCTCCCCAGTATGATTCATATGATGTCAAGTCTGGAGTAGCAGTAGGAGG
ACTCGCAGGCTATCCTGGACCAGCTGGCCCCCCAGGCCCTCCCGGTCCCCCTGGTACATCTG
GTCATCCTGGTTCCCCTGGATCTCCAGGATACCAAGGACCCCCTGGTGAACCTGGGCAAGCT
GGTCCTTCAGGCCCTCCAGGACCTCCTGGTGCTATAGGTCCATCTGGTCCTGCTGGAAAAGA
TGGAGAATCAGGTAGACCCGGACGACCTGGAGAGCGAGGATTGCCTGGACCTCCAGGTATCA
AAGGTCCAGCTGGGATACCTGGATTCCCTGGTATGAAAGGACACAGAGGCTTCGATGGACGA
AATGGAGAAAAGGGTGAAACAGGTGCTCCTGGATTAAAGGGTGAAAATGGTCTTCCAGGCGA
AAATGGAGCTCCTGGACCCATGGGTCCAAGAGGGGCTCCTGGTGAGCGAGGACGGCCAGGAC
TTCCTGGGGCTGCAGGTGCTCGGGGTAATGACGGTGCTCGAGGCAGTGATGGTCAACCAGGC
CCTCCTGGTCCTCCTGGAACTGCCGGATTCCCTGGATCCCCTGGTGCTAAGGGTGAAGTTGG
ACCTGCAGGGTCTCCTGGTTCAAATGGTGCCCCTGGACAAAGAGGAGAACCTGGACCTCAGG
GACACGCTGGTGCTCAAGGTCCTCCTGGCCCTCCTGGGATTAATGGTAGTCCTGGTGGTAAA
GGCGAAATGGGTCCCGCTGGCATTCCTGGAGCTCCTGGACTGATGGGAGCCCGGGGTCCTCC
AGGACCAGCCGGTGCTAATGGTGCTCCTGGACTGCGAGGTGGTGCAGGTGAGCCTGGTAAGA
ATGGTGCCAAAGGAGAGCCCGGACCACGTGGTGAACGCGGTGAGGCTGGTATTCCAGGTGTT
CCAGGAGCTAAAGGCGAAGATGGCAAGGATGGATCACCTGGAGAACCTGGTGCAAATGGGCT
TCCAGGAGCTGCAGGAGAAAGGGGTGCCCCTGGGTTCCGAGGACCTGCTGGACCAAATGGCA
TCCCAGGAGAAAAGGGTCCTGCTGGAGAGCGTGGTGCTCCAGGCCCTGCAGGGCCCAGAGGA
GCTGCTGGAGAACCTGGCAGAGATGGCGTCCCTGGAGGTCCAGGAATGAGGGGCATGCCCGG
AAGTCCAGGAGGACCAGGAAGTGATGGGAAACCAGGGCCTCCCGGAAGTCAAGGAGAAAGTG
GTCGACCAGGTCCTCCTGGGCCATCTGGTCCCCGAGGTCAGCCTGGTGTCATGGGCTTCCCC
GGTCCTAAAGGAAATGATGGTGCTCCTGGTAAGAATGGAGAACGAGGTGGCCCTGGAGGACC
TGGCCCTCAGGGTCCTCCTGGAAAGAATGGTGAAACTGGACCTCAGGGACCCCCAGGGCCTA
CTGGGCCTGGTGGTGACAAAGGAGACACAGGACCCCCTGGTCCACAAGGATTACAAGGCTTG
CCTGGTACAGGTGGTCCTCCAGGAGAAAATGGAAAACCTGGGGAACCAGGTCCAAAGGGTGA
TGCCGGTGCACCTGGAGCTCCAGGAGGCAAGGGTGATGCTGGTGCCCCTGGTGAACGTGGAC
CTCCTGGATTGGCAGGGGCCCCAGGACTTAGAGGTGGAGCTGGTCCCCCTGGTCCCGAAGGA
GGAAAGGGTGCTGCTGGTCCTCCTGGGCCACCTGGTGCTGCTGGTACTCCTGGTCTGCAAGG
AATGCCTGGAGAAAGAGGAGGTCTTGGAAGTCCTGGTCCAAAGGGTGACAAGGGTGAACCAG
GCGGTCCAGGTGCTGATGGTGTCCCAGGGAAAGATGGCCCAAGGGGTCCTACTGGTCCTATT
GGTCCTCCTGGCCCAGCTGGCCAGCCTGGAGATAAGGGTGAAGGTGGTGCCCCCGGACTTCC
AGGTATAGCTGGACCTCGTGGTAGCCCTGGTGAGAGAGGTGAAACTGGCCCTCCAGGACCTG
CTGGTTTCCCTGGTGCTCCTGGACAGAATGGTGAACCTGGTGGTAAAGGAGAAAGAGGGGCT
CCGGGTGAGAAAGGTGAAGGAGGCCCTCCTGGAGTTGCAGGACCCCCTGGAGGTTCTGGACC
TGCTGGTCCTCCTGGTCCCCAAGGTGTCAAAGGTGAACGTGGCAGTCCTGGTGGACCTGGTG
CTGCTGGCTTCCCTGGTGCTCGTGGTCTTCCTGGTCCTCCTGGTAGTAATGGTAACCCAGGA
CCCCCAGGTCCCAGCGGTTCTCCAGGCAAGGATGGGCCCCCAGGTCCTGCGGGTAACACTGG
TGCTCCTGGCAGCCCTGGAGTGTCTGGACCAAAAGGTGATGCTGGCCAACCAGGAGAGAAGG
GATCGCCTGGTGCCCAGGGCCCACCAGGAGCTCCAGGCCCACTTGGGATTGCTGGGATCACT
GGAGCACGGGGTCTTGCAGGACCACCAGGCATGCCAGGTCCTAGGGGAAGCCCTGGCCCTCA
GGGTGTCAAGGGTGAAAGTGGGAAACCAGGAGCTAACGGTCTCAGTGGAGAACGTGGTCCCC
CTGGACCCCAGGGTCTTCCTGGTCTGGCTGGTACAGCTGGTGAACCTGGAAGAGATGGAAAC
CCTGGATCAGATGGTCTTCCAGGCCGAGATGGATCTCCTGGTGGCAAGGGTGATCGTGGTGA
AAATGGCTCTCCTGGTGCCCCTGGCGCTCCTGGTCATCCAGGCCCACCTGGTCCTGTCGGTC
CAGCTGGAAAGAGTGGTGACAGAGGAGAAAGTGGCCCTGCTGGCCCTGCTGGTGCTCCCGGT
CCTGCTGGTTCCCGAGGTGCTCCTGGTCCTCAAGGCCCACGTGGTGACAAAGGTGAAACAGG
TGAACGTGGAGCTGCTGGCATCAAAGGACATCGAGGATTCCCTGGTAATCCAGGTGCCCCAG
GTTCTCCAGGCCCTGCTGGTCAGCAGGGTGCAATCGGCAGTCCAGGACCTGCAGGCCCCAGA
GGACCTGTTGGACCCAGTGGACCTCCTGGCAAAGATGGAACCAGTGGACATCCAGGTCCCAT
TGGACCACCAGGGCCTCGAGGTAACAGAGGTGAAAGAGGATCTGAGGGCTCCCCAGGCCACC
CAGGGCAACCAGGCCCTCCTGGACCTCCTGGTGCCCCTGGTCCTTGCTGTGGTGGTGTTGGA
GCCGCTGCCATTGCTGGGATTGGAGGTGAAAAAGCTGGCGGTTTTGCCCCGTATTATGGAGA
TGAACCAATGGATTTCAAAATCAACACCGATGAGATTATGACTTCACTCAAGTCTGTTAATG
GACAAATAGAAAGCCTCATTAGTCCTGATGGTTCTCGTAAAAACCCCGCTAGAAACTGCAGA
GACCTGAAATTCTGCCATCCTGAACTCAAGAGTGGAGAATACTGGGTTGACCCTAACCAAGG
ATGCAAATTGGATGCTATCAAGGTATTCTGTAATATGGAAACTGGGGAAACATGCATAAGTG
CCAATCCTTTGAATGTTCCACGGAAACACTGGTGGACAGATTCTAGTGCTGAGAAGAAACAC
GTTTGGTTTGGAGAGTCCATGGATGGTGGTTTTCAGTTTAGCTACGGCAATCCTGAACTTCC
TGAAGATGTCCTTGATGTGCATCTGGCATTCCTTCGACTTCTCTCCAGCCGAGCTTCCCAGA
ACATCACATATCACTGCAAAAATAGCATTGCATACATGGATCAGGCCAGTGGAAATGTAAAG
AAGGCCCTGAAGCTGATGGGGTCAAATGAAGGTGAATTCAAGGCTGAAGGAAATAGCAAATT
CACCTACACAGTTCTGGAGGATGGTTGCACGAAACACACTGGGGAATGGAGCAAAACAGTCT
TTGAATATCGAACACGCAAGGCTGTGAGACTACCTATTGTAGATATTGCACCCTATGACATT
GGTGGTCCTGATCAAGAATTTGGTGTGGACGTTGGCCCTGTTTGCTTTTTATAAACCAAACT
CTATCTGAAATCCCAACAAAAAAAATTTAACTCCATATGTGTTCCTCTTGTTCTAATCTTGT
CAACCAGTGCAAGTGACCGACAAAATTCCAGTTATTTATTTCCAAAATGTTTGGAAACAGTA
TAATTTGACAAAGAAAAATGATACTTCTCTTTTTTTGCTGTTCCACCAAATACAATTCAAAT
GCTTTTTGTTTTATTTTTTTACCAATTCCAATTTCAAAATGTCTCAATGGTGCTATAATAAA
TAAACTTCAACACTCTTTATGATAACAACACTGTGTTATATTCTTTGAATCCTAGCCCATCT
GCAGAGCAATGACTGTGCTCACCAGTAAAAGATAACCTTTCTTTCTGAAATAGTCAAATACG
AAATTAGAAAAGCCCTCCCTATTTTAACTACCTCAACTGGTCAGAAACACAGATTGTATTCT
ATGAGTCCCAGAAGATGAAAAAAATTTTATACGTTGATAAAACTTATAAATTTCATTGATTA
ATCTCCTGGAAGATTGGTTTAAAAAGAAAAGTGTAATGCAAGAATTTAAAGAAATATTTTTA
AAGCCACAATTATTTTAATATTGGATATCAACTGCTTGTAAAGGTGCTCCTCTTTTTTCTTG
TCATTGCTGGTCAAGATTACTAATATTTGGGAAGGCTTTAAAGACGCATGTTATGGTGCTAA
TGTACTTTCACTTTTAAACTCTAGATCAGAATTGTTGACTTGCATTCAGAACATAAATGCAC
AAAATCTGTACATGTCTCCCATCAGAAAGATTCATTGGCATGCCACAGGGGATTCTCCTCCT
TCATCCTGTAAAGGTCAACAATAAAAACCAAATTATGGGGCTGCTTTTGTCACACTAGCATA
GAGAATGTGTTGAAATTTAACTTTGTAAGCTTGTATGTGGTTGTTGATCTTTTTTTTCCTTA
CAGACACCCATAATAAAATATCATATTAAAATTC

An exemplary COL3A1 polypeptide amino acid sequence is provided herein in SEQ ID NO: 6.

(SEQ ID NO: 6)
MMSFVQKGSWLLLALLHPTIILAQQEAVEGGCSHLGQSYADRDVWKPEPCQICVCDSGSVLC
DDIICDDQELDCPNPEIPFGECCAVCPQPPTAPTRPPNGQGPQGPKGDPGPPGIPGRNGDPG
IPGQPGSPGSPGPPGICESCPTGPQNYSPQYDSYDVKSGVAVGGLAGYPGPAGPPGPPGPPG
TSGHPGSPGSPGYQGPPGEPGQAGPSGPPGPPGAIGPSGPAGKDGESGRPGRPGERGLPGPP
GIKGPAGIPGFPGMKGHRGFDGRNGEKGETGAPGLKGENGLPGENGAPGPMGPRGAPGERGR
PGLPGAAGARGNDGARGSDGQPGPPGPPGTAGFPGSPGAKGEVGPAGSPGSNGAPGQRGEPG
PQGHAGAQGPPGPPGINGSPGGKGEMGPAGIPGAPGLMGARGPPGPAGANGAPGLRGGAGEP
GKNGAKGEPGPRGERGEAGIPGVPGAKGEDGKDGSPGEPGANGLPGAAGERGAPGERGPAGP
NGIPGEKGPAGERGAPGPAGPRGAAGEPGRDGVPGGPGMRGMPGSPGGPGSDGKPGPPGSQG
ESGRPGPPGPSGPRGQPGVMGFPGPKGNDGAPGKNGERGGPGGPGPQGPPGKNGETGPQGPP
GPTGPGGDKGDTGPPGPQGLQGLPGTGGPPGENGKPGEPGPKGDAGAPGAPGGKGDAGAPGE
RGPPGLAGAPGLRGGAGPPGPEGGKGAAGPPGPPGAAGTPGLQGMPGERGGLGSPGPKGDKG
EPGGPGADGVPGKDGPRGPTGPIGPPGPAGQPGDKGEGGAPGLPGIAGPRGSPGERGETGPP
GPAGFPGAPGQNGEPGGKGERGAPGEKGEGGPPGVAGPPGGSGPAGPPGPQGVKGERGSPGG
PGAAGFPGARGLPGPPGSNGNPGPPGPSGSPGKDGPPGPAGNTGAPGSPGVSGPKGDAGQPG
EKGSPGAQGPPGAPGPLGIAGITGARGLAGPPGMPGPRGSPGPQGVKGESGKPGANGLSGER
GPPGPQGLPGLAGTAGEPGRDGNPGSDGLPGRDGSPGGKGDRGENGSPGAPGAPGHPGPPGP
VGPAGKSGDRGESGPAGPAGAPGPAGSRGAPGPQGPRGDKGETGERGAAGIKGHRGFPGNPG
APGSPGPAGQQGAIGSPGPAGPRGPVGPSGPPGKDGTSGHPGPIGPPGPRGNRGERGSEGSP
GHPGQPGPPGPPGAPGPCCGGVGAAAIAGIGGEKAGGFAPYYGDEPMDFKINTDEIMTSLKS
VNGQIESLISPDGSRKNPARNCRDLKFCHPELKSGEYWVDPNQGCKLDAIKVFCNMETGETC
ISANPLNVPRKHWWTDSSAEKKHVWFGESMDGGFQFSYGNPELPEDVLDVHLAFLRLLSSRA
SQNITYHCKNSIAYMDQASGNVKKALKLMGSNEGEFKAEGNSKFTYTVLEDGCTKHTGEWSK
TVFEYRTRKAVRLPIVDIAPYDIGGPDQEFGVDVGPVCFL

COL5A2-ALK Fusion Nucleic Acid Molecules

The present disclosure is based, at least in part, on the discovery of COL5A2-ALK gene fusions in cancers, such as in rhabdomyosarcoma. Thus, in some aspects, provided herein are COL5A2-ALK fusion nucleic acid molecules.

In some embodiments of the COL5A2-ALK fusion nucleic acid molecules provided herein, an intron or an exon of COL5A2, or a portion or fragment thereof, is directly fused to an intron or an exon of ALK, or a portion or fragment thereof, thereby establishing a COL5A2-ALK breakpoint between the COL5A2 sequence and the ALK sequence.

In some embodiments, a COL5A2-ALK fusion nucleic acid molecule provided herein comprises at least one exon of COL5A2 or a portion thereof and at least one exon of ALK or a portion thereof.

In some embodiments, a COL5A2-ALK fusion nucleic acid molecule provided herein comprises an exon or a portion thereof of COL5A2 and an intron or a portion thereof of ALK, and a COL5A2-ALK breakpoint that fuses the exon or a portion thereof of COL5A2 to the intron or a portion thereof of ALK In some embodiments, a COL5A2-ALK fusion nucleic acid molecule provided herein comprises an intron or a portion thereof of COL5A2 and an intron or a portion thereof of ALK, and a COL5A2-ALK breakpoint that fuses the intron or a portion thereof of COL5A2 to the intron or a portion thereof of ALK In some embodiments, a COL5A2-ALK fusion nucleic acid molecule provided herein comprises an exon or a portion thereof of COL5A2 and an exon or a portion thereof of ALK, and a COL5A2-ALK breakpoint that fuses the exon or a portion thereof of COL5A2 to the exon or a portion thereof of ALK In some embodiments, a COL5A2-ALK fusion nucleic acid molecule provided herein comprises an intron or a portion thereof of COL5A2 and an exon or a portion thereof of ALK, and a COL5A2-ALK breakpoint that fuses the intron or a portion thereof of COL5A2 to the exon or a portion thereof of ALK.

In some embodiments, the COL5A2 breakpoint occurs within an intron or within an exon of COL5A2, e.g., within exon 1 or intron 1 of COL5A2. In some embodiments, the COL5A2 breakpoint occurs at the 3′ end or at the 5′ end of an intron, or at the 3′ end or at the 5′ end of an exon of COL5A2, e.g., at the 3′ end of exon 1, at the 5′ end of intron 1, or at the 3′ end of intron 1 of COL5A2. In some embodiments, the ALK breakpoint occurs within an intron or within an exon of ALK, e.g., within intron 5 or exon 6 of ALK. In some embodiments, the ALK breakpoint occurs at the 3′ end or at the 5′ end of an intron, or at the 3′ end or at the 5′ end of an exon of ALK, e.g., at the 5′ end of intron 5, at the 3′ end of intron 5, or at the 5′ end of exon 6 of ALK.

In certain embodiments, exon 1 or intron 1, or a portion of exon 1 or intron 1, of COL5A2 is directly fused to intron 5 or exon 6, or a portion of intron 5 or exon 6, of ALK, thereby establishing a COL5A2-ALK breakpoint between the COL5A2 sequence and the ALK sequence.

In some embodiments, a COL5A2-ALK fusion nucleic acid molecule provided herein comprises exon 1 or a portion thereof of COL5A2 fused to intron 5 or a portion thereof of ALK. In some embodiments, a COL5A2-ALK fusion nucleic acid molecule provided herein comprises intron 1 or a portion thereof of COL5A2 fused to intron 5 or a portion thereof of ALK. In some embodiments, a COL5A2-ALK fusion nucleic acid molecule provided herein comprises exon 1 or a portion thereof of COL5A2 fused to exon 6 or a portion thereof of ALK. In some embodiments, a COL5A2-ALK fusion nucleic acid molecule provided herein comprises intron 1 or a portion thereof of COL5A2 fused to exon 6 or a portion thereof of ALK.

In some embodiments, a COL5A2-ALK fusion nucleic acid molecule provided herein comprises a COL5A2-ALK breakpoint that fuses exon 1 or a portion thereof of COL5A2 to intron 5 or a portion thereof of ALK. In some embodiments, the COL5A2-ALK breakpoint fuses the 3′ end of exon 1 or the portion thereof of COL5A2 to the 5′ end of intron 5 or the portion thereof of ALK. In some embodiments, a COL5A2-ALK fusion nucleic acid molecule provided herein comprises a COL5A2-ALK breakpoint that fuses intron 1 or a portion thereof of COL5A2 to intron 5 or a portion thereof of ALK. In some embodiments, the COL5A2-ALK breakpoint fuses the 3′ end of intron 1 or a portion thereof of COL5A2 to the 5′ end of intron 5 or a portion thereof of ALK. In some embodiments, a COL5A2-ALK fusion nucleic acid molecule provided herein comprises a COL5A2-ALK breakpoint that fuses exon 1 or a portion thereof of COL5A2 to exon 6 or a portion thereof of ALK. In some embodiments, the COL5A2-ALK breakpoint fuses the 3′ end of exon 1 or a portion thereof of COL5A2 to the 5′ end of exon 6 or a portion thereof of ALK. In some embodiments, a COL5A2-ALK fusion nucleic acid molecule provided herein comprises a COL5A2-ALK breakpoint that fuses intron 1 or a portion thereof of COL5A2 to exon 6 or a portion thereof of ALK. In some embodiments, the COL5A2-ALK breakpoint fuses the 3′ end of intron 1 or a portion thereof of COL5A2 to the 5′ end of exon 6 or a portion thereof of ALK.

In some embodiments, a COL5A2-ALK fusion nucleic acid molecule provided herein comprises exon 1 or a portion thereof of COL5A2 fused to an intron or a portion thereof between exon 5 and exon 6 of ALK. In some embodiments, a COL5A2-ALK fusion nucleic acid molecule provided herein comprises an intron or a portion thereof between exon 1 and exon 2 of COL5A2 fused to an intron or a portion thereof between exon 5 and exon 6 of ALK. In some embodiments, a COL5A2-ALK fusion nucleic acid molecule provided herein comprises an intron or a portion thereof between exon 1 and exon 2 of COL5A2 fused to exon 6 or a portion thereof of ALK.

In some embodiments, a COL5A2-ALK fusion nucleic acid molecule provided herein comprises exon 1 or a portion thereof of COL5A2 and intron 5 or a portion thereof of ALK, and a COL5A2-ALK breakpoint that fuses exon 1 or the portion thereof of COL5A2 and intron 5 or the portion thereof of ALK. In some embodiments, the 3′ end of exon 1 or of a portion of exon 1 of COL5A2 is fused to the 5′ end of intron 5 or of a portion of intron 5 of ALK.

In some embodiments, a COL5A2-ALK fusion nucleic acid molecule provided herein comprises intron 1 or a portion thereof of COL5A2 and intron 5 or a portion thereof of ALK, and a COL5A2-ALK breakpoint that fuses intron 1 or the portion thereof of COL5A2 and intron 5 or the portion thereof of ALK. In some embodiments, the 3′ end of intron 1 or of a portion of intron 1 of COL5A2 is fused to the 5′ end of intron 5 or of a portion of intron 5 of ALK.

In some embodiments, a COL5A2-ALK fusion nucleic acid molecule provided herein comprises exon 1 or a portion thereof of COL5A2 and exon 6 or a portion thereof of ALK, and a COL5A2-ALK breakpoint that fuses exon 1 or the portion thereof of COL5A2 and exon 6 or the portion thereof of ALK. In some embodiments, the 3′ end of exon 1 or of a portion of exon 1 of COL5A2 is fused to the 5′ end of exon 6 or of a portion of exon 6 of ALK.

In some embodiments, a COL5A2-ALK fusion nucleic acid molecule provided herein comprises intron 1 or a portion thereof of COL5A2 and exon 6 or a portion thereof of ALK, and a COL5A2-ALK breakpoint that fuses intron 1 or the portion thereof of COL5A2 and exon 6 or the portion thereof of ALK. In some embodiments, the 3′ end of intron 1 or of a portion of intron 1 of COL5A2 is fused to the 5′ end of exon 6 or of a portion of exon 6 of ALK.

In some embodiments, the exon-exon fusions or exon-intron fusions are in-frame fusions. The fusion breakpoint may occur anywhere within an exon or an intron of COL5A2 (e.g., exon 1 or intron 1), and anywhere within an exon or an intron of ALK (e.g., intron 5 or exon 6). When a breakpoint occurs in an intron of COL5A2, the resulting mRNA sequence, and the resulting amino acid sequence, has a breakpoint or fusion junction between the preceding exon of COL5A2 and the sequence of ALK. When a breakpoint occurs in an intron of ALK, the resulting mRNA sequence, and the resulting amino acid sequence, has a breakpoint or fusion junction between the following exon of ALK and the sequence of COL5A2. When the breakpoint or fusion junction occurs between an intron of COL5A2 and an intron of ALK, the resulting mRNA sequence, and the resulting amino acid sequence, has a breakpoint or fusion junction between the preceding exon of COL5A2 and the following exon of ALK. For example, a fusion of intron 1 of COL5A2 and intron 5 of ALK in the DNA sequence would result in an mRNA sequence and in an amino acid sequence having a breakpoint or fusion junction between exon 1 of COL5A2 and exon 6 of ALK. One skilled in the art could readily determine the exon and intron sequences within the COL5A2 and ALK genes, and the corresponding mRNA and amino acid sequences, for example using an NCBI database (e.g., GenBank).

In some embodiments, the COL5A2-ALK fusion nucleic acid molecule comprises 5 or more, 10 or more, or 20 or more nucleotides on the 5′ end of the COL5A2-ALK breakpoint, and 5 or more, 10 or more, or 20 or more nucleotides on the 3′ end of the COL5A2-ALK breakpoint. In some embodiments, the COL5A2-ALK fusion nucleic acid molecule comprises 5 or more nucleotides from exon 1 or intron 1 of COL5A2 on the 5′ end of the COL5A2-ALK breakpoint, and 5 or more nucleotides from intron 5 or exon 6 of ALK on the 3′ end of the COL5A2-ALK breakpoint.

In some embodiments, a COL5A2-ALK fusion nucleic acid molecule provided herein, e.g., a DNA molecule, results in an mRNA molecule comprising a fusion, e.g., an in-frame fusion, of exon 1 or a portion thereof of COL5A2 fused to exon 6 or a portion thereof of ALK.

In some embodiments, a COL5A2-ALK fusion nucleic acid molecule provided herein comprises a COL5A2-ALK breakpoint resulting in an in-frame fusion of an exon described herein or a portion thereof of COL5A2 with an exon described herein or a portion thereof of ALK, e.g., resulting in an RNA molecule, such as an mRNA molecule, comprising an in-frame fusion of an exon described herein or a portion thereof of COL5A2 to an exon described herein or a portion thereof of ALK. In some embodiments, a COL5A2-ALK fusion nucleic acid molecule provided herein is an mRNA molecule comprising a fusion, e.g., an in-frame fusion, of exon 1 or a portion thereof of COL5A2 fused to exon 6 or a portion thereof of ALK. In some embodiments, a COL5A2-ALK fusion nucleic acid molecule provided herein is cDNA molecule comprising a fusion, e.g., an in-frame fusion, of exon 1 or a portion thereof of COL5A2 fused to exon 6 or a portion thereof of ALK.

In some embodiments, the COL5A2-ALK fusion nucleic acid molecule comprises a nucleotide sequence comprising, in the 5′ to 3′ direction, a fusion of exon 1 or a portion thereof of COL5A2 to exon 6 or a portion thereof of ALK. In some embodiments, the COL5A2-ALK fusion nucleic acid molecule comprises a nucleotide sequence comprising, in the 5′ to 3′ direction, exon 1 or a portion thereof of COL5A2, and exon 6 or a portion thereof and exons 7-29 of ALK. In some embodiments, the COL5A2-ALK fusion nucleic acid molecule results from a breakpoint in exon 1 or in intron 1 of COL5A2, and in intron 5 or in exon 6 of ALK.

In some embodiments, a COL5A2-ALK fusion nucleic acid molecule comprises at least a portion of a COL5A2 sequence of SEQ ID NO: 2 and at least a portion of an ALK sequence of SEQ ID NO: 1, or a sequence having at least about 85% (e.g., any of at least about 85%, at least about 90%, at least about 95%, at least about 96%, at least about 97%, at least about 98%, at least about 99%, or 100%) sequence identity to the portion of the COL5A2 sequence of SEQ ID NO: 2 and/or the portion of the ALK sequence of SEQ ID NO: 1.

In some embodiments, the COL5A2-ALK fusion nucleic acid molecule comprises the nucleotide sequence of SEQ ID NO: 7, or a nucleotide sequence at least 70%, 75%, 80%, 85%, 90%, 91%, 92%, 93%, 94%, 95%, 96%, 97%, 98%, 99%, or 99.5% identical to the nucleotide sequence of SEQ ID NO: 7.

(SEQ ID NO: 7)
GCAGACTGTGCTGGAGCTGGTGCTGAAAAAGGGGGTTTGCAGAGGCTGC
CCTGGGGCTGGTGCTGAAAGAAGAGCCCACAGCTGACTTCATGGTGCTA
CAATAACCTCAGAATCTACTTTTCACTCTCAGGAGAACCCACATGTCTA
ATATTTAGACATGATGGCAAACTGGGCGGAAGCAAGACCTCTCCTCATT
CTTATTGTTTTATTAGGGCAATTTGTCTCAATAAAAGCCCAGGAAGAAG
ACGAGGATGGAACATCCCCAGGCTCCAAGATGGCCCTGCAGAGCTCCTT
CACTTGTTGGAATGGGACAGTCCTCCAGCTTGGGCAGGCCTGTGACTTC
CACCAGGACTGTGCCCAGGGAGAAGATGAGAGCCAGATGTGCCGGAAAC
TGCCTGTGGGTTTTTACTGCAACTTTGAAGATGGCTTCTGTGGCTGGAC
CCAAGGCACACTGTCACCCCACACTCCTCAATGGCAGGTCAGGACCCTA
AAGGATGCCCGGTTCCAGGACCACCAAGACCATGCTCTATTGCTCAGTA
CCACTGATGTCCCCGCTTCTGAAAGTGCTACAGTGACCAGTGCTACGTT
TCCTGCACCGATCAAGAGCTCTCCATGTGAGCTCCGAATGTCCTGGCTC
ATTCGTGGAGTCTTGAGGGGAAACGTGTCCTTGGTGCTAGTGGAGAACA
AAACCGGGAAGGAGCAAGGCAGGATGGTCTGGCATGTCGCCGCCTATGA
AGGCTTGAGCCTGTGGCAGTGGATGGTGTTGCCTCTCCTCGATGTGTCT
GACAGGTTCTGGCTGCAGATGGTCGCATGGTGGGGACAAGGATCCAGAG
CCATCGTGGCTTTTGACAATATCTCCATCAGCCTGGACTGCTACCTCAC
CATTAGCGGAGAGGACAAGATCCTGCAGAATACAGCACCCAAATCAAGA
AACCTGTTTGAGAGAAACCCAAACAAGGAGCTGAAACCCGGGGAAAATT
CACCAAGACAGACCCCCATCTTTGACCCTACAGTTCATTGGCTGTTCAC
CACATGTGGGGCCAGCGGGCCCCATGGCCCCACCCAGGCACAGTGCAAC
AACGCCTACCAGAACTCCAACCTGAGCGTGGAGGTGGGGAGCGAGGGCC
CCCTGAAAGGCATCCAGATCTGGAAGGTGCCAGCCACCGACACCTACAG
CATCTCGGGCTACGGAGCTGCTGGCGGGAAAGGCGGGAAGAACACCATG
ATGCGGTCCCACGGCGTGTCTGTGCTGGGCATCTTCAACCTGGAGAAGG
ATGACATGCTGTACATCCTGGTTGGGCAGCAGGGAGAGGACGCCTGCCC
CAGTACAAACCAGTTAATCCAGAAAGTCTGCATTGGAGAGAACAATGTG
ATAGAAGAAGAAATCCGTGTGAACAGAAGCGTGCATGAGTGGGCAGGAG
GCGGAGGAGGAGGGGGTGGAGCCACCTACGTATTTAAGATGAAGGATGG
AGTGCCGGTGCCCCTGATCATTGCAGCCGGAGGTGGTGGCAGGGCCTAC
GGGGCCAAGACAGACACGTTCCACCCAGAGAGACTGGAGAATAACTCCT
CGGTTCTAGGGCTAAACGGCAATTCCGGAGCCGCAGGTGGTGGAGGTGG
CTGGAATGATAACACTTCCTTGCTCTGGGCCGGAAAATCTTTGCAGGAG
GGTGCCACCGGAGGACATTCCTGCCCCCAGGCCATGAAGAAGTGGGGGT
GGGAGACAAGAGGGGGTTTCGGAGGGGGTGGAGGGGGGTGCTCCTCAGG
TGGAGGAGGCGGAGGATATATAGGCGGCAATGCAGCCTCAAACAATGAC
CCCGAAATGGATGGGGAAGATGGGGTTTCCTTCATCAGTCCACTGGGCA
TCCTGTACACCCCAGCTTTAAAAGTGATGGAAGGCCACGGGGAAGTGAA
TATTAAGCATTATCTAAACTGCAGTCACTGTGAGGTAGACGAATGTCAC
ATGGACCCTGAAAGCCACAAGGTCATCTGCTTCTGTGACCACGGGACGG
TGCTGGCTGAGGATGGCGTCTCCTGCATTGTGTCACCCACCCCGGAGCC
ACACCTGCCACTCTCGCTGATCCTCTCTGTGGTGACCTCTGCCCTCGTG
GCCGCCCTGGTCCTGGCTTTCTCCGGCATCATGATTGTGTACCGCCGGA
AGCACCAGGAGCTGCAAGCCATGCAGATGGAGCTGCAGAGCCCTGAGTA
CAAGCTGAGCAAGCTCCGCACCTCGACCATCATGACCGACTACAACCCC
AACTACTGCTTTGCTGGCAAGACCTCCTCCATCAGTGACCTGAAGGAGG
TGCCGCGGAAAAACATCACCCTCATTCGGGGTCTGGGCCATGGCGCCTT
TGGGGAGGTGTATGAAGGCCAGGTGTCCGGAATGCCCAACGACCCAAGC
CCCCTGCAAGTGGCTGTGAAGACGCTGCCTGAAGTGTGCTCTGAACAGG
ACGAACTGGATTTCCTCATGGAAGCCCTGATCATCAGCAAATTCAACCA
CCAGAACATTGTTCGCTGCATTGGGGTGAGCCTGCAATCCCTGCCCCGG
TTCATCCTGCTGGAGCTCATGGCGGGGGGAGACCTCAAGTCCTTCCTCC
GAGAGACCCGCCCTCGCCCGAGCCAGCCCTCCTCCCTGGCCATGCTGGA
CCTTCTGCACGTGGCTCGGGACATTGCCTGTGGCTGTCAGTATTTGGAG
GAAAACCACTTCATCCACCGAGACATTGCTGCCAGAAACTGCCTCTTGA
CCTGTCCAGGCCCTGGAAGAGTGGCCAAGATTGGAGACTTCGGGATGGC
CCGAGACATCTACAGGGCGAGCTACTATAGAAAGGGAGGCTGTGCCATG
CTGCCAGTTAAGTGGATGCCCCCAGAGGCCTTCATGGAAGGAATATTCA
CTTCTAAAACAGACACATGGTCCTTTGGAGTGCTGCTATGGGAAATCTT
TTCTCTTGGATATATGCCATACCCCAGCAAAAGCAACCAGGAAGTTCTG
GAGTTTGTCACCAGTGGAGGCCGGATGGACCCACCCAAGAACTGCCCTG
GGCCTGTATACCGGATAATGACTCAGTGCTGGCAACATCAGCCTGAAGA
CAGGCCCAACTTTGCCATCATTTTGGAGAGGATTGAATACTGCACCCAG
GACCCGGATGTAATCAACACCGCTTTGCCGATAGAATATGGTCCACTTG
TGGAAGAGGAAGAGAAAGTGCCTGTGAGGCCCAAGGACCCTGAGGGGGT
TCCTCCTCTCCTGGTCTCTCAACAGGCAAAACGGGAGGAGGAGCGCAGC
CCAGCTGCCCCACCACCTCTGCCTACCACCTCCTCTGGCAAGGCTGCAA
AGAAACCCACAGCTGCAGAGATCTCTGTTCGAGTCCCTAGAGGGCCGGC
CGTGGAAGGGGGACACGTGAATATGGCATTCTCTCAGTCCAACCCTCCT
TCGGAGTTGCACAAGGTCCACGGATCCAGAAACAAGCCCACCAGCTTGT
GGAACCCAACGTACGGCTCCTGGTTTACAGAGAAACCCACCAAAAAGAA
TAATCCTATAGCAAAGAAGGAGCCACACGACAGGGGTAACCTGGGGCTG
GAGGGAAGCTGTACTGTCCCACCTAACGTTGCAACTGGGAGACTTCCGG
GGGCCTCACTGCTCCTAGAGCCCTCTTCGCTGACTGCCAATATGAAGGA
GGTACCTCTGTTCAGGCTACGTCACTTCCCTTGTGGGAATGTCAATTAC
GGCTACCAGCAACAGGGCTTGCCCTTAGAAGCCGCTACTGCCCCTGGAG
CTGGTCATTACGAGGATACCATTCTGAAAAGCAAGAATAGCATGAACCA
GCCTGGGCCCTGAGCTCGGTCGCACACTCACTTCTCTTCCTTGGGATCC
CTAAGACCGTGGAGGAGAGAGAGGCAATGGCTCCTTCACAAACCAGAGA
CCAAATGTCACGTTTTGTTTTGTGCCAACCTATTTTGAAGTACCACCAA
AAAAGCTGTATTTTGAAAATGCTTTAGAAAGGTTTTGAGCATGGGTTCA
TCCTATTCTTTCGAAAGAAGAAAATATCATAAAAATGAGTGATAAATAC
AAGGCCCAGATGTGGTTGCATAAGGTTTTTATGCATGTTTGTTGTATAC
TTCCTTATGCTTCTTTCAAATTGTGTGTGCTCTGCTTCAATGTAGTCAG
AATTAGCTGCTTCTATGTTTCATAGTTGGGGTCATAGATGTTTCCTTGC
CTTGTTGATGTGGACATGAGCCATTTGAGGGGAGAGGGAACGGAAATAA
AGGAGTTATTTGTAATGACTAA

In the sequence of SEQ ID NO: 7 provided above, nucleotide sequences corresponding to ALK are underlined, and bolded sequences correspond to a novel codon formed by the fusion (wherein the first base corresponds to COL5A2 and the second and third bases are from ALK).

In some embodiments, the COL5A2-ALK fusion nucleic acid molecule is an isolated nucleic acid molecule. The isolated nucleic acid molecule may be free of sequences (such as protein-encoding sequences) that naturally flank the nucleic acid (i.e., sequences located at the 5′ and 3′ ends of the nucleic acid) in the genomic DNA of the organism from which the nucleic acid is derived. For example, in various embodiments, the fusion nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived.

COL3A1-ALK Fusion Nucleic Acid Molecules

The present disclosure is based, at least in part, on the discovery of COL3A1-ALK gene fusions in cancers, such as in leiomyosarcoma, inflammatory myofibroblastic tumor (IMT), and sarcoma not otherwise specified (NOS). Thus, in some aspects, provided herein are COL3A1-ALK fusion nucleic acid molecules.

The present disclosure is based, at least in part, on the discovery of COL3A1-ALK gene fusions in sarcomas, such as in uterus leiomyosarcoma, soft tissue inflammatory myofibroblastic tumor, or soft tissue sarcoma not otherwise specified (NOS), as demonstrated in Example 1. Thus, in some aspects, provided herein are COL3A1-ALK fusion nucleic acid molecules. The COL3A1-ALK fusion nucleic acid molecules identified in Example 1 are provided in Table 1 below.

TABLE 1
COL3A1-ALK fusion nucleic acid molecules detected in sarcomas.

| | | | 5′ | | 3′ |
|---|---|---|---|---|---|
| | | | breakpoint/ | | breakpoint/ |
| | Detected | | Exons | | Exons |
| Cancer | from | 5′ gene | included | 3′ gene | included |
| uterus leiomyosarcoma | DNA/RNA | COL3A1 | ex1-48 | ALK | ex19-29 |
| soft tissue inflammatory | DNA/RNA | COL3A1 | ex1-2 | ALK | ex19-29 |
| myofibroblastic tumor | | | | | |
| soft tissue sarcoma (nos) | DNA/RNA | COL3A1 | ex1-48 | ALK | ex19-29 |

In some embodiments of the COL3A1-ALK fusion nucleic acid molecules provided herein, an intron or an exon of COL3A1, or a portion or fragment thereof, is directly fused to an intron or an exon of ALK, or a portion or fragment thereof, thereby establishing a COL3A1-ALK breakpoint between the COL3A1 sequence and the ALK sequence.

In some embodiments, a COL3A1-ALK fusion nucleic acid molecule provided herein comprises at least one exon of COL3A1 or a portion thereof and at least one exon of ALK or a portion thereof.

In some embodiments, a COL3A1-ALK fusion nucleic acid molecule provided herein comprises an exon or a portion thereof of COL3A1 and an intron or a portion thereof of ALK, and a COL3A1-ALK breakpoint that fuses the exon or a portion thereof of COL3A1 to the intron or a portion thereof of ALK. In some embodiments, a COL3A1-ALK fusion nucleic acid molecule provided herein comprises an intron or a portion thereof of COL3A1 and an intron or a portion thereof of ALK, and a COL3A1-ALK breakpoint that fuses the intron or a portion thereof of COL3A1 to the intron or a portion thereof of ALK. In some embodiments, a COL3A1-ALK fusion nucleic acid molecule provided herein comprises an exon or a portion thereof of COL3A1 and an exon or a portion thereof of ALK, and a COL3A1-ALK breakpoint that fuses the exon or a portion thereof of COL3A1 to the exon or a portion thereof of ALK. In some embodiments, a COL3A1-ALK fusion nucleic acid molecule provided herein comprises an intron or a portion thereof of COL3A1 and an exon or a portion thereof of ALK, and a COL3A1-ALK breakpoint that fuses the intron or a portion thereof of COL3A1 to the exon or a portion thereof of ALK.

In some embodiments, the COL3A1 breakpoint occurs within an intron or within an exon of COL3A1, e.g., within exon 48, intron 48, exon 2, or intron 2 of COL3A1. In some embodiments, the COL3A1 breakpoint occurs at the 3′ end or at the 5′ end of an intron, or at the 3′ end or at the 5′ end of an exon, of COL3A1, e.g., at the 3′ end of exon 48, at the 5′ end of intron 48, at the 3′ end of intron 48, at the 3′ end of exon 2, at the 5′ end of intron 2, or at the 3′ end of intron 2 of COL3A1. In some embodiments, the ALK breakpoint occurs within an intron or within an exon of ALK, e.g., within intron 18 or exon 19 of ALK. In some embodiments, the ALK breakpoint occurs at the 3′ end or at the 5′ end of an intron, or at the 3′ end or at the 5′ end of an exon, of ALK, e.g., at the 5′ end of intron 18, at the 3′ end of intron 18, or at the 5′ end of exon 19 of ALK.

In certain embodiments, exon 48 or intron 48, or a portion of exon 48 or intron 48, of COL3A1 is directly fused to intron 18 or exon 19, or a portion of intron 18 or exon 19, of ALK, thereby establishing a COL3A1-ALK breakpoint between the COL3A1 sequence and the ALK sequence. In certain embodiments, exon 2 or intron 2, or a portion of exon 2 or intron 2, of COL3A1 is directly fused to intron 18 or exon 19, or a portion of intron 18 or exon 19, of ALK, thereby establishing a COL3A1-ALK breakpoint between the COL3A1 sequence and the ALK sequence.

In some embodiments, a COL3A1-ALK fusion nucleic acid molecule provided herein comprises exon 48 or a portion thereof of COL3A1 fused to intron 18 or a portion thereof of ALK. In some embodiments, a COL3A1-ALK fusion nucleic acid molecule provided herein comprises exon 48 or a portion thereof of COL3A1 fused to exon 19 or a portion thereof of ALK. In some embodiments, a COL3A1-ALK fusion nucleic acid molecule provided herein comprises intron 48 or a portion thereof of COL3A1 fused to intron 18 or a portion thereof of ALK. In some embodiments, a COL3A1-ALK fusion nucleic acid molecule provided herein comprises intron 48 or a portion thereof of COL3A1 fused to exon 19 or a portion thereof of ALK. In some embodiments, a COL3A1-ALK fusion nucleic acid molecule provided herein comprises exon 2 or a portion thereof of COL3A1 fused to intron 18 or a portion thereof of ALK. In some embodiments, a COL3A1-ALK fusion nucleic acid molecule provided herein comprises exon 2 or a portion thereof of COL3A1 fused to exon 19 or a portion thereof of ALK. In some embodiments, a COL3A1-ALK fusion nucleic acid molecule provided herein comprises intron 2 or a portion thereof of COL3A1 fused to intron 18 or a portion thereof of ALK. In some embodiments, a COL3A1-ALK fusion nucleic acid molecule provided herein comprises intron 2 or a portion thereof of COL3A1 fused to exon 19 or a portion thereof of ALK.

In some embodiments, a COL3A1-ALK fusion nucleic acid molecule provided herein comprises exon 48 or a portion thereof of COL3A1 fused to exon 19 or a portion thereof of ALK. In some embodiments, a COL3A1-ALK fusion nucleic acid molecule provided herein comprises exons 1-48 or a portion thereof of COL3A1 fused to exons 19-29 or a portion thereof of ALK. In some embodiments, the cancer is uterus leiomyosarcoma.

In some embodiments, a COL3A1-ALK fusion nucleic acid molecule provided herein comprises exon 2 or a portion thereof of COL3A1 fused to exon 19 or a portion thereof of ALK. In some embodiments, a COL3A1-ALK fusion nucleic acid molecule provided herein comprises exons 1 and 2 or a portion thereof of COL3A1 fused to exons 19-29 or a portion thereof of ALK. In some embodiments, the cancer is soft tissue inflammatory myofibroblastic tumor.

In some embodiments, a COL3A1-ALK fusion nucleic acid molecule provided herein comprises exon 48 or a portion thereof of COL3A1 fused to exon 19 or a portion thereof of ALK. In some embodiments, a COL3A1-ALK fusion nucleic acid molecule provided herein comprises exons 1-48 or a portion thereof of COL3A1 fused to exons 19-29 or a portion thereof of ALK. In some embodiments, the cancer is soft tissue sarcoma (nos).

In some embodiments, a COL3A1-ALK fusion nucleic acid molecule provided herein comprises a COL3A1-ALK breakpoint that fuses exon 48 or a portion thereof of COL3A1 to intron 18 or a portion thereof of ALK. In some embodiments, the COL3A1-ALK breakpoint fuses the 3′ end of exon 48 or of the portion thereof of COL3A1 to the 5′ end of intron 18 or of the portion thereof of ALK. In some embodiments, a COL3A1-ALK fusion nucleic acid molecule provided herein comprises a COL3A1-ALK breakpoint that fuses exon 48 or a portion thereof of COL3A1 to exon 19 or a portion thereof of ALK. In some embodiments, the COL3A1-ALK breakpoint fuses the 3′ end of exon 48 or of the portion thereof of COL3A1 to the 5′ end of exon 19 or of the portion thereof of ALK. In some embodiments, a COL3A1-ALK fusion nucleic acid molecule provided herein comprises a COL3A1-ALK breakpoint that fuses intron 48 or a portion thereof of COL3A1 to intron 18 or a portion thereof of ALK. In some embodiments, the COL3A1-ALK breakpoint fuses the 3′ end of intron 48 or of the portion thereof of COL3A1 to the 5′ end of intron 18 or of the portion thereof of ALK. In some embodiments, a COL3A1-ALK fusion nucleic acid molecule provided herein comprises a COL3A1-ALK breakpoint that fuses intron 48 or a portion thereof of COL3A1 to exon 19 or a portion thereof of ALK. In some embodiments, the COL3A1-ALK breakpoint fuses the 3′ end of intron 48 or of the portion thereof of COL3A1 to the 5′ end of exon 19 or of the portion thereof of ALK. In some embodiments, a COL3A1-ALK fusion nucleic acid molecule provided herein comprises a COL3A1-ALK breakpoint that fuses exon 2 or a portion thereof of COL3A1 to intron 18 or a portion thereof of ALK. In some embodiments, the COL3A1-ALK breakpoint fuses the 3′ end of exon 2 or of the portion thereof of COL3A1 to the 5′ end of intron 18 or of the portion thereof of ALK. In some embodiments, a COL3A1-ALK fusion nucleic acid molecule provided herein comprises a COL3A1-ALK breakpoint that fuses exon 2 or a portion thereof of COL3A1 to exon 19 or a portion thereof of ALK. In some embodiments, the COL3A1-ALK breakpoint fuses the 3′ end of exon 2 or of the portion thereof of COL3A1 to the 5′ end of exon 19 or of the portion thereof of ALK. In some embodiments, a COL3A1-ALK fusion nucleic acid molecule provided herein comprises a COL3A1-ALK breakpoint that fuses intron 2 or a portion thereof of COL3A1 to intron 18 or a portion thereof of ALK. In some embodiments, the COL3A1-ALK breakpoint fuses the 3′ end of intron 2 or of the portion thereof of COL3A1 to the 5′ end of intron 18 or of the portion thereof of ALK. In some embodiments, a COL3A1-ALK fusion nucleic acid molecule provided herein comprises a COL3A1-ALK breakpoint that fuses intron 2 or a portion thereof of COL3A1 to exon 19 or a portion thereof of ALK. In some embodiments, the COL3A1-ALK breakpoint fuses the 3′ end of intron 2 or of the portion thereof of COL3A1 to the 5′ end of exon 19 or of the portion thereof of ALK.

In some embodiments, a COL3A1-ALK fusion nucleic acid molecule provided herein comprises exon 48 or a portion thereof of COL3A1 fused to an intron or a portion thereof between exon 18 and exon 19 of ALK. In some embodiments, a COL3A1-ALK fusion nucleic acid molecule provided herein comprises an intron or a portion thereof between exon 48 and exon 49 of COL3A1 fused to an intron or a portion thereof between exon 18 and exon 19 of ALK. In some embodiments, a COL3A1-ALK fusion nucleic acid molecule provided herein comprises an intron or a portion thereof between exon 48 and exon 49 of COL3A1 fused to exon 19 or a portion thereof of ALK. In some embodiments, a COL3A1-ALK fusion nucleic acid molecule provided herein comprises exon 2 or a portion thereof of COL3A1 fused to an intron or a portion thereof between exon 18 and exon 19 of ALK. In some embodiments, a COL3A1-ALK fusion nucleic acid molecule provided herein comprises an intron or a portion thereof between exon 2 and exon 3 of COL3A1 fused to an intron or a portion thereof between exon 18 and exon 19 of ALK. In some embodiments, a COL3A1-ALK fusion nucleic acid molecule provided herein comprises an intron or a portion thereof between exon 2 and exon 3 of COL3A1 fused to exon 19 or a portion thereof of ALK.

In some embodiments, a COL3A1-ALK fusion nucleic acid molecule provided herein comprises exon 48 or a portion thereof of COL3A1 and intron 18 or a portion thereof of ALK, and a COL3A1-ALK breakpoint that fuses exon 48 or a portion thereof of COL3A1 and intron 18 or a portion thereof of ALK. In some embodiments, the 3′ end of exon 48 or of a portion of exon 48 of COL3A1 is fused to the 5′ end of intron 18 or of a portion of intron 18 of ALK.

In some embodiments, a COL3A1-ALK fusion nucleic acid molecule provided herein comprises exon 48 or a portion thereof of COL3A1 fused to exon 19 or a portion thereof of ALK, and a COL3A1-ALK breakpoint that fuses exon 48 or a portion thereof of COL3A1 and exon 19 or a portion thereof of ALK. In some embodiments, the 3′ end of exon 48 or of a portion of exon 48 of COL3A1 is fused to the 5′ end of exon 19 or of a portion of exon 19 of ALK.

In some embodiments, a COL3A1-ALK fusion nucleic acid molecule provided herein comprises intron 48 or a portion thereof of COL3A1 fused to intron 18 or a portion thereof of ALK, and a COL3A1-ALK breakpoint that fuses intron 48 or a portion thereof of COL3A1 and intron 18 or a portion thereof of ALK. In some embodiments, the 3′ end of intron 48 or of a portion of intron 48 of COL3A1 is fused to the 5′ end of intron 18 or of a portion of intron 18 of ALK.

In some embodiments, a COL3A1-ALK fusion nucleic acid molecule provided herein comprises intron 48 or a portion thereof of COL3A1 fused to exon 19 or a portion thereof of ALK, and a COL3A1-ALK breakpoint that fuses intron 48 or a portion thereof of COL3A1 and exon 19 or a portion thereof of ALK. In some embodiments, the 3′ end of intron 48 or of a portion of intron 48 of COL3A1 is fused to the 5′ end of exon 19 or of a portion of exon 19 of ALK.

In some embodiments, a COL3A1-ALK fusion nucleic acid molecule provided herein comprises exon 2 or a portion thereof of COL3A1 fused to intron 18 or a portion thereof of ALK, and a COL3A1-ALK breakpoint that fuses exon 2 or a portion thereof of COL3A1 and intron 18 or a portion thereof of ALK. In some embodiments, the 3′ end of exon 2 or of a portion of exon 2 of COL3A1 is fused to the 5′ end of intron 18 or of a portion of intron 18 of ALK.

In some embodiments, a COL3A1-ALK fusion nucleic acid molecule provided herein comprises exon 2 or a portion thereof of COL3A1 fused to exon 19 or a portion thereof of ALK, and a COL3A1-ALK breakpoint that fuses exon 2 or a portion thereof of COL3A1 and exon 19 or a portion thereof of ALK. In some embodiments, the 3′ end of exon 2 or of a portion of exon 2 of COL3A1 is fused to the 5′ end of exon 19 or of a portion of exon 19 of ALK.

In some embodiments, a COL3A1-ALK fusion nucleic acid molecule provided herein comprises intron 2 or a portion thereof of COL3A1 fused to intron 18 or a portion thereof of ALK, and a COL3A1-ALK breakpoint that fuses intron 2 or a portion thereof of COL3A1 and intron 18 or a portion thereof of ALK. In some embodiments, the 3′ end of intron 2 or of a portion of intron 2 of COL3A1 is fused to the 5′ end of intron 18 or of a portion of intron 18 of ALK.

In some embodiments, a COL3A1-ALK fusion nucleic acid molecule provided herein comprises intron 2 or a portion thereof of COL3A1 fused to exon 19 or a portion thereof of ALK, and a COL3A1-ALK breakpoint that fuses intron 2 or a portion thereof of COL3A1 and exon 19 or a portion thereof of ALK. In some embodiments, the 3′ end of intron 2 or of a portion of intron 2 of COL3A1 is fused to the 5′ end of exon 19 or of a portion of exon 19 of ALK.

In some embodiments, the exon-exon fusions or exon-intron fusions are in-frame fusions. The fusion breakpoint may occur anywhere within an exon or an intron of COL3A1 (e.g., exon 48, intron 48, exon 2, or intron 2), and anywhere within an exon or an intron of ALK (e.g., intron 18 or exon 19). When a breakpoint occurs in an intron of COL3A1, the resulting mRNA sequence, and the resulting amino acid sequence, has a breakpoint or fusion junction between the preceding exon of COL3A1 and the sequence of ALK. When a breakpoint occurs in an intron of ALK, the resulting mRNA sequence, and the resulting amino acid sequence, has a breakpoint or fusion junction between the following exon of ALK and the sequence of COL3A1. When the breakpoint or fusion junction occurs between an intron of COL3A1 and an intron of ALK, the resulting mRNA sequence, and the resulting amino acid sequence, has a breakpoint or fusion junction between the preceding exon of COL3A1 and the following exon of ALK. For example, a fusion of intron 48 of COL3A1 and intron 18 of ALK in the DNA sequence would result in an mRNA sequence, and in an amino acid sequence, having a breakpoint or fusion junction between exon 48 of COL3A1 and exon 19 of ALK. In another example, a fusion of intron 2 of COL3A1 and intron 18 of ALK in the DNA sequence would result in an mRNA sequence, and in an amino acid sequence, having a breakpoint or fusion junction between exon 2 of COL3A1 and exon 19 of ALK. One skilled in the art could readily determine the exon and intron sequences within the COL3A1 and ALK genes, for example using an NCBI database (e.g., GenBank).

In some embodiments, the COL3A1-ALK fusion nucleic acid molecule comprises 5 or more, 10 or more, or 20 or more nucleotides on the 5′ end of the COL3A1-ALK breakpoint, and 5 or more, 10 or more, or 20 or more nucleotides on the 3′ end of the COL3A1-ALK breakpoint. In some embodiments, the COL3A1-ALK fusion nucleic acid molecule comprises 5 or more nucleotides from exon 48 or intron 48 of COL3A1 on the 5′ end of the COL3A1-ALK breakpoint, and 5 or more nucleotides from intron 18 or exon 19 of ALK on the 3′ end of the COL3A1-ALK breakpoint. In some embodiments, the COL3A1-ALK fusion nucleic acid molecule comprises 5 or more nucleotides from exon 2 or intron 2 of COL3A1 on the 5′ end of the COL3A1-ALK breakpoint, and 5 or more nucleotides from intron 18 or exon 19 of ALK on the 3′ end of the COL3A1-ALK breakpoint.

In some embodiments, a COL3A1-ALK fusion nucleic acid molecule provided herein, e.g., a DNA molecule, results in an mRNA molecule comprising a fusion, e.g., an in-frame fusion, of exon 48 or a portion thereof of COL3A1 fused to exon 19 or a portion thereof of ALK. In some embodiments, a COL3A1-ALK fusion nucleic acid molecule provided herein, e.g., a DNA molecule, results in an mRNA molecule comprising a fusion, e.g., an in-frame fusion, of exon 2 or a portion thereof of COL3A1 fused to exon 19 or a portion thereof of ALK.

In some embodiments, a COL3A1-ALK fusion nucleic acid molecule provided herein comprises a COL3A1-ALK breakpoint resulting in an in-frame fusion of an exon described herein or a portion thereof of COL3A1 with an exon described herein or a portion thereof of ALK, e.g., resulting in an RNA molecule, such as an mRNA molecule, comprising an in-frame fusion of an exon described herein or a portion thereof of COL3A1 to an exon described herein or a portion thereof of ALK. In some embodiments, a COL3A1-ALK fusion nucleic acid molecule provided herein is an mRNA molecule comprising a fusion, e.g., an in-frame fusion, of exon 48 or a portion thereof of COL3A1 fused to exon 19 or a portion thereof of ALK. In some embodiments, a COL3A1-ALK fusion nucleic acid molecule provided herein is an mRNA molecule comprising a fusion, e.g., an in-frame fusion, of exon 2 or a portion thereof of COL3A1 fused to exon 19 or a portion thereof of ALK. In some embodiments, a COL3A1-ALK fusion nucleic acid molecule provided herein is a cDNA molecule comprising a fusion, e.g., an in-frame fusion, of exon 48 or a portion thereof of COL3A1 fused to exon 19 or a portion thereof of ALK. In some embodiments, a COL3A1-ALK fusion nucleic acid molecule provided herein is a cDNA molecule comprising a fusion, e.g., an in-frame fusion, of exon 2 or a portion thereof of COL3A1 fused to exon 19 or a portion thereof of ALK.

In some embodiments, the COL3A1-ALK fusion nucleic acid molecule comprises a nucleotide sequence comprising, in the 5′ to 3′ direction, a fusion of exon 48 or a portion thereof of COL3A1 to exon 19 or a portion thereof of ALK. In some embodiments, the COL3A1-ALK fusion nucleic acid molecule comprises a nucleotide sequence comprising, in the 5′ to 3′ direction, a fusion of exon 2 or a portion thereof of COL3A1 to exon 19 or a portion thereof of ALK.

In some embodiments, the COL3A1-ALK fusion nucleic acid molecule comprises a nucleotide sequence comprising, in the 5′ to 3′ direction, exons 1-47 and exon 48 or a portion thereof of COL3A1, and exon 19 or a portion thereof and exons 20-29 of ALK. In some embodiments, the COL3A1-ALK fusion nucleic acid molecule comprises a nucleotide sequence comprising, in the 5′ to 3′ direction, exon 1 and exon 2 or a portion thereof of COL3A1, and exon 19 or a portion thereof and exons 20-29 of ALK. In some embodiments, the COL3A1-ALK fusion nucleic acid molecule results from a breakpoint in exon 2 or intron 2 of COL3A1, and in intron 18 or exon 19 of ALK. In some embodiments, the COL3A1-ALK fusion nucleic acid molecule results from a breakpoint joining Chr2:189849674 with Chr2:29448496. In some embodiments, the COL3A1-ALK fusion nucleic acid molecule results from a breakpoint in exon 48 or intron 48 of COL3A1, and in intron 18 or exon 19 of ALK. In some embodiments, the COL3A1-ALK fusion nucleic acid molecule results from a breakpoint joining Chr2:189874528 with Chr2:29448490. In some embodiments, the COL3A1-ALK fusion nucleic acid molecule results from a breakpoint joining Chr2:189874814 with Chr2:29449440. In some embodiments, the chromosome positions correspond to chromosome positions of human genome version hg19.

In some embodiments, a COL3A1-ALK fusion nucleic acid molecule comprises at least a portion of a COL3A1 sequence of SEQ ID NO: 3 and at least a portion of an ALK sequence of SEQ ID NO: 1, or a sequence having at least about 85% (e.g., any of at least about 85%, at least about 90%, at least about 95%, at least about 96%, at least about 97%, at least about 98%, at least about 99%, or 100%) sequence identity to the portion of the COL3A1 sequence of SEQ ID NO: 3 and/or the portion of the ALK sequence of SEQ ID NO: 1.

In some embodiments, the COL3A1-ALK fusion nucleic acid molecule comprises the nucleotide sequence of SEQ ID NO: 8, or a nucleotide sequence at least 70%, 75%, 80%, 85%, 90%, 91%, 92%, 93%, 94%, 95%, 96%, 97%, 98%, 99%, or 99.5% identical to the nucleotide sequence of SEQ ID NO: 8.

(SEQ ID NO: 8)
GGCTGAGTTTTATGACGGGCCCGGTGCTGAAGGGCAGGGAACAACTTGA
TGGTGCTACTTTGAACTGCTTTTCTTTTCTCCTTTTTGCACAAAGAGTC
TCATGTCTGATATTTAGACATGATGAGCTTTGTGCAAAAGGGGAGCTGG
CTACTTCTCGCTCTGCTTCATCCCACTATTATTTTGGCACAACAGGAAG
CTGTTGAAGGAGGATGTTCCCATCTTGGTCAGTCCTATGCGGATAGAGA
TGTCTGGAAGCCAGAACCATGCCAAATATGTGTCTGTGACTCAGGATCC
GTTCTCTGCGATGACATAATATGTGACGATCAAGAATTAGACTGCCCCA
ACCCAGAAATTCCATTTGGAGAATGTTGTGCAGTTTGCCCACTGTCACC
CACCCCGGAGCCACACCTGCCACTCTCGCTGATCCTCTCTGTGGTGACC
TCTGCCCTCGTGGCCGCCCTGGTCCTGGCTTTCTCCGGCATCATGATTG
TGTACCGCCGGAAGCACCAGGAGCTGCAAGCCATGCAGATGGAGCTGCA
GAGCCCTGAGTACAAGCTGAGCAAGCTCCGCACCTCGACCATCATGACC
GACTACAACCCCAACTACTGCTTTGCTGGCAAGACCTCCTCCATCAGTG
ACCTGAAGGAGGTGCCGCGGAAAAACATCACCCTCATTCGGGGTCTGGG
CCATGGCGCCTTTGGGGAGGTGTATGAAGGCCAGGTGTCCGGAATGCCC
AACGACCCAAGCCCCCTGCAAGTGGCTGTGAAGACGCTGCCTGAAGTGT
GCTCTGAACAGGACGAACTGGATTTCCTCATGGAAGCCCTGATCATCAG
CAAATTCAACCACCAGAACATTGTTCGCTGCATTGGGGTGAGCCTGCAA
TCCCTGCCCCGGTTCATCCTGCTGGAGCTCATGGCGGGGGGAGACCTCA
AGTCCTTCCTCCGAGAGACCCGCCCTCGCCCGAGCCAGCCCTCCTCCCT
GGCCATGCTGGACCTTCTGCACGTGGCTCGGGACATTGCCTGTGGCTGT
CAGTATTTGGAGGAAAACCACTTCATCCACCGAGACATTGCTGCCAGAA
ACTGCCTCTTGACCTGTCCAGGCCCTGGAAGAGTGGCCAAGATTGGAGA
CTTCGGGATGGCCCGAGACATCTACAGGGCGAGCTACTATAGAAAGGGA
GGCTGTGCCATGCTGCCAGTTAAGTGGATGCCCCCAGAGGCCTTCATGG
AAGGAATATTCACTTCTAAAACAGACACATGGTCCTTTGGAGTGCTGCT
ATGGGAAATCTTTTCTCTTGGATATATGCCATACCCCAGCAAAAGCAAC
CAGGAAGTTCTGGAGTTTGTCACCAGTGGAGGCCGGATGGACCCACCCA
AGAACTGCCCTGGGCCTGTATACCGGATAATGACTCAGTGCTGGCAACA
TCAGCCTGAAGACAGGCCCAACTTTGCCATCATTTTGGAGAGGATTGAA
TACTGCACCCAGGACCCGGATGTAATCAACACCGCTTTGCCGATAGAAT
ATGGTCCACTTGTGGAAGAGGAAGAGAAAGTGCCTGTGAGGCCCAAGGA
CCCTGAGGGGGTTCCTCCTCTCCTGGTCTCTCAACAGGCAAAACGGGAG
GAGGAGCGCAGCCCAGCTGCCCCACCACCTCTGCCTACCACCTCCTCTG
GCAAGGCTGCAAAGAAACCCACAGCTGCAGAGATCTCTGTTCGAGTCCC
TAGAGGGCCGGCCGTGGAAGGGGGACACGTGAATATGGCATTCTCTCAG
TCCAACCCTCCTTCGGAGTTGCACAAGGTCCACGGATCCAGAAACAAGC
CCACCAGCTTGTGGAACCCAACGTACGGCTCCTGGTTTACAGAGAAACC
CACCAAAAAGAATAATCCTATAGCAAAGAAGGAGCCACACGACAGGGGT
AACCTGGGGCTGGAGGGAAGCTGTACTGTCCCACCTAACGTTGCAACTG
GGAGACTTCCGGGGGCCTCACTGCTCCTAGAGCCCTCTTCGCTGACTGC
CAATATGAAGGAGGTACCTCTGTTCAGGCTACGTCACTTCCCTTGTGGG
AATGTCAATTACGGCTACCAGCAACAGGGCTTGCCCTTAGAAGCCGCTA
CTGCCCCTGGAGCTGGTCATTACGAGGATACCATTCTGAAAAGCAAGAA
TAGCATGAACCAGCCTGGGCCCTGAGCTCGGTCGCACACTCACTTCTCT
TCCTTGGGATCCCTAAGACCGTGGAGGAGAGAGAGGCAATGGCTCCTTC
ACAAACCAGAGACCAAATGTCACGTTTTGTTTTGTGCCAACCTATTTTG
AAGTACCACCAAAAAAGCTGTATTTTGAAAATGCTTTAGAAAGGTTTTG
AGCATGGGTTCATCCTATTCTTTCGAAAGAAGAAAATATCATAAAAATG
AGTGATAAATACAAGGCCCAGATGTGGTTGCATAAGGTTTTTATGCATG
TTTGTTGTATACTTCCTTATGCTTCTTTCAAATTGTGTGTGCTCTGCTT
CAATGTAGTCAGAATTAGCTGCTTCTATGTTTCATAGTTGGGGTCATAG
ATGTTTCCTTGCCTTGTTGATGTGGACATGAGCCATTTGAGGGGAGAGG
GAACGGAAATAAAGGAGTTATTTGTAATGACTAA

In the sequence of SEQ ID NO: 8 provided above, nucleotide sequences corresponding to ALK are underlined, and bolded sequences correspond to a novel codon formed by the fusion (wherein the first base corresponds to COL3A1 and the second and third bases are from ALK).

In some embodiments, the COL3A1-ALK fusion nucleic acid molecule comprises the nucleotide sequence of SEQ ID NO: 9, or a nucleotide sequence at least 70%, 75%, 80%, 85%, 90%, 91%, 92%, 93%, 94%, 95%, 96%, 97%, 98%, 99%, or 99.5% identical to the nucleotide sequence of SEQ ID NO: 9.

(SEQ ID NO: 9)
GGCTGAGTTTTATGACGGGCCCGGTGCTGAAGGGCAGGGAACAACTTGATGGTGCTACTTTG
AACTGCTTTTCTTTTCTCCTTTTTGCACAAAGAGTCTCATGTCTGATATTTAGACATGATGA
GCTTTGTGCAAAAGGGGAGCTGGCTACTTCTCGCTCTGCTTCATCCCACTATTATTTTGGCA
CAACAGGAAGCTGTTGAAGGAGGATGTTCCCATCTTGGTCAGTCCTATGCGGATAGAGATGT
CTGGAAGCCAGAACCATGCCAAATATGTGTCTGTGACTCAGGATCCGTTCTCTGCGATGACA
TAATATGTGACGATCAAGAATTAGACTGCCCCAACCCAGAAATTCCATTTGGAGAATGTTGT
GCAGTTTGCCCACAGCCTCCAACTGCTCCTACTCGCCCTCCTAATGGTCAAGGACCTCAAGG
CCCCAAGGGAGATCCAGGCCCTCCTGGTATTCCTGGGAGAAATGGTGACCCTGGTATTCCAG
GACAACCAGGGTCCCCTGGTTCTCCTGGCCCCCCTGGAATCTGTGAATCATGCCCTACTGGT
CCTCAGAACTATTCTCCCCAGTATGATTCATATGATGTCAAGTCTGGAGTAGCAGTAGGAGG
ACTCGCAGGCTATCCTGGACCAGCTGGCCCCCCAGGCCCTCCCGGTCCCCCTGGTACATCTG
GTCATCCTGGTTCCCCTGGATCTCCAGGATACCAAGGACCCCCTGGTGAACCTGGGCAAGCT
GGTCCTTCAGGCCCTCCAGGACCTCCTGGTGCTATAGGTCCATCTGGTCCTGCTGGAAAAGA
TGGAGAATCAGGTAGACCCGGACGACCTGGAGAGCGAGGATTGCCTGGACCTCCAGGTATCA
AAGGTCCAGCTGGGATACCTGGATTCCCTGGTATGAAAGGACACAGAGGCTTCGATGGACGA
AATGGAGAAAAGGGTGAAACAGGTGCTCCTGGATTAAAGGGTGAAAATGGTCTTCCAGGCGA
AAATGGAGCTCCTGGACCCATGGGTCCAAGAGGGGCTCCTGGTGAGCGAGGACGGCCAGGAC
TTCCTGGGGCTGCAGGTGCTCGGGGTAATGACGGTGCTCGAGGCAGTGATGGTCAACCAGGC
CCTCCTGGTCCTCCTGGAACTGCCGGATTCCCTGGATCCCCTGGTGCTAAGGGTGAAGTTGG
ACCTGCAGGGTCTCCTGGTTCAAATGGTGCCCCTGGACAAAGAGGAGAACCTGGACCTCAGG
GACACGCTGGTGCTCAAGGTCCTCCTGGCCCTCCTGGGATTAATGGTAGTCCTGGTGGTAAA
GGCGAAATGGGTCCCGCTGGCATTCCTGGAGCTCCTGGACTGATGGGAGCCCGGGGTCCTCC
AGGACCAGCCGGTGCTAATGGTGCTCCTGGACTGCGAGGTGGTGCAGGTGAGCCTGGTAAGA
ATGGTGCCAAAGGAGAGCCCGGACCACGTGGTGAACGCGGTGAGGCTGGTATTCCAGGTGTT
CCAGGAGCTAAAGGCGAAGATGGCAAGGATGGATCACCTGGAGAACCTGGTGCAAATGGGCT
TCCAGGAGCTGCAGGAGAAAGGGGTGCCCCTGGGTTCCGAGGACCTGCTGGACCAAATGGCA
TCCCAGGAGAAAAGGGTCCTGCTGGAGAGCGTGGTGCTCCAGGCCCTGCAGGGCCCAGAGGA
GCTGCTGGAGAACCTGGCAGAGATGGCGTCCCTGGAGGTCCAGGAATGAGGGGCATGCCCGG
AAGTCCAGGAGGACCAGGAAGTGATGGGAAACCAGGGCCTCCCGGAAGTCAAGGAGAAAGTG
GTCGACCAGGTCCTCCTGGGCCATCTGGTCCCCGAGGTCAGCCTGGTGTCATGGGCTTCCCC
GGTCCTAAAGGAAATGATGGTGCTCCTGGTAAGAATGGAGAACGAGGTGGCCCTGGAGGACC
TGGCCCTCAGGGTCCTCCTGGAAAGAATGGTGAAACTGGACCTCAGGGACCCCCAGGGCCTA
CTGGGCCTGGTGGTGACAAAGGAGACACAGGACCCCCTGGTCCACAAGGATTACAAGGCTTG
CCTGGTACAGGTGGTCCTCCAGGAGAAAATGGAAAACCTGGGGAACCAGGTCCAAAGGGTGA
TGCCGGTGCACCTGGAGCTCCAGGAGGCAAGGGTGATGCTGGTGCCCCTGGTGAACGTGGAC
CTCCTGGATTGGCAGGGGCCCCAGGACTTAGAGGTGGAGCTGGTCCCCCTGGTCCCGAAGGA
GGAAAGGGTGCTGCTGGTCCTCCTGGGCCACCTGGTGCTGCTGGTACTCCTGGTCTGCAAGG
AATGCCTGGAGAAAGAGGAGGTCTTGGAAGTCCTGGTCCAAAGGGTGACAAGGGTGAACCAG
GCGGTCCAGGTGCTGATGGTGTCCCAGGGAAAGATGGCCCAAGGGGTCCTACTGGTCCTATT
GGTCCTCCTGGCCCAGCTGGCCAGCCTGGAGATAAGGGTGAAGGTGGTGCCCCCGGACTTCC
AGGTATAGCTGGACCTCGTGGTAGCCCTGGTGAGAGAGGTGAAACTGGCCCTCCAGGACCTG
CTGGTTTCCCTGGTGCTCCTGGACAGAATGGTGAACCTGGTGGTAAAGGAGAAAGAGGGGCT
CCGGGTGAGAAAGGTGAAGGAGGCCCTCCTGGAGTTGCAGGACCCCCTGGAGGTTCTGGACC
TGCTGGTCCTCCTGGTCCCCAAGGTGTCAAAGGTGAACGTGGCAGTCCTGGTGGACCTGGTG
CTGCTGGCTTCCCTGGTGCTCGTGGTCTTCCTGGTCCTCCTGGTAGTAATGGTAACCCAGGA
CCCCCAGGTCCCAGCGGTTCTCCAGGCAAGGATGGGCCCCCAGGTCCTGCGGGTAACACTGG
TGCTCCTGGCAGCCCTGGAGTGTCTGGACCAAAAGGTGATGCTGGCCAACCAGGAGAGAAGG
GATCGCCTGGTGCCCAGGGCCCACCAGGAGCTCCAGGCCCACTTGGGATTGCTGGGATCACT
GGAGCACGGGGTCTTGCAGGACCACCAGGCATGCCAGGTCCTAGGGGAAGCCCTGGCCCTCA
GGGTGTCAAGGGTGAAAGTGGGAAACCAGGAGCTAACGGTCTCAGTGGAGAACGTGGTCCCC
CTGGACCCCAGGGTCTTCCTGGTCTGGCTGGTACAGCTGGTGAACCTGGAAGAGATGGAAAC
CCTGGATCAGATGGTCTTCCAGGCCGAGATGGATCTCCTGGTGGCAAGGGTGATCGTGGTGA
AAATGGCTCTCCTGGTGCCCCTGGCGCTCCTGGTCATCCAGGCCCACCTGGTCCTGTCGGTC
CAGCTGGAAAGAGTGGTGACAGAGGAGAAAGTGGCCCTGCTGGCCCTGCTGGTGCTCCCGGT
CCTGCTGGTTCCCGAGGTGCTCCTGGTCCTCAAGGCCCACGTGGTGACAAAGGTGAAACAGG
TGAACGTGGAGCTGCTGGCATCAAAGGACATCGAGGATTCCCTGGTAATCCAGGTGCCCCAG
GTTCTCCAGGCCCTGCTGGTCAGCAGGGTGCAATCGGCAGTCCAGGACCTGCAGGCCCCAGA
GGACCTGTTGGACCCAGTGGACCTCCTGGCAAAGATGGAACCAGTGGACATCCAGGTCCCAT
TGGACCACCAGGGCCTCGAGGTAACAGAGGTGAAAGAGGATCTGAGGGCTCCCCAGGCCACC
CAGGGCAACCAGGCCCTCCTGGACCTCCTGGTGCCCCTGGTCCTTGCTGTGGTGGTGTTGGA
GCCGCTGCCATTGCTGGGATTGGAGGTGAAAAAGCTGGCGGTTTTGCCCCGTATTATGGAGA
TGAACCAATGGATTTCAAAATCAACACCGATGAGATTATGACTTCACTCAAGTCTGTTAATG
GACAAATAGAAAGCCTCATTAGTCCTGATGGTTCTCGTAAAAACCCCGCTAGAAACTGCAGA
GACCTGAAATTCTGCCATCCTGAACTCAAGAGTGTGTCACCCACCCCGGAGCCACACCTGCC
ACTCTCGCTGATCCTCTCTGTGGTGACCTCTGCCCTCGTGGCCGCCCTGGTCCTGGCTTTCT
CCGGCATCATGATTGTGTACCGCCGGAAGCACCAGGAGCTGCAAGCCATGCAGATGGAGCTG
CAGAGCCCTGAGTACAAGCTGAGCAAGCTCCGCACCTCGACCATCATGACCGACTACAACCC
CAACTACTGCTTTGCTGGCAAGACCTCCTCCATCAGTGACCTGAAGGAGGTGCCGCGGAAAA
ACATCACCCTCATTCGGGGTCTGGGCCATGGCGCCTTTGGGGAGGTGTATGAAGGCCAGGTG
TCCGGAATGCCCAACGACCCAAGCCCCCTGCAAGTGGCTGTGAAGACGCTGCCTGAAGTGTG
CTCTGAACAGGACGAACTGGATTTCCTCATGGAAGCCCTGATCATCAGCAAATTCAACCACC
AGAACATTGTTCGCTGCATTGGGGTGAGCCTGCAATCCCTGCCCCGGTTCATCCTGCTGGAG
CTCATGGCGGGGGGAGACCTCAAGTCCTTCCTCCGAGAGACCCGCCCTCGCCCGAGCCAGCC
CTCCTCCCTGGCCATGCTGGACCTTCTGCACGTGGCTCGGGACATTGCCTGTGGCTGTCAGT
ATTTGGAGGAAAACCACTTCATCCACCGAGACATTGCTGCCAGAAACTGCCTCTTGACCTGT
CCAGGCCCTGGAAGAGTGGCCAAGATTGGAGACTTCGGGATGGCCCGAGACATCTACAGGGC
GAGCTACTATAGAAAGGGAGGCTGTGCCATGCTGCCAGTTAAGTGGATGCCCCCAGAGGCCT
TCATGGAAGGAATATTCACTTCTAAAACAGACACATGGTCCTTTGGAGTGCTGCTATGGGAA
ATCTTTTCTCTTGGATATATGCCATACCCCAGCAAAAGCAACCAGGAAGTTCTGGAGTTTGT
CACCAGTGGAGGCCGGATGGACCCACCCAAGAACTGCCCTGGGCCTGTATACCGGATAATGA
CTCAGTGCTGGCAACATCAGCCTGAAGACAGGCCCAACTTTGCCATCATTTTGGAGAGGATT
GAATACTGCACCCAGGACCCGGATGTAATCAACACCGCTTTGCCGATAGAATATGGTCCACT
TGTGGAAGAGGAAGAGAAAGTGCCTGTGAGGCCCAAGGACCCTGAGGGGGTTCCTCCTCTCC
TGGTCTCTCAACAGGCAAAACGGGAGGAGGAGCGCAGCCCAGCTGCCCCACCACCTCTGCCT
ACCACCTCCTCTGGCAAGGCTGCAAAGAAACCCACAGCTGCAGAGATCTCTGTTCGAGTCCC
TAGAGGGCCGGCCGTGGAAGGGGGACACGTGAATATGGCATTCTCTCAGTCCAACCCTCCTT
CGGAGTTGCACAAGGTCCACGGATCCAGAAACAAGCCCACCAGCTTGTGGAACCCAACGTAC
GGCTCCTGGTTTACAGAGAAACCCACCAAAAAGAATAATCCTATAGCAAAGAAGGAGCCACA
CGACAGGGGTAACCTGGGGCTGGAGGGAAGCTGTACTGTCCCACCTAACGTTGCAACTGGGA
GACTTCCGGGGGCCTCACTGCTCCTAGAGCCCTCTTCGCTGACTGCCAATATGAAGGAGGTA
CCTCTGTTCAGGCTACGTCACTTCCCTTGTGGGAATGTCAATTACGGCTACCAGCAACAGGG
CTTGCCCTTAGAAGCCGCTACTGCCCCTGGAGCTGGTCATTACGAGGATACCATTCTGAAAA
GCAAGAATAGCATGAACCAGCCTGGGCCCTGAGCTCGGTCGCACACTCACTTCTCTTCCTTG
GGATCCCTAAGACCGTGGAGGAGAGAGAGGCAATGGCTCCTTCACAAACCAGAGACCAAATG
TCACGTTTTGTTTTGTGCCAACCTATTTTGAAGTACCACCAAAAAAGCTGTATTTTGAAAAT
GCTTTAGAAAGGTTTTGAGCATGGGTTCATCCTATTCTTTCGAAAGAAGAAAATATCATAAA
AATGAGTGATAAATACAAGGCCCAGATGTGGTTGCATAAGGTTTTTATGCATGTTTGTTGTA
TACTTCCTTATGCTTCTTTCAAATTGTGTGTGCTCTGCTTCAATGTAGTCAGAATTAGCTGC
TTCTATGTTTCATAGTTGGGGTCATAGATGTTTCCTTGCCTTGTTGATGTGGACATGAGCCA
TTTGAGGGGAGAGGGAACGGAAATAAAGGAGTTATTTGTAATGACTAA

In the sequence of SEQ ID NO: 9 provided above, nucleotide sequences corresponding to ALK are underlined, and bolded sequences correspond to a novel codon formed by the fusion (wherein the first base corresponds to COL3A1 and the second and third bases are from ALK).

In some embodiments, the COL3A1-ALK fusion nucleic acid molecule is an isolated nucleic acid molecule. The isolated nucleic acid molecule may be free of sequences (such as protein-encoding sequences) that naturally flank the nucleic acid (i.e., sequences located at the 5′ and 3′ ends of the nucleic acid) in the genomic DNA of the organism from which the nucleic acid is derived. For example, in various embodiments, the fusion nucleic acid molecule can contain less than about 5 kB, less than about 4 kB, less than about 3 kB, less than about 2 kB, less than about 1 kB, less than about 0.5 kB or less than about 0.1 kB of nucleotide sequences which naturally flank the nucleic acid molecule in genomic DNA of the cell from which the nucleic acid is derived.

ALK Fusion Polypeptides

In certain aspects, provided herein are COL5A2-ALK or COL3A1-ALK fusion polypeptides.

COL5A2-ALK Fusion Polypeptides

In some aspects, provided herein are COL5A2-ALK fusion polypeptides. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a COL5A2-ALK fusion nucleic acid molecule described herein or a fragment thereof.

In some embodiments, the COL5A2-ALK fusion polypeptides provided herein comprise an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or an exon of COL5A2, or a portion or fragment thereof, directly fused to an intron or an exon of ALK, or a portion or fragment thereof.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising at least one exon of COL5A2 or a portion thereof and at least one exon of ALK or a portion thereof.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an exon or a portion thereof of COL5A2 and an intron or a portion thereof of ALK, and a COL5A2-ALK breakpoint that fuses the exon or a portion thereof of COL5A2 to the intron or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof of COL5A2 and an intron or a portion thereof of ALK, and a COL5A2-ALK breakpoint that fuses the intron or a portion thereof of COL5A2 to the intron or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an exon or a portion thereof of COL5A2 and an exon or a portion thereof of ALK, and a COL5A2-ALK breakpoint that fuses the exon or a portion thereof of COL5A2 to the exon or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof of COL5A2 and an exon or a portion thereof of ALK, and a COL5A2-ALK breakpoint that fuses the intron or a portion thereof of COL5A2 to the exon or a portion thereof of ALK. In some embodiments, the COL5A2 breakpoint occurs within an intron or within an exon of COL5A2, e.g., within exon 1 or intron 1 of COL5A2. In some embodiments, the COL5A2 breakpoint occurs at the 3′ end or at the 5′ end of an intron, or at the 3′ end or at the 5′ end of an exon, of COL5A2, e.g., at the 3′ end of exon 1, at the 5′ end of intron 1, or at the 3′ end of intron 1 of COL5A2. In some embodiments, the ALK breakpoint occurs within an intron or within an exon of ALK, e.g., within intron 5 or exon 6 of ALK. In some embodiments, the ALK breakpoint occurs at the 3′ end or at the 5′ end of an intron, or at the 3′ end or at the 5′ end of an exon, of ALK, e.g., at the 5′ end of intron 5, at the 3′ end of intron 5, or at the 5′ end of exon 6 of ALK. In certain embodiments, exon 1 or intron 1, or a portion of exon 1 or intron 1, of COL5A2 is directly fused to intron 5 or exon 6, or a portion of intron 5 or exon 6, of ALK, thereby establishing a COL5A2-ALK breakpoint between the COL5A2 sequence and the ALK sequence.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 1 or a portion thereof of COL5A2 fused to intron 5 or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 1 or a portion thereof of COL5A2 fused to intron 5 or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 1 or a portion thereof of COL5A2 fused to exon 6 or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 1 or a portion thereof of COL5A2 fused to exon 6 or a portion thereof of ALK.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL5A2-ALK breakpoint that fuses exon 1 or a portion thereof of COL5A2 to intron 5 or a portion thereof of ALK. In some embodiments, the COL5A2-ALK breakpoint fuses the 3′ end of exon 1 or the portion thereof of COL5A2 to the 5′ end of intron 5 or the portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL5A2-ALK breakpoint that fuses intron 1 or a portion thereof of COL5A2 to intron 5 or a portion thereof of ALK. In some embodiments, the COL5A2-ALK breakpoint fuses the 3′ end of intron 1 or a portion thereof of COL5A2 to the 5′ end of intron 5 or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL5A2-ALK breakpoint that fuses exon 1 or a portion thereof of COL5A2 to exon 6 or a portion thereof of ALK. In some embodiments, the COL5A2-ALK breakpoint fuses the 3′ end of exon 1 or a portion thereof of COL5A2 to the 5′ end of exon 6 or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL5A2-ALK breakpoint that fuses intron 1 or a portion thereof of COL5A2 to exon 6 or a portion thereof of ALK. In some embodiments, the COL5A2-ALK breakpoint fuses the 3′ end of intron 1 or a portion thereof of COL5A2 to the 5′ end of exon 6 or a portion thereof of ALK.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 1 or a portion thereof of COL5A2 fused to an intron or a portion thereof between exon 5 and exon 6 of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof between exon 1 and exon 2 of COL5A2 fused to an intron or a portion thereof between exon 5 and exon 6 of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof between exon 1 and exon 2 of COL5A2 fused to exon 6 or a portion thereof of ALK.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 1 or a portion thereof of COL5A2 and intron 5 or a portion thereof of ALK, and a COL5A2-ALK breakpoint that fuses exon 1 or the portion thereof of COL5A2 and intron 5 or the portion thereof of ALK. In some embodiments, the 3′ end of exon 1 or of a portion of exon 1 of COL5A2 is fused to the 5′ end of intron 5 or of a portion of intron 5 of ALK.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 1 or a portion thereof of COL5A2 and intron 5 or a portion thereof of ALK, and a COL5A2-ALK breakpoint that fuses intron 1 or the portion thereof of COL5A2 and intron 5 or the portion thereof of ALK. In some embodiments, the 3′ end of intron 1 or of a portion of intron 1 of COL5A2 is fused to the 5′ end of intron 5 or of a portion of intron 5 of ALK.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 1 or a portion thereof of COL5A2 and exon 6 or a portion thereof of ALK, and a COL5A2-ALK breakpoint that fuses exon 1 or the portion thereof of COL5A2 and exon 6 or the portion thereof of ALK. In some embodiments, the 3′ end of exon 1 or of a portion of exon 1 of COL5A2 is fused to the 5′ end of exon 6 or of a portion of exon 6 of ALK.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 1 or a portion thereof of COL5A2 and exon 6 or a portion thereof of ALK, and a COL5A2-ALK breakpoint that fuses intron 1 or the portion thereof of COL5A2 and exon 6 or the portion thereof of ALK. In some embodiments, the 3′ end of intron 1 or of a portion of intron 1 of COL5A2 is fused to the 5′ end of exon 6 or of a portion of exon 6 of ALK.

In some embodiments, the exon-exon fusions or exon-intron fusions are in-frame fusions. The fusion breakpoint may occur anywhere within an exon or an intron of COL5A2 (e.g., exon 1 or intron 1), and anywhere within an exon or an intron of ALK (e.g., intron 5 or exon 6). When a breakpoint occurs in an intron of COL5A2, the resulting mRNA sequence, and the resulting amino acid sequence, has a breakpoint or fusion junction between the preceding exon of COL5A2 and the sequence of ALK. When a breakpoint occurs in an intron of ALK, the resulting mRNA sequence, and the resulting amino acid sequence, has a breakpoint or fusion junction between the following exon of ALK and the sequence of COL5A2. When the breakpoint or fusion junction occurs between an intron of COL5A2 and an intron of ALK, the resulting mRNA sequence, and the resulting amino acid sequence, has a breakpoint or fusion junction between the preceding exon of COL5A2 and the following exon of ALK. For example, a fusion of intron 1 of COL5A2 and intron 5 of ALK in the DNA sequence would result in an mRNA sequence and in an amino acid sequence having a breakpoint or fusion junction between exon 1 of COL5A2 and exon 6 of ALK. One skilled in the art could readily determine the exon and intron sequences within the COL5A2 and ALK genes, and the corresponding mRNA and amino acid sequences, for example using an NCBI database (e.g., GenBank).

In some embodiments, the COL5A2-ALK fusion polypeptide comprises an amino acid sequence encoded by a nucleic acid comprising 5 or more, 10 or more, or 20 or more nucleotides on the 5′ end of the COL5A2-ALK breakpoint, and 5 or more, 10 or more, or 20 or more nucleotides on the 3′ end of the COL5A2-ALK breakpoint. In some embodiments, the COL5A2-ALK fusion polypeptide comprises an amino acid sequence encoded by a nucleic acid comprising 5 or more nucleotides from exon 1 or intron 1 of COL5A2 on the 5′ end of the COL5A2-ALK breakpoint, and 5 or more nucleotides from intron 5 or exon 6 of ALK on the 3′ end of the COL5A2-ALK breakpoint.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by an mRNA molecule comprising a fusion, e.g., an in-frame fusion, of exon 1 or a portion thereof of COL5A2 fused to exon 6 or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a cDNA molecule comprising a fusion, e.g., an in-frame fusion, of exon 1 or a portion thereof of COL5A2 fused to exon 6 or a portion thereof of ALK.

In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL5A2-ALK breakpoint resulting in an in-frame fusion of an exon described herein or a portion thereof of COL5A2 with an exon described herein or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by an mRNA molecule comprising an in-frame fusion of an exon described herein or a portion thereof of COL5A2 to an exon described herein or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a cDNA molecule comprising an in-frame fusion of an exon described herein or a portion thereof of COL5A2 to an exon described herein or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by an mRNA molecule comprising a fusion, e.g., an in-frame fusion, of exon 1 or a portion thereof of COL5A2 fused to exon 6 or a portion thereof of ALK. In some embodiments, a COL5A2-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a cDNA molecule comprising a fusion, e.g., an in-frame fusion, of exon 1 or a portion thereof of COL5A2 fused to exon 6 or a portion thereof of ALK.

In some embodiments, the COL5A2-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a nucleotide sequence comprising, in the 5′ to 3′ direction, a fusion of exon 1 or a portion thereof of COL5A2 to exon 6 or a portion thereof of ALK. In some embodiments, the COL5A2-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a nucleotide sequence comprising, in the 5′ to 3′ direction, exon 1 or a portion thereof of COL5A2, and exon 6 or a portion thereof and exons 7-29 of ALK. In some embodiments, the COL5A2-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid resulting from a breakpoint in exon 1 or in intron 1 of COL5A2, and in intron 5 or in exon 6 of ALK.

In some embodiments, the COL5A2-ALK fusion polypeptide comprises 5 or more amino acids (e.g., any of 5 or more, 10 or more, 15 or more, or 20 or more amino acids) encoded by the 3′ end of exon 1 or a portion thereof of COL5A2, fused to 5 or more amino acids (e.g., any of 5 or more, 10 or more, 15 or more, or 20 or more amino acids) encoded by the 5′ end of exon 6 of ALK or a portion thereof.

In some embodiments, a COL5A2-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising at least a portion of a COL5A2 sequence of SEQ ID NO: 2 and at least a portion of an ALK sequence of SEQ ID NO: 1, or a sequence having at least about 85% (e.g., any of at least about 85%, at least about 90%, at least about 95%, at least about 96%, at least about 97%, at least about 98%, at least about 99%, or 100%) sequence identity to the portion of the COL5A2 sequence of SEQ ID NO: 2 and/or the portion of the ALK sequence of SEQ ID NO: 1.

In some embodiments, a COL5A2-ALK fusion polypeptide comprises an amino acid sequence comprising at least a portion of a COL5A2 sequence of SEQ ID NO: 5 and at least a portion of an ALK sequence of SEQ ID NO: 4, or a sequence having at least about 85% (e.g., any of at least about 85%, at least about 90%, at least about 95%, at least about 96%, at least about 97%, at least about 98%, at least about 99%, or 100%) sequence identity to the portion of the COL5A2 sequence of SEQ ID NO: 5 and the portion of the ALK sequence of SEQ ID NO: 4.

In some embodiments, the COL5A2-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by the nucleotide sequence of SEQ ID NO: 7, or a nucleotide sequence at least 70%, 75%, 80%, 85%, 90%, 91%, 92%, 93%, 94%, 95%, 96%, 97%, 98%, 99%, or 99.5% identical to the nucleotide sequence of SEQ ID NO: 7.

In some embodiments, the COL5A2-ALK fusion polypeptide comprises an amino acid sequence at least about 85%, at least about 90%, at least about 95%, at least about 96%, at least about 97%, at least about 98%, at least about 99%, or 100% identical to the amino acid sequence of SEQ ID NO: 10. In some embodiments, the COL5A2-ALK fusion polypeptide comprises the amino acid sequence of SEQ ID NO: 10.

(SEQ ID NO: 10)
MMANWAEARPLLILIVLLGQFVSIKAQEEDEDGTSPGSKMALQSSFTCW
NGTVLQLGQACDFHQDCAQGEDESQMCRKLPVGFYCNFEDGFCGWTQGT
LSPHTPQWQVRTLKDARFQDHQDHALLLSTTDVPASESATVTSATFPAP
IKSSPCELRMSWLIRGVLRGNVSLVLVENKTGKEQGRMVWHVAAYEGLS
LWQWMVLPLLDVSDRFWLQMVAWWGQGSRAIVAFDNISISLDCYLTISG
EDKILQNTAPKSRNLFERNPNKELKPGENSPRQTPIFDPTVHWLFTTCG
ASGPHGPTQAQCNNAYQNSNLSVEVGSEGPLKGIQIWKVPATDTYSISG
YGAAGGKGGKNTMMRSHGVSVLGIFNLFKDDMLYILVGQQGEDACPSTN
QLIQKVCIGENNVIEEEIRVNRSVHEWAGGGGGGGGATYVEKMKDGVPV
PLIIAAGGGGRAYGAKTDTFHPERLENNSSVLGLNGNSGAAGGGGGWND
NTSLLWAGKSLQEGATGGHSCPQAMKKWGWETRGGFGGGGGGCSSGGGG
GGYIGGNAASNNDPEMDGEDGVSFISPLGILYTPALKVMEGHGEVNIKH
YLNCSHCEVDECHMDPESHKVICFCDHGTVLAEDGVSCIVSPTPEPHLP
LSLILSVVTSALVAALVLAFSGIMIVYRRKHQELQAMQMELQSPEYKLS
KLRTSTIMTDYNPNYCFAGKTSSISDLKEVPRKNITLIRGLGHGAFGEV
YEGQVSGMPNDPSPLQVAVKTLPEVCSEQDELDELMEALIISKENHQNI
VRCIGVSLQSLPRFILLELMAGGDLKSFLRETRPRPSQPSSLAMLDLLH
VARDIACGCQYLEENHFIHRDIAARNCLLTCPGPGRVAKIGDFGMARDI
YRASYYRKGGCAMLPVKWMPPEAFMEGIFTSKTDTWSFGVLLWEIFSLG
YMPYPSKSNQEVLEFVTSGGRMDPPKNCPGPVYRIMTQCWQHQPEDRPN
FAIILERIEYCTQDPDVINTALPIEYGPLVEEEEKVPVRPKDPEGVPPL
LVSQQAKREEERSPAAPPPLPTTSSGKAAKKPTAAEISVRVPRGPAVEG
GHVNMAFSQSNPPSELHKVHGSRNKPTSLWNPTYGSWFTEKPTKKNNPI
AKKEPHDRGNLGLEGSCTVPPNVATGRLPGASLLLEPSSLTANMKEVPL
FRLRHFPCGNVNYGYQQQGLPLEAATAPGAGHYEDTILKSKNSMNQPGP

In the sequence of SEQ ID NO: 10 provided above, amino acid sequences corresponding to ALK are underlined, and bolded sequences correspond to a novel amino acid formed by the fusion.

In some embodiments, the COL5A2-ALK fusion polypeptide is isolated from cells or tissue sources according to methods known in the art. In some embodiments, a fusion polypeptide provided herein can be synthesized chemically using standard peptide synthesis techniques. In some embodiments, a fusion polypeptide provided herein is isolated or purified such that it is substantially free of cellular material or other contaminating proteins from the cell or tissue source from which the protein is derived, or substantially free of chemical precursors or other chemicals when chemically synthesized.

In some embodiments, the COL5A2-ALK fusion polypeptide is fused to a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, Green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, Strep-tag, and Nus-tag. In some embodiments, the label or tag comprises a detection agent, such as a fluorescent molecule or an affinity reagent or tag.

In some embodiments, the COL5A2-ALK fusion polypeptide has a kinase activity, e.g., an ALK kinase activity, or a tyrosine kinase activity. Methods of assessing kinase activity are known in the art and include, without limitation, using radioactivity-based assays (e.g., using ³²P-orthophosphate or other suitable reagents) in combination with SDS-PAGE, 2-dimensional gel electrophoresis, phosphorylation-state specific antibodies, Western blots, enzyme-linked immunosorbent assays (ELISA), mass spectrometry, immunohistochemistry, and flow cytometry.

COL3A1-ALK Fusion Polypeptides

In some aspects, provided herein are COL3A1-ALK fusion polypeptides. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a COL3A1-ALK fusion nucleic acid molecule described herein or a fragment thereof.

In some embodiments, the COL3A1-ALK fusion polypeptides provided herein comprise an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or an exon of COL3A1, or a portion or fragment thereof, directly fused to an intron or an exon of ALK, or a portion or fragment thereof.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising at least one exon of COL3A1 or a portion thereof and at least one exon of ALK or a portion thereof.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an exon or a portion thereof of COL3A1 and an intron or a portion thereof of ALK, and a COL3A1-ALK breakpoint that fuses the exon or a portion thereof of COL3A1 to the intron or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof of COL3A1 and an intron or a portion thereof of ALK, and a COL3A1-ALK breakpoint that fuses the intron or a portion thereof of COL3A1 to the intron or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an exon or a portion thereof of COL3A1 and an exon or a portion thereof of ALK, and a COL3A1-ALK breakpoint that fuses the exon or a portion thereof of COL3A1 to the exon or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof of COL3A1 and an exon or a portion thereof of ALK, and a COL3A1-ALK breakpoint that fuses the intron or a portion thereof of COL3A1 to the exon or a portion thereof of ALK. In some embodiments, the COL3A1 breakpoint occurs within an intron or within an exon of COL3A1, e.g., within exon 48, intron 48, exon 2, or intron 2 of COL3A1. In some embodiments, the COL3A1 breakpoint occurs at the 3′ end or at the 5′ end of an intron, or at the 3′ end or at the 5′ end of an exon, of COL3A1, e.g., at the 3′ end of exon 48, at the 5′ end of intron 48, at the 3′ end of intron 48, at the 3′ end of exon 2, at the 5′ end of intron 2, or at the 3′ end of intron 2 of COL3A1. In some embodiments, the ALK breakpoint occurs within an intron or within an exon of ALK, e.g., within intron 18 or exon 19 of ALK. In some embodiments, the ALK breakpoint occurs at the 3′ end or at the 5′ end of an intron, or at the 3′ end or at the 5′ end of an exon of ALK, e.g., at the 5′ end of intron 18, at the 3′ end of intron 18, or at the 5′ end of exon 19 of ALK. In certain embodiments, exon 48 or intron 48, or a portion of exon 48 or intron 48, of COL3A1 is directly fused to intron 18 or exon 19, or a portion of intron 18 or exon 19, of ALK, thereby establishing a COL3A1-ALK breakpoint between the COL3A1 sequence and the ALK sequence. In certain embodiments, exon 2 or intron 2, or a portion of exon 2 or intron 2, of COL3A1 is directly fused to intron 18 or exon 19, or a portion of intron 18 or exon 19, of ALK, thereby establishing a COL3A1-ALK breakpoint between the COL3A1 sequence and the ALK sequence.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 48 or a portion thereof of COL3A1 fused to intron 18 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 48 or a portion thereof of COL3A1 fused to exon 19 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 48 or a portion thereof of COL3A1 fused to intron 18 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 48 or a portion thereof of COL3A1 fused to exon 19 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 2 or a portion thereof of COL3A1 fused to intron 18 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 2 or a portion thereof of COL3A1 fused to exon 19 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 2 or a portion thereof of COL3A1 fused to intron 18 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 2 or a portion thereof of COL3A1 fused to exon 19 or a portion thereof of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL3A1-ALK breakpoint that fuses exon 48 or a portion thereof of COL3A1 to intron 18 or a portion thereof of ALK. In some embodiments, the COL3A1-ALK breakpoint fuses the 3′ end of exon 48 or of the portion thereof of COL3A1 to the 5′ end of intron 18 or of the portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL3A1-ALK breakpoint that fuses exon 48 or a portion thereof of COL3A1 to exon 19 or a portion thereof of ALK. In some embodiments, the COL3A1-ALK breakpoint fuses the 3′ end of exon 48 or of the portion thereof of COL3A1 to the 5′ end of exon 19 or of the portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL3A1-ALK breakpoint that fuses intron 48 or a portion thereof of COL3A1 to intron 18 or a portion thereof of ALK. In some embodiments, the COL3A1-ALK breakpoint fuses the 3′ end of intron 48 or of the portion thereof of COL3A1 to the 5′ end of intron 18 or of the portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL3A1-ALK breakpoint that fuses intron 48 or a portion thereof of COL3A1 to exon 19 or a portion thereof of ALK. In some embodiments, the COL3A1-ALK breakpoint fuses the 3′ end of intron 48 or of the portion thereof of COL3A1 to the 5′ end of exon 19 or of the portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL3A1-ALK breakpoint that fuses exon 2 or a portion thereof of COL3A1 to intron 18 or a portion thereof of ALK. In some embodiments, the COL3A1-ALK breakpoint fuses the 3′ end of exon 2 or of the portion thereof of COL3A1 to the 5′ end of intron 18 or of the portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL3A1-ALK breakpoint that fuses exon 2 or a portion thereof of COL3A1 to exon 19 or a portion thereof of ALK. In some embodiments, the COL3A1-ALK breakpoint fuses the 3′ end of exon 2 or of the portion thereof of COL3A1 to the 5′ end of exon 19 or of the portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL3A1-ALK breakpoint that fuses intron 2 or a portion thereof of COL3A1 to intron 18 or a portion thereof of ALK. In some embodiments, the COL3A1-ALK breakpoint fuses the 3′ end of intron 2 or of the portion thereof of COL3A1 to the 5′ end of intron 18 or of the portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL3A1-ALK breakpoint that fuses intron 2 or a portion thereof of COL3A1 to exon 19 or a portion thereof of ALK. In some embodiments, the COL3A1-ALK breakpoint fuses the 3′ end of intron 2 or of the portion thereof of COL3A1 to the 5′ end of exon 19 or of the portion thereof of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 48 or a portion thereof of COL3A1 fused to an intron or a portion thereof between exon 18 and exon 19 of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof between exon 48 and exon 49 of COL3A1 fused to an intron or a portion thereof between exon 18 and exon 19 of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof between exon 48 and exon 49 of COL3A1 fused to exon 19 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 2 or a portion thereof of COL3A1 fused to an intron or a portion thereof between exon 18 and exon 19 of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof between exon 2 and exon 3 of COL3A1 fused to an intron or a portion thereof between exon 18 and exon 19 of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising an intron or a portion thereof between exon 2 and exon 3 of COL3A1 fused to exon 19 or a portion thereof of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 48 or a portion thereof of COL3A1 and intron 18 or a portion thereof of ALK, and a COL3A1-ALK breakpoint that fuses exon 48 or a portion thereof of COL3A1 and intron 18 or a portion thereof of ALK. In some embodiments, the 3′ end of exon 48 or of a portion of exon 48 of COL3A1 is fused to the 5′ end of intron 18 or of a portion of intron 18 of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 48 or a portion thereof of COL3A1 fused to exon 19 or a portion thereof of ALK, and a COL3A1-ALK breakpoint that fuses exon 48 or a portion thereof of COL3A1 and exon 19 or a portion thereof of ALK. In some embodiments, the 3′ end of exon 48 or of a portion of exon 48 of COL3A1 is fused to the 5′ end of exon 19 or of a portion of exon 19 of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 48 or a portion thereof of COL3A1 fused to intron 18 or a portion thereof of ALK, and a COL3A1-ALK breakpoint that fuses intron 48 or a portion thereof of COL3A1 and intron 18 or a portion thereof of ALK. In some embodiments, the 3′ end of intron 48 or of a portion of intron 48 of COL3A1 is fused to the 5′ end of intron 18 or of a portion of intron 18 of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 48 or a portion thereof of COL3A1 fused to exon 19 or a portion thereof of ALK, and a COL3A1-ALK breakpoint that fuses intron 48 or a portion thereof of COL3A1 and exon 19 or a portion thereof of ALK. In some embodiments, the 3′ end of intron 48 or of a portion of intron 48 of COL3A1 is fused to the 5′ end of exon 19 or of a portion of exon 19 of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 2 or a portion thereof of COL3A1 fused to intron 18 or a portion thereof of ALK, and a COL3A1-ALK breakpoint that fuses exon 2 or a portion thereof of COL3A1 and intron 18 or a portion thereof of ALK. In some embodiments, the 3′ end of exon 2 or of a portion of exon 2 of COL3A1 is fused to the 5′ end of intron 18 or of a portion of intron 18 of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising exon 2 or a portion thereof of COL3A1 fused to exon 19 or a portion thereof of ALK, and a COL3A1-ALK breakpoint that fuses exon 2 or a portion thereof of COL3A1 and exon 19 or a portion thereof of ALK. In some embodiments, the 3′ end of exon 2 or of a portion of exon 2 of COL3A1 is fused to the 5′ end of exon 19 or of a portion of exon 19 of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 2 or a portion thereof of COL3A1 fused to intron 18 or a portion thereof of ALK, and a COL3A1-ALK breakpoint that fuses intron 2 or a portion thereof of COL3A1 and intron 18 or a portion thereof of ALK. In some embodiments, the 3′ end of intron 2 or of a portion of intron 2 of COL3A1 is fused to the 5′ end of intron 18 or of a portion of intron 18 of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising intron 2 or a portion thereof of COL3A1 fused to exon 19 or a portion thereof of ALK, and a COL3A1-ALK breakpoint that fuses intron 2 or a portion thereof of COL3A1 and exon 19 or a portion thereof of ALK. In some embodiments, the 3′ end of intron 2 or of a portion of intron 2 of COL3A1 is fused to the 5′ end of exon 19 or of a portion of exon 19 of ALK.

In some embodiments, the exon-exon fusions or exon-intron fusions are in-frame fusions. The fusion breakpoint may occur anywhere within an exon or an intron of COL3A1 (e.g., exon 48, intron 48, exon 2, or intron 2), and anywhere within an exon or an intron of ALK (e.g., intron 18 or exon 19). When a breakpoint occurs in an intron of COL3A1, the resulting mRNA sequence, and the resulting amino acid sequence, has a breakpoint or fusion junction between the preceding exon of COL3A1 and the sequence of ALK. When a breakpoint occurs in an intron of ALK, the resulting mRNA sequence, and the resulting amino acid sequence, has a breakpoint or fusion junction between the following exon of ALK and the sequence of COL3A1. When the breakpoint or fusion junction occurs between an intron of COL3A1 and an intron of ALK, the resulting mRNA sequence, and the resulting amino acid sequence, has a breakpoint or fusion junction between the preceding exon of COL3A1 and the following exon of ALK. For example, a fusion of intron 48 of COL3A1 and intron 18 of ALK in the DNA sequence would result in an mRNA sequence, and in an amino acid sequence, having a breakpoint or fusion junction between exon 48 of COL3A1 and exon 19 of ALK. In another example, a fusion of intron 2 of COL3A1 and intron 18 of ALK in the DNA sequence would result in an mRNA sequence, and in an amino acid sequence, having a breakpoint or fusion junction between exon 2 of COL3A1 and exon 19 of ALK. One skilled in the art could readily determine the exon and intron sequences within the COL3A1 and ALK genes, and the corresponding mRNA and amino acid sequences, for example using an NCBI database (e.g., GenBank).

In some embodiments, the COL3A1-ALK fusion polypeptide comprises an amino acid sequence encoded by a nucleic acid comprising 5 or more, 10 or more, or 20 or more nucleotides on the 5′ end of the COL3A1-ALK breakpoint, and 5 or more, 10 or more, or 20 or more nucleotides on the 3′ end of the COL3A1-ALK breakpoint. In some embodiments, the COL3A1-ALK fusion polypeptide comprises an amino acid sequence encoded by a nucleic acid comprising 5 or more nucleotides from exon 48 or intron 48 of COL3A1 on the 5′ end of the COL3A1-ALK breakpoint, and 5 or more nucleotides from intron 18 or exon 19 of ALK on the 3′ end of the COL3A1-ALK breakpoint. In some embodiments, the COL3A1-ALK fusion polypeptide comprises an amino acid sequence encoded by a nucleic acid comprising 5 or more nucleotides from exon 2 or intron 2 of COL3A1 on the 5′ end of the COL3A1-ALK breakpoint, and 5 or more nucleotides from intron 18 or exon 19 of ALK on the 3′ end of the COL3A1-ALK breakpoint.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by an mRNA molecule comprising a fusion, e.g., an in-frame fusion, of exon 48 or a portion thereof of COL3A1 fused to exon 19 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by an mRNA molecule comprising a fusion, e.g., an in-frame fusion, of exon 2 or a portion thereof of COL3A1 fused to exon 19 or a portion thereof of ALK.

In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a COL3A1-ALK breakpoint resulting in an in-frame fusion of an exon described herein or a portion thereof of COL3A1 with an exon described herein or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by an mRNA molecule comprising an in-frame fusion of an exon described herein or a portion thereof of COL3A1 to an exon described herein or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by an mRNA molecule comprising a fusion, e.g., an in-frame fusion, of exon 48 or a portion thereof of COL3A1 fused to exon 19 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by an mRNA molecule comprising a fusion, e.g., an in-frame fusion, of exon 2 or a portion thereof of COL3A1 fused to exon 19 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a cDNA molecule comprising a fusion, e.g., an in-frame fusion, of exon 48 or a portion thereof of COL3A1 fused to exon 19 or a portion thereof of ALK. In some embodiments, a COL3A1-ALK fusion polypeptide provided herein comprises an amino acid sequence at least about 85%, at least about 90%, at least about 95%, at least about 96%, at least about 97%, at least about 98%, at least about 99%, or 100% identical to an amino acid sequence encoded by a cDNA molecule comprising a fusion, e.g., an in-frame fusion, of exon 2 or a portion thereof of COL3A1 fused to exon 19 or a portion thereof of ALK.

In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a nucleotide sequence comprising, in the 5′ to 3′ direction, a fusion of exon 48 or a portion thereof of COL3A1 to exon 19 or a portion thereof of ALK. In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a nucleotide sequence comprising, in the 5′ to 3′ direction, a fusion of exon 2 or a portion thereof of COL3A1 to exon 19 or a portion thereof of ALK.

In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a nucleotide sequence comprising, in the 5′ to 3′ direction, exons 1-47 and exon 48 or a portion thereof of COL3A1, and exon 19 or a portion thereof and exons 20-29 of ALK. In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising a nucleotide sequence comprising, in the 5′ to 3′ direction, exon 1 and exon 2 or a portion thereof of COL3A1, and exon 19 or a portion thereof and exons 20-29 of ALK.

In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a fusion nucleic acid molecule resulting from a breakpoint in exon 2 or intron 2 of COL3A1, and in intron 18 or exon 19 of ALK. In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a fusion nucleic acid molecule resulting from a breakpoint joining Chr2:189849674 with Chr2:29448496. In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a fusion nucleic acid molecule resulting from a breakpoint in exon 48 or intron 48 of COL3A1, and in intron 18 or exon 19 of ALK. In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a fusion nucleic acid molecule resulting from a breakpoint joining Chr2:189874528 with Chr2:29448490. In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a fusion nucleic acid molecule resulting from a breakpoint joining Chr2:189874814 with Chr2:29449440. In some embodiments, the chromosome positions correspond to chromosome positions of human genome version hg19.

In some embodiments, the COL3A1-ALK fusion polypeptide comprises 5 or more amino acids (e.g., any of 5 or more, 10 or more, 15 or more, or 20 or more amino acids) encoded by the 3′ end of exon 48 or a portion thereof of COL3A1, fused to 5 or more amino acids (e.g., any of 5 or more, 10 or more, 15 or more, or 20 or more amino acids) encoded by the 5′ end of exon 19 or a portion thereof of ALK. In some embodiments, the COL3A1-ALK fusion polypeptide comprises 5 or more amino acids (e.g., any of 5 or more, 10 or more, 15 or more, or 20 or more amino acids) encoded by the 3′ end of exon 2 or a portion thereof of COL3A1, fused to 5 or more amino acids (e.g., any of 5 or more, 10 or more, 15 or more, or 20 or more amino acids) encoded by the 5′ end of exon 19 or a portion thereof of ALK.

In some embodiments, a COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising at least a portion of a COL3A1 sequence of SEQ ID NO: 3 and at least a portion of an ALK sequence of SEQ ID NO: 1, or a sequence having at least about 85% (e.g., any of at least about 85%, at least about 90%, at least about 95%, at least about 96%, at least about 97%, at least about 98%, at least about 99%, or 100%) sequence identity to the portion of the COL3A1 sequence of SEQ ID NO: 3 and/or the portion of the ALK sequence of SEQ ID NO: 1.

In some embodiments, a COL3A1-ALK fusion polypeptide comprises an amino acid sequence comprising at least a portion of a COL3A1 sequence of SEQ ID NO: 6 and at least a portion of an ALK sequence of SEQ ID NO: 4, or a sequence having at least about 85% (e.g., any of at least about 85%, at least about 90%, at least about 95%, at least about 96%, at least about 97%, at least about 98%, at least about 99%, or 100%) sequence identity to the portion of the COL3A1 sequence of SEQ ID NO: 6 and/or the portion of the ALK sequence of SEQ ID NO: 4.

In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising the nucleotide sequence of SEQ ID NO: 8, or a nucleotide sequence at least 70%, 75%, 80%, 85%, 90%, 91%, 92%, 93%, 94%, 95%, 96%, 97%, 98%, 99%, or 99.5% identical to the nucleotide sequence of SEQ ID NO: 8.

In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to an amino acid sequence encoded by a nucleic acid comprising the nucleotide sequence of SEQ ID NO: 9, or a nucleotide sequence at least 70%, 75%, 80%, 85%, 90%, 91%, 92%, 93%, 94%, 95%, 96%, 97%, 98%, 99%, or 99.5% identical to the nucleotide sequence of SEQ ID NO: 9.

In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to the amino acid sequence of SEQ ID NO: 11. In some embodiments, the COL3A1-ALK fusion polypeptide comprises the amino acid sequence of SEQ ID NO: 11.

(SEQ ID NO: 11)
MMSFVQKGSWLLLALLHPTIILAQQEAVEGGCSHLGQSYADRDVWKPEP
CQICVCDSGSVLCDDIICDDQELDCPNPEIPFGECCAVCPLSPTPEPHL
PLSLILSVVTSALVAALVLAFSGIMIVYRRKHQELQAMQMELQSPEYKL
SKIRTSTIMTDYNPNYCFAGKTSSISDLKEVPRKNITLIRGLGHGAFGE
VYEGQVSGMPNDPSPLQVAVKTLPEVCSEQDELDELMEALIISKFNHQN
IVRCIGVSLQSLPRFILLELMAGGDLKSFLRETRPRPSQPSSLAMLDLL
HVARDIACGCQYLEENHFIHRDIAARNCLLTCPGPGRVAKIGDFGMARD
IYRASYYRKGGCAMLPVKWMPPEAFMEGIFTSKTDTWSFGVLLWEIFSL
GYMPYPSKSNQEVLEFVTSGGRMDPPKNCPGPVYRIMTQCWQHQPEDRP
NFAIILERIEYCTQDPDVINTALPIEYGPLVEEEEKVPVRPKDPEGVPP
LLVSQQAKREEERSPAAPPPLPTTSSGKAAKKPTAAEISVRVPRGPAVE
GGHVNMAFSQSNPPSELHKVHGSRNKPTSLWNPTYGSWFTEKPTKKNNP
IAKKEPHDRGNLGLEGSCTVPPNVATGRLPGASLLLEPSSLTANMKEVP
LFRLRHFPCGNVNYGYQQQGLPLEAATAPGAGHYEDTILKSKNSMNQPG
P

In the sequence of SEQ ID NO: 11 provided above, amino acid sequences corresponding to ALK are underlined, and bolded sequences correspond to a novel amino acid formed by the fusion.

In some embodiments, the COL3A1-ALK fusion polypeptide comprises an amino acid sequence at least about 85%, at least about 90%, at least about 95%, at least about 96%, at least about 97%, at least about 98%, at least about 99%, or 100% identical to the amino acid sequence of SEQ ID NO: 12. In some embodiments, the COL3A1-ALK fusion polypeptide comprises the amino acid sequence of SEQ ID NO: 12.

(SEQ ID NO: 12)
MMSFVQKGSWLLLALLHPTIILAQQEAVEGGCSHLGQSYADRDVWKPEP
CQICVCDSGSVLCDDIICDDQELDCPNPEIPFGECCAVCPQPPTAPTRP
PNGQGPQGPKGDPGPPGIPGRNGDPGIPGQPGSPGSPGPPGICESCPTG
PQNYSPQYDSYDVKSGVAVGGLAGYPGPAGPPGPPGPPGTSGHPGSPGS
PGYQGPPGEPGQAGPSGPPGPPGAIGPSGPAGKDGESGRPGRPGERGLP
GPPGIKGPAGIPGFPGMKGHRGFDGRNGEKGETGAPGLKGENGLPGENG
APGPMGPRGAPGERGRPGLPGAAGARGNDGARGSDGQPGPPGPPGTAGF
PGSPGAKGEVGPAGSPGSNGAPGQRGEPGPQGHAGAQGPPGPPGINGSP
GGKGEMGPAGIPGAPGLMGARGPPGPAGANGAPGLRGGAGEPGKNGAKG
EPGPRGERGEAGIPGVPGAKGEDGKDGSPGEPGANGLPGAAGERGAPGF
RGPAGPNGIPGEKGPAGERGAPGPAGPRGAAGEPGRDGVPGGPGMRGMP
GSPGGPGSDGKPGPPGSQGESGRPGPPGPSGPRGQPGVMGFPGPKGNDG
APGKNGERGGPGGPGPQGPPGKNGETGPQGPPGPTGPGGDKGDTGPPGP
QGLQGLPGTGGPPGENGKPGEPGPKGDAGAPGAPGGKGDAGAPGERGPP
GLAGAPGLRGGAGPPGPEGGKGAAGPPGPPGAAGTPGLQGMPGERGGLG
SPGPKGDKGEPGGPGADGVPGKDGPRGPTGPIGPPGPAGQPGDKGEGGA
PGLPGIAGPRGSPGERGETGPPGPAGFPGAPGQNGEPGGKGERGAPGEK
GEGGPPGVAGPPGGSGPAGPPGPQGVKGERGSPGGPGAAGFPGARGLPG
PPGSNGNPGPPGPSGSPGKDGPPGPAGNTGAPGSPGVSGPKGDAGQPGE
KGSPGAQGPPGAPGPLGIAGITGARGLAGPPGMPGPRGSPGPQGVKGES
GKPGANGLSGERGPPGPQGLPGLAGTAGEPGRDGNPGSDGLPGRDGSPG
GKGDRGENGSPGAPGAPGHPGPPGPVGPAGKSGDRGESGPAGPAGAPGP
AGSRGAPGPQGPRGDKGETGERGAAGIKGHRGFPGNPGAPGSPGPAGQQ
GAIGSPGPAGPRGPVGPSGPPGKDGTSGHPGPIGPPGPRGNRGERGSEG
SPGHPGQPGPPGPPGAPGPCCGGVGAAAIAGIGGEKAGGFAPYYGDEPM
DFKINTDEIMTSLKSVNGQIESLISPDGSRKNPARNCRDLKFCHPELKS
VSPTPEPHLPLSLILSVVTSALVAALVLAFSGIMIVYRRKHQELQAMQM
ELQSPEYKLSKLRTSTIMTDYNPNYCFAGKTSSISDLKEVPRKNITLIR
GLGHGAFGEVYEGQVSGMPNDPSPLQVAVKTLPEVCSEQDELDFLMEAL
IISKFNHQNIVRCIGVSLQSLPRFILLELMAGGDLKSFLRETRPRPSQP
SSLAMLDLLHVARDIACGCQYLEENHFIHRDIAARNCLLTCPGPGRVAK
IGDFGMARDIYRASYYRKGGCAMLPVKWMPPEAFMEGIFTSKTDTWSFG
VLLWEIFSLGYMPYPSKSNQEVLEFVTSGGRMDPPKNCPGPVYRIMTQC
WQHQPEDRPNFAIILERIEYCTQDPDVINTALPIEYGPLVEEEEKVPVR
PKDPEGVPPLLVSQQAKREEERSPAAPPPLPTTSSGKAAKKPTAAEISV
RVPRGPAVEGGHVNMAFSQSNPPSELHKVHGSRNKPTSLWNPTYGSWFT
EKPTKKNNPIAKKEPHDRGNLGLEGSCTVPPNVATGRLPGASLLLEPSS
LTANMKEVPLFRLRHFPCGNVNYGYQQQGLPLEAATAPGAGHYEDTILK
SKNSMNQPGP

In the sequence of SEQ ID NO: 12 provided above, amino acid sequences corresponding to ALK are underlined, and bolded sequences correspond to a novel amino acid formed by the fusion.

In some embodiments, the COL3A1-ALK fusion polypeptide is isolated from cells or tissue sources according to methods known in the art. In some embodiments, a fusion polypeptide provided herein can be synthesized chemically using standard peptide synthesis techniques. In some embodiments, a fusion polypeptide provided herein is isolated or purified such that it is substantially free of cellular material or other contaminating proteins from the cell or tissue source from which the protein is derived, or substantially free of chemical precursors or other chemicals when chemically synthesized.

In some embodiments, the COL3A1-ALK fusion polypeptide is fused to a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, Green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, Strep-tag, and Nus-tag. In some embodiments, the label or tag comprises a detection agent, such as a fluorescent molecule or an affinity reagent or tag.

In some embodiments, the COL3A1-ALK fusion polypeptide has a kinase activity, such as an ALK kinase activity or a tyrosine kinase activity. Methods of assessing kinase activity are known in the art and include, without limitation, using radioactivity-based assays (e.g., using ³²P-orthophosphate or other suitable reagents) in combination with SDS-PAGE, 2-dimensional gel electrophoresis, phosphorylation state-specific antibodies, Western blots, enzyme-linked immunosorbent assays (ELISA), mass spectrometry, immunohistochemistry, and flow cytometry.

### Methods of Detecting ALK Fusions

In some aspects, provided herein are methods of detecting the presence of a COL5A2-ALK fusion or a COL3A1-ALK fusion described herein, e.g., in a sample. In some embodiments, the methods of detecting the presence of a COL5A2-ALK fusion or a COL3A1-ALK fusion described herein comprise detecting a COL5A2-ALK fusion nucleic acid molecule or a COL3A1-ALK fusion nucleic acid molecule described herein in a sample. In some embodiments, the methods of detecting the presence of a COL5A2-ALK fusion or a COL3A1-ALK fusion described herein comprise detecting a COL5A2-ALK fusion polypeptide or a COL3A1-ALK fusion polypeptide described herein in a sample. In some embodiments, the sample is obtained from an individual, such as an individual having a cancer, e.g., a cancer described herein. In some embodiments, the methods of detecting the presence of a COL5A2-ALK fusion nucleic acid molecule or a COL3A1-ALK fusion nucleic acid molecule described herein comprise selectively enriching for one or more nucleic acids comprising COL5A2, COL3A1, or ALK nucleotide sequences to produce an enriched sample, e.g., using a reagent known in the art or provided herein, such as a bait, probe, or oligonucleotide described herein. In some embodiments, the methods of detecting the presence of a COL5A2-ALK fusion polypeptide or a COL3A1-ALK fusion polypeptide described herein comprise selectively enriching for one or more polypeptides comprising COL5A2, COL3A1, or ALK amino acid sequences to produce an enriched sample, e.g., using a reagent known in the art or provided herein, such as an antibody described herein.

Detection of ALK Fusion Nucleic Acids

Provided herein are methods of detecting a COL5A2-ALK fusion nucleic acid molecule of the disclosure, or a COL3A1-ALK fusion nucleic acid molecule of the disclosure.

In some embodiments, a COL5A2-ALK fusion nucleic acid molecule of the disclosure, or a COL3A1-ALK fusion nucleic acid molecule of the disclosure is detected using any suitable method known in the art, such as a nucleic acid hybridization assay, an amplification-based assay (e.g., polymerase chain reaction, PCR), a PCR-RFLP assay, real-time PCR, sequencing (e.g., Sanger sequencing or next-generation sequencing), a screening analysis (e.g., using karyotype methods), fluorescence in situ hybridization (FISH), break away FISH, spectral karyotyping, multiplex-FISH, comparative genomic hybridization, in situ hybridization, single specific primer-polymerase chain reaction (SSP-PCR), high performance liquid chromatography (HPLC), or mass-spectrometric genotyping. Methods of analyzing samples, e.g., to detect a fusion nucleic acid molecule, are described in U.S. Pat. No. 9,340,830 and in WO2012092426A1, which are hereby incorporated by reference in their entirety.

In Situ Hybridization Methods

In some embodiments, a COL5A2-ALK fusion nucleic acid molecule of the disclosure, or a COL3A1-ALK fusion nucleic acid molecule of the disclosure is detected using an in situ hybridization method, such as a fluorescence in situ hybridization (FISH) method.

In some embodiments, FISH analysis is used to identify the chromosomal rearrangement resulting in the fusions as described herein. In some embodiments, FISH analysis is used to identify an RNA molecule comprising a COL5A2-ALK or a COL3A1-ALK breakpoint described herein. Methods for performing FISH are known in the art and can be used in nearly any type of tissue. In FISH analysis, nucleic acid probes which are detectably labeled, e.g. fluorescently labeled, are allowed to bind to specific regions of DNA, e.g., a chromosome, or an RNA, e.g., an mRNA, and then examined, e.g., through a microscope. See, for example, U.S. Pat. No. 5,776,688. DNA or RNA molecules are first fixed onto a slide, the labeled probe is then hybridized to the DNA or RNA molecules, and then visualization is achieved, e.g., using enzyme-linked label-based detection methods known in the art. Generally, the resolution of FISH analysis is on the order of detection of 60 to 100000 nucleotides, e.g., 60 base pairs (bp) up to 100 kilobase pairs of DNA. Nucleic acid probes used in FISH analysis comprise single stranded nucleic acids. Such probes are typically at least about 50 nucleotides in length. In some embodiments, probes comprise about 100 to about 500 nucleotides. Probes that hybridize with centromeric DNA and locus-specific DNA or RNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA or other sources of nucleic acids through standard techniques. Examples of probes, labeling and hybridization methods are known in the art.

Several variations of FISH methods are known in the art and are suitable for use according to the methods of the disclosure, including single-molecule RNA FISH, Fiber FISH, Q-FISH, Flow-FISH, MA-FISH, break-away FISH, hybrid fusion-FISH, and multi-fluor FISH or mFISH. In some embodiments, “break-away FISH” is used in the methods provided herein. In break-away FISH, at least one probe targeting a fusion junction or breakpoint and at least one probe targeting an individual gene of the fusion, e.g., at one or more exons and or introns of the gene, are utilized. In normal cells (i.e., cells not having a fusion nucleic acid molecule described herein), both probes are observed (or a secondary color is observed due to the close proximity of the two genes of the gene fusion); and in cells having a fusion nucleic acid molecule described herein, only a single gene probe is observed due to the presence of a rearrangement resulting in the fusion nucleic acid molecule.

Array-Based Methods

In some embodiments, a COL5A2-ALK fusion nucleic acid molecule of the disclosure, or a COL3A1-ALK fusion nucleic acid molecule of the disclosure is detected using an array-based method, such as array-based comparative genomic hybridization (CGH) methods. In array-based CGH methods, a first sample of nucleic acids (e.g., from a sample, such as from a tumor) is labeled with a first label, while a second sample of nucleic acids (e.g., a control, such as from a healthy cell/tissue) is labeled with a second label. In some embodiments, equal quantities of the two samples are mixed and co-hybridized to a DNA microarray of several thousand evenly spaced cloned DNA fragments or oligonucleotides, which have been spotted in triplicate on the array. After hybridization, digital imaging systems are used to capture and quantify the relative fluorescence intensities of each of the hybridized fluorophores. The resulting ratio of the fluorescence intensities is proportional to the ratio of the copy numbers of DNA sequences in the two samples. In some embodiments, where there are chromosomal deletions or multiplications, differences in the ratio of the signals from the two labels are detected and the ratio provides a measure of the copy number. Array-based CGH can also be performed with single-color labeling. In single color CGH, a control (e.g., control nucleic acid sample, such as from a healthy cell/tissue) is labeled and hybridized to one array and absolute signals are read, and a test sample (e.g., a nucleic acid sample obtained from an individual or from a tumor) is labeled and hybridized to a second array (with identical content) and absolute signals are read. Copy number differences are calculated based on absolute signals from the two arrays.

Amplification-Based Methods

In some embodiments, a COL5A2-ALK fusion nucleic acid molecule of the disclosure, or a COL3A1-ALK fusion nucleic acid molecule of the disclosure is detected using an amplification-based method. As is known in the art, in such amplification-based methods, a sample of nucleic acids, such as a sample obtained from an individual or from a tumor, is used as a template in an amplification reaction (e.g., Polymerase Chain Reaction (PCR)) using one or more oligonucleotides or primers, e.g., such as one or more oligonucleotides or primers provided herein. The presence of a COL5A2-ALK fusion nucleic acid molecule of the disclosure, or of a COL3A1-ALK fusion nucleic acid molecule of the disclosure in the sample can be determined based on the presence or absence of an amplification product. Quantitative amplification methods are also known in the art and may be used according to the methods provided herein. Methods of measurement of DNA copy number at microsatellite loci using quantitative PCR analysis are known in the art. The known nucleotide sequence for genes is sufficient to enable one of skill in the art to routinely select primers to amplify any portion of the gene. Fluorogenic quantitative PCR can also be used. In fluorogenic quantitative PCR, quantitation is based on the amount of fluorescence signals, e.g., TaqMan and Sybr green.

Other amplification methods suitable for use according to the methods provided herein include, e.g., ligase chain reaction (LCR), transcription amplification, self-sustained sequence replication, dot PCR, and linker adapter PCR.

Sequencing

In some embodiments, a COL5A2-ALK fusion nucleic acid molecule of the disclosure, or a COL3A1-ALK fusion nucleic acid molecule of the disclosure is detected using a sequencing method. Any method of sequencing known in the art can be used to detect a fusion nucleic acid molecule provided herein. Exemplary sequencing methods that may be used to detect a fusion nucleic acid molecule provided herein include those based on techniques developed by Maxam and Gilbert or Sanger. Automated sequencing procedures may also be used, e.g., including sequencing by mass spectrometry.

In some embodiments, a COL5A2-ALK fusion nucleic acid molecule of the disclosure, or a COL3A1-ALK fusion nucleic acid molecule of the disclosure is detected using hybrid capture-based sequencing (hybrid capture-based NGS), e.g., using adaptor ligation-based libraries. See, e.g., Frampton, G. M. et al. (2013) Nat. Biotech. 31:1023-1031. In some embodiments, a COL5A2-ALK fusion nucleic acid molecule of the disclosure, or a COL3A1-ALK fusion nucleic acid molecule of the disclosure is detected using next-generation sequencing (NGS). Next-generation sequencing includes any sequencing method that determines the nucleotide sequence of either individual nucleic acid molecules or clonally expanded proxies for individual nucleic acid molecules in a highly parallel fashion (e.g., greater than 105 molecules may be sequenced simultaneously). Next generation sequencing methods suitable for use according to the methods provided herein are known in the art and include, without limitation, massively parallel short-read sequencing, template-based sequencing, pyrosequencing, real-time sequencing comprising imaging the continuous incorporation of dye-labeling nucleotides during DNA synthesis, nanopore sequencing, sequencing by hybridization, nano-transistor array based sequencing, polony sequencing, scanning tunneling microscopy (STM)-based sequencing, or nanowire-molecule sensor based sequencing. See, e.g., Metzker, M. (2010) Nature Biotechnology Reviews 11:31-46, which is hereby incorporated by reference. Exemplary NGS methods and platforms that may be used to detect a fusion nucleic acid molecule provided herein include, without limitation, the HeliScope Gene Sequencing system from Helicos BioSciences (Cambridge, MA., USA), the PacBio RS system from Pacific Biosciences (Menlo Park, CA, USA), massively parallel short-read sequencing such as the Solexa sequencer and other methods and platforms from Illumina Inc. (San Diego, CA, USA), 454 sequencing from 454 LifeSciences (Branford, CT, USA), Ion Torrent sequencing from ThermoFisher (Waltham, MA, USA), or the SOLiD sequencer from Applied Biosystems (Foster City, CA, USA). Additional exemplary methods and platforms that may be used to detect a fusion nucleic acid molecule provided herein include, without limitation, the Genome Sequencer (GS) FLX System from Roche (Basel, CHE), the G.007 polonator system, the Solexa Genome Analyzer, HiSeq 2500, HiSeq3000, HiSeq 4000, and NovaSeq 6000 platforms from Illumina Inc. (San Diego, CA, USA).

Detection Reagents

In some aspects, provided herein are reagents for detecting a COL5A2-ALK fusion nucleic acid molecule of the disclosure or a COL3A1-ALK fusion nucleic acid molecule of the disclosure, or a fragment thereof, e.g., according to the methods of detection provided herein. In some embodiments, a detection reagent provided herein comprises a nucleic acid molecule, e.g., a DNA, RNA, or mixed DNA/RNA molecule, comprising a nucleotide sequence that is complementary to a nucleotide sequence on a target nucleic acid, e.g., a nucleic acid that comprises a fusion nucleic acid molecule described herein or a fragment or portion thereof.

Baits

Provided herein are baits suitable for the detection of a COL5A2-ALK fusion nucleic acid molecule or a COL3A1-ALK fusion nucleic acid molecule of the disclosure.

In some embodiments, the bait comprises a capture nucleic acid molecule configured to hybridize to a target nucleic acid molecule comprising a COL5A2-ALK fusion nucleic acid molecule or a COL3A1-ALK fusion nucleic acid molecule provided herein, or a fragment or portion thereof. In some embodiments, the capture nucleic acid molecule is configured to hybridize to the COL5A2-ALK fusion nucleic acid molecule or to the COL3A1-ALK fusion nucleic acid molecule of the target nucleic acid molecule.

In some embodiments, the capture nucleic acid molecule is configured to hybridize to a fragment of the COL5A2-ALK fusion nucleic acid molecule or the COL3A1-ALK fusion nucleic acid molecule of the target nucleic acid molecule. In some embodiments, the fragment comprises (or is) between about 5 and about 25 nucleotides, between about 5 and about 300 nucleotides, between about 100 and about 300 nucleotides, between about 130 and about 230 nucleotides, or between about 150 and about 200 nucleotides. In some embodiments, the capture nucleic acid molecule is between about 5 and about 25 nucleotides, between about 5 and about 300 nucleotides, between about 100 and about 300 nucleotides, between about 130 and about 230 nucleotides, or between about 150 and about 200 nucleotides. In some embodiments, the fragment comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length. In some embodiments, the capture nucleic acid molecule comprises (or is) about 100 nucleotides, about 125 nucleotides, about 150 nucleotides, about 175 nucleotides, about 200 nucleotides, about 225 nucleotides, about 250 nucleotides, about 275 nucleotides, or about 300 nucleotides in length.

In some embodiments, the capture nucleic acid molecule is configured to hybridize to a COL5A2-ALK breakpoint or a COL3A1-ALK breakpoint, and may further hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the COL5A2-ALK breakpoint or the COL3A1-ALK breakpoint.

In some embodiments, the capture nucleic acid molecule is configured to hybridize to a nucleotide sequence in an intron or an exon of COL5A2 or ALK, or in a COL5A2-ALK breakpoint joining the introns or exons of COL5A2 and ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid molecule is configured to hybridize to the COL5A2-ALK breakpoint joining an intron of COL5A2 and an intron of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid molecule is configured to hybridize to the COL5A2-ALK breakpoint joining an intron of COL5A2 and an exon of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid molecule is configured to hybridize to the COL5A2-ALK breakpoint joining an exon of COL5A2 and an exon of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid molecule is configured to hybridize to the COL5A2-ALK breakpoint joining an exon of COL5A2 and an intron of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the capture nucleic acid molecule is configured to hybridize to a nucleotide sequence in an intron or an exon of COL3A1 or ALK, or in a COL3A1-ALK breakpoint joining the introns or exons of COL3A1 and ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid molecule is configured to hybridize to the COL3A1-ALK breakpoint joining an intron of COL3A1 and an intron of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid molecule is configured to hybridize to the COL3A1-ALK breakpoint joining an intron of COL3A1 and an exon of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid molecule is configured to hybridize to the COL3A1-ALK breakpoint joining an exon of COL3A1 and an exon of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the capture nucleic acid molecule is configured to hybridize to the COL3A1-ALK breakpoint joining an exon of COL3A1 and an intron of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, a capture nucleic acid molecule provided herein hybridizes to the COL5A2-ALK breakpoint between exon 1 of COL5A2 and intron 5 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL5A2-ALK breakpoint between intron 1 of COL5A2 and intron 5 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL5A2-ALK breakpoint between exon 1 of COL5A2 and exon 6 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL5A2-ALK breakpoint between intron 1 of COL5A2 and exon 6 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, a capture nucleic acid molecule provided herein hybridizes to the COL3A1-ALK breakpoint between exon 48 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL3A1-ALK breakpoint between exon 48 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL3A1-ALK breakpoint between intron 48 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL3A1-ALK breakpoint between intron 48 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL3A1-ALK breakpoint between exon 2 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL3A1-ALK breakpoint between exon 2 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL3A1-ALK breakpoint between intron 2 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a capture nucleic acid molecule provided herein hybridizes to the COL3A1-ALK breakpoint between intron 2 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the capture nucleic acid molecule is a DNA, RNA, or a DNA/RNA molecule. In some embodiments, the capture nucleic acid molecule comprises any of between about 50 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the capture nucleic acid molecule comprises any of between about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the capture nucleic acid molecule comprises about 150 nucleotides. In some embodiments, the capture nucleic acid molecule is about 150 nucleotides. In some embodiments, the capture nucleic acid molecule comprises about 170 nucleotides. In some embodiments, the capture nucleic acid molecule is about 170 nucleotides.

In some embodiments, a bait provided herein comprises a DNA, RNA, or a DNA/RNA molecule. In some embodiments, a bait provided herein includes a label or a tag. In some embodiments, the label or tag is a radiolabel, a fluorescent label, an enzymatic label, a sequence tag, biotin, or another ligand. In some embodiments, a bait provided herein includes a detection reagent such as a fluorescent marker. In some embodiments, a bait provided herein includes (e.g., is conjugated to) an affinity tag, e.g., that allows capture and isolation of a hybrid formed by a bait and a nucleic acid hybridized to the bait. In some embodiments, the affinity tag is an antibody, an antibody fragment, biotin, or any other suitable affinity tag or reagent known in the art. In some embodiments, a bait is suitable for solution phase hybridization.

Baits can be produced and used according to methods known in the art, e.g., as described in WO2012092426A1 and/or or in Frampton et al (2013) Nat Biotechnol, 31:1023-1031, incorporated herein by reference. For example, biotinylated baits (e.g., RNA baits) can be produced by obtaining a pool of synthetic long oligonucleotides, originally synthesized on a microarray, and amplifying the oligonucleotides to produce the bait sequences. In some embodiments, the baits are produced by adding an RNA polymerase promoter sequence at one end of the bait sequences, and synthesizing RNA sequences using RNA polymerase. In one embodiment, libraries of synthetic oligodeoxynucleotides can be obtained from commercial suppliers, such as Agilent Technologies, Inc., and amplified using known nucleic acid amplification methods.

In some embodiments, a bait provided herein is between about 100 nucleotides and about 300 nucleotides. In some embodiments, a bait provided herein is between about 130 nucleotides and about 230 nucleotides. In some embodiments, a bait provided herein is between about 150 nucleotides and about 200 nucleotides. In some embodiments, a bait provided herein comprises a target-specific bait sequence (e.g., a capture nucleic acid molecule described herein) and universal tails on each end. In some embodiments, the target-specific sequence, e.g., a capture nucleic acid molecule described herein, is between about 40 nucleotides and about 300 nucleotides. In some embodiments, the target-specific sequence, e.g., a capture nucleic acid molecule described herein, is between about 100 nucleotides and about 200 nucleotides. In some embodiments, the target-specific sequence, e.g., a capture nucleic acid molecule described herein, is between about 120 nucleotides and about 170 nucleotides. In some embodiments, the target-specific sequence, e.g., a capture nucleic acid molecule described herein, is about 150 nucleotides or about 170 nucleotides. In some embodiments, a bait provided herein comprises an oligonucleotide comprising about 200 nucleotides, of which about 150 nucleotides or about 170 nucleotides are target-specific (e.g., a capture nucleic acid molecule described herein), and the other 50 nucleotides or 30 nucleotides (e.g., 25 or 15 nucleotides on each end of the bait) are universal arbitrary tails, e.g., suitable for PCR amplification.

In some embodiments, a bait provided herein hybridizes to a nucleotide sequence comprising a nucleotide sequence in an intron or an exon of one gene of a fusion molecule described herein (e.g., COL5A2 or COL3A1), in an intron or an exon of the other gene of a fusion molecule described herein (e.g., ALK), and/or a COL5A2-ALK or COL3A1-ALK breakpoint joining the introns and/or exons. In some embodiments, a bait provided herein hybridizes to a nucleotide sequence comprising a nucleotide sequence in an intron of one gene of a fusion molecule described herein (e.g., COL5A2 or COL3A1), in an intron of the other gene of a fusion molecule described herein (e.g., ALK), or a COL5A2-ALK or COL3A1-ALK breakpoint joining the introns. In some embodiments, a bait provided herein hybridizes to a nucleotide sequence comprising a nucleotide sequence in an intron of one gene of a fusion molecule described herein (e.g., COL5A2 or COL3A1), in an exon of the other gene of a fusion molecule described herein (e.g., ALK), or a COL5A2-ALK or COL3A1-ALK breakpoint joining the intron and exon. In some embodiments, a bait provided herein hybridizes to a nucleotide sequence comprising a nucleotide sequence in an exon of one gene of a fusion molecule described herein (e.g., COL5A2 or COL3A1), in an exon of the other gene of a fusion molecule described herein (e.g., ALK), or a COL5A2-ALK or COL3A1-ALK breakpoint joining the exons. In some embodiments, a bait provided herein hybridizes to a nucleotide sequence comprising a nucleotide sequence in an exon of one gene of a fusion molecule described herein (e.g., COL5A2 or COL3A1), in an intron of the other gene of a fusion molecule described herein (e.g., ALK), or a COL5A2-ALK or COL3A1-ALK breakpoint joining the intron and the exon.

In some embodiments, a bait provided herein hybridizes to the COL5A2-ALK breakpoint between exon 1 of COL5A2 and intron 5 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL5A2-ALK breakpoint between intron 1 of COL5A2 and intron 5 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL5A2-ALK breakpoint between exon 1 of COL5A2 and exon 6 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL5A2-ALK breakpoint between intron 1 of COL5A2 and exon 6 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, a bait provided herein hybridizes to the COL3A1-ALK breakpoint between exon 48 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL3A1-ALK breakpoint between exon 48 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL3A1-ALK breakpoint between intron 48 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL3A1-ALK breakpoint between intron 48 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL3A1-ALK breakpoint between exon 2 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL3A1-ALK breakpoint between exon 2 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL3A1-ALK breakpoint between intron 2 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, a bait provided herein hybridizes to the COL3A1-ALK breakpoint between intron 2 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

The baits described herein can be used for selection of exons and short target sequences.

In some embodiments, a bait of the disclosure distinguishes a nucleic acid, e.g., a genomic or transcribed nucleic acid, e.g., a cDNA or RNA, having a COL3A1-ALK or a COL5A2-ALK breakpoint described herein, from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL5A2-ALK breakpoint between exon 1 of COL5A2 and intron 5 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL5A2-ALK breakpoint between intron 1 of COL5A2 and intron 5 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL5A2-ALK breakpoint between exon 1 of COL5A2 and exon 6 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL5A2-ALK breakpoint between intron 1 of COL5A2 and exon 6 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL3A1-ALK breakpoint between exon 48 of COL3A1 and intron 18 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL3A1-ALK breakpoint between exon 48 of COL3A1 and exon 19 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL3A1-ALK breakpoint between intron 48 of COL3A1 and intron 18 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL3A1-ALK breakpoint between intron 48 of COL3A1 and exon 19 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL3A1-ALK breakpoint between exon 2 of COL3A1 and intron 18 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL3A1-ALK breakpoint between exon 2 of COL3A1 and exon 19 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL3A1-ALK breakpoint between intron 2 of COL3A1 and intron 18 of ALK from a reference nucleotide sequence, e.g., a nucleotide sequence not having the breakpoint. In some embodiments, a bait of the disclosure distinguishes a nucleic acid, e.g., a genomic or transcribed nucleic acid, e.g., a cDNA or RNA, having a COL3A1-ALK breakpoint between intron 2 of COL3A1 and exon 19 of ALK from a reference nucleotide sequence, e.g., a nucleotide sequence not having the breakpoint.

In some embodiments, the bait hybridizes to the COL3A1-ALK breakpoint or the COL5A2-ALK breakpoint, and a sequence on either side of the COL3A1-ALK breakpoint or the COL5A2-ALK breakpoint (e.g., any of 1, 2, 3, 4, 5, 6, 7, 8, 9, or 10 nucleotides on either side of the COL3A1-ALK breakpoint or the COL5A2-ALK breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the COL3A1-ALK breakpoint or the COL5A2-ALK breakpoint).

Probes

Also provided herein are probes, e.g., nucleic acid molecules, suitable for the detection of a COL3A1-ALK fusion nucleic acid molecule or a COL5A2-ALK fusion nucleic acid molecule provided herein. In some embodiments, a probe provided herein comprises a nucleic acid sequence configured to hybridize to a target nucleic acid molecule comprising a COL3A1-ALK fusion nucleic acid molecule or a COL5A2-ALK fusion nucleic acid molecule provided herein, or a fragment or portion thereof. In some embodiments, the probe comprises a nucleic acid sequence configured to hybridize to the COL3A1-ALK fusion nucleic acid molecule or the COL5A2-ALK fusion nucleic acid molecule, or the fragment or portion thereof, of the target nucleic acid molecule. In some embodiments, the probe comprises a nucleic acid sequence configured to hybridize to a fragment or portion of the COL3A1-ALK fusion nucleic acid molecule or the COL5A2-ALK fusion nucleic acid molecule of the target nucleic acid molecule. In some embodiments, the fragment or portion comprises between about 5 and about 25 nucleotides, between about 5 and about 300 nucleotides, between about 100 and about 300 nucleotides, between about 130 and about 230 nucleotides, or between about 150 and about 200 nucleotides.

In some embodiments, the probe comprises a nucleotide sequence configured to hybridize to a COL5A2-ALK breakpoint or a COL3A1-ALK breakpoint, and may be further configured to hybridize to between about 10 and about 100 nucleotides or more, e.g., any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides flanking either side of the COL5A2-ALK breakpoint or the COL3A1-ALK breakpoint.

In some embodiments, the probe comprises a nucleotide sequence configured to hybridize to a nucleotide sequence in an intron or an exon of COL5A2 or ALK, or in a COL5A2-ALK breakpoint joining the introns or exons of COL5A2 and ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL5A2-ALK breakpoint joining an intron of COL5A2 and an intron of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL5A2-ALK breakpoint joining an intron of COL5A2 and an exon of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL5A2-ALK breakpoint joining an exon of COL5A2 and an exon of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL5A2-ALK breakpoint joining an exon of COL5A2 and an intron of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the probe comprises a nucleotide sequence configured to hybridize to a nucleotide sequence in an intron or an exon of COL3A1 or ALK, or in a COL3A1-ALK breakpoint joining the introns or exons of COL3A1 and ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint joining an intron of COL3A1 and an intron of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint joining an intron of COL3A1 and an exon of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint joining an exon of COL3A1 and an exon of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint joining an exon of COL3A1 and an intron of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the probe comprises a nucleotide sequence configured to hybridize to the COL5A2-ALK breakpoint between exon 1 of COL5A2 and intron 5 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL5A2-ALK breakpoint between intron 1 of COL5A2 and intron 5 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL5A2-ALK breakpoint between exon 1 of COL5A2 and exon 6 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL5A2-ALK breakpoint between intron 1 of COL5A2 and exon 6 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the probe comprises a nucleotide sequence configured to hybridize to the COL3A1-ALK breakpoint between exon 48 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint between exon 48 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint between intron 48 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint between intron 48 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint between exon 2 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint between exon 2 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint between intron 2 of COL3A1 and intron 18 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides). In some embodiments, the probe comprises a nucleotide sequence configured to hybridize to the COL3A1-ALK breakpoint between intron 2 of COL3A1 and exon 19 of ALK (e.g., plus or minus any of between about 10 and about 20, about 20 and about 30, about 30 and about 40, about 40 and about 50, about 50 and about 60, about 60 and about 70, about 70 and about 80, about 80 and about 90, or about 90 and about 100, or more nucleotides).

In some embodiments, the probe comprises a nucleic acid molecule which is a DNA, RNA, or a DNA/RNA molecule. In some embodiments, the probe comprises a nucleic acid molecule comprising any of between about 10 and about 20 nucleotides, between about 12 and about 20 nucleotides, between about 10 and about 1000 nucleotides, between about 50 and about 500 nucleotides, between about 100 and about 500 nucleotides, between about 100 and about 300 nucleotides, between about 130 and about 230 nucleotides, or between about 150 and about 200 nucleotides. In some embodiments, the probe comprises a nucleic acid molecule comprising any of 10 nucleotides, 11 nucleotides, 12 nucleotides, 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides. In some embodiments, the probe comprises a nucleic acid molecule comprising any of between about 40 nucleotides and about 50 nucleotides, about 50 nucleotides and about 100 nucleotides, about 100 nucleotides and about 150 nucleotides, about 150 nucleotides and about 200 nucleotides, about 200 nucleotides and about 250 nucleotides, about 250 nucleotides and about 300 nucleotides, about 300 nucleotides and about 350 nucleotides, about 350 nucleotides and about 400 nucleotides, about 400 nucleotides and about 450 nucleotides, about 450 nucleotides and about 500 nucleotides, about 500 nucleotides and about 550 nucleotides, about 550 nucleotides and about 600 nucleotides, about 600 nucleotides and about 650 nucleotides, about 650 nucleotides and about 700 nucleotides, about 700 nucleotides and about 750 nucleotides, about 750 nucleotides and about 800 nucleotides, about 800 nucleotides and about 850 nucleotides, about 850 nucleotides and about 900 nucleotides, about 900 nucleotides and about 950 nucleotides, or about 950 nucleotides and about 1000 nucleotides. In some embodiments, the probe comprises a nucleic acid molecule comprising between about 12 and about 20 nucleotides.

In some embodiments, a probe provided herein comprises a DNA, RNA, or a DNA/RNA molecule. In some embodiments, a probe provided herein includes a label or a tag. In some embodiments, the label or tag is a radiolabel (e.g., a radioisotope), a fluorescent label (e.g., a fluorescent compound), an enzymatic label, an enzyme co-factor, a sequence tag, biotin, or another ligand. In some embodiments, a probe provided herein includes a detection reagent such as a fluorescent marker. In some embodiments, a probe provided herein includes (e.g., is conjugated to) an affinity tag, e.g., that allows capture and isolation of a hybrid formed by a probe and a nucleic acid hybridized to the probe. In some embodiments, the affinity tag is an antibody, an antibody fragment, biotin, or any other suitable affinity tag or reagent known in the art. In some embodiments, a probe is suitable for solution phase hybridization.

In some embodiments, probes provided herein may be used according to the methods of detection of COL5A2-ALK or COL3A1-ALK fusion nucleic acid molecules provided herein. For example, a probe provided herein may be used for detecting a COL5A2-ALK fusion nucleic acid molecule or a COL3A1-ALK fusion nucleic acid molecule provided herein in a sample, e.g., a sample obtained from an individual. In some embodiments, the probe may be used for identifying cells or tissues that express a COL5A2-ALK fusion nucleic acid molecule or a COL3A1-ALK fusion nucleic acid molecule provided herein, e.g., by measuring levels of the COL5A2-ALK fusion nucleic acid molecule or the COL3A1-ALK fusion nucleic acid molecule. In some embodiments, the probe may be used for detecting levels of a COL5A2-ALK fusion nucleic acid molecule or a COL3A1-ALK fusion nucleic acid molecule, e.g., mRNA levels, in a sample of cells from an individual.

In some embodiments, a probe provided herein specifically hybridizes to a nucleic acid comprising a rearrangement (e.g., a deletion, inversion, insertion, duplication, or other rearrangement) resulting in a COL5A2-ALK fusion nucleic acid molecule or a COL3A1-ALK fusion nucleic acid molecule described herein.

In some embodiments, a probe of the disclosure distinguishes a nucleic acid, e.g., a genomic or transcribed nucleic acid, e.g., a cDNA or RNA, having a COL3A1-ALK or a COL5A2-ALK breakpoint described herein, from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL5A2-ALK breakpoint between exon 1 of COL5A2 and intron 5 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL5A2-ALK breakpoint between intron 1 of COL5A2 and intron 5 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL5A2-ALK breakpoint between exon 1 of COL5A2 and exon 6 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL5A2-ALK breakpoint between intron 1 of COL5A2 and exon 6 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL3A1-ALK breakpoint between exon 48 of COL3A1 and intron 18 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL3A1-ALK breakpoint between exon 48 of COL3A1 and exon 19 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL3A1-ALK breakpoint between intron 48 of COL3A1 and intron 18 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL3A1-ALK breakpoint between intron 48 of COL3A1 and exon 19 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL3A1-ALK breakpoint between exon 2 of COL3A1 and intron 18 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL3A1-ALK breakpoint between exon 2 of COL3A1 and exon 19 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL3A1-ALK breakpoint between intron 2 of COL3A1 and intron 18 of ALK from a reference nucleotide sequence, e.g., a nucleotide sequence not having the breakpoint. In some embodiments, a probe of the disclosure distinguishes a nucleic acid, e.g., a genomic or transcribed nucleic acid, e.g., a cDNA or RNA, having a COL3A1-ALK breakpoint between intron 2 of COL3A1 and exon 19 of ALK from a reference nucleotide sequence, e.g., a nucleotide sequence not having the breakpoint.

Also provided herein are isolated pairs of allele-specific probes, wherein, for example, the first probe of the pair specifically hybridizes to a COL5A2-ALK fusion nucleic acid molecule or a COL3A1-ALK fusion nucleic acid molecule described herein, e.g., to the COL5A2-ALK breakpoint or the COL3A1-ALK breakpoint, and the second probe of the pair specifically hybridizes to a corresponding wild type sequence (e.g., a wild type COL5A2, COL3A1 or ALK nucleic acid molecule). Probe pairs can be designed and produced for any of the fusion nucleic acid molecules described herein and are useful in detecting a somatic mutation in a sample. In some embodiments, a first probe of a pair specifically hybridizes to a mutation (e.g., the COL5A2-ALK breakpoint or the COL3A1-ALK breakpoint of an inversion, duplication, deletion, insertion or translocation resulting in a COL5A2-ALK fusion nucleic acid molecule or a COL3A1-ALK fusion nucleic acid molecule described herein), and a second probe of a pair specifically hybridizes to a sequence upstream or downstream of the mutation.

In some embodiments, one or more probes provided herein are suitable for use in in situ hybridization methods, e.g., as described above, such as FISH.

Chromosomal probes, e.g., for use in the FISH methods described herein, are typically about 50 to about 105 nucleotides in length. Longer probes typically comprise smaller fragments of about 100 to about 500 nucleotides. Probes that hybridize with centromeric DNA and locus-specific DNA are available commercially, for example, from Vysis, Inc. (Downers Grove, Ill.), Molecular Probes, Inc. (Eugene, Oreg.) or from Cytocell (Oxfordshire, UK). Alternatively, probes can be made non-commercially from chromosomal or genomic DNA through standard techniques. For example, sources of DNA that can be used include genomic DNA, cloned DNA sequences, somatic cell hybrids that contain one, or a part of one, chromosome (e.g., human chromosome) along with the normal chromosome complement of the host, and chromosomes purified by flow cytometry or microdissection. The region of interest can be isolated through cloning, or by site-specific amplification via the polymerase chain reaction (PCR). Probes of the disclosure may also hybridize to RNA molecules, e.g., mRNA, such as an RNA comprising a COL5A2-ALK breakpoint or a COL3A1-ALK breakpoint provided herein.

In some embodiments, probes, such as probes for use in the FISH methods described herein, are used for determining whether a cytogenetic abnormality is present in one or more cells, e.g., in a region of a chromosome or an RNA bound by one or more probes provided herein. The cytogenetic abnormality may be a cytogenetic abnormality that results in a COL5A2-ALK fusion nucleic acid molecule or a COL3A1-ALK fusion nucleic acid molecule described herein. Examples of such cytogenetic abnormalities include, without limitation, deletions (e.g., deletions of entire chromosomes or deletions of fragments of one or more chromosomes), duplications (e.g., of entire chromosomes, or of regions smaller than an entire chromosome), translocations (e.g., non-reciprocal translocations, balanced translocations), intra-chromosomal inversions, point mutations, deletions, gene copy number changes, germ-line mutations, and gene expression level changes.

In some embodiments, probes, such as probes for use in the FISH methods described herein, are labeled such that a chromosomal region or a region on an RNA to which the probes hybridize can be detected. Probes typically are directly labeled with a fluorophore, allowing the probe to be visualized without a secondary detection molecule. Probes can also be labeled by nick translation, random primer labeling or PCR labeling. Labeling may be accomplished using fluorescent (direct)- or haptene (indirect)-labeled nucleotides. Representative, non-limiting examples of labels include: AMCA-6-dUTP, CascadeBlue-4-dUTP, Fluorescein-12-dUTP, Rhodamine-6-dUTP, TexasRed-6-dUTP, Cy3-6-dUTP, Cy5-dUTP, Biotin(BIO)-11-dUTP, Digoxygenin(DIG)-11-dUTP and Dinitrophenyl (DNP)-11-dUTP. Probes can also be indirectly labeled with biotin or digoxygenin, or labeled with radioactive isotopes such as ³²P and ³H, and secondary detection molecules are used, or further processing is performed, to visualize the probes. For example, a probe labeled with biotin can be detected by avidin conjugated to a detectable marker, e.g., avidin can be conjugated to an enzymatic marker such as alkaline phosphatase or horseradish peroxidase. Enzymatic markers can be detected in standard colorimetric reactions using a substrate and/or a catalyst for the enzyme. Catalysts for alkaline phosphatase include 5-bromo-4-chloro-3-indolylphosphate and nitro blue tetrazolium. Diaminobenzoate can be used as a catalyst for horseradish peroxidase. Probes can also be prepared such that a fluorescent or other label is added after hybridization of the probe to its target to detect that the probe hybridized to the target. For example, probes can be used that have antigenic molecules incorporated into the nucleotide sequence. After hybridization, these antigenic molecules are detected, for example, using specific antibodies reactive with the antigenic molecules. Such antibodies can, for example, themselves incorporate a fluorochrome, or can be detected using a second antibody with a bound fluorochrome. For fluorescent probes, e.g., used in FISH techniques, fluorescence can be viewed with a fluorescence microscope equipped with an appropriate filter for each fluorophore, or by using dual or triple band-pass filter sets to observe multiple fluorophores. Alternatively, techniques such as flow cytometry can be used to examine the hybridization pattern of the chromosomal probes.

In some embodiments, the probe hybridizes to the COL3A1-ALK breakpoint or the COL5A2-ALK breakpoint, and a sequence on either side of the COL3A1-ALK breakpoint or the COL5A2-ALK breakpoint (e.g., any of 1, 2, 3, 4, 5, 6, 7, 8, 9, or 10 nucleotides on either side of the COL3A1-ALK breakpoint or the COL5A2-ALK breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the COL3A1-ALK breakpoint or the COL5A2-ALK breakpoint).

Oligonucleotides

In some aspects, provided herein are oligonucleotides, e.g., useful as primers. In some embodiments, an oligonucleotide, e.g., a primer, provided herein comprises a nucleotide sequence configured to hybridize to a target nucleic acid molecule comprising a COL3A1-ALK fusion nucleic acid molecule or a COL5A2-ALK fusion nucleic acid molecule provided herein, or a fragment or portion thereof. In some embodiments, the oligonucleotide comprises a nucleotide sequence configured to hybridize to the COL3A1-ALK fusion nucleic acid molecule or the COL5A2-ALK fusion nucleic acid molecule of the target nucleic acid molecule. In some embodiments, the oligonucleotide comprises a nucleotide sequence configured to hybridize to a fragment or portion of the COL3A1-ALK fusion nucleic acid molecule or the COL5A2-ALK fusion nucleic acid molecule of the target nucleic acid molecule.

In some embodiments, the oligonucleotide, e.g., the primer, comprises a nucleotide sequence configured to hybridize to a COL5A2-ALK breakpoint or a COL3A1-ALK breakpoint, and may be further configured to hybridize to between about 10 and about 12, about 12 and about 15, about 15 and about 17, about 17 and about 20, about 20 and about 25, or about 25 and about 30, or more nucleotides flanking either side of the COL5A2-ALK breakpoint or the COL3A1-ALK breakpoint.

In some embodiments, the oligonucleotide, e.g., the primer, comprises a nucleotide sequence configured to hybridize to a nucleotide sequence in an intron or an exon of COL5A2 or ALK, or in a COL5A2-ALK breakpoint joining the introns or exons of COL5A2 and ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL5A2-ALK breakpoint joining an intron of COL5A2 and an intron of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL5A2-ALK breakpoint joining an intron of COL5A2 and an exon of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL5A2-ALK breakpoint joining an exon of COL5A2 and an exon of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL5A2-ALK breakpoint joining an exon of COL5A2 and an intron of ALK (e.g., plus or minus any of between about 10 and about 12, about 12 and about 15, about 15 and about 17, about 17 and about 20, about 20 and about 25, or about 25 and about 30, or more nucleotides).

In some embodiments, the oligonucleotide, e.g., the primer, comprises a nucleotide sequence configured to hybridize to a nucleotide sequence in an intron or an exon of COL3A1 or ALK, or in a COL3A1-ALK breakpoint joining the introns or exons of COL3A1 and ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint joining an intron of COL3A1 and an intron of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint joining an intron of COL3A1 and an exon of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint joining an exon of COL3A1 and an exon of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint joining an exon of COL3A1 and an intron of ALK (e.g., plus or minus any of between about 10 and about 12, about 12 and about 15, about 15 and about 17, about 17 and about 20, about 20 and about 25, or about 25 and about 30, or more nucleotides).

In some embodiments, the oligonucleotide, e.g., the primer, comprises a nucleotide sequence configured to hybridize to the COL5A2-ALK breakpoint between exon 1 of COL5A2 and intron 5 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL5A2-ALK breakpoint between intron 1 of COL5A2 and intron 5 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL5A2-ALK breakpoint between exon 1 of COL5A2 and exon 6 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL5A2-ALK breakpoint between intron 1 of COL5A2 and exon 6 of ALK (e.g., plus or minus any of between about 10 and about 12, about 12 and about 15, about 15 and about 17, about 17 and about 20, about 20 and about 25, or about 25 and about 30, or more nucleotides).

In some embodiments, the oligonucleotide, e.g., the primer, comprises a nucleotide sequence configured to hybridize to the COL3A1-ALK breakpoint between exon 48 of COL3A1 and intron 18 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint between exon 48 of COL3A1 and exon 19 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint between intron 48 of COL3A1 and intron 18 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint between intron 48 of COL3A1 and exon 19 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint between exon 2 of COL3A1 and intron 18 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint between exon 2 of COL3A1 and exon 19 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint between intron 2 of COL3A1 and intron 18 of ALK (e.g., plus or minus any of between about 10 and about 12, about 12 and about 15, about 15 and about 17, about 17 and about 20, about 20 and about 25, or about 25 and about 30, or more nucleotides). In some embodiments, the oligonucleotide, e.g., the primer, comprises a nucleotide sequence configured to hybridize to the COL3A1-ALK breakpoint between intron 2 of COL3A1 and exon 19 of ALK (e.g., plus or minus any of between about 10 and about 12, about 12 and about 15, about 15 and about 17, about 17 and about 20, about 20 and about 25, or about 25 and about 30, or more nucleotides).

In some embodiments, the oligonucleotide comprises a nucleotide sequence corresponding to a COL5A2-ALK fusion nucleic acid molecule or a COL3A1-ALK fusion nucleic acid molecule provided herein. In some embodiments, the oligonucleotide comprises a nucleotide sequence corresponding to a fragment or a portion of a COL5A2-ALK fusion nucleic acid molecule or a COL3A1-ALK fusion nucleic acid molecule provided herein. In some embodiments, the fragment or portion comprises between about 10 and about 30 nucleotides, between about 12 and about 20 nucleotides, or between about 12 and about 17 nucleotides. In some embodiments, the oligonucleotide comprises a nucleotide sequence complementary to a COL5A2-ALK fusion nucleic acid molecule or a COL3A1-ALK fusion nucleic acid molecule provided herein. In some embodiments, the oligonucleotide comprises a nucleotide sequence complementary to a fragment or a portion of a COL5A2-ALK fusion nucleic acid molecule or a COL3A1-ALK fusion nucleic acid molecule provided herein. In some embodiments, the fragment or portion comprises between about 10 and about 30 nucleotides, between about 12 and about 20 nucleotides, or between about 12 and about 17 nucleotides.

In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence, e.g., under high stringency conditions. In some embodiments, an oligonucleotide, e.g., a primer, provided herein comprises a nucleotide sequence that is sufficiently complementary to its target nucleotide sequence such that the oligonucleotide specifically hybridizes to a nucleic acid molecule comprising the target nucleotide sequence under conditions that allow a polymerization reaction (e.g., PCR) to occur.

In some embodiments, an oligonucleotide, e.g., a primer, provided herein may be useful for initiating DNA synthesis via PCR (polymerase chain reaction) or a sequencing method. In some embodiments, the oligonucleotide may be used to amplify a nucleic acid molecule comprising a COL3A1-ALK fusion nucleic acid molecule or a COL5A2-ALK fusion nucleic acid molecule provided herein, or a fragment thereof, e.g., using PCR. In some embodiments, the oligonucleotide may be used to sequence a nucleic acid molecule comprising a COL3A1-ALK fusion nucleic acid molecule or a COL5A2-ALK fusion nucleic acid molecule provided herein, or a fragment thereof. In some embodiments, the oligonucleotide may be used to amplify a nucleic acid molecule comprising a COL3A1-ALK breakpoint or a COL5A2-ALK breakpoint provided herein, e.g., using PCR. In some embodiments, the oligonucleotide may be used to sequence a nucleic acid molecule comprising a COL3A1-ALK breakpoint or a COL5A2-ALK breakpoint.

In some embodiments, pairs of oligonucleotides, e.g., pairs of primers, are provided herein, which are configured to hybridize to a nucleic acid molecule comprising a COL3A1-ALK fusion nucleic acid molecule or a COL5A2-ALK fusion nucleic acid molecule provided herein, or a fragment thereof. In some embodiments, a pair of oligonucleotides of the disclosure may be used for directing amplification of the fusion nucleic acid molecule or fragment thereof, e.g., using a PCR reaction. In some embodiments, pairs of oligonucleotides, e.g., pairs of primers, are provided herein, which are configured to hybridize to a nucleic acid molecule comprising a COL3A1-ALK breakpoint or a COL5A2-ALK breakpoint provided herein, e.g., for use in directing amplification of the fusion nucleic acid molecule or fragment thereof, e.g., using a PCR reaction.

In some embodiments, an oligonucleotide, e.g., a primer, provided herein is a single stranded nucleic acid molecule, e.g., for use in sequencing or amplification methods. In some embodiments, an oligonucleotide provided herein is a double stranded nucleic acid molecule. In some embodiments, a double stranded oligonucleotide is treated, e.g., denatured, to separate its two strands prior to use, e.g., in sequencing or amplification methods. Oligonucleotides provided herein comprise a nucleotide sequence of sufficient length to hybridize to their target, e.g., a COL3A1-ALK fusion nucleic acid molecule or a COL5A2-ALK fusion nucleic acid molecule provided herein, or a fragment thereof, and to prime the synthesis of extension products, e.g., during PCR or sequencing.

In some embodiments, an oligonucleotide, e.g., a primer, provided herein comprises 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, or more deoxyribonucleotides or ribonucleotides. In some embodiments, an oligonucleotide provided herein comprises at least about 8 deoxyribonucleotides or ribonucleotides. In some embodiments, an oligonucleotide provided herein comprises at least about 10 deoxyribonucleotides or ribonucleotides. In some embodiments, an oligonucleotide provided herein comprises at least about 12 deoxyribonucleotides or ribonucleotides. In some embodiments, an oligonucleotide provided herein comprises at least about 15 deoxyribonucleotides or ribonucleotides. In some embodiments, an oligonucleotide provided herein comprises at least about 20 deoxyribonucleotides or ribonucleotides. In some embodiments, an oligonucleotide provided herein comprises at least about 30 deoxyribonucleotides or ribonucleotides. In some embodiments, an oligonucleotide provided herein comprises between about 10 and about 30 deoxyribonucleotides or ribonucleotides. In some embodiments, an oligonucleotide provided herein comprises between about 10 and about 25 deoxyribonucleotides or ribonucleotides. In some embodiments, an oligonucleotide provided herein comprises between about 10 and about 20 deoxyribonucleotides or ribonucleotides. In some embodiments, an oligonucleotide provided herein comprises between about 10 and about 15 deoxyribonucleotides or ribonucleotides. In some embodiments, an oligonucleotide provided herein comprises between about 12 and about 20 deoxyribonucleotides or ribonucleotides. In some embodiments, an oligonucleotide provided herein comprises between about 17 and about 20 deoxyribonucleotides or ribonucleotides. In some embodiments, the length and nucleotide sequence of an oligonucleotide provided herein is determined according to methods known in the art, e.g., based on factors such as the specific application (e.g., PCR, sequencing library preparation, sequencing), reaction conditions (e.g., buffers, temperature), and the nucleotide composition of the nucleotide sequence of the oligonucleotide or of its target complementary sequence.

In some embodiments, an oligonucleotide, e.g., a primer, of the disclosure distinguishes a nucleic acid, e.g., a genomic or transcribed nucleic acid, e.g., a cDNA or RNA, having a COL3A1-ALK or a COL5A2-ALK breakpoint described herein, from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL5A2-ALK breakpoint between exon 1 of COL5A2 and intron 5 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL5A2-ALK breakpoint between intron 1 of COL5A2 and intron 5 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL5A2-ALK breakpoint between exon 1 of COL5A2 and exon 6 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL5A2-ALK breakpoint between intron 1 of COL5A2 and exon 6 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL3A1-ALK breakpoint between exon 48 of COL3A1 and intron 18 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL3A1-ALK breakpoint between exon 48 of COL3A1 and exon 19 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL3A1-ALK breakpoint between intron 48 of COL3A1 and intron 18 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL3A1-ALK breakpoint between intron 48 of COL3A1 and exon 19 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL3A1-ALK breakpoint between exon 2 of COL3A1 and intron 18 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL3A1-ALK breakpoint between exon 2 of COL3A1 and exon 19 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL3A1-ALK breakpoint between intron 2 of COL3A1 and intron 18 of ALK from a reference nucleotide sequence, e.g., a nucleotide sequence not having the breakpoint. In some embodiments, an oligonucleotide, e.g., a primer, of the disclosure distinguishes a nucleic acid, e.g., a genomic or transcribed nucleic acid, e.g., a cDNA or RNA, having a COL3A1-ALK breakpoint between intron 2 of COL3A1 and exon 19 of ALK from a reference nucleotide sequence, e.g., a nucleotide sequence not having the breakpoint.

In one aspect, provided herein is a primer or primer set for amplifying a nucleic acid molecule comprising a cytogenetic abnormality such as a chromosomal inversion, deletion, translocation, duplication, or other rearrangement resulting in a fusion nucleic acid molecule described herein (e.g., a COL5A2-ALK fusion nucleic acid molecule or a COL3A1-ALK fusion nucleic acid molecule described herein). In another aspect, provided herein is a primer or primer set for amplifying a nucleic acid molecule comprising a chromosomal inversion, insertion, deletion, translocation, duplication or other rearrangement resulting in a fusion nucleic acid molecule described herein (e.g., a COL5A2-ALK fusion nucleic acid molecule or a COL3A1-ALK fusion nucleic acid molecule described herein). In certain aspects, provided herein are allele-specific oligonucleotides, e.g., primers, wherein a first oligonucleotide of a pair specifically hybridizes to a mutation (e.g., the COL5A2-ALK breakpoint or the COL3A1-ALK breakpoint of an inversion, duplication, deletion, insertion, translocation, or other rearrangement resulting in a COL5A2-ALK fusion nucleic acid molecule or a COL3A1-ALK fusion nucleic acid molecule described herein), and a second oligonucleotide of a pair specifically hybridizes to a sequence upstream or downstream of the mutation. In certain aspects, provided herein are pairs of oligonucleotides, e.g., primers, wherein a first oligonucleotide of a pair specifically hybridizes to a sequence upstream of a mutation (e.g., the COL5A2-ALK breakpoint or the COL3A1-ALK breakpoint of an inversion, duplication, deletion, insertion, translocation, or other rearrangement resulting in a COL5A2-ALK fusion nucleic acid molecule or a COL3A1-ALK fusion nucleic acid molecule described herein), and a second oligonucleotide of the pair specifically hybridizes to a sequence downstream of the mutation.

In some embodiments, the oligonucleotide, e.g., the primer, hybridizes to the COL3A1-ALK breakpoint or the COL5A2-ALK breakpoint, and a sequence on either side of the COL3A1-ALK breakpoint or the COL5A2-ALK breakpoint (e.g., any of 1, 2, 3, 4, 5, 6, 7, 8, 9, or 10 nucleotides on either side of the COL3A1-ALK breakpoint or the COL5A2-ALK breakpoint, or any of between 1 and about 5, about 5 and about 10, about 10 and about 15, about 15 and about 20, about 20 and about 25, about 25 and about 30, about 30 and about 35, about 35 and about 40, about 40 and about 45, about 45 and about 50, about 50 and about 55, about 55 and about 60, about 60 and about 65, about 70 and about 75, about 75 and about 80, about 80 and about 85, about 85 and about 90, about 90 and about 95, or about 95 and about 100, or more nucleotides on either side of the COL3A1-ALK breakpoint or the COL5A2-ALK breakpoint).

Nucleic Acid Samples

In some embodiments, a COL5A2-ALK fusion nucleic acid molecule of the disclosure, or a COL3A1-ALK fusion nucleic acid molecule of the disclosure is detected in a sample comprising nucleic acids, e.g., genomic DNA, cDNA, or mRNA. In some embodiments, the sample is obtained from an individual having a cancer, such as a cancer described herein. A variety of materials (such as tissues) can be the source of the nucleic acid samples used in the methods provided herein. For example, the source of the sample can be solid tissue as from a fresh, frozen and/or preserved organ, tissue sample, biopsy, resection, smear, or aspirate; blood or any blood constituents; bodily fluids such as cerebrospinal fluid, amniotic fluid, urine, saliva, sputum, peritoneal fluid or interstitial fluid; or cells from any time in gestation or development of an individual. In some embodiments, the source of the sample is blood or blood constituents. In some embodiments, the source of the sample is a tumor sample. In some embodiments, the sample is or comprises biological tissue or fluid. In some embodiments, the sample can contain compounds that are not naturally intermixed with the tissue in nature, such as preservatives, anticoagulants, buffers, fixatives, nutrients, antibiotics or the like. In some embodiments, a fusion nucleic acid molecule is detected in a sample comprising genomic or subgenomic DNA fragments, or RNA, such as mRNA isolated from a sample, e.g., a tumor sample, a normal adjacent tissue (NAT) sample, a tissue sample, or a blood sample obtained from an individual. In some embodiments, the sample comprises cDNA derived from an mRNA sample or from a sample comprising mRNA. In some embodiments, the tissue is preserved as a frozen sample or as a formaldehyde- or paraformaldehyde-fixed paraffin-embedded (FFPE) tissue preparation. For example, the sample can be embedded in a matrix, e.g., an FFPE block or a frozen sample.

In some embodiments, the sample comprises cell-free DNA (cfDNA). In some embodiments, the sample comprises cell-free RNA (cfRNA). In some embodiments, the sample comprises circulating tumor DNA (ctDNA).

In some embodiments, a sample may be or comprise bone marrow; a bone marrow aspirate; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; other body fluids, secretions, and/or excretions; and/or cells therefrom. In some embodiments, a biological sample is or comprises cells obtained from an individual.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example nucleic acids or proteins extracted from a sample or obtained by subjecting a primary sample to techniques such as amplification or reverse transcription of mRNA, or isolation and/or purification of certain components.

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In another embodiment, the sample includes one or more circulating tumor cells (CTCs) (e.g., a CTC acquired from a blood sample). In one embodiment, the sample is a cell not associated with a tumor, e.g., a non-tumor cell or a peripheral blood lymphocyte.

In some embodiments, the sample comprises tumor nucleic acids, such as nucleic acids from a tumor or a cancer sample, e.g., genomic DNA, RNA, or cDNA derived from RNA, from a tumor or cancer sample. In certain embodiments, a tumor nucleic acid sample is purified or isolated (e.g., it is removed from its natural state).

In some embodiments, the sample is a control nucleic acid sample or a reference nucleic acid sample, e.g., genomic DNA, RNA, or cDNA derived from RNA, not containing a gene fusion described herein. In certain embodiments, the reference or control nucleic acid sample comprises a wild type or a non-mutated sequence. In certain embodiments, the reference nucleic acid sample is purified or isolated (e.g., it is removed from its natural state). In other embodiments, the reference nucleic acid sample is from a non-tumor sample, e.g., a blood control, a normal adjacent tumor (NAT), or any other non-cancerous sample from the same or a different subject.

In some embodiments, a fusion nucleic acid molecule of the disclosure is detected in a sample comprising cell-free DNA (cfDNA), cell-free RNA, or circulating tumor DNA (ctDNA).

Detection of ALK Fusion Polypeptides

Also provided herein are methods of detecting a COL5A2-ALK fusion polypeptide of the disclosure or a COL3A1-ALK fusion polypeptide of the disclosure, or a fragment thereof. A fusion polypeptide provided herein, or a fragment thereof, may be detected or measured, e.g., in a sample obtained from an individual, using any method known in the art, such as using antibodies (e.g., an antibody described herein), mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography).

In some embodiments, a COL5A2-ALK fusion polypeptide of the disclosure or a COL3A1-ALK fusion polypeptide of the disclosure, or a fragment thereof, can be distinguished from a reference polypeptide, e.g., a non-mutant or wild type COL5A2, COL3A1, and/or ALK protein or polypeptide, with an antibody or antibody fragment that reacts differentially with a mutant protein or polypeptide (e.g., a fusion polypeptide provided herein or a fragment thereof) as compared to a reference protein or polypeptide. In some embodiments, a COL5A2-ALK fusion polypeptide of the disclosure or a COL3A1-ALK fusion polypeptide of the disclosure, or a fragment thereof, can be distinguished from a reference polypeptide, e.g., a non-mutant or wild type COL5A2, COL3A1, and/or ALK protein or polypeptide, by reaction with a detection reagent, e.g., a substrate, e.g., a substrate for catalytic activity, e.g., phosphorylation.

In some aspects, methods of detection of a COL5A2-ALK fusion polypeptide of the disclosure or a COL3A1-ALK fusion polypeptide of the disclosure, or a fragment thereof, are provided, comprising contacting a sample, e.g., a sample described herein, comprising a fusion polypeptide described herein, with a detection reagent provided herein (e.g., an antibody of the disclosure), and determining if the fusion polypeptide is present in the sample.

Protein Samples

In some embodiments, a sample for use according to the methods of detection of a COL5A2-ALK fusion polypeptide of the disclosure, or of a COL3A1-ALK fusion polypeptide of the disclosure, is a solid tissue, e.g., from a fresh, frozen and/or preserved organ, tissue sample, biopsy (e.g., a tumor biopsy), resection, smear, or aspirate; blood or any blood constituents; bodily fluids such as cerebrospinal fluid, amniotic fluid, urine, saliva, sputum, peritoneal fluid or interstitial fluid; or cells such as tumor cells. In some embodiments, the source of the sample is blood or blood constituents. In some embodiments, the source of the sample is a tumor sample. In some embodiments, the sample is or comprises biological tissue or fluid. In some embodiments, the sample is preserved as a frozen sample or as a formaldehyde- or paraformaldehyde-fixed paraffin-embedded (FFPE) tissue preparation. In some embodiments, the sample comprises circulating tumor cells (CTCs).

In some embodiments, a sample for use according to the methods of detection of a fusion polypeptide described herein is a sample of proteins isolated or obtained from a solid tissue, e.g., from a fresh, frozen and/or preserved organ, tissue sample, biopsy (e.g., a tumor biopsy), resection, smear, or aspirate; from blood or any blood constituents; from bodily fluids such as cerebrospinal fluid, amniotic fluid, urine, saliva, sputum, peritoneal fluid or interstitial fluid; or from cells such as tumor cells. In some embodiments, the sample is a sample of proteins isolated or obtained from a preserved sample, such as a frozen sample or a formaldehyde- or paraformaldehyde-fixed paraffin-embedded (FFPE) tissue preparation. In some embodiments, the sample is a sample of proteins isolated or obtained from circulating tumor cells (CTCs). In some embodiments, the sample can contain compounds that are not naturally intermixed with the tissue in nature, such as preservatives, anticoagulants, buffers, fixatives, nutrients, antibiotics or the like.

In some embodiments, a sample may be or comprise bone marrow; a bone marrow aspirate; blood; blood cells; ascites; tissue or fine needle biopsy samples; cell-containing body fluids; free floating nucleic acids; sputum; saliva; urine; cerebrospinal fluid, peritoneal fluid; pleural fluid; feces; lymph; gynecological fluids; skin swabs; vaginal swabs; oral swabs; nasal swabs; washings or lavages such as ductal lavages or bronchoalveolar lavages; aspirates; scrapings; bone marrow specimens; tissue biopsy specimens; surgical specimens; other body fluids, secretions, and/or excretions; and/or cells therefrom. In some embodiments, a biological sample is or comprises cells obtained from an individual.

In some embodiments, a sample is a primary sample obtained directly from a source of interest by any appropriate means. For example, in some embodiments, a primary biological sample is obtained by a method chosen from biopsy (e.g., fine needle aspiration or tissue biopsy), surgery, or collection of body fluid (e.g., blood, lymph, or feces). In some embodiments, as will be clear from context, the term “sample” refers to a preparation that is obtained by processing (e.g., by removing one or more components of and/or by adding one or more agents to) a primary sample. Such a processed sample may comprise, for example, proteins extracted from a sample or obtained by subjecting a primary sample to techniques such as isolation and/or purification of certain components.

In one embodiment, the sample comprises one or more cells associated with a tumor, e.g., tumor cells or tumor-infiltrating lymphocytes (TIL). In one embodiment, the sample includes one or more premalignant or malignant cells. In one embodiment, the sample is acquired from a hematologic malignancy (or pre-malignancy), e.g., a hematologic malignancy (or pre-malignancy) described herein. In one embodiment, the sample is acquired from a cancer, such as a cancer described herein. In some embodiments, the sample is acquired from a solid tumor, a soft tissue tumor or a metastatic lesion. In other embodiments, the sample includes tissue or cells from a surgical margin. In another embodiment, the sample includes one or more circulating tumor cells (CTCs) (e.g., a CTC acquired from a blood sample). In one embodiment, the sample is a cell not associated with a tumor, e.g., a non-tumor cell or a peripheral blood lymphocyte.

In some embodiments, the sample comprises tumor proteins or polypeptides, such as proteins or polypeptides from a tumor or a cancer sample. In certain embodiments, the proteins are purified or isolated (e.g., removed from their natural state).

In some embodiments, the sample is a control sample or a reference sample, e.g., not containing a fusion polypeptide described herein. In certain embodiments, the reference sample is purified or isolated (e.g., it is removed from its natural state). In other embodiments, the reference sample is from a non-tumor sample, e.g., a blood control, a normal adjacent tumor (NAT), or any other non-cancerous sample from the same or a different subject.

### Antibodies

Provided herein are antibodies or antibody fragments that specifically bind to a COL5A2-ALK fusion polypeptide of the disclosure or a COL3AT-ALK fusion polypeptide of the disclosure, or a portion thereof. The antibody may be of any suitable type of antibody, including, but not limited to, a monoclonal antibody, a polyclonal antibody, a multi-specific antibody (e.g., a bispecific antibody), or an antibody fragment, so long as the antibody or antibody fragment exhibits a specific antigen binding activity (e.g., binding to a fusion polypeptide of the disclosure, or a portion thereof).

In some embodiments, a COL5A2-ALK fusion polypeptide of the disclosure or a COL3A1-ALK fusion polypeptide of the disclosure, or a fragment thereof, is used as an immunogen to generate one or more antibodies of the disclosure, e.g., using standard techniques for polyclonal and monoclonal antibody preparation. In some embodiments, a fusion polypeptide provided herein, is used to provide antigenic peptide fragments (e.g., comprising any of at least about 8, at least about 10, at least about 15, at least about 20, at least about 30 or more amino acids) for use as immunogens to generate one or more antibodies of the disclosure, e.g., using standard techniques for polyclonal and monoclonal antibody preparation. As is known in the art, an antibody of the disclosure may be prepared by immunizing a suitable (i.e., immunocompetent) subject such as a rabbit, goat, mouse, or other mammal or vertebrate. An appropriate immunogenic preparation can contain, for example, recombinantly-expressed or chemically-synthesized polypeptides, e.g., a fusion polypeptide provided herein, or a fragment thereof. The preparation can further include an adjuvant, such as Freund's complete or incomplete adjuvant, or a similar immunostimulatory agent.

In some embodiments, an antibody provided herein is a polyclonal antibody. Methods of producing polyclonal antibodies are known in the art. In some embodiments, an antibody provided herein is a monoclonal antibody, wherein a population of the antibody molecules contain only one species of an antigen binding site capable of immunoreacting or binding with a particular epitope, e.g., an epitope on a fusion polypeptide provided herein. Methods of preparation of monoclonal antibodies are known in the art, e.g., using standard hybridoma techniques originally described by Kohler and Milstein (1975) Nature 256:495-497, human B cell hybridoma techniques (see Kozbor et al., 1983, Immunol. Today 4:72), EBV-hybridoma techniques (see Cole et al., pp. 77-96 In Monoclonal Antibodies and Cancer Therapy, Alan R. Liss, Inc., 1985), or trioma techniques. The technology for producing hybridomas is well known (see generally Current Protocols in Immunology, Coligan et al. ed., John Wiley & Sons, New York, 1994). A monoclonal antibody of the disclosure may also be identified and isolated by screening a recombinant combinatorial immunoglobulin library (e.g., an antibody phage display library) with the polypeptide of interest, e.g., a fusion polypeptide provided herein or a fragment thereof. Kits for generating and screening phage display libraries are commercially available (e.g., the Pharmacia Recombinant Phage Antibody System, Catalog No. 27-9400-01; and the Stratagene SurfZAP Phage Display Kit, Catalog No. 240612). Additionally, examples of methods and reagents particularly amenable for use in generating and screening antibody display libraries can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; and Griffiths et al. (1993) EMBO J. 12:725-734. In some embodiments, monoclonal antibodies of the disclosure are recombinant antibodies, such as chimeric and humanized monoclonal antibodies, comprising both human and non-human portions. Such chimeric and/or humanized monoclonal antibodies can be produced by recombinant DNA techniques known in the art, for example, using methods described in PCT Publication No. WO 87/02671; European Patent Application 184,187; European Patent Application 171,496; European Patent Application 173,494; PCT Publication No. WO 86/01533; U.S. Pat. No. 4,816,567; European Patent Application 125,023; Better et al. (1988) Science 240:1041-1043; Liu et al. (1987) Proc. Natl. Acad. Sci. USA 84:3439-3443; Liu et al. (1987) J. Immunol. 139:3521-3526; Sun et al. (1987) Proc. Natl. Acad. Sci. USA 84:214-218; Nishimura et al. (1987) Cancer Res. 47:999-1005; Wood et al. (1985) Nature 314:446-449; Shaw et al. (1988) J. Natl. Cancer Inst. 80:1553-1559; Morrison (1985) Science 229:1202-1207; Oi et al. (1986) Bio/Techniques 4:214; U.S. Pat. No. 5,225,539; Jones et al. (1986) Nature 321:552-525; Verhoeyan et al. (1988) Science 239:1534; and Beidler et al. (1988) J. Immunol. 141:4053-4060. In some embodiments, a monoclonal antibody of the disclosure is a human monoclonal antibody. In some embodiments, human monoclonal antibodies are prepared using methods known in the art, e.g., using transgenic mice which are incapable of expressing endogenous immunoglobulin heavy and light chains genes, but which can express human heavy and light chain genes. For an overview of this technology for producing human antibodies, see Lonberg and Huszar (1995) Int. Rev. Immunol. 13:65-93. For a detailed discussion of this technology for producing human antibodies and human monoclonal antibodies, and protocols for producing such antibodies, see, e.g., U.S. Pat. Nos. 5,625,126; 5,633,425; 5,569,825; 5,661,016; and 5,545,806.

In some embodiments, the antibody or antibody fragment of the disclosure is an isolated antibody or antibody fragment, which has been separated from a component of its natural environment or a cell culture used to produce the antibody or antibody fragment. In some embodiments, an antibody of the disclosure is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods.

In some embodiments, an antibody of the disclosure can be used to isolate a COL5A2-ALK fusion polypeptide or a COL3AT-ALK fusion polypeptide provided herein, or a fragment thereof, by standard techniques, such as affinity chromatography or immunoprecipitation. In some embodiments, an antibody of the disclosure can be used to detect a COL5A2-ALK fusion polypeptide or a COL3AT-ALK fusion polypeptide provided herein, or a fragment thereof, e.g., in a tissue sample, cellular lysate, or cell supernatant, in order to evaluate the level and/or pattern of expression of the fusion polypeptide. Detection can be facilitated by coupling the antibody to a detectable substance. Thus, in some embodiments, an antibody of the disclosure is coupled to a detectable substance, such as enzymes, prosthetic groups, fluorescent materials, luminescent materials, bioluminescent materials, and radioactive materials. Non-limiting examples of suitable enzymes include, e.g., horseradish peroxidase, alkaline phosphatase, β-galactosidase, or acetylcholinesterase; examples of suitable prosthetic group complexes include, e.g., streptavidin/biotin and avidin/biotin; examples of suitable fluorescent materials include, e.g., umbelliferone, fluorescein, fluorescein isothiocyanate, rhodamine, dichlorotriazinylamine fluorescein, dansyl chloride or phycoerythrin; an example of a luminescent material includes, but is not limited to, luminol; examples of bioluminescent materials include, e.g., luciferase, luciferin, and aequorin; and examples of suitable radioactive materials include, e.g., ¹²⁵I, ¹³¹I, ³⁵S or ³H.

An antibody or antibody fragment of the disclosure may also be used diagnostically, e.g., to detect and/or monitor protein levels (e.g., protein levels of a fusion polypeptide provided herein) in tissues or body fluids (e.g., in a tumor cell-containing tissue or body fluid), e.g., according to the methods provided herein.

Antibody Affinity

In certain embodiments, an antibody provided herein has a dissociation constant (Kd) of ≤1 μM, ≤100 nM, ≤10 nM, ≤1 nM, ≤0.1 nM, ≤0.01 nM, or ≤0.001 nM (e.g. 10⁻⁸ M or less, e.g., from 10⁻⁸ M to 10⁻¹³ M, e.g., from 10⁻⁹ M to 10⁻¹³ M). Methods of measuring antibody affinity (e.g., Kd) are known in the art, and include, without limitation, a radiolabeled antigen binding assay (RIA) and a BIACORE® surface plasmon resonance assay. In some embodiments, antibody affinity (e.g., Kd) is determined using the Fab version of an antibody of the disclosure and its antigen (e.g., a fusion polypeptide provided herein). In some embodiments, a RIA is performed with the Fab version of an antibody of the disclosure and its antigen (e.g., a fusion polypeptide provided herein).

Antibody Fragments

In certain embodiments, an antibody provided herein is an antibody fragment. Antibody fragments include, but are not limited to, Fab, Fab′, Fab′-SH, F(ab′)2, Fv, and single-chain antibody molecules (e.g., scFv) fragments, and other fragments described herein.

In certain embodiments, an antibody provided herein is a diabody. Diabodies are antibody fragments with two antigen-binding sites that may be bivalent or bispecific. In certain embodiments, an antibody provided herein is a triabody or a tetrabody.

In certain embodiments, an antibody provided herein is a single-domain antibody. Single-domain antibodies are antibody fragments comprising all or a portion of the heavy chain variable domain or all or a portion of the light chain variable domain of an antibody. In certain embodiments, a single-domain antibody is a human single-domain antibody.

Antibody fragments can be made by various techniques, including but not limited to proteolytic digestion of an intact antibody, as well as production by recombinant host cells (e.g., E. coli or phage), as known in the art and as described herein.

Chimeric and Humanized Antibodies

In certain embodiments, an antibody provided herein is a chimeric antibody. In one example, a chimeric antibody comprises a non-human variable region (e.g., a variable region derived from a mouse, rat, hamster, rabbit, or non-human primate, such as a monkey), and a human constant region. In a further example, a chimeric antibody is a “class switched” antibody, in which the class or subclass of the antibody has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof), are derived from a non-human antibody, and framework regions (FRs) (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity. Humanized antibodies and methods of making them are known in the art. Human framework regions that may be used for humanization include but are not limited to: framework regions selected using the “best-fit” method; framework regions derived from the consensus sequence of human antibodies of a particular subgroup of light or heavy chain variable regions; human mature (somatically mutated) framework regions or human germline framework regions; and framework regions derived from screening FR libraries.

Human Antibodies

In certain embodiments, an antibody provided herein is a human antibody. Human antibodies can be produced using various techniques known in the art. For example, human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or are present extrachromosomally or integrated randomly into the animal's chromosomes. In such transgenic animals, e.g., mice, the endogenous immunoglobulin loci have generally been inactivated. Human variable regions from intact antibodies generated by such animals may be further modified, e.g., by combining with a different human constant region. Human antibodies can also be made by hybridoma-based methods known in the art, e.g., using known human myeloma and mouse-human heteromyeloma cell lines for the production of human monoclonal antibodies. Human antibodies may also be generated by isolating Fv clone variable domain sequences selected from human-derived phage display libraries. Such variable domain sequences may then be combined with a desired human constant domain. Techniques for selecting human antibodies from antibody libraries are described below.

Library-Derived Antibodies

Antibodies of the disclosure may be isolated by screening combinatorial libraries for antibodies with the desired activity or activities. For example, a variety of methods are known in the art for generating phage display libraries and screening such libraries for antibodies possessing the desired binding characteristics. In certain phage display methods, repertoires of VH and VL genes are separately cloned by polymerase chain reaction (PCR) and recombined randomly in phage libraries, which can then be screened for antigen-binding phage. Phage typically display antibody fragments, either as single-chain Fv (scFv) fragments or as Fab fragments. Libraries from immunized sources provide high-affinity antibodies to the immunogen without the requirement of constructing hybridomas. Alternatively, a naive antibody repertoire can be cloned (e.g., from human) to provide a single source of antibodies to a wide range of non-self and also self antigens without any immunization. Naive libraries can also be made synthetically by cloning un-rearranged V-gene segments from stem cells, and using PCR primers containing random sequences to amplify the highly variable CDR3 regions and to accomplish rearrangement in vitro. Antibodies or antibody fragments isolated from human antibody libraries are considered human antibodies or human antibody fragments herein.

Multispecific Antibodies

In certain embodiments, an antibody provided herein is a multispecific antibody, e.g., a bispecific antibody. Multispecific antibodies are monoclonal antibodies that have binding specificities for at least two different sites or at least two different antigens. For example, one of the binding specificities can be to an immune checkpoint protein of the present disclosure, and the other can be to any other antigen, e.g., a fusion polypeptide provided herein. Multispecific antibodies can be prepared as full length antibodies or as antibody fragments. Techniques for making multispecific antibodies are known in the art and include, but are not limited to, recombinant co-expression of two immunoglobulin heavy chain-light chain pairs having different specificities, and “knob-in-hole” engineering. Multispecific antibodies may also be made by engineering electrostatic steering effects (e.g., by introducing mutations in the constant region) for making heterodimeric Fcs; cross-linking two or more antibodies or fragments; using leucine zippers to produce bispecific antibodies; using “diabody” technology for making bispecific antibody fragments; using single-chain Fv (scFv) dimers; and preparing trispecific antibodies. Engineered antibodies with three or more functional antigen binding sites, including “Octopus antibodies,” are also included in the disclosure. Antibodies or antibody fragments of the disclosure also include “Dual Acting FAbs” or “DAF,” e.g., comprising an antigen binding site that binds to an immune checkpoint protein as well as another, different antigen.

Antibody Variants

In certain embodiments, amino acid sequence variants of the antibodies provided herein are contemplated. For example, it may be desirable to improve the binding affinity and/or other biological properties of the antibody. Amino acid sequence variants of an antibody of the disclosure may be prepared by introducing appropriate modifications into the nucleotide sequence encoding the antibody, or by peptide synthesis. Such modifications include, for example, deletions, and/or insertions, and/or substitutions of residues within the amino acid sequences of the antibody. Any combination of deletions, insertions, and substitutions can be made to arrive at the final antibody, provided that the final antibody possesses the desired characteristics, e.g., antigen-binding.

In certain embodiments, antibody variants having one or more amino acid substitutions are provided. Sites of interest for substitutional mutagenesis include the HVRs and FRs. Amino acid substitutions may be introduced into an antibody of interest, and the products may be screened for a desired activity, e.g., retained/improved antigen binding, decreased immunogenicity, or improved or reduced antibody-dependent cell-mediated cytotoxicity (ADCC) and/or complement-dependent cytotoxicity (CDC).

In certain embodiments, an antibody of the present disclosure is altered to increase or to decrease the extent to which the antibody is glycosylated. Addition or deletion of glycosylation sites to an antibody may be conveniently accomplished by altering the amino acid sequence of the antibody, such that one or more glycosylation sites is created or removed. Antibody variants having bisected oligosaccharides are further provided, e.g., in which a biantennary oligosaccharide attached to the Fc region of the antibody is bisected by GlcNAc. In some embodiments, antibody variants of the disclosure may have increased fucosylation. In some embodiments, antibody variants of the disclosure may have reduced fucosylation. In some embodiments, antibody variants of the disclosure may have improved ADCC function. In some embodiments, antibody variants of the disclosure may have decreased ADCC function. Antibody variants with at least one galactose residue in the oligosaccharide attached to the Fc region are also provided. Such antibody variants may have improved CDC function. In some embodiments, antibody variants of the disclosure may have increased CDC function. In some embodiments, antibody variants of the disclosure may have decreased CDC function.

In certain embodiments, one or more amino acid modifications may be introduced into the Fc region of an antibody of the present disclosure, thereby generating an Fc region variant. The Fc region variant may comprise a human Fc region sequence (e.g., a human IgG1, IgG2, IgG3 or IgG4 Fc region) comprising an amino acid modification (e.g. a substitution) at one or more amino acid positions.

In certain embodiments, the present disclosure contemplates an antibody variant that possesses some but not all effector functions, which make it a desirable candidate for applications in which the half-life of the antibody in vivo is important, yet certain effector functions (such as CDC and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks Fc-gamma-R binding (hence likely lacking ADCC activity), but retains FcRn binding ability. The primary cells that mediate ADCC, e.g., NK cells, express Fc-gamma-RIII only, whereas monocytes express Fc-gamma-RI, Fc-gamma-RII and Fc-gamma-RIII. Antibodies with reduced effector function include those with substitution of one or more of Fc region residues 238, 265, 269, 270, 297, 327 and 329. Such Fc mutants include Fc mutants with substitutions at two or more of amino acid positions 265, 269, 270, 297 and 327, including the so-called “DANA” Fc mutant with substitutions of residues 265 and 297 to alanine. Antibody variants with improved or diminished binding to FcRs are also included in the disclosure. In certain embodiments, an antibody variant comprises an Fc region with one or more amino acid substitutions that improve ADCC, e.g., substitutions at positions 298, 333, and/or 334 of the Fc region. In some embodiments, number of Fc region residues is according to EU numbering of residues. In some embodiments, alterations are made in the Fc region that result in altered (i.e., either improved or diminished) C1q binding and/or CDC. In some embodiments, antibodies of the disclosure include antibodies with increased half-lives and improved binding to the neonatal Fc receptor (FcRn), e.g., comprising one or more substitutions that improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434. See, also, Duncan & Winter, Nature 322:738-40 (1988); U.S. Pat. Nos. 5,648,260; 5,624,821; and WO 94/29351 for other examples of Fc region variants.

In certain embodiments, an antibody provided herein is a cysteine-engineered antibody, e.g., “thioMAb,” in which one or more residues of the antibody are substituted with cysteine residues. In some embodiments, the substituted residues occur at accessible sites of the antibody. By substituting those residues with cysteine, reactive thiol groups are thereby positioned at accessible sites of the antibody, and may be used to conjugate the antibody to other moieties, such as drug moieties or linker-drug moieties, e.g., to create an immunoconjugate, as described further herein. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine-engineered antibodies may be generated using any suitable method known in the art.

In some embodiments, an antibody or antibody fragment provided herein comprises a label or a tag. In some embodiments, the label or tag is a radiolabel, a fluorescent label, an enzymatic label, a sequence tag, biotin, or other ligands. Examples of labels or tags include, but are not limited to, 6×His-tag, biotin-tag, Glutathione-S-transferase (GST)-tag, green fluorescent protein (GFP)-tag, c-myc-tag, FLAG-tag, Thioredoxin-tag, Glu-tag, Nus-tag, V5-tag, calmodulin-binding protein (CBP)-tag, Maltose binding protein (MBP)-tag, Chitin-tag, alkaline phosphatase (AP)-tag, HRP-tag, Biotin Caboxyl Carrier Protein (BCCP)-tag, Calmodulin-tag, S-tag, Strep-tag, haemoglutinin (HA)-tag, digoxigenin (DIG)-tag, DsRed, RFP, Luciferase, Short Tetracysteine Tags, Halo-tag, and Nus-tag. In some embodiments, the label or tag comprises a detection agent, such as a fluorescent molecule or an affinity reagent or tag.

In some embodiments, an antibody or antibody fragment provided herein is conjugated to a drug molecule, e.g., an anti-cancer agent described herein, or a cytotoxic agent such as mertansine or monomethyl auristatin E (MMAE).

In certain embodiments, an antibody or antibody fragment provided herein may be further modified to contain additional nonproteinaceous moieties. Such moieties may be suitable for derivatization of the antibody, e.g., including but not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyamino acids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, polyethylene glycol propionaldehyde, and mixtures thereof. The polymers may be of any molecular weight, and may be branched or unbranched. The number of polymers attached to the antibody may vary, and if more than one polymer is attached, the polymers can be the same or different molecules. In general, the number and/or type of polymers used for derivatization can be determined based on considerations including, but not limited to, the particular properties or functions of the antibody to be improved, or whether the antibody derivative will be used in a therapy under defined conditions. In some embodiments, provided herein are antibodies conjugated to carbon nanotubes, e.g., for use in methods to selectively heat the antibody using radiation to a temperature at which cells proximal to the antibody are killed.

### Cancers

In some embodiments, a cancer of the disclosure, e.g., comprising a fusion nucleic acid molecule or polypeptide described herein, is acute lymphoblastic leukemia (“ALL”), acute myeloid leukemia (“AML”), adenocarcinoma, adenocarcinoma of the lung, adrenocortical cancer, adrenocortical carcinoma, anal cancer, appendiceal cancer, B-cell derived leukemia, B-cell derived lymphoma, B-cell lymphoma, bladder cancer, brain cancer, breast cancer (e.g., triple negative breast cancer (TNBC) or non-triple negative breast cancer), cancer of the fallopian tube(s), cancer of the testes, carcinoma, cerebral cancer, cervical cancer, cholangiocarcinoma, choriocarcinoma, chronic myelogenous leukemia, central nervous system (CNS) tumor, CNS cancer, colon cancer, colorectal cancer (e.g., colon adenocarcinoma), diffuse intrinsic pontine glioma (DIPG), diffuse large B cell lymphoma (“DLBCL”), embryonal rhabdomyosarcoma (ERMS), endometrial cancer, epithelial cancer, epithelial neoplasm, thymoma, esophageal cancer, Ewing's sarcoma, eye cancer (e.g., uveal melanoma), eyelid cancer, follicular lymphoma (“FL”), gall bladder cancer, gastric cancer, gastrointestinal cancer, glioblastoma, polycythemia vera, glioblastoma multiforme, glioma (e.g., lower grade glioma), gullet cancer, head and neck cancer, a hematological cancer, hepatocellular cancer, hepatocellular carcinoma, Hodgkin's lymphoma (HL), a heavy chain disease, intestinum rectum cancer, renal cancer, kidney cancer (e.g., kidney clear cell cancer, kidney chromophobe cancer, kidney clear cell cancer, kidney papillary cancer), large B-cell lymphoma, large intestine cancer, laryngeal cancer, leucosis, leukemia, liver cancer, lung cancer (e.g., lung adenocarcinoma, or non-small cell lung cancer), lymphoma, mammary gland cancer, melanoma (e.g., metastatic malignant melanoma), Hodgkin's disease, Waldenstrom's macroglobulinemia, Merkel cell carcinoma, mesothelioma, monocytic leukemia, multiple myeloma, myeloma, myogenic sarcoma, nasopharyngeal cancer, neuroblastic-derived CNS tumor (e.g., neuroblastoma (NB)), neuroma, astrocytoma, pilocytic astrocytoma, anaplastic astrocytoma, medulloblastoma, craniopharyngioma, ependymoma, pinealoma, hemangioblastoma, acoustic neuroma, oligodendroglioma, meningioma, vestibular schwannoma, adenoma, metastatic brain tumor, spinal tumor, non-Hodgkin's lymphoma (NHL), oral cancer, oral cavity cancer, osteosarcoma, ovarian cancer, ovarian carcinoma, pancreatic adenocarcinoma, pancreatic cancer, peritoneal cancer, pheochromocytoma, primary mediastinal B-cell lymphoma, primary peritoneal cancer, prostate cancer (e.g., hormone refractory prostate adenocarcinoma), rectal cancer (rectum carcinoma), relapsed or refractory classic Hodgkin's Lymphoma (cHL), salivary gland cancer (e.g., salivary gland tumor), skin cancer, small cell lung cancer, small intestine cancer, soft tissue sarcoma, squamous carcinoma, squamous cell carcinoma (e.g., squamous cell carcinoma of the anogenital region, squamous cell carcinoma of the anus, squamous cell carcinoma of the cervix, squamous cell carcinoma of the esophagus, squamous cell carcinoma of the head and neck (SCHNC), squamous cell carcinoma of the lung, squamous cell carcinoma of the penis, squamous cell carcinoma of the vagina, or squamous cell carcinoma of the vulva), stomach cancer, T-cell derived leukemia, T-cell lymphoma, testicular cancer, testicular tumor, thymic cancer, thyroid cancer (thyroid carcinoma), tongue cancer, tunica conjunctiva cancer, urinary bladder cancer, urothelial cell carcinoma, uterine cancer (e.g., uterine endometrial cancer or uterine sarcoma such as uterine carcinosarcoma), uterine endometrial cancer, uterus cancer, vaginal cancer, vulvar cancer, or Wilms' tumor.

In some embodiments, a cancer of the disclosure, e.g., comprising a fusion nucleic acid molecule or polypeptide described herein, is a hematologic cancer (e.g., a hematologic malignancy), such as diffuse large B cell lymphoma (“DLBCL”), Hodgkin's lymphoma (“HL”), Non-Hodgkin's lymphoma (“NHL”), follicular lymphoma (“FL”), acute myeloid leukemia (“AML”), acute lymphoblastic leukemia (“ALL”), multiple myeloma (“MM”), acute lymphoblastic B-cell leukemia, acute lymphoblastic T-cell leukemia, acute myeloblastic leukemia, acute promyelocytic leukemia (“APL”), acute monoblastic leukemia, acute erythroleukemic leukemia, acute megakaryoblastic leukemia, acute myelomonocytic leukemia, acute nonlymphocyctic leukemia, acute undifferentiated leukemia, chronic myelocytic leukemia (“CML”), chronic lymphocytic leukemia (“CLL”), or hairy cell leukemia. In some embodiments, a hematologic cancer of the disclosure, e.g., comprising a fusion nucleic acid molecule or polypeptide described herein, is an acute or a chronic leukemia, such as a lymphoblastic, myelogenous, lymphocytic, or myelocytic leukemia. In some embodiments, a hematologic cancer of the disclosure, e.g., comprising a fusion nucleic acid molecule or polypeptide described herein, is a lymphoma (e.g., Hodgkin's lymphoma, such as relapsed or refractory classic Hodgkin's Lymphoma (cHL), a non-Hodgkin's lymphoma, a diffuse large B-cell lymphoma, or a precursor T-lymphoblastic lymphoma), a lymphoepithelial carcinoma, or a malignant histiocytosis.

In some embodiments, a cancer of the disclosure, e.g., comprising a fusion nucleic acid molecule or polypeptide described herein, is a solid tumor (e.g., a solid malignancy), such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, osteosarcoma, colon cancer, colorectal cancer, kidney cancer, pancreatic cancer, bone cancer, breast cancer, ovarian cancer, prostate cancer, esophageal cancer, stomach cancer, oral cancer, nasal cancer, throat cancer, squamous cell carcinoma, basal cell carcinoma, adenocarcinoma, sweat gland carcinoma, sebaceous gland carcinoma, papillary carcinoma, papillary adenocarcinoma, cystadenocarcinoma, medullary carcinoma, bronchogenic carcinoma, renal cell carcinoma, hepatoma, bile duct carcinoma, choriocarcinoma, seminoma, embryonal carcinoma, Wilms tumor, cervical cancer, uterine cancer, testicular cancer, non-small cell lung cancer (NSCLC), small cell lung carcinoma, bladder carcinoma, lung cancer, epithelial carcinoma, skin cancer, melanoma, neuroblastoma (NB), or retinoblastoma.

In some embodiments, a cancer of the disclosure, e.g., comprising a fusion nucleic acid molecule or polypeptide described herein, is anaplastic large cell lymphoma (ALCL), non-small cell lung cancer (NSCLC), colorectal cancer (CRC), sarcoma, sarcoma not otherwise specified (NOS), inflammatory myofibroblastic tumor (IMT), rhabdomyosarcoma, acute myeloid leukemia, histiocytosis, leiomyosarcoma, ALK-positive large B-cell lymphoma, epithelioid fibrous histiocytoma, a pulmonary carcinoma, a renal cell carcinoma, a thyroid carcinoma, a pancreatic carcinoma, carcinoma of unknown primary, ovarian carcinoma, glioma, mesothelioma, melanoma or a Spitzoid tumor.

In certain embodiments, a cancer of the disclosure, e.g., comprising a fusion nucleic acid molecule or polypeptide described herein, is a cancer of the adrenal glands (such as neuroblastoma), bladder cancer (such as urothelial (transitional cell) carcinoma), brain cancer (such as anaplastic astrocytoma or glioblastoma), bone cancer (such as osteosarcoma), bone marrow cancer (such as B-cell acute leukemia (B-ALL) or multiple myeloma), breast cancer (such as invasive ductal carcinoma), head and neck cancer (such as adenocarcinoma, mucoepidermoid carcinoma, squamous cell carcinoma), lymph node cancer, lung cancer (e.g., mucoepidermoid carcinoma, sarcoma, small cell undifferentiated carcinoma, adenocarcinoma, adenosquamous carcinoma, large cell carcinoma, large cell neuroendocrine carcinoma, non-small cell lung carcinoma, non-small cell lung carcinoma not otherwise specified, or squamous cell carcinoma), female reproductive cancer (e.g., cancer of the fallopian tubes such as fallopian tube serous carcinoma; ovarian cancer, such as epithelial carcinoma, epithelial carcinoma not otherwise specified, high grade serous carcinoma, low grade serous carcinoma, serous carcinoma; and uterine cancer, such as carcinosarcoma, endometrial adenocarcinoma, endometrial adenocarcinoma not otherwise specified, papillary serous endometrial adenocarcinoma, leiomyosarcoma, sarcoma, sarcoma not otherwise specified, or smooth muscle tumor of uncertain malignant potential (STUMP)), gallbladder cancer (such as adenocarcinoma), cancer of the gastroesophageal junction (such as adenocarcinoma), lymph node cancer (such as anaplastic large cell lymphoma, B-cell lymphoma, B-cell lymphoma not otherwise specified, diffuse large B cell lymphoma, non-Hodgkin's lymphoma, non-Hodgkin's lymphoma not otherwise specified), colon cancer (such as adenocarcinoma), colorectal cancer, skin cancer (such as melanoma or squamous cell carcinoma), small intestine cancer (adenocarcinoma), soft tissue cancer (such as Ewing sarcoma, fibrosarcoma, histiocytosis, histiocytosis not otherwise specified, juvenile xanthogranuloma or non-Langerhans cell histiocytosis, inflammatory myofibroblastic tumor, leiomyosarcoma, neurofibroma, neuroblastoma, sarcoma not otherwise specified, sarcoma, undifferentiated sarcoma, or an undifferentiated soft tissue cancer), pancreatic cancer (such as carcinoma, carcinoma not otherwise specified, ductal adenocarcinoma, or mucinous cystadenocarcinoma), prostate cancer (such as acinar adenocarcinoma), pericardium cancer (such as mesothelioma), peritoneum cancer (such as mesothelioma), salivary gland cancer (such as carcinoma or carcinoma not otherwise specified), stomach cancer (such as adenocarcinoma, adenocarcinoma not otherwise specified, or diffuse type cancer), kidney cancer (such as renal cell carcinoma or renal cell carcinoma not otherwise specified), thyroid cancer (such as carcinoma, carcinoma not otherwise specified, or papillary carcinoma), or a cancer of unknown primary origin (such as adenocarcinoma, carcinoma, carcinoma not otherwise specified, leiomyosarcoma, malignant neoplasm, malignant neoplasm not otherwise specified, melanoma, myoepithelial carcinoma, squamous cell carcinoma (SCC), or undifferentiated neuroendocrine carcinoma).

In some embodiments, a cancer of the disclosure is rhabdomyosarcoma comprising a COL5A2-ALK fusion nucleic acid molecule or polypeptide described herein. In some embodiments, the rhabdomyosarcoma comprises a COL5A2-ALK fusion nucleic acid molecule comprising: exon 1 or a portion thereof of COL5A2 fused to intron 5 or a portion thereof of ALK; intron 1 or a portion thereof of COL5A2 fused to intron 5 or a portion thereof of ALK; exon 1 or a portion thereof of COL5A2 fused to exon 6 or a portion thereof of ALK; or intron 1 or a portion thereof of COL5A2 fused to exon 6 or a portion thereof of ALK. In some embodiments, the rhabdomyosarcoma comprises a COL5A2-ALK fusion nucleic acid molecule comprising a nucleotide sequence comprising, in the 5′ to 3′ direction, exon 1 or a portion thereof of COL5A2, and exon 6 or a portion thereof and exons 7-29 of ALK. In some embodiments, the rhabdomyosarcoma comprises a COL5A2-ALK fusion nucleic acid molecule that results from a breakpoint in exon 1 or in intron 1 of COL5A2, and in intron 5 or in exon 6 of ALK. In some embodiments, the rhabdomyosarcoma comprises a COL5A2-ALK fusion nucleic acid molecule comprising the nucleotide sequence of SEQ ID NO: 7, or a nucleotide sequence at least 70%, 75%, 80%, 85%, 90%, 91%, 92%, 93%, 94%, 95%, 96%, 97%, 98%, 99%, or 99.5% identical thereto. In some embodiments, the rhabdomyosarcoma comprises a COL5A2-ALK fusion polypeptide comprising: an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK; or an amino acid sequence at least about 85% (e.g., any of at least about 85%, at least about 90%, at least about 95%, at least about 96%, at least about 97%, at least about 98%, at least about 99%, or 100%) identical to an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK. In some embodiments, the rhabdomyosarcoma comprises a COL5A2-ALK fusion polypeptide comprising the amino acid sequence of SEQ ID NO: 10, or an amino acid sequence at least 70%, 75%, 80%, 85%, 90%, 91%, 92%, 93%, 94%, 95%, 96%, 97%, 98%, 99%, or 99.5% identical thereto. In some embodiments, the COL5A2-ALK fusion polypeptide has kinase activity, e.g., ALK kinase activity or a tyrosine kinase activity.

In some embodiments, a cancer of the disclosure is a leiomyosarcoma, an inflammatory myofibroblastic tumor (IMT), or a sarcoma not otherwise specified (NOS) comprising a COL3A1-ALK fusion nucleic acid molecule or polypeptide described herein. In some embodiments, the leiomyosarcoma, inflammatory myofibroblastic tumor (IMT), or sarcoma not otherwise specified (NOS) comprises a COL3A1-ALK fusion nucleic acid molecule comprising: (a) exon 48 or a portion thereof of COL3A1 fused to intron 18 or a portion thereof of ALK; exon 48 or a portion thereof of COL3A1 fused to exon 19 or a portion thereof of ALK; intron 48 or a portion thereof of COL3A1 fused to intron 18 or a portion thereof of ALK; or intron 48 or a portion thereof of COL3A1 fused to exon 19 or a portion thereof of ALK; or (b) exon 2 or a portion thereof of COL3A1 fused to intron 18 or a portion thereof of ALK; exon 2 or a portion thereof of COL3A1 fused to exon 19 or a portion thereof of ALK; intron 2 or a portion thereof of COL3A1 fused to intron 18 or a portion thereof of ALK; or intron 2 or a portion thereof of COL3A1 fused to exon 19 or a portion thereof of ALK. In some embodiments, the leiomyosarcoma, inflammatory myofibroblastic tumor (IMT), or sarcoma not otherwise specified (NOS) comprises a COL3A1-ALK fusion nucleic acid molecule comprising a nucleotide sequence comprising, in the 5′ to 3′ direction: (a) exons 1-47 and exon 48 or a portion thereof of COL3A1, and exon 19 or a portion thereof and exons 20-29 of ALK; or (b) exon 1 and exon 2 or a portion thereof of COL3A1, and exon 19 or a portion thereof and exons 20-29 of ALK. In some embodiments, the leiomyosarcoma, inflammatory myofibroblastic tumor (IMT), or sarcoma not otherwise specified (NOS) comprises a COL3A1-ALK fusion nucleic acid molecule resulting from: (a) a breakpoint in exon 2 or intron 2 of COL3A1, and in intron 18 or exon 19 of ALK; or a breakpoint joining Chr2:189849674 with Chr2:29448496; or (b) a breakpoint in exon 48 or intron 48 of COL3A1, and in intron 18 or exon 19 of ALK; a breakpoint joining Chr2:189874528 with Chr2:29448490; or a breakpoint joining Chr2:189874814 with Chr2:29449440. In some embodiments, the chromosome positions correspond to chromosome positions of human genome version hg19. In some embodiments, the leiomyosarcoma, inflammatory myofibroblastic tumor (IMT), or sarcoma not otherwise specified (NOS) comprises a COL3A1-ALK fusion nucleic acid molecule comprising the nucleotide sequence of SEQ ID NO: 8 or 9, or a nucleotide sequence at least 70%, 75%, 80%, 85%, 90%, 91%, 92%, 93%, 94%, 95%, 96%, 97%, 98%, 99%, or 99.5% identical thereto. In some embodiments, the leiomyosarcoma, inflammatory myofibroblastic tumor (IMT), or sarcoma not otherwise specified (NOS) comprises a COL3A1-ALK fusion polypeptide comprising: (a) an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK; or an amino acid sequence at least about 85% (e.g., any of at least about 85%, at least about 90%, at least about 95%, at least about 96%, at least about 97%, at least about 98%, at least about 99%, or 100%) identical to an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK; or (b) an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK; or an amino acid sequence at least about 85% (e.g., any of at least about 85%, at least about 90%, at least about 95%, at least about 96%, at least about 97%, at least about 98%, at least about 99%, or 100%) identical to an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK. In some embodiments, the leiomyosarcoma, inflammatory myofibroblastic tumor (IMT), or sarcoma not otherwise specified (NOS) comprises a COL3A1-ALK fusion polypeptide comprising the amino acid sequence of SEQ ID NO: 11 or 12, or an amino acid sequence at least 70%, 75%, 80%, 85%, 90%, 91%, 92%, 93%, 94%, 95%, 96%, 97%, 98%, 99%, or 99.5% identical thereto. In some embodiments, the COL3A1-ALK fusion polypeptide has kinase activity, e.g., ALK kinase activity or a tyrosine kinase activity.

In some embodiments, a cancer of the disclosure, e.g., comprising a fusion nucleic acid molecule or polypeptide described herein, is a cancer that is recurrent or refractory to one or more prior anti-cancer therapies.

In some embodiments, a cancer of the disclosure, e.g., comprising a fusion nucleic acid molecule or polypeptide described herein, is any cancer type provided in Ross et al., Oncologist (2017) 22(12):1444-1450, which is incorporated herein by reference.

In some embodiments, a cancer of the disclosure, e.g., comprising a fusion nucleic acid molecule or polypeptide described herein, has low tumor mutational burden (TMB).

### Methods of Diagnosing, Assessing, Screening, Monitoring or Predicting

In some aspects, provided herein are methods of diagnosing or assessing a COL5A2-ALK fusion or a COL3A1-ALK fusion in a cancer, such as a cancer provided herein, in an individual. In some embodiments, the methods comprise acquiring knowledge of the presence of a COL5A2-ALK fusion nucleic acid molecule or polypeptide or a COL3A1-ALK fusion nucleic acid molecule or polypeptide provided herein in a sample obtained from the individual. In some embodiments, the methods comprise detecting a COL5A2-ALK fusion nucleic acid molecule or polypeptide or a COL3A1-ALK fusion nucleic acid molecule or polypeptide provided herein in a sample obtained from the individual. In some embodiments, the fusion nucleic acid molecule or polypeptide is detected in a sample obtained from the individual using any method known in the art, such as one or more of the methods of detection of fusion nucleic acid molecules or polypeptides described herein. In some embodiments, the methods further comprise providing a diagnosis or an assessment of the fusion nucleic acid molecule or polypeptide. In some embodiments, the diagnosis or assessment identifies the presence or absence of the fusion nucleic acid molecule or polypeptide in the sample. In some embodiments, the diagnosis or assessment identifies the cancer, such as a cancer provided herein, as likely to respond to an anti-cancer therapy, e.g., an anti-cancer therapy provided herein. In some embodiments, the presence of the fusion nucleic acid molecule or polypeptide in the sample identifies the cancer as likely to respond to an anti-cancer therapy, e.g., an anti-cancer therapy provided herein. In some embodiments, the sample is a sample described herein. In some embodiments, the sample comprises cells from the cancer or is obtained from cells from the cancer. In some embodiments, the individual has a cancer, is suspected of having a cancer, is being tested for a cancer, is being treated for a cancer, or is being tested for a susceptibility to a cancer, e.g., a cancer described herein.

In some aspects, provided herein are methods of diagnosing or assessing a cancer in an individual, e.g., a cancer provided herein. In some embodiments, the methods of diagnosing or assessing cancer comprise detecting a COL5A2-ALK fusion nucleic acid molecule or polypeptide or a COL3A1-ALK fusion nucleic acid molecule or polypeptide provided herein in a sample obtained from the individual, e.g., a sample comprising cells from the cancer. In some embodiments, the methods comprise detecting a fusion nucleic acid molecule or polypeptide described herein in a sample obtained from the individual using any method known in the art, such as one or more of the methods of detection of fusion nucleic acid molecules or polypeptides described herein. In some embodiments, detection of a fusion nucleic acid molecule or polypeptide described herein, or a fragment thereof, in a sample obtained from the individual identifies the cancer as likely to respond to an anti-cancer therapy, e.g., an anti-cancer therapy provided herein. In some embodiments, the presence of a fusion nucleic acid molecule or polypeptide described herein, or a fragment thereof, in a sample obtained from the individual identifies the cancer as likely to respond to an anti-cancer therapy, e.g., an anti-cancer therapy provided herein. In some embodiments, the methods further comprise providing a diagnosis or an assessment of the cancer or of the fusion nucleic acid molecule or polypeptide. In some embodiments, the diagnosis or assessment identifies the cancer as likely to respond to an anti-cancer therapy, e.g., an anti-cancer therapy provided herein. In some embodiments, the diagnosis or assessment identifies the presence or absence of the fusion nucleic acid molecule or polypeptide in the sample.

In some aspects, provided herein are methods of predicting survival of an individual having a cancer, e.g., a cancer provided herein. In some embodiments, the individual is being treated with an anti-cancer therapy, such as an anti-cancer therapy described herein. In some embodiments, the methods comprise acquiring knowledge of a COL5A2-ALK fusion nucleic acid molecule or polypeptide or a COL3A1-ALK fusion nucleic acid molecule or polypeptide provided herein in a sample from the individual. In some embodiments, the methods comprise detecting a COL5A2-ALK fusion nucleic acid molecule or polypeptide or a COL3A1-ALK fusion nucleic acid molecule or polypeptide provided herein in a sample from the individual. In some embodiments, responsive to acquiring knowledge of a COL5A2-ALK fusion nucleic acid molecule or polypeptide or a COL3A1-ALK fusion nucleic acid molecule or polypeptide provided herein in the sample, the individual is predicted to have longer survival after treatment with an anti-cancer therapy, e.g., an anti-cancer therapy provided herein, for example, as compared to an individual whose cancer does not exhibit the COL5A2-ALK fusion nucleic acid molecule or polypeptide, or the COL3A1-ALK fusion nucleic acid molecule or polypeptide. In some embodiments, responsive to detecting a COL5A2-ALK fusion nucleic acid molecule or polypeptide or a COL3A1-ALK fusion nucleic acid molecule or polypeptide provided herein in the sample, the individual is predicted to have longer survival after treatment with an anti-cancer therapy, e.g., an anti-cancer therapy provided herein, for example, as compared to an individual whose cancer does not exhibit the COL5A2-ALK fusion nucleic acid molecule or polypeptide, or the COL3A1-ALK fusion nucleic acid molecule or polypeptide. In some embodiments, the methods further comprise providing a diagnosis or an assessment. In some embodiments, the diagnosis or assessment identifies the presence or absence of the fusion nucleic acid molecule or polypeptide in the sample. In some embodiments, the diagnosis or assessment identifies the individual as being predicted to have longer survival after treatment with an anti-cancer therapy, e.g., an anti-cancer therapy provided herein, for example, as compared to an individual whose cancer does not exhibit the COL5A2-ALK fusion nucleic acid molecule or polypeptide, or the COL3A1-ALK fusion nucleic acid molecule or polypeptide. In some embodiments, the sample is a sample described herein. In some embodiments, the sample comprises cells from the cancer or is obtained from cells from the cancer.

In some aspects, provided herein are methods of screening an individual having cancer, suspected of having cancer, being tested for cancer, being treated for cancer, or being tested for a susceptibility to cancer, e.g., a cancer provided herein. In some embodiments, the individual is being treated with an anti-cancer therapy, such as an anti-cancer therapy described herein. In some embodiments, the methods comprise acquiring knowledge of a COL5A2-ALK fusion nucleic acid molecule or polypeptide or a COL3A1-ALK fusion nucleic acid molecule or polypeptide provided herein in a sample from the individual. In some embodiments, the methods comprise detecting a COL5A2-ALK fusion nucleic acid molecule or polypeptide or a COL3A1-ALK fusion nucleic acid molecule or polypeptide provided herein in a sample from the individual. In some embodiments, responsive to acquiring knowledge of a COL5A2-ALK fusion nucleic acid molecule or polypeptide or a COL3A1-ALK fusion nucleic acid molecule or polypeptide provided herein in the sample, the individual is predicted to have increased risk of cancer recurrence, aggressive cancer, anti-cancer therapy resistance, or poor prognosis, for example, as compared to an individual whose cancer does not exhibit the COL5A2-ALK fusion nucleic acid molecule or polypeptide, or the COL3A1-ALK fusion nucleic acid molecule or polypeptide. In some embodiments, responsive to detecting a COL5A2-ALK fusion nucleic acid molecule or polypeptide or a COL3A1-ALK fusion nucleic acid molecule or polypeptide provided herein in the sample, the individual is predicted to have increased risk of cancer recurrence, aggressive cancer, anti-cancer therapy resistance, or poor prognosis, for example, as compared to an individual whose cancer does not exhibit the COL5A2-ALK fusion nucleic acid molecule or polypeptide, or the COL3A1-ALK fusion nucleic acid molecule or polypeptide. In some embodiments, the methods further comprise providing a diagnosis or an assessment. In some embodiments, the diagnosis or assessment identifies the presence or absence of the fusion nucleic acid molecule or polypeptide in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the COL5A2-ALK fusion nucleic acid molecule or polypeptide, or the COL3A1-ALK fusion nucleic acid molecule or polypeptide. In some embodiments, the sample is a sample described herein. In some embodiments, the sample comprises cells from the cancer or is obtained from cells from the cancer.

In some aspects, provided herein are methods of monitoring an individual having cancer, e.g., a cancer provided herein. In some embodiments, the individual is being treated with an anti-cancer therapy, such as an anti-cancer therapy described herein. In some embodiments, the methods comprise acquiring knowledge of a COL5A2-ALK fusion nucleic acid molecule or polypeptide or a COL3A1-ALK fusion nucleic acid molecule or polypeptide provided herein in a sample from the individual. In some embodiments, the methods comprise detecting a COL5A2-ALK fusion nucleic acid molecule or polypeptide or a COL3A1-ALK fusion nucleic acid molecule or polypeptide provided herein in a sample from the individual. In some embodiments, responsive to acquiring knowledge of a COL5A2-ALK fusion nucleic acid molecule or polypeptide or a COL3A1-ALK fusion nucleic acid molecule or polypeptide provided herein in the sample, the individual is predicted to have increased risk of cancer recurrence, aggressive cancer, anti-cancer therapy resistance, or poor prognosis, for example, as compared to an individual whose cancer does not exhibit the COL5A2-ALK fusion nucleic acid molecule or polypeptide, or the COL3A1-ALK fusion nucleic acid molecule or polypeptide. In some embodiments, responsive to detecting a COL5A2-ALK fusion nucleic acid molecule or polypeptide or a COL3A1-ALK fusion nucleic acid molecule or polypeptide provided herein in the sample, the individual is predicted to have increased risk of cancer recurrence, aggressive cancer, anti-cancer therapy resistance, or poor prognosis, for example, as compared to an individual whose cancer does not exhibit the COL5A2-ALK fusion nucleic acid molecule or polypeptide, or the COL3A1-ALK fusion nucleic acid molecule or polypeptide. In some embodiments, the methods further comprise providing a diagnosis or an assessment. In some embodiments, the diagnosis or assessment identifies the presence or absence of the fusion nucleic acid molecule or polypeptide in the sample. In some embodiments, the diagnosis or assessment identifies the individual as being predicted to have increased risk of cancer recurrence, aggressive cancer, anti-cancer therapy resistance, or poor prognosis, for example, as compared to an individual whose cancer does not exhibit the COL5A2-ALK fusion nucleic acid molecule or polypeptide, or the COL3A1-ALK fusion nucleic acid molecule or polypeptide. In some embodiments, the sample is a sample described herein. In some embodiments, the sample comprises cells from the cancer or is obtained from cells from the cancer.

In some embodiments, the methods further comprise selectively enriching for one or more nucleic acids comprising COL5A2, COL3A1, or ALK nucleotide sequences to produce an enriched sample, e.g., using a reagent known in the art or provided herein, such as a bait, probe, or oligonucleotide described herein. In some embodiments, the methods further comprise selectively enriching for one or more polypeptides comprising COL5A2, COL3A1, or ALK amino acid sequences to produce an enriched sample, e.g., using a reagent known in the art or provided herein, such as an antibody described herein.

### Methods Selecting or Identifying a Treatment

In some aspects, provided herein are methods of identifying an individual having cancer, e.g., a cancer provided herein, who may benefit from an anti-cancer therapy, e.g., an anti-cancer therapy provided herein. In some embodiments, the methods comprise detecting a COL5A2-ALK fusion nucleic acid molecule or polypeptide or a COL3A1-ALK fusion nucleic acid molecule or polypeptide provided herein in a sample obtained from the individual. In some embodiments, the methods comprise acquiring knowledge of the presence of a COL5A2-ALK fusion nucleic acid molecule or polypeptide or a COL3A1-ALK fusion nucleic acid molecule or polypeptide provided herein in a sample obtained from the individual. In some embodiments, the presence of the fusion nucleic acid molecule or polypeptide in the sample identifies the individual as one who may benefit from a treatment comprising an anti-cancer therapy, e.g., an anti-cancer therapy provided herein. In some embodiments, detection of the fusion nucleic acid molecule or polypeptide in the sample identifies the individual as one who may benefit from a treatment comprising an anti-cancer therapy, e.g., an anti-cancer therapy provided herein. In some embodiments, responsive to knowledge of the fusion nucleic acid molecule or polypeptide in the sample, the individual is identified as one who may benefit from a treatment comprising an anti-cancer therapy, e.g., an anti-cancer therapy provided herein. In some embodiments, the fusion nucleic acid molecule or polypeptide is detected using any suitable method known in the art or described herein. In some embodiments, the sample is a sample described herein. In some embodiments, the sample comprises cells from the cancer or is obtained from cells from the cancer.

Also provided herein are methods of identifying or selecting a treatment, a therapy, or one or more treatment options for an individual having a cancer, such as a cancer described herein. In some embodiments, the cancer comprises a COL5A2-ALK fusion nucleic acid molecule or polypeptide or a COL3A1-ALK fusion nucleic acid molecule or polypeptide provided herein. In some embodiments, the methods comprise detecting the fusion nucleic acid molecule or polypeptide provided herein in a sample obtained from an individual having a cancer, such as a cancer described herein. In some embodiments, the fusion nucleic acid molecule or polypeptide is detected using any suitable method known in the art or described herein. In some embodiments, detection of a fusion nucleic acid molecule or polypeptide provided herein in a sample obtained from an individual having a cancer, e.g., a cancer described herein, identifies the individual as one who may benefit from an anti-cancer therapy, e.g., an anti-cancer therapy provided herein. In some embodiments, the presence of a fusion nucleic acid molecule or polypeptide provided herein in a sample obtained from an individual having a cancer, e.g., a cancer provided herein, identifies the individual as one who may benefit from an anti-cancer therapy, such as an anti-cancer therapy provided herein. In some embodiments, responsive to detection of a fusion nucleic acid molecule or polypeptide provided herein in a sample obtained from an individual having a cancer, e.g., a cancer provided herein, the individual is classified as a candidate to receive an anti-cancer therapy, such as an anti-cancer therapy provided herein. In some embodiments, responsive to detection of a fusion nucleic acid molecule or polypeptide provided herein in a sample obtained from an individual having a cancer, e.g., a cancer provided herein, the individual is classified or identified as likely to respond to an anti-cancer therapy, such as an anti-cancer therapy provided herein. In some embodiments, the sample is a sample described herein. In some embodiments, the sample comprises cells from the cancer or is obtained from cells from the cancer. In some embodiments, the methods further comprise generating a report, e.g., as described herein. In some embodiments, the report comprises a treatment, a therapy, or one or more treatment options identified or selected for the individual, e.g., based at least in part on detection of a COL5A2-ALK fusion nucleic acid molecule or polypeptide or a COL3A1-ALK fusion nucleic acid molecule or polypeptide provided herein in the sample.

In some embodiments, the methods of selecting or identifying a treatment, a therapy, or one or more treatment options for an individual having a cancer, e.g., a cancer described herein, comprise acquiring knowledge of a COL5A2-ALK fusion nucleic acid molecule or polypeptide or a COL3A1-ALK fusion nucleic acid molecule or polypeptide provided herein in a sample obtained from the individual. In some embodiments, the methods of selecting or identifying a treatment, a therapy, or one or more treatment options for an individual having a cancer, e.g., a cancer described herein, comprise acquiring knowledge of the presence of a fusion nucleic acid molecule or polypeptide provided herein in a sample obtained from the individual. In some embodiments, the knowledge of the presence of the fusion nucleic acid molecule or polypeptide is acquired using any suitable method known in the art or described herein. In some embodiments, acquiring knowledge of the presence of a fusion nucleic acid molecule or polypeptide provided herein in a sample obtained from the individual identifies the individual as one who may benefit from an anti-cancer therapy, such as an anti-cancer therapy provided herein. In some embodiments, responsive to acquisition of knowledge of a fusion nucleic acid molecule or polypeptide provided herein in a sample obtained from the individual, the individual is classified as a candidate to receive an anti-cancer therapy, such as an anti-cancer therapy provided herein. In some embodiments, responsive to acquisition of knowledge of a fusion nucleic acid molecule or polypeptide provided herein in a sample obtained from the individual, the individual is classified or identified as likely to respond to an anti-cancer therapy, such as an anti-cancer therapy provided herein. In some embodiments, responsive to acquisition of knowledge of the presence of a fusion nucleic acid molecule or polypeptide provided herein in a sample obtained from the individual, the individual is classified as a candidate to receive an anti-cancer therapy, such as an anti-cancer therapy provided herein. In some embodiments, responsive to acquisition of knowledge of the presence of a fusion nucleic acid molecule or polypeptide provided herein in a sample obtained from the individual, the individual is classified or identified as likely to respond to an anti-cancer therapy, such as an anti-cancer therapy provided herein. In some embodiments, the sample is a sample described herein. In some embodiments, the sample comprises cells from the cancer or is obtained from cells from the cancer. In some embodiments, the methods further comprise generating a report, e.g., as described herein. In some embodiments, the report comprises a treatment, a therapy, or one or more treatment options identified or selected for the individual, e.g., based at least in part on knowledge of a COL5A2-ALK fusion nucleic acid molecule or polypeptide or a COL3A1-ALK fusion nucleic acid molecule or polypeptide provided herein in the sample.

### Methods of Treatment

Also provided herein are methods of treating or delaying progression of a cancer in an individual, such as a cancer provided herein. In some embodiments, the individual has a cancer comprising a COL5A2-ALK fusion nucleic acid molecule or polypeptide or a COL3A1-ALK fusion nucleic acid molecule or polypeptide provided herein. In some embodiments, the methods of treating or delaying progression of a cancer of the disclosure in an individual, e.g., comprising a COL5A2-ALK fusion nucleic acid molecule or polypeptide or a COL3A1-ALK fusion nucleic acid molecule or polypeptide provided herein, comprise administering to the individual a therapeutically effective amount of an anti-cancer therapy, such as an anti-cancer therapy provided herein. In some embodiments, the methods of treating or delaying progression of a cancer of the disclosure in an individual, e.g., comprising a COL5A2-ALK fusion nucleic acid molecule or polypeptide or a COL3A1-ALK fusion nucleic acid molecule or polypeptide provided herein, comprise administering to the individual an effective amount of an anti-cancer therapy, such as an anti-cancer therapy provided herein, responsive to knowledge of the presence of the fusion nucleic acid molecule or polypeptide in a sample obtained from the individual. In some embodiments, the sample is a sample described herein. In some embodiments, the sample comprises cells from the cancer or is obtained from cells from the cancer.

In some embodiments, the methods of treating or delaying progression of a cancer of the disclosure, e.g., comprising a fusion nucleic acid molecule or polypeptide provided herein, in an individual comprise detecting a COL5A2-ALK fusion nucleic acid molecule or polypeptide or a COL3A1-ALK fusion nucleic acid molecule or polypeptide provided herein in a sample obtained from the individual. In some embodiments, the methods further comprise administering to the individual an effective amount of an anti-cancer therapy, such as an anti-cancer therapy provided herein. In some embodiments, the anti-cancer therapy is administered to the individual responsive to detecting the fusion nucleic acid molecule or polypeptide in the sample. In some embodiments, the fusion nucleic acid molecule or polypeptide is detected using any suitable method known in the art or described herein. In some embodiments, responsive to detection of the fusion nucleic acid molecule or polypeptide provided herein in a sample obtained from the individual, the individual is administered a therapeutically effective amount of an anti-cancer therapy, such as an anti-cancer therapy provided herein. In some embodiments, the sample is a sample described herein. In some embodiments, the sample comprises cells from the cancer or is obtained from cells from the cancer.

In some embodiments, the methods of treating or delaying progression of a cancer of the disclosure in an individual, e.g., comprising a COL5A2-ALK fusion nucleic acid molecule or polypeptide or a COL3A1-ALK fusion nucleic acid molecule or polypeptide provided herein, comprise acquiring knowledge of the presence of a COL5A2-ALK fusion nucleic acid molecule or polypeptide or a COL3A1-ALK fusion nucleic acid molecule or polypeptide of the disclosure in a sample obtained from the individual. In some embodiments, the methods further comprise administering to the individual an effective amount of an anti-cancer therapy, such as an anti-cancer therapy provided herein. In some embodiments, the anti-cancer therapy is administered to the individual responsive to acquiring knowledge of the presence of the fusion nucleic acid molecule or polypeptide in the sample. In some embodiments, the knowledge of the presence of the fusion nucleic acid molecule or polypeptide is acquired using any suitable method known in the art or described herein. In some embodiments, responsive to acquisition of knowledge of the presence of the fusion nucleic acid molecule or polypeptide provided herein in a sample obtained from the individual, the individual is administered an effective amount of an anti-cancer therapy, such as an anti-cancer therapy provided herein. In some embodiments, the sample is a sample described herein. In some embodiments, the sample comprises cells from the cancer or is obtained from cells from the cancer.

### Reporting

In some embodiments, the methods provided herein comprise generating a report, and/or providing a report to party.

In some embodiments, a report according to the present disclosure comprises information about one or more of: a COL5A2-ALK fusion nucleic acid molecule or polypeptide or a COL3A1-ALK fusion nucleic acid molecule or polypeptide of the disclosure; a cancer of the disclosure, e.g., comprising a fusion nucleic acid molecule or polypeptide of the disclosure; or a treatment, a therapy, or one or more treatment options for an individual having a cancer, such as a cancer of the disclosure (e.g., comprising a fusion nucleic acid molecule or polypeptide described herein).

In some embodiments, a report according to the present disclosure comprises information about the presence or absence of a COL5A2-ALK fusion nucleic acid molecule or polypeptide or a COL3A1-ALK fusion nucleic acid molecule or polypeptide of the disclosure in a sample obtained from an individual, such as an individual having a cancer, e.g., a cancer provided herein. In one embodiment, a report according to the present disclosure indicates that a fusion nucleic acid molecule or polypeptide of the disclosure is present in a sample obtained from the individual. In one embodiment, a report according to the present disclosure indicates that a fusion nucleic acid molecule or polypeptide of the disclosure is not present in a sample obtained from the individual. In one embodiment, a report according to the present disclosure indicates that a fusion nucleic acid molecule or polypeptide of the disclosure has been detected in a sample obtained from the individual. In one embodiment, a report according to the present disclosure indicates that a fusion nucleic acid molecule or polypeptide of the disclosure has not been detected in a sample obtained from the individual. In some embodiments, the report comprises an identifier for the individual from which the sample was obtained.

In some embodiments, the report includes information on the role of a COL5A2-ALK fusion nucleic acid molecule or polypeptide or a COL3A1-ALK fusion nucleic acid molecule or polypeptide of the disclosure, or its wild type counterparts, in disease, such as in cancer. Such information can include one or more of: information on prognosis of a cancer, such as a cancer provided herein, e.g., comprising a fusion nucleic acid molecule or polypeptide described herein; information on resistance of a cancer, such as a cancer provided herein, e.g., comprising a fusion nucleic acid molecule or polypeptide described herein, to one or more treatments; information on potential or suggested therapeutic options (e.g., such as an anti-cancer therapy provided herein, or a treatment selected or identified according to the methods provided herein); or information on therapeutic options that should be avoided. In some embodiments, the report includes information on the likely effectiveness, acceptability, and/or advisability of applying a therapeutic option (e.g., such as an anti-cancer therapy provided herein, or a treatment selected or identified according to the methods provided herein) to an individual having a cancer, such as a cancer provided herein, e.g., comprising a fusion nucleic acid molecule or polypeptide described herein and identified in the report. In some embodiments, the report includes information or a recommendation on the administration of a treatment (e.g., an anti-cancer therapy provided herein, or a treatment selected or identified according to the methods provided herein). In some embodiments, the information or recommendation includes the dosage of the treatment and/or a treatment regimen (e.g., in combination with other treatments, such as a second therapeutic agent). In some embodiments, the report comprises information or a recommendation for at least one, at least two, at least three, at least four, at least five, at least six, at least seven, at least eight, at least nine, at least ten, or more treatments.

Also provided herein are methods of generating a report according to the present disclosure. In some embodiments, a report according to the present disclosure is generated by a method comprising one or more of the following steps: obtaining a sample, such as a sample described herein, from an individual, e.g., an individual having a cancer, such as a cancer provided herein; detecting a COL5A2-ALK fusion nucleic acid molecule or polypeptide or a COL3A1-ALK fusion nucleic acid molecule or polypeptide of the disclosure in the sample, or acquiring knowledge of the presence of a COL5A2-ALK fusion nucleic acid molecule or polypeptide or a COL3A1-ALK fusion nucleic acid molecule or polypeptide of the disclosure in the sample; and generating a report. In some embodiments, a report generated according to the methods provided herein comprises one or more of: information about the presence or absence of a fusion nucleic acid molecule or polypeptide of the disclosure in the sample; an identifier for the individual from which the sample was obtained; information on the role of a fusion nucleic acid molecule or polypeptide of the disclosure, or its wild type counterparts, in disease (e.g., such as in cancer); information on prognosis, resistance, or potential or suggested therapeutic options (such as an anti-cancer therapy provided herein, or a treatment selected or identified according to the methods provided herein); information on the likely effectiveness, acceptability, or the advisability of applying a therapeutic option (such as an anti-cancer therapy provided herein, or a treatment selected or identified according to the methods provided herein) to the individual; a recommendation or information on the administration of a treatment (such as an anti-cancer therapy provided herein, or a treatment selected or identified according to the methods provided herein); or a recommendation or information on the dosage or treatment regimen of a treatment (such as an anti-cancer therapy provided herein, or a treatment selected or identified according to the methods provided herein), e.g., in combination with other treatments (e.g., a second therapeutic agent). In some embodiments, the report generated is a personalized cancer report.

A report according to the present disclosure may be in an electronic, web-based, or paper form. The report may be provided to an individual or a patient (e.g., an individual or a patient with a cancer, such as a cancer provided herein, e.g., comprising a fusion nucleic acid molecule or polypeptide of the disclosure), or to an individual or entity other than the individual or patient (e.g., other than the individual or patient with the cancer), such as one or more of a caregiver, a physician, an oncologist, a hospital, a clinic, a third party payor, an insurance company, or a government entity. In some embodiments, the report is provided or delivered to the individual or entity within any of about 1 day or more, about 7 days or more, about 14 days or more, about 21 days or more, about 30 days or more, about 45 days or more, or about 60 days or more from obtaining a sample from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from detecting a fusion nucleic acid molecule or polypeptide of the disclosure in a sample obtained from an individual (e.g., an individual having a cancer). In some embodiments, the report is provided or delivered to an individual or entity within any of about 1 day or more, about 7 days or more, about 14 days or more, about 21 days or more, about 30 days or more, about 45 days or more, or about 60 days or more from acquiring knowledge of the presence of a fusion nucleic acid molecule or polypeptide of the disclosure in a sample obtained from an individual (e.g., an individual having a cancer).

The method steps of the methods described herein are intended to include any suitable method of causing one or more other parties or entities to perform the steps, unless a different meaning is expressly provided or otherwise clear from the context. Such parties or entities need not be under the direction or control of any other party or entity, and need not be located within a particular jurisdiction. Thus, for example, a description or recitation of “adding a first number to a second number” includes causing one or more parties or entities to add the two numbers together. For example, if person X engages in an arm's length transaction with person Y to add the two numbers, and person Y indeed adds the two numbers, then both persons X and Y perform the step as recited: person Y by virtue of the fact that he actually added the numbers, and person X by virtue of the fact that he caused person Y to add the numbers. Furthermore, if person X is located within the United States and person Y is located outside the United States, then the method is performed in the United States by virtue of person X's participation in causing the step to be performed.

### Anti-Cancer Therapies and Formulations

Certain aspects of the present disclosure relate to anti-cancer therapies. In some embodiments, an anti-cancer therapy of the disclosure includes one or more therapeutic agents, e.g., for treating a disease, disorder, or injury associated with a COL5A2-ALK fusion nucleic acid molecule or polypeptide or a COL3AT-ALK fusion nucleic acid molecule or polypeptide described herein, such as a cancer provided herein.

In some embodiments, the anti-cancer therapy is a small molecule inhibitor, an antibody, a cellular therapy (i.e., a cell-based therapy), or a nucleic acid. In some embodiments, the anti-cancer therapy is a chemotherapeutic agent, an anti-hormonal agent, an antimetabolite chemotherapeutic agent, a kinase inhibitor, a peptide, a gene therapy, a vaccine, a platinum-based chemotherapeutic agent, an immunotherapy, an antibody, or a checkpoint inhibitor. In some embodiments, the anti-cancer therapy is an ALK-targeted therapy. In some embodiments, an anti-cancer therapy comprises an anti-cancer agent that inhibits activity or expression of a COL5A2-ALK fusion nucleic acid molecule or polypeptide or a COL3AT-ALK fusion nucleic acid molecule or polypeptide of the disclosure. In some embodiments, the anti-cancer therapy inhibits the kinase activity of ALK, or a tyrosine kinase activity. In some embodiments, the anti-cancer therapy comprises a second agent, such as a second anti-cancer agent. In some embodiments, the anti-cancer therapy is administered in combination with a second anti-cancer therapy or agent.

In some embodiments, the anti-cancer therapy comprises a heat shock protein (HSP) inhibitor, a MYC inhibitor, an HDAC inhibitor, an immunotherapy, an ALK neoantigen, a vaccine, or a cellular therapy.

In some embodiments, the second anti-cancer agent includes one or more of an immune checkpoint inhibitor, a chemotherapy, a VEGF inhibitor, an Integrin 33 inhibitor, a statin, an EGFR inhibitor, an mTOR inhibitor, a PI3K inhibitor, a MAPK inhibitor, or a CDK4/6 inhibitor.

In some embodiments, the anti-cancer therapy comprises a kinase inhibitor. In some embodiments, the ALK-targeted therapy comprises a kinase inhibitor. In some embodiments, the methods provided herein comprise administering to the individual a kinase inhibitor, e.g., in combination with another anti-cancer therapy. In some embodiments, the kinase inhibitor is crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684). Additional examples of ALK kinase inhibitors that may be used according to any of the methods provided herein are described in examples 3-39 of WO2005016894, which is incorporated herein by reference.

In some embodiments, the anti-cancer therapy comprises a heat shock protein (HSP) inhibitor. In some embodiments, the methods provided herein comprise administering to the individual an HSP inhibitor, e.g., in combination with another anti-cancer therapy. In some embodiments, the HSP inhibitor is a Pan-HSP inhibitor, such as KNK423. In some embodiments, the HSP inhibitor is an HSP70 inhibitor, such as cmHsp70.1, quercetin, VER155008, or 17-AAD. In some embodiments, the HSP inhibitor is a HSP90 inhibitor. In some embodiments, the HSP90 inhibitor is 17-AAD, Debio0932, ganetespib (STA-9090), retaspimycin hydrochloride (retaspimycin, IPI-504), AUY922, alvespimycin (KOS-1022, 17-DMAG), tanespimycin (KOS-953, 17-AAG), DS 2248, or AT13387 (onalespib). In some embodiments, the HSP inhibitor is an HSP27 inhibitor, such as Apatorsen (OGX-427).

In some embodiments, the anti-cancer therapy comprises a MYC inhibitor. In some embodiments, the methods provided herein comprise administering to the individual a MYC inhibitor, e.g., in combination with another anti-cancer therapy. In some embodiments, the MYC inhibitor is MYCi361 (NUCC-0196361), MYCi975 (NUCC-0200975), Omomyc (dominant negative peptide), ZINC16293153 (Min9), 10058-F4, JKY-2-169, 7594-0035, or inhibitors of MYC/MAX dimerization and/or MYC/MAX/DNA complex formation.

In some embodiments, the anti-cancer therapy comprises a histone deacetylase (HDAC) inhibitor. In some embodiments, the methods provided herein comprise administering to the individual an HDAC inhibitor, e.g., in combination with another anti-cancer therapy. In some embodiments, the HDAC inhibitor is belinostat (PXD101, Beleodaq®), SAHA (vorinostat, suberoylanilide hydroxamine, Zolinza®), panobinostat (LBH589, LAQ-824), ACY1215 (Rocilinostat), quisinostat (JNJ-26481585), abexinostat (PCI-24781), pracinostat (SB939), givinostat (ITF2357), resminostat (4SC-201), trichostatin A (TSA), MS-275 (etinostat), Romidepsin (depsipeptide, FK228), MGCD0103 (mocetinostat), BML-210, CAY10603, valproic acid, MC1568, CUDC-907, CI-994 (Tacedinaline), Pivanex (AN-9), AR-42, Chidamide (CS055, HBI-8000), CUDC-101, CHR-3996, MPTOE028, BRD8430, MRLB-223, apicidin, RGFP966, BG45, PCI-34051, C149 (NCC149), TMP269, Cpd2, T247, T326, LMK235, CIA, HPOB, Nexturastat A, Befexamac, CBHA, Phenylbutyrate, MC1568, SNDX275, Scriptaid, Merck60, PX089344, PX105684, PX117735, PX117792, PX117245, PX105844, compound 12 as described by Li et al., Cold Spring Harb Perspect Med (2016) 6(10):a026831, or PX117445.

In some embodiments, the anti-cancer therapy comprises a VEGF inhibitor. In some embodiments, the methods provided herein comprise administering to the individual a VEGF inhibitor, e.g., in combination with another anti-cancer therapy. In some embodiments, the VEGF inhibitor is Bevacizumab (Avastin®), BMS-690514, ramucirumab, pazopanib, sorafenib, sunitinib, golvatinib, vandetanib, cabozantinib, levantinib, axitinib, cediranib, tivozanib, lucitanib, semaxanib, nindentanib, regorafinib, or aflibercept.

In some embodiments, the anti-cancer therapy comprises an integrin β3 inhibitor. In some embodiments, the methods provided herein comprise administering to the individual an integrin β3 inhibitor, e.g., in combination with another anti-cancer therapy. In some embodiments, the integrin β3 inhibitor is anti-avb3 (clone LM609), cilengitide (EMD121974, NSC, 707544), an siRNA, GLPG0187, MK-0429, CNTO95, TN-161, etaracizumab (MEDI-522), intetumumab (CNTO95) (anti-alphaV subunit antibody), abituzumab (EMD 525797/DI17E6) (anti-alphaV subunit antibody), JSM6427, SJ749, BCH-15046, SCH221153, or SC56631. In some embodiments, the anti-cancer therapy comprises an uIIbP3 integrin inhibitor. In some embodiments, the methods provided herein comprise administering to the individual an αIIbβ3 integrin inhibitor, e.g., in combination with another anti-cancer therapy. In some embodiments, the αIIbβ3 integrin inhibitor is abciximab, eptifibatide (Integrilin®), or tirofiban (Aggrastat®).

In some embodiments, the anti-cancer therapy comprises a statin or a statin-based agent. In some embodiments, the methods provided herein comprise administering to the individual a statin or a statin-based agent, e.g., in combination with another anti-cancer therapy. In some embodiments, the statin or statin-based agent is simvastatin, atorvastatin, fluvastatin, pitavastatin, pravastatin, rosuvastatin, or cerivastatin.

In some embodiments, the anti-cancer therapy comprises an mTOR inhibitor. In some embodiments, the methods provided herein comprise administering to the individual an mTOR inhibitor, e.g., in combination with another anti-cancer therapy. In some embodiments, the mTOR inhibitor is temsirolimus (CCI-779), KU-006379, PP242, Torin1, Torin2, ICSN3250, Rapalink-1, CC-223, sirolimus (rapamycin), everolimus (RAD001), dactosilib (NVP-BEZ235), GSK2126458, WAY-001, WAY-600, WYE-687, WYE-354, SF1126, XL765, INK128 (MLN012), AZD8055, OSI027, AZD2014, or AP-23573.

In some embodiments, the anti-cancer therapy comprises a PI3K inhibitor. In some embodiments, the methods provided herein comprise administering to the individual a PI3K inhibitor, e.g., in combination with another anti-cancer therapy. In some embodiments, the PI3K inhibitor is GSK2636771, buparlisib (BKM120), AZD8186, copanlisib (BAY80-6946), LY294002, PX-866, TGX115, TGX126, BEZ235, SF1126, idelalisib (GS-1101, CAL-101), pictilisib (GDC-094), GDCO0032, IP1145, INK1117 (MLN1117), SAR260301, KIN-193 (AZD6482), duvelisib, GS-9820, GSK2636771, GDC-0980, AMG319, pazobanib, or alpelisib (BYL719, Piqray).

In some embodiments, the anti-cancer therapy comprises a MAPK inhibitor. In some embodiments, the methods provided herein comprise administering to the individual a MAPK inhibitor, e.g., in combination with another anti-cancer therapy. In some embodiments, the MAPK inhibitor is SB203580, SKF-86002, BIRB-796, SC-409, RJW-67657, BIRB-796, VX-745, R03201195, SB-242235, or MW181.

In some embodiments, the anti-cancer therapy comprises a CDK4/6 inhibitor. In some embodiments, the methods provided herein comprise administering to the individual a CDK4/6 inhibitor, e.g., in combination with another anti-cancer therapy. In some embodiments, the CDK4/6 inhibitor is ribociclib (Kisqali®, LEE011), palbociclib (PD0332991, Ibrance®), or abemaciclib (LY2835219).

In some embodiments, the anti-cancer therapy comprises an EGFR inhibitor. In some embodiments, the methods provided herein comprise administering to the individual an EGFR inhibitor, e.g., in combination with another anti-cancer therapy. In some embodiments, the EGFR inhibitor is cetuximab, panitumumab, lapatinib, gefitinib, vandetanib, dacomitinib, icotinib, osimertinib (AZD9291), afatanib, olmutinib, EGF816 (nazartinib), avitinib (ACO0010), rociletinib (CO-1686), BMS-690514, YH5448, PF-06747775, ASP8273, PF299804, AP26113, or erlotinib. In some embodiments, the EGFR inhibitor is gefitinib or cetuximab.

In some embodiments, the anti-cancer therapy comprises a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy. In some embodiments, the methods provided herein comprise administering to the individual a cancer immunotherapy, such as a checkpoint inhibitor, cancer vaccine, cell-based therapy, T cell receptor (TCR)-based therapy, adjuvant immunotherapy, cytokine immunotherapy, and oncolytic virus therapy, e.g., in combination with another anti-cancer therapy. In some embodiments, the cancer immunotherapy comprises a small molecule, nucleic acid, polypeptide, carbohydrate, toxin, cell-based agent, or cell-binding agent. Examples of cancer immunotherapies are described in greater detail herein but are not intended to be limiting. In some embodiments, the cancer immunotherapy activates one or more aspects of the immune system to attack a cell (e.g., a tumor cell) that expresses a neoantigen, e.g., a neoantigen expressed by a cancer of the disclosure (e.g., a neoantigen corresponding to a COL5A2-ALK fusion nucleic acid molecule or polypeptide or a COL3A1-ALK fusion nucleic acid molecule or polypeptide described herein, such as an ALK neoantigen). The cancer immunotherapies of the present disclosure are contemplated for use as monotherapies, or in combination approaches comprising two or more in any combination or number, subject to medical judgement. Any of the cancer immunotherapies (optionally as monotherapies or in combination with another cancer immunotherapy or other therapeutic agent described herein) may find use in any of the methods described herein.

In some embodiments, the cancer immunotherapy comprises a cancer vaccine. A range of cancer vaccines have been tested that employ different approaches to promoting an immune response against a cancer (see, e.g., Emens L A, Expert Opin Emerg Drugs 13(2): 295-308 (2008) and US20190367613). Approaches have been designed to enhance the response of B cells, T cells, or professional antigen-presenting cells against tumors. Exemplary types of cancer vaccines include, but are not limited to, DNA-based vaccines, RNA-based vaccines, virus transduced vaccines, peptide-based vaccines, dendritic cell vaccines, oncolytic viruses, whole tumor cell vaccines, tumor antigen vaccines, etc. In some embodiments, the cancer vaccine can be prophylactic or therapeutic. In some embodiments, the cancer vaccine is formulated as a peptide-based vaccine, a nucleic acid-based vaccine, an antibody based vaccine, or a cell based vaccine. For example, a vaccine composition can include naked cDNA in cationic lipid formulations; lipopeptides (e.g., Vitiello, A. et ah, J. Clin. Invest. 95:341, 1995), naked cDNA or peptides, encapsulated e.g., in poly(DL-lactide-co-glycolide) (“PLG”) microspheres (see, e.g., Eldridge, et ah, Molec. Immunol. 28:287-294, 1991: Alonso et al, Vaccine 12:299-306, 1994; Jones et al, Vaccine 13:675-681, 1995); peptide composition contained in immune stimulating complexes (ISCOMS) (e.g., Takahashi et al, Nature 344:873-875, 1990; Hu et al, Clin. Exp. Immunol. 113:235-243, 1998); or multiple antigen peptide systems (MAPs) (see e.g., Tam, J. P., Proc. Natl Acad. Sci. U.S.A. 85:5409-5413, 1988; Tam, J. P., J. Immunol. Methods 196: 17-32, 1996). In some embodiments, a cancer vaccine is formulated as a peptide-based vaccine, or nucleic acid based vaccine in which the nucleic acid encodes the polypeptides. In some embodiments, a cancer vaccine is formulated as an antibody-based vaccine. In some embodiments, a cancer vaccine is formulated as a cell based vaccine. In some embodiments, the cancer vaccine is a peptide cancer vaccine, which in some embodiments is a personalized peptide vaccine. In some embodiments, the cancer vaccine is a multivalent long peptide, a multiple peptide, a peptide mixture, a hybrid peptide, or a peptide pulsed dendritic cell vaccine (see, e.g., Yamada et al, Cancer Sci, 104: 14-21), 2013). In some embodiments, such cancer vaccines augment the anti-cancer response.

In some embodiments, the cancer vaccine comprises a polynucleotide that encodes a neoantigen, e.g., a neoantigen expressed by a cancer of the disclosure (e.g., a neoantigen corresponding to a COL5A2-ALK fusion nucleic acid molecule or polypeptide or a COL3A1-ALK fusion nucleic acid molecule or polypeptide described herein, such as an ALK neoantigen). In some embodiments, the cancer vaccine comprises DNA that encodes a neoantigen, e.g., a neoantigen expressed by a cancer of the disclosure (e.g., a neoantigen corresponding to a COL5A2-ALK fusion nucleic acid molecule or polypeptide or a COL3A1-ALK fusion nucleic acid molecule or polypeptide described herein, such as an ALK neoantigen). In some embodiments, the cancer vaccine comprises RNA that encodes a neoantigen, e.g., a neoantigen expressed by a cancer of the disclosure (e.g., a neoantigen corresponding to a COL5A2-ALK fusion nucleic acid molecule or polypeptide or a COL3A1-ALK fusion nucleic acid molecule or polypeptide described herein, such as an ALK neoantigen). In some embodiments, the cancer vaccine comprises a polynucleotide that encodes a neoantigen, e.g., a neoantigen expressed by a cancer of the disclosure (e.g., a neoantigen corresponding to a COL5A2-ALK fusion nucleic acid molecule or polypeptide or a COL3A1-ALK fusion nucleic acid molecule or polypeptide described herein, such as an ALK neoantigen), as well as one or more additional antigens, neoantigens, or other sequences that promote antigen presentation and/or an immune response. In some embodiments, the polynucleotide is complexed with one or more additional agents, such as a liposome or lipoplex. In some embodiments, the polynucleotide(s) are taken up and translated by antigen presenting cells (APCs), which then present the neoantigen(s) via MHC class I on the APC cell surface.

In some embodiments, the cancer vaccine is selected from sipuleucel-T (Provenge®, Dendreon/Valeant Pharmaceuticals), which has been approved for treatment of asymptomatic, or minimally symptomatic metastatic castrate-resistant (hormone-refractory) prostate cancer; and talimogene laherparepvec (Imlygic®, BioVex/Amgen, previously known as T-VEC), a genetically modified oncolytic viral therapy approved for treatment of unresectable cutaneous, subcutaneous and nodal lesions in melanoma. In some embodiments, the cancer vaccine is selected from an oncolytic viral therapy such as pexastimogene devacirepvec (PexaVec/JX-594, SillaJen/formerly Jennerex Biotherapeutics), a thymidine kinase- (TK-) deficient vaccinia virus engineered to express GM-CSF, for hepatocellular carcinoma (NCT02562755) and melanoma (NCT00429312); pelareorep (Reolysin®, Oncolytics Biotech), a variant of respiratory enteric orphan virus (reovirus) which does not replicate in cells that are not RAS-activated, in numerous cancers, including colorectal cancer (NCT01622543). prostate cancer (NCT01619813), head and neck squamous cell cancer (NCT01166542), pancreatic adenocarcinoma (NCT00998322), and non-small cell lung cancer (NSCLC) (NCT 00861627); enadenotucirev (NG-348, PsiOxus, formerly known as ColoAdl), an adenovirus engineered to express a full length CD80 and an antibody fragment specific for the T-cell receptor CD3 protein, in ovarian cancer (NCT02028117), metastatic or advanced epithelial tumors such as in colorectal cancer, bladder cancer, head and neck squamous cell carcinoma and salivary gland cancer (NCT02636036); ONCOS-102 (Targovax/formerly Oncos), an adenovirus engineered to express GM-CSF, in melanoma (NCT03003676), and peritoneal disease, colorectal cancer or ovarian cancer (NCT02963831); GL-ONC1 (GLV-1h68/GLV-1h153, Genelux GmbH), vaccinia viruses engineered to express beta-galactosidase (beta-gal)/beta-glucoronidase or beta-gal/human sodium iodide symporter (hNIS), respectively, were studied in peritoneal carcinomatosis (NCT01443260), fallopian tube cancer, ovarian cancer (NCT 02759588); or CG0070 (Cold Genesys), an adenovirus engineered to express GM-CSF in bladder cancer (NCT02365818); anti-gp100; STINGVAX; GVAX; DCVaxL; and DNX-2401. In some embodiments, the cancer vaccine is selected from JX-929 (SillaJen/formerly Jennerex Biotherapeutics), a TK- and vaccinia growth factor-deficient vaccinia virus engineered to express cytosine deaminase, which is able to convert the prodrug 5-fluorocytosine to the cytotoxic drug 5-fluorouracil; TGO1 and TG02 (Targovax/formerly Oncos), peptide-based immunotherapy agents targeted for difficult-to-treat RAS mutations; and TILT-123 (TILT Biotherapeutics), an engineered adenovirus designated: Ad5/3-E2F-delta24-hTNFα-IRES-hIL20; and VSV-GP (ViraTherapeutics) a vesicular stomatitis virus (VSV) engineered to express the glycoprotein (GP) of lymphocytic choriomeningitis virus (LCMV), which can be further engineered to express antigens designed to raise an antigen-specific CD8+ T cell response. In some embodiments, the cancer vaccine comprises a vector-based tumor antigen vaccine. Vector-based tumor antigen vaccines can be used as a way to provide a steady supply of antigens to stimulate an anti-tumor immune response. In some embodiments, vectors encoding for tumor antigens are injected into an individual (possibly with pro-inflammatory or other attractants such as GM-CSF), taken up by cells in vivo to make the specific antigens, which then provoke the desired immune response. In some embodiments, vectors may be used to deliver more than one tumor antigen at a time, to increase the immune response. In addition, recombinant virus, bacteria or yeast vectors can trigger their own immune responses, which may also enhance the overall immune response.

In some embodiments, the cancer vaccine comprises a DNA-based vaccine. In some embodiments, DNA-based vaccines can be employed to stimulate an anti-tumor response. The ability of directly injected DNA that encodes an antigenic protein, to elicit a protective immune response has been demonstrated in numerous experimental systems. Vaccination through directly injecting DNA that encodes an antigenic protein, to elicit a protective immune response often produces both cell-mediated and humoral responses. Moreover, reproducible immune responses to DNA encoding various antigens have been reported in mice that last essentially for the lifetime of the animal (see, e.g., Yankauckas et al. (1993) DNA Cell Biol., 12: 771-776). In some embodiments, plasmid (or other vector) DNA that includes a sequence encoding a protein operably linked to regulatory elements required for gene expression is administered to individuals (e.g. human patients, non-human mammals, etc.). In some embodiments, the cells of the individual take up the administered DNA and the coding sequence is expressed. In some embodiments, the antigen so produced becomes a target against which an immune response is directed.

In some embodiments, the cancer vaccine comprises an RNA-based vaccine. In some embodiments, RNA-based vaccines can be employed to stimulate an anti-tumor response. In some embodiments, RNA-based vaccines comprise a self-replicating RNA molecule. In some embodiments, the self-replicating RNA molecule may be an alphavirus-derived RNA replicon. Self-replicating RNA (or “SAM”) molecules are well known in the art and can be produced by using replication elements derived from, e.g., alphaviruses, and substituting the structural viral proteins with a nucleotide sequence encoding a protein of interest. A self-replicating RNA molecule is typically a +-strand molecule which can be directly translated after delivery to a cell, and this translation provides a RNA-dependent RNA polymerase which then produces both antisense and sense transcripts from the delivered RNA. Thus, the delivered RNA leads to the production of multiple daughter RNAs. These daughter RNAs, as well as collinear subgenomic transcripts, may be translated themselves to provide in situ expression of an encoded polypeptide, or may be transcribed to provide further transcripts with the same sense as the delivered RNA which are translated to provide in situ expression of the antigen.

In some embodiments, the cancer immunotherapy comprises a cell-based therapy. In some embodiments, the cancer immunotherapy comprises a T cell-based therapy. In some embodiments, the cancer immunotherapy comprises an adoptive therapy, e.g., an adoptive T cell-based therapy. In some embodiments, the T cells are autologous or allogeneic to the recipient. In some embodiments, the T cells are CD8+ T cells. In some embodiments, the T cells are CD4+ T cells. Adoptive immunotherapy refers to a therapeutic approach for treating cancer or infectious diseases in which immune cells are administered to a host with the aim that the cells mediate either directly or indirectly specific immunity to (i.e., mount an immune response directed against) cancer cells. In some embodiments, the immune response results in inhibition of tumor and/or metastatic cell growth and/or proliferation, and in related embodiments, results in neoplastic cell death and/or resorption. The immune cells can be derived from a different organism/host (exogenous immune cells) or can be cells obtained from the subject organism (autologous immune cells). In some embodiments, the immune cells (e.g., autologous or allogeneic T cells (e.g., regulatory T cells, CD4+ T cells, CD8+ T cells, or gamma-delta T cells), NK cells, invariant NK cells, or NKT cells) can be genetically engineered to express antigen receptors such as engineered TCRs and/or chimeric antigen receptors (CARs). For example, the host cells (e.g., autologous or allogeneic T-cells) are modified to express a T cell receptor (TCR) having antigenic specificity for a cancer antigen. In some embodiments, NK cells are engineered to express a TCR. The NK cells may be further engineered to express a CAR. Multiple CARs and/or TCRs, such as to different antigens, may be added to a single cell type, such as T cells or NK cells. In some embodiments, the cells comprise one or more nucleic acids/expression constructs/vectors introduced via genetic engineering that encode one or more antigen receptors, and genetically engineered products of such nucleic acids. In some embodiments, the nucleic acids are heterologous, i.e., normally not present in a cell or sample obtained from the cell, such as one obtained from another organism or cell, which for example, is not ordinarily found in the cell being engineered and/or an organism from which such cell is derived. In some embodiments, the nucleic acids are not naturally occurring, such as a nucleic acid not found in nature (e.g. chimeric). In some embodiments, a population of immune cells can be obtained from a subject in need of therapy or suffering from a disease associated with reduced immune cell activity. Thus, the cells will be autologous to the subject in need of therapy. In some embodiments, a population of immune cells can be obtained from a donor, such as a histocompatibility-matched donor. In some embodiments, the immune cell population can be harvested from the peripheral blood, cord blood, bone marrow, spleen, or any other organ/tissue in which immune cells reside in said subject or donor. In some embodiments, the immune cells can be isolated from a pool of subjects and/or donors, such as from pooled cord blood. In some embodiments, when the population of immune cells is obtained from a donor distinct from the subject, the donor may be allogeneic, provided the cells obtained are subject-compatible, in that they can be introduced into the subject. In some embodiments, allogeneic donor cells may or may not be human-leukocyte-antigen (HLA)-compatible. In some embodiments, to be rendered subject-compatible, allogeneic cells can be treated to reduce immunogenicity.

In some embodiments, the cell-based therapy comprises a T cell-based therapy, such as autologous cells, e.g., tumor-infiltrating lymphocytes (TILs); T cells activated ex-vivo using autologous DCs, lymphocytes, artificial antigen-presenting cells (APCs) or beads coated with T cell ligands and activating antibodies, or cells isolated by virtue of capturing target cell membrane; allogeneic cells naturally expressing anti-host tumor T cell receptor (TCR); and non-tumor-specific autologous or allogeneic cells genetically reprogrammed or “redirected” to express tumor-reactive TCR or chimeric TCR molecules displaying antibody-like tumor recognition capacity known as “T-bodies”. Several approaches for the isolation, derivation, engineering or modification, activation, and expansion of functional anti-tumor effector cells have been described in the last two decades and may be used according to any of the methods provided herein. In some embodiments, the T cells are derived from the blood, bone marrow, lymph, umbilical cord, or lymphoid organs. In some embodiments, the cells are human cells. In some embodiments, the cells are primary cells, such as those isolated directly from a subject and/or isolated from a subject and frozen. In some embodiments, the cells include one or more subsets of T cells or other cell types, such as whole T cell populations, CD4⁺ cells, CD8⁺ cells, and subpopulations thereof, such as those defined by function, activation state, maturity, potential for differentiation, expansion, recirculation, localization, and/or persistence capacities, antigen-specificity, type of antigen receptor, presence in a particular organ or compartment, marker or cytokine secretion profile, and/or degree of differentiation. In some embodiments, the cells may be allogeneic and/or autologous. In some embodiments, such as for off-the-shelf technologies, the cells are pluripotent and/or multipotent, such as stem cells, such as induced pluripotent stem cells (iPSCs).

In some embodiments, the T cell-based therapy comprises a chimeric antigen receptor (CAR)-T cell-based therapy. This approach involves engineering a CAR that specifically binds to an antigen of interest and comprises one or more intracellular signaling domains for T cell activation. The CAR is then expressed on the surface of engineered T cells (CAR-T) and administered to a patient, leading to a T-cell-specific immune response against cancer cells expressing the antigen. In some embodiments, the CAR specifically binds a neoantigen, such as a neoantigen corresponding to a fusion (e.g., a fusion polypeptide) provided herein, e.g., an ALK neoantigen.

In some embodiments, the T cell-based therapy comprises T cells expressing a recombinant T cell receptor (TCR). This approach involves identifying a TCR that specifically binds to an antigen of interest, which is then used to replace the endogenous or native TCR on the surface of engineered T cells that are administered to a patient, leading to a T-cell-specific immune response against cancer cells expressing the antigen. In some embodiments, the recombinant TCR specifically binds a neoantigen corresponding to a fusion (e.g., a fusion polypeptide) provided herein, e.g., an ALK neoantigen.

In some embodiments, the T cell-based therapy comprises tumor-infiltrating lymphocytes (TILs). For example, TILs can be isolated from a tumor or cancer of the present disclosure, then isolated and expanded in vitro. Some or all of these TILs may specifically recognize an antigen expressed by the tumor or cancer of the present disclosure. In some embodiments, the TILs are exposed to one or more neoantigens, e.g., a neoantigen corresponding to a fusion (e.g., a fusion polypeptide) provided herein, e.g., an ALK neoantigen, in vitro after isolation. TILs are then administered to the patient (optionally in combination with one or more cytokines or other immune-stimulating substances).

In some embodiments, the cell-based therapy comprises a natural killer (NK) cell-based therapy. Natural killer (NK) cells are a subpopulation of lymphocytes that have spontaneous cytotoxicity against a variety of tumor cells, virus-infected cells, and some normal cells in the bone marrow and thymus. NK cells are critical effectors of the early innate immune response toward transformed and virus-infected cells. NK cells can be detected by specific surface markers, such as CD16, CD56, and CD8 in humans. NK cells do not express T-cell antigen receptors, the pan T marker CD3, or surface immunoglobulin B cell receptors. In some embodiments, NK cells are derived from human peripheral blood mononuclear cells (PBMC), unstimulated leukapheresis products (PBSC), human embryonic stem cells (hESCs), induced pluripotent stem cells (iPSCs), bone marrow, or umbilical cord blood by methods well known in the art.

In some embodiments, the cell-based therapy comprises a dendritic cell (DC)-based therapy, e.g., a dendritic cell vaccine. In some embodiments, the DC vaccine comprises antigen-presenting cells that are able to induce specific T cell immunity, which are harvested from the patient or from a donor. In some embodiments, the DC vaccine can then be exposed in vitro to a peptide antigen, for which T cells are to be generated in the patient. In some embodiments, dendritic cells loaded with the antigen are then injected back into the patient. In some embodiments, immunization may be repeated multiple times if desired. Methods for harvesting, expanding, and administering dendritic cells are known in the art; see, e.g., WO2019178081. Dendritic cell vaccines (such as Sipuleucel-T, also known as APC8015 and PROVENGE®) are vaccines that involve administration of dendritic cells that act as APCs to present one or more cancer-specific antigens to the patient's immune system. In some embodiments, the dendritic cells are autologous or allogeneic to the recipient.

In some embodiments, the cancer immunotherapy comprises a TCR-based therapy. In some embodiments, the cancer immunotherapy comprises administration of one or more TCRs or TCR-based therapeutics that specifically bind an antigen expressed by a cancer of the present disclosure, e.g., an antigen corresponding to a fusion polypeptide of the disclosure. In some embodiments, the TCR-based therapeutic may further include a moiety that binds an immune cell (e.g., a T cell), such as an antibody or antibody fragment that specifically binds a T cell surface protein or receptor (e.g., an anti-CD3 antibody or antibody fragment).

In some embodiments, the immunotherapy comprises adjuvant immunotherapy. Adjuvant immunotherapy comprises the use of one or more agents that activate components of the innate immune system, e.g., HILTONOL® (imiquimod), which targets the TLR7 pathway.

In some embodiments, the immunotherapy comprises cytokine immunotherapy. Cytokine immunotherapy comprises the use of one or more cytokines that activate components of the immune system. Examples include, but are not limited to, aldesleukin (PROLEUKIN®; interleukin-2), interferon alfa-2a (ROFERON®-A), interferon alfa-2b (INTRON®-A), and peginterferon alfa-2b (PEGINTRON®).

In some embodiments, the immunotherapy comprises oncolytic virus therapy. Oncolytic virus therapy uses genetically modified viruses to replicate in and kill cancer cells, leading to the release of antigens that stimulate an immune response. In some embodiments, replication-competent oncolytic viruses expressing a tumor antigen comprise any naturally occurring (e.g., from a “field source”) or modified replication-competent oncolytic virus. In some embodiments, the oncolytic virus, in addition to expressing a tumor antigen, may be modified to increase selectivity of the virus for cancer cells. In some embodiments, replication-competent oncolytic viruses include, but are not limited to, oncolytic viruses that are a member in the family of myoviridae, siphoviridae, podpviridae, teciviridae, corticoviridae, plasmaviridae, lipothrixviridae, fuselloviridae, poxyiridae, iridoviridae, phycodnaviridae, baculoviridae, herpesviridae, adnoviridae, papovaviridae, polydnaviridae, inoviridae, microviridae, geminiviridae, circoviridae, parvoviridae, hcpadnaviridae, retroviridae, cyctoviridae, reoviridae, birnaviridae, paramyxoviridae, rhabdoviridae, filoviridae, orthomyxoviridae, bunyaviridae, arenaviridae, Leviviridae, picornaviridae, sequiviridae, comoviridae, potyviridae, caliciviridae, astroviridae, nodaviridae, tetraviridae, tombusviridae, coronaviridae, glaviviridae, togaviridae, and bamaviridae. In some embodiments, replication-competent oncolytic viruses include adenovirus, retrovirus, reovirus, rhabdovirus, Newcastle Disease virus (NDV), polyoma virus, vaccinia virus (VacV), herpes simplex virus, picornavirus, coxsackie virus and parvovirus. In some embodiments, a replicative oncolytic vaccinia virus expressing a tumor antigen may be engineered to lack one or more functional genes in order to increase the cancer selectivity of the virus. In some embodiments, an oncolytic vaccinia virus is engineered to lack thymidine kinase (TK) activity. In some embodiments, the oncolytic vaccinia virus may be engineered to lack vaccinia virus growth factor (VGF). In some embodiments, an oncolytic vaccinia virus may be engineered to lack both VGF and TK activity. In some embodiments, an oncolytic vaccinia virus may be engineered to lack one or more genes involved in evading host interferon (IFN) response such as E3L, K3L, B18R, or B8R. In some embodiments, a replicative oncolytic vaccinia virus is a Western Reserve, Copenhagen, Lister or Wyeth strain and lacks a functional TK gene. In some embodiments, the oncolytic vaccinia virus is a Western Reserve, Copenhagen, Lister or Wyeth strain lacking a functional B18R and/or B8R gene. In some embodiments, a replicative oncolytic vaccinia virus expressing a tumor antigen may be locally or systemically administered to a subject, e.g. via intratumoral, intraperitoneal, intravenous, intra-arterial, intramuscular, intradermal, intracranial, subcutaneous, or intranasal administration.

In some embodiments, the anti-cancer therapy comprises an immune checkpoint inhibitor. In some embodiments, the methods provided herein comprise administering to the individual an immune checkpoint inhibitor, e.g., in combination with another anti-cancer therapy. In some embodiments, the methods provided herein comprise administering to an individual an effective amount of an immune checkpoint inhibitor. As is known in the art, a checkpoint inhibitor targets at least one immune checkpoint protein to alter the regulation of an immune response. Immune checkpoint proteins include, e.g., CTLA4, PD-L1, PD-1, PD-L2, VISTA, B7-H2, B7-H3, B7-H4, B7-H6, 2B4, ICOS, HVEM, CEACAM, LAIR1, CD80, CD86, CD276, VTCN1, MHC class I, MHC class II, GALS, adenosine, TGFR, CSF1R, MICA/B, arginase, CD160, gp49B, PIR-B, KIR family receptors, TIM-1, TIM-3, TIM-4, LAG-3, BTLA, SIRPalpha (CD47), CD48, 2B4 (CD244), B7.1, B7.2, ILT-2, ILT-4, TIGIT, LAG-3, BTLA, IDO, OX40, and A2aR. In some embodiments, molecules involved in regulating immune checkpoints include, but are not limited to: PD-1 (CD279), PD-L1 (B7-H1, CD274), PD-L2 (B7-CD, CD273), CTLA-4 (CD152), HVEM, BTLA (CD272), a killer-cell immunoglobulin-like receptor (KIR), LAG-3 (CD223), TIM-3 (HAVCR2), CEACAM, CEACAM-1, CEACAM-3, CEACAM-5, GAL9, VISTA (PD-1H), TIGIT, LAIR1, CD160, 2B4, TGFRbeta, A2AR, GITR (CD357), CD80 (B7-1), CD86 (B7-2), CD276 (B7-H3), VTCNI (B7-H4), MHC class I, MHC class II, GALS, adenosine, TGFR, B7-H1, OX40 (CD134), CD94 (KLRD1), CD137 (4-1BB), CD137L (4-1BBL), CD40, IDO, CSF1R, CD40L, CD47, CD70 (CD27L), CD226, HHLA2, ICOS (CD278), ICOSL (CD275), LIGHT (TNFSFi4, CD258), NKG2a, NKG2d, OX40L (CD134L), PVR (NECL5, CD155), SIRPa, MICA/B, and/or arginase. In some embodiments, an immune checkpoint inhibitor (i.e., a checkpoint inhibitor) decreases the activity of a checkpoint protein that negatively regulates immune cell function, e.g., in order to enhance T cell activation and/or an anti-cancer immune response. In other embodiments, a checkpoint inhibitor increases the activity of a checkpoint protein that positively regulates immune cell function, e.g., in order to enhance T cell activation and/or an anti-cancer immune response. In some embodiments, the checkpoint inhibitor is an antibody. Examples of checkpoint inhibitors include, without limitation, a PD-1 axis binding antagonist, a PD-L1 axis binding antagonist (e.g., an anti-PD-L1 antibody, e.g., atezolizumab (MPDL3280A)), an antagonist directed against a co-inhibitory molecule (e.g., a CTLA4 antagonist (e.g., an anti-CTLA4 antibody), a TIM-3 antagonist (e.g., an anti-TIM-3 antibody), or a LAG-3 antagonist (e.g., an anti-LAG-3 antibody)), or any combination thereof. In some embodiments, the immune checkpoint inhibitors comprise drugs such as small molecules, recombinant forms of ligand or receptors, or antibodies, such as human antibodies (see, e.g., International Patent Publication WO2015016718; Pardoll, Nat Rev Cancer, 12(4): 252-64, 2012; both incorporated herein by reference). In some embodiments, known inhibitors of immune checkpoint proteins or analogs thereof may be used, in particular chimerized, humanized or human forms of antibodies may be used.

In some embodiments, the checkpoint inhibitor is a PD-L1 axis binding antagonist, e.g., a PD-1 binding antagonist, a PD-L1 binding antagonist, or a PD-L2 binding antagonist. PD-1 (programmed death 1) is also referred to in the art as “programmed cell death 1,” “PDCD1,” “CD279,” and “SLEB2.” An exemplary human PD-1 is shown in UniProtKB/Swiss-Prot Accession No. Q15116. PD-L1 (programmed death ligand 1) is also referred to in the art as “programmed cell death 1 ligand 1,” “PDCD1 LG1,” “CD274,” “B7-H,” and “PDL1.” An exemplary human PD-L1 is shown in UniProtKB/Swiss-Prot Accession No. Q9NZQ7.1. PD-L2 (programmed death ligand 2) is also referred to in the art as “programmed cell death 1 ligand 2,” “PDCD1 LG2,” “CD273,” “B7-DC,” “Btdc,” and “PDL2.” An exemplary human PD-L2 is shown in UniProtKB/Swiss-Prot Accession No. Q9BQ51. In some instances, PD-1, PD-L1, and PD-L2 are human PD-1, PD-L1 and PD-L2.

In some instances, the PD-1 binding antagonist is a molecule that inhibits the binding of PD-1 to its ligand binding partners. In a specific embodiment, the PD-1 ligand binding partners are PD-L1 and/or PD-L2. In another instance, a PD-L1 binding antagonist is a molecule that inhibits the binding of PD-L1 to its binding ligands. In a specific embodiment, PD-L1 binding partners are PD-1 and/or B7-1. In another instance, the PD-L2 binding antagonist is a molecule that inhibits the binding of PD-L2 to its ligand binding partners. In a specific embodiment, the PD-L2 binding ligand partner is PD-1. The antagonist may be an antibody, an antigen binding fragment thereof, an immunoadhesin, a fusion protein, or an oligopeptide. In some embodiments, the PD-1 binding antagonist is a small molecule, a nucleic acid, a polypeptide (e.g., antibody), a carbohydrate, a lipid, a metal, or a toxin.

In some instances, the PD-1 binding antagonist is an anti-PD-1 antibody (e.g., a human antibody, a humanized antibody, or a chimeric antibody), for example, as described below. In some instances, the anti-PD-1 antibody is selected from the group consisting of MDX-1 106 (nivolumab), MK-3475 (pembrolizumab, Keytruda®), MEDI-0680 (AMP-514), PDR001, REGN2810, MGA-012, JNJ-63723283, BI 754091, and BGB-108. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence)). In some instances, the PD-1 binding antagonist is AMP-224. Other examples of anti-PD-1 antibodies include, but are not limited to, MEDI-0680 (AMP-514; AstraZeneca), PDR001 (CAS Registry No. 1859072-53-9; Novartis), REGN2810 (LIBTAYO® or cemiplimab-rwlc; Regeneron), BGB-108 (BeiGene), BGB-A317 (BeiGene), BI 754091, JS-001 (Shanghai Junshi), STI-A1110 (Sorrento), INCSHR-1210 (Incyte), PF-06801591 (Pfizer), TSR-042 (also known as ANB011; Tesaro/AnaptysBio), AM0001 (ARMO Biosciences), ENUM 244C8 (Enumeral Biomedical Holdings), or ENUM 388D4 (Enumeral Biomedical Holdings). In some embodiments, the PD-1 axis binding antagonist comprises tislelizumab (BGB-A317), BGB-108, STI-A1110, AM0001, BI 754091, sintilimab (IBI308), cetrelimab (JNJ-63723283), toripalimab (JS-001), camrelizumab (SHR-1210, INCSHR-1210, HR-301210), MEDI-0680 (AMP-514), MGA-012 (INCMGA 0012), nivolumab (BMS-936558, MDX1106, ONO-4538), spartalizumab (PDR001), pembrolizumab (MK-3475, SCH 900475, Keytruda®), PF-06801591, cemiplimab (REGN-2810, REGEN2810), dostarlimab (TSR-042, ANB011), FITC-YT-16 (PD-1 binding peptide), APL-501 or CBT-501 or genolimzumab (GB-226), AB-122, AK105, AMG 404, BCD-100, F520, HLX10, HX008, JTX-4014, LZM009, Sym021, PSB205, AMP-224 (fusion protein targeting PD-1), CX-188 (PD-1 probody), AGEN-2034, GLS-010, budigalimab (ABBV-181), AK-103, BAT-1306, CS-1003, AM-0001, TILT-123, BH-2922, BH-2941, BH-2950, ENUM-244C8, ENUM-388D4, HAB-21, H EISCOI 11-003, IKT-202, MCLA-134, MT-17000, PEGMP-7, PRS-332, RXI-762, STI-1110, VXM-10, XmAb-23104, AK-112, HLX-20, SSI-361, AT-16201, SNA-01, AB122, PD1-PIK, PF-06936308, RG-7769, CAB PD-1 Abs, AK-123, MEDI-3387, MEDI-5771, 4H1128Z-E27, REMD-288, SG-001, BY-24.3, CB-201, IBI-319, ONCR-177, Max-1, CS-4100, JBI-426, CCC-0701, or CCX-4503, or derivatives thereof.

In some embodiments, the PD-L1 binding antagonist is a small molecule that inhibits PD-1. In some embodiments, the PD-L1 binding antagonist is a small molecule that inhibits PD-L1. In some embodiments, the PD-L1 binding antagonist is a small molecule that inhibits PD-L1 and VISTA or PD-L1 and TIM3. In some embodiments, the PD-L1 binding antagonist is CA-170 (also known as AUPM-170). In some embodiments, the PD-L1 binding antagonist is an anti-PD-L1 antibody. In some embodiments, the anti-PD-L1 antibody can bind to a human PD-L1, for example a human PD-L1 as shown in UniProtKB/Swiss-Prot Accession No. Q9NZQ7.1, or a variant thereof. In some embodiments, the PD-L1 binding antagonist is a small molecule, a nucleic acid, a polypeptide (e.g., antibody), a carbohydrate, a lipid, a metal, or a toxin.

In some instances, the PD-L1 binding antagonist is an anti-PD-L1 antibody, for example, as described below. In some instances, the anti-PD-L1 antibody is capable of inhibiting the binding between PD-L1 and PD-1, and/or between PD-L1 and B7-1. In some instances, the anti-PD-L1 antibody is a monoclonal antibody. In some instances, the anti-PD-L1 antibody is an antibody fragment selected from a Fab, Fab′-SH, Fv, scFv, or (Fab′)2 fragment. In some instances, the anti-PD-L1 antibody is a humanized antibody. In some instances, the anti-PD-L1 antibody is a human antibody. In some instances, the anti-PD-L1 antibody is selected from YW243.55.570, MPDL3280A (atezolizumab), MDX-1 105, MEDI4736 (durvalumab), or MSB0010718C (avelumab). In some embodiments, the PD-L1 axis binding antagonist comprises atezolizumab, avelumab, durvalumab (imfinzi), BGB-A333, SHR-1316 (HTI-1088), CK-301, BMS-936559, envafolimab (KN035, ASC22), CS1001, MDX-1105 (BMS-936559), LY3300054, STI-A1014, FAZ053, CX-072, INCB086550, GNS-1480, CA-170, CK-301, M-7824, HTI-1088 (HTI-131, SHR-1316), MSB-2311, AK-106, AVA-004, BBI-801, CA-327, CBA-0710, CBT-502, FPT-155, IKT-201, IKT-703, 10-103, JS-003, KD-033, KY-1003, MCLA-145, MT-5050, SNA-02, BCD-135, APL-502 (CBT-402 or TQB2450), IMC-001, KD-045, INBRX-105, KN-046, IMC-2102, IMC-2101, KD-005, IMM-2502, 89Zr-CX-072, 89Zr-DFO-6E11, KY-1055, MEDI-1109, MT-5594, SL-279252, DSP-106, Gensci-047, REMD-290, N-809, PRS-344, FS-222, GEN-1046, BH-29xx, or FS-118, or a derivative thereof.

In some embodiments, the checkpoint inhibitor is an antagonist of CTLA4. In some embodiments, the checkpoint inhibitor is a small molecule antagonist of CTLA4. In some embodiments, the checkpoint inhibitor is an anti-CTLA4 antibody. CTLA4 is part of the CD28-B7 immunoglobulin superfamily of immune checkpoint molecules that acts to negatively regulate T cell activation, particularly CD28-dependent T cell responses. CTLA4 competes for binding to common ligands with CD28, such as CD80 (B7-1) and CD86 (B7-2), and binds to these ligands with higher affinity than CD28. Blocking CTLA4 activity (e.g., using an anti-CTLA4 antibody) is thought to enhance CD28-mediated costimulation (leading to increased T cell activation/priming), affect T cell development, and/or deplete Tregs (such as intratumoral Tregs). In some embodiments, the CTLA4 antagonist is a small molecule, a nucleic acid, a polypeptide (e.g., antibody), a carbohydrate, a lipid, a metal, or a toxin. In some embodiments, the CTLA-4 inhibitor comprises ipilimumab (1B1310, BMS-734016, MDXO10, MDX-CTLA4, MEDI4736), tremelimumab (CP-675, CP-675,206), APL-509, AGEN1884, CS1002, AGEN1181, Abatacept (Orencia, BMS-188667, RG2077), BCD-145, ONC-392, ADU-1604, REGN4659, ADG116, KN044, KN046, or a derivative thereof.

In some embodiments, the anti-PD-1 antibody or antibody fragment is MDX-1106 (nivolumab), MK-3475 (pembrolizumab, Keytruda®), MEDI-0680 (AMP-514), PDR001, REGN2810, MGA-012, JNJ-63723283, BI 754091, BGB-108, BGB-A317, JS-001, STI-A1110, INCSHR-1210, PF-06801591, TSR-042, AM0001, ENUM 244C8, or ENUM 388D4. In some embodiments, the PD-1 binding antagonist is an anti-PD-1 immunoadhesin. In some embodiments, the anti-PD-1 immunoadhesin is AMP-224. In some embodiments, the anti-PD-L1 antibody or antibody fragment is YW243.55.S70, MPDL3280A (atezolizumab), MDX-1105, MEDI4736 (durvalumab), MSB0010718C (avelumab), LY3300054, STI-A1014, KN035, FAZ053, or CX-072.

In some embodiments, the immune checkpoint inhibitor comprises a LAG-3 inhibitor (e.g., an antibody, an antibody conjugate, or an antigen-binding fragment thereof). In some embodiments, the LAG-3 inhibitor comprises a small molecule, a nucleic acid, a polypeptide (e.g., an antibody), a carbohydrate, a lipid, a metal, or a toxin. In some embodiments, the LAG-3 inhibitor comprises a small molecule. In some embodiments, the LAG-3 inhibitor comprises a LAG-3 binding agent. In some embodiments, the LAG-3 inhibitor comprises an antibody, an antibody conjugate, or an antigen-binding fragment thereof. In some embodiments, the LAG-3 inhibitor comprises eftilagimod alpha (IMP321, IMP-321, EDDP-202, EOC-202), relatlimab (BMS-986016), GSK2831781 (IMP-731), LAG525 (IMP701), TSR-033, EVIP321 (soluble LAG-3 protein), BI 754111, IMP761, REGN3767, MK-4280, MGD-013, XmAb22841, INCAGN-2385, ENUM-006, AVA-017, AM-0003, iOnctura anti-LAG-3 antibody, Arcus Biosciences LAG-3 antibody, Sym022, a derivative thereof, or an antibody that competes with any of the preceding.

In some embodiments, the anti-cancer therapy comprises an immunoregulatory molecule or a cytokine. In some embodiments, the methods provided herein comprise administering to the individual an immunoregulatory molecule or a cytokine, e.g., in combination with another anti-cancer therapy. An immunoregulatory profile is required to trigger an efficient immune response and balance the immunity in a subject. Examples of suitable immunoregulatory cytokines include, but are not limited to, interferons (e.g., IFNα, IFNβ and IFNγ), interleukins (e.g., IL-1, IL-2, IL-3, IL-4, IL-5, IL-6, IL-7, IL-8, IL-9, IL-10, IL-12 and IL-20), tumor necrosis factors (e.g., TNFα and TNFβ), erythropoietin (EPO), FLT-3 ligand, gIp10, TCA-3, MCP-1, MIF, MIP-lW, MIP-10, Rantes, macrophage colony stimulating factor (M-CSF), granulocyte colony stimulating factor (G-CSF), or granulocyte-macrophage colony stimulating factor (GM-CSF), as well as functional fragments thereof. In some embodiments, any immunomodulatory chemokine that binds to a chemokine receptor, i.e., a CXC, CC, C, or CX3C chemokine receptor, can be used in the context of the present disclosure. Examples of chemokines include, but are not limited to, MIP-3α (Lax), MIP-3β, Hcc-1, MPIF-1, MPIF-2, MCP-2, MCP-3, MCP-4, MCP-5, Eotaxin, Tarc, Elc, 1309, IL-8, GCP-2 Groα, Gro-β, Nap-2, Ena-78, Ip-10, MIG, I-Tac, SDF-1, or BCA-1 (Blc), as well as functional fragments thereof. In some embodiments, the immunoregulatory molecule is included with any of the treatments provided herein.

In some embodiments, the immune checkpoint inhibitor is monovalent and/or monospecific. In some embodiments, the immune checkpoint inhibitor is multivalent and/or multispecific.

In some embodiments, the anti-cancer therapy comprises an anti-cancer agent that inhibits expression of a COL5A2-ALK fusion nucleic acid molecule or polypeptide or a COL3A1-ALK fusion nucleic acid molecule or polypeptide. In some embodiments, the methods provided herein comprise administering to the individual an anti-cancer agent that inhibits expression of a COL5A2-ALK fusion nucleic acid molecule or polypeptide or a COL3A1-ALK fusion nucleic acid molecule or polypeptide, e.g., in combination with another anti-cancer therapy.

In some embodiments, the anti-cancer therapy comprises a nucleic acid molecule, such as a dsRNA, an siRNA, or an shRNA. In some embodiments, the methods provided herein comprise administering to the individual a nucleic acid molecule, such as a dsRNA, an siRNA, or an shRNA, e.g., in combination with another anti-cancer therapy. As is known in the art, dsRNAs having a duplex structure are effective at inducing RNA interference (RNAi). In some embodiments, the anti-cancer therapy comprises a small interfering RNA molecule (siRNA). dsRNAs and siRNAs can be used to silence gene expression in mammalian cells (e.g., human cells). In some embodiments, a dsRNA of the disclosure comprises any of between about 5 and about 10 base pairs, between about 10 and about 12 base pairs, between about 12 and about 15 base pairs, between about 15 and about 20 base pairs, between about 20 and 23 base pairs, between about 23 and about 25 base pairs, between about 25 and about 27 base pairs, or between about 27 and about 30 base pairs. As is known in the art, siRNAs are small dsRNAs that optionally include overhangs. In some embodiments, the duplex region of an siRNA is between about 18 and 25 nucleotides, e.g., any of 18, 19, 20, 21, 22, 23, 24, or 25 nucleotides. siRNAs may also include short hairpin RNAs (shRNAs), e.g., with approximately 29-base-pair stems and 2-nucleotide 3′ overhangs. In some embodiments, a dsRNA, an siRNA, or an shRNA of the disclosure comprises a nucleotide sequence that is configured to hybridize to a COL5A2-ALK fusion nucleic acid molecule or a COL3A1-ALK fusion nucleic acid molecule provided herein. In some embodiments, a dsRNA, an siRNA, or an shRNA of the disclosure comprises a nucleotide sequence that is configured to hybridize to the COL5A2-ALK breakpoint or the COL3A1-ALK breakpoint of a fusion nucleic acid molecule provided herein. Methods for designing, optimizing, producing, and using dsRNAs, siRNAs, or shRNAs, are known in the art.

In some embodiments, the anti-cancer therapy comprises a chemotherapy. In some embodiments, the methods provided herein comprise administering to the individual a chemotherapy, e.g., in combination with another anti-cancer therapy. Examples of chemotherapeutic agents include alkylating agents, such as thiotepa and cyclosphosphamide; alkyl sulfonates, such as busulfan, improsulfan, and piposulfan; aziridines, such as benzodopa, carboquone, meturedopa, and uredopa; ethylenimines and methylamelamines, including altretamine, triethylenemelamine, trietylenephosphoramide, triethiylenethiophosphoramide, and trimethylolomelamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CBT-TM1); eleutherobin; pancratistatin; a sarcodictyin; spongistatin; nitrogen mustards, such as chlorambucil, chlomaphazine, cholophosphamide, estramustine, ifosfamide, mechlorethamine, mechlorethamine oxide hydrochloride, melphalan, novembichin, phenesterine, prednimustine, trofosfamide, and uracil mustard; nitrosureas, such as carmustine, chlorozotocin, fotemustine, lomustine, nimustine, and ranimnustine; antibiotics, such as the enediyne antibiotics (e.g., calicheamicin, especially calicheamicin gammall and calicheamicin omegall); dynemicin, including dynemicin A; bisphosphonates, such as clodronate; an esperamicin; as well as neocarzinostatin chromophore and related chromoprotein enediyne antiobiotic chromophores, aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins, such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, and zorubicin; anti-metabolites, such as methotrexate and 5-fluorouracil (5-FU); folic acid analogues, such as denopterin, pteropterin, and trimetrexate; purine analogs, such as fludarabine, 6-mercaptopurine, thiamiprine, and thioguanine; pyrimidine analogs, such as ancitabine, azacitidine, 6-azauridine, carmofur, cytarabine, dideoxyuridine, doxifluridine, enocitabine, and floxuridine; androgens, such as calusterone, dromostanolone propionate, epitiostanol, mepitiostane, and testolactone; anti-adrenals, such as mitotane and trilostane; folic acid replenishers such as folinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids, such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK polysaccharide complex; razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; taxoids, e.g., paclitaxel and docetaxel gemcitabine; 6-thioguanine; mercaptopurine; platinum coordination complexes, such as cisplatin, oxaliplatin, and carboplatin; vinblastine; platinum; etoposide (VP-16); ifosfamide; mitoxantrone; vincristine; vinorelbine; novantrone; teniposide; edatrexate; daunomycin; aminopterin; xeloda; ibandronate; irinotecan (e.g., CPT-11); topoisomerase inhibitor RFS 2000; difluorometlhylomithine (DMFO); retinoids, such as retinoic acid; capecitabine; carboplatin, procarbazine, plicomycin, gemcitabine, navelbine, famesyl-protein tansferase inhibitors, transplatinum, and pharmaceutically acceptable salts, acids, or derivatives of any of the above.

Some non-limiting examples of chemotherapeutic drugs which can be combined with anti-cancer therapies of the present disclosure are carboplatin (Paraplatin), cisplatin (Platinol, Platinol-AQ), cyclophosphamide (Cytoxan, Neosar), docetaxel (Taxotere), doxorubicin (Adriamycin), erlotinib (Tarceva), etoposide (VePesid), fluorouracil (5-FU), gemcitabine (Gemzar), imatinib mesylate (Gleevec), irinotecan (Camptosar), methotrexate (Folex, Mexate, Amethopterin), paclitaxel (Taxol, Abraxane), sorafinib (Nexavar), sunitinib (Sutent), topotecan (Hycamtin), vincristine (Oncovin, Vincasar PFS), and vinblastine (Velban).

In some embodiments, the anti-cancer therapy comprises a kinase inhibitor. In some embodiments, the methods provided herein comprise administering to the individual a kinase inhibitor, e.g., in combination with another anti-cancer therapy. Examples of kinase inhibitors include those that target one or more receptor tyrosine kinases, e.g., BCR-ABL, B-Raf, EGFR, HER-2/ErbB2, IGF-IR, PDGFR-a, PDGFR-β, cKit, Flt-4, Flt3, FGFR1, FGFR3, FGFR4, CSF1R, c-Met, RON, c-Ret, or ALK; one or more cytoplasmic tyrosine kinases, e.g., c-SRC, c-YES, Abl, or JAK-2; one or more serine/threonine kinases, e.g., ATM, Aurora A & B, CDKs, mTOR, PKCi, PLKs, b-Raf, S6K, or STK11/LKB1; or one or more lipid kinases, e.g., PI3K or SKI. Small molecule kinase inhibitors include PHA-739358, nilotinib, dasatinib, PD166326, NSC 743411, lapatinib (GW-572016), canertinib (CI-1033), semaxinib (SU5416), vatalanib (PTK787/ZK222584), sutent (SU1 1248), sorafenib (BAY 43-9006), or leflunomide (SU101). Additional non-limiting examples of tyrosine kinase inhibitors include imatinib (Gleevec/Glivec) and gefitinib (Iressa).

In some embodiments, the anti-cancer therapy comprises an anti-angiogenic agent. In some embodiments, the methods provided herein comprise administering to the individual an anti-angiogenic agent, e.g., in combination with another anti-cancer therapy. Angiogenesis inhibitors prevent the extensive growth of blood vessels (angiogenesis) that tumors require to survive. Non-limiting examples of angiogenesis-mediating molecules or angiogenesis inhibitors which may be used in the methods of the present disclosure include soluble VEGF (for example: VEGF isoforms, e.g., VEGF121 and VEGF165; VEGF receptors, e.g., VEGFR1, VEGFR2; and co-receptors, e.g., Neuropilin-1 and Neuropilin-2), NRP-1, angiopoietin 2, TSP-1 and TSP-2, angiostatin and related molecules, endostatin, vasostatin, calreticulin, platelet factor-4, TIMP and CDAI, Meth-1 and Meth-2, IFNα, IFN-β and IFN-γ, CXCL10, IL-4, IL-12 and IL-18, prothrombin (kringle domain-2), antithrombin III fragment, prolactin, VEGI, SPARC, osteopontin, maspin, canstatin, proliferin-related protein, restin and drugs such as bevacizumab, itraconazole, carboxyamidotriazole, TNP-470, CM101, IFN-α platelet factor-4, suramin, SU5416, thrombospondin, VEGFR antagonists, angiostatic steroids and heparin, cartilage-derived angiogenesis inhibitory factor, matrix metalloproteinase inhibitors, 2-methoxyestradiol, tecogalan, tetrathiomolybdate, thalidomide, thrombospondin, prolactina v β3 inhibitors, linomide, or tasquinimod. In some embodiments, known therapeutic candidates that may be used according to the methods of the disclosure include naturally occurring angiogenic inhibitors, including without limitation, angiostatin, endostatin, or platelet factor-4. In another embodiment, therapeutic candidates that may be used according to the methods of the disclosure include, without limitation, specific inhibitors of endothelial cell growth, such as TNP-470, thalidomide, and interleukin-12. Still other anti-angiogenic agents that may be used according to the methods of the disclosure include those that neutralize angiogenic molecules, including without limitation, antibodies to fibroblast growth factor, antibodies to vascular endothelial growth factor, antibodies to platelet derived growth factor, or antibodies or other types of inhibitors of the receptors of EGF, VEGF or PDGF. In some embodiments, anti-angiogenic agents that may be used according to the methods of the disclosure include, without limitation, suramin and its analogs, and tecogalan. In other embodiments, anti-angiogenic agents that may be used according to the methods of the disclosure include, without limitation, agents that neutralize receptors for angiogenic factors or agents that interfere with vascular basement membrane and extracellular matrix, including, without limitation, metalloprotease inhibitors and angiostatic steroids. Another group of anti-angiogenic compounds that may be used according to the methods of the disclosure includes, without limitation, anti-adhesion molecules, such as antibodies to integrin alpha v beta 3. Still other anti-angiogenic compounds or compositions that may be used according to the methods of the disclosure include, without limitation, kinase inhibitors, thalidomide, itraconazole, carboxyamidotriazole, CM101, IFN-α, IL-12, SU5416, thrombospondin, cartilage-derived angiogenesis inhibitory factor, 2-methoxyestradiol, tetrathiomolybdate, thrombospondin, prolactin, and linomide. In one particular embodiment, the anti-angiogenic compound that may be used according to the methods of the disclosure is an antibody to VEGF, such as Avastin®/bevacizumab (Genentech).

In some embodiments, the anti-cancer therapy comprises an anti-DNA repair therapy. In some embodiments, the methods provided herein comprise administering to the individual an anti-DNA repair therapy, e.g., in combination with another anti-cancer therapy. In some embodiments, the anti-DNA repair therapy is a PARP inhibitor (e.g., talazoparib, rucaparib, olaparib), a RAD51 inhibitor (e.g., RI-1), or an inhibitor of a DNA damage response kinase, e.g., CHCK1 (e.g., AZD7762), ATM (e.g., KU-55933, KU-60019, NU7026, or VE-821), and ATR (e.g., NU7026).

In some embodiments, the anti-cancer therapy comprises a radiosensitizer. In some embodiments, the methods provided herein comprise administering to the individual a radiosensitizer, e.g., in combination with another anti-cancer therapy. Exemplary radiosensitizers include hypoxia radiosensitizers such as misonidazole, metronidazole, and trans-sodium crocetinate, a compound that helps to increase the diffusion of oxygen into hypoxic tumor tissue. The radiosensitizer can also be a DNA damage response inhibitor interfering with base excision repair (BER), nucleotide excision repair (NER), mismatch repair (MMR), recombinational repair comprising homologous recombination (HR) and non-homologous end-joining (NHEJ), and direct repair mechanisms. Single strand break (SSB) repair mechanisms include BER, NER, or MMR pathways, while double stranded break (DSB) repair mechanisms consist of HR and NHEJ pathways. Radiation causes DNA breaks that, if not repaired, are lethal. SSBs are repaired through a combination of BER, NER and MMR mechanisms using the intact DNA strand as a template. The predominant pathway of SSB repair is BER, utilizing a family of related enzymes termed poly-(ADP-ribose) polymerases (PARP). Thus, the radiosensitizer can include DNA damage response inhibitors such as PARP inhibitors.

In some embodiments, the anti-cancer therapy comprises an anti-inflammatory agent. In some embodiments, the methods provided herein comprise administering to the individual an anti-inflammatory agent, e.g., in combination with another anti-cancer therapy. In some embodiments, the anti-inflammatory agent is an agent that blocks, inhibits, or reduces inflammation or signaling from an inflammatory signaling pathway. In some embodiments, the anti-inflammatory agent inhibits or reduces the activity of one or more of any of the following: IL-1, IL-2, IL-3, IL-4, IL-5, IL-6, IL-7, IL-8, IL-9, IL-10, IL-12, IL-13, IL-15, IL-18, IL-23; interferons (IFNs), e.g., IFNα, IFNβ, IFNγ, IFN-γ inducing factor (IGIF); transforming growth factor-β (TGF-β); transforming growth factor-α (TGF-α); tumor necrosis factors, e.g., TNF-α, TNF-β, TNF-RI, TNF-RII; CD23; CD30; CD40L; EGF; G-CSF; GDNF; PDGF-BB; RANTES/CCL5; IKK; NF-κB; TLR2; TLR3; TLR4; TL5; TLR6; TLR7; TLR8; TLR8; TLR9; and/or any cognate receptors thereof. In some embodiments, the anti-inflammatory agent is an IL-1 or IL-1 receptor antagonist, such as anakinra (Kineret®), rilonacept, or canakinumab. In some embodiments, the anti-inflammatory agent is an IL-6 or IL-6 receptor antagonist, e.g., an anti-IL-6 antibody or an anti-IL-6 receptor antibody, such as tocilizumab (ACTEMRA®), olokizumab, clazakizumab, sarilumab, sirukumab, siltuximab, or ALX-0061. In some embodiments, the anti-inflammatory agent is a TNF-α antagonist, e.g., an anti-TNFα antibody, such as infliximab (Remicade®), golimumab (Simponi®), adalimumab (Humira®), certolizumab pegol (Cimzia®) or etanercept. In some embodiments, the anti-inflammatory agent is a corticosteroid. Exemplary corticosteroids include, but are not limited to, cortisone (hydrocortisone, hydrocortisone sodium phosphate, hydrocortisone sodium succinate, Ala-Cort®, Hydrocort Acetate®, hydrocortone phosphate Lanacort®, Solu-Cortef®), decadron (dexamethasone, dexamethasone acetate, dexamethasone sodium phosphate, Dexasone®, Diodex®, Hexadrol®, Maxidex®), methylprednisolone (6-methylprednisolone, methylprednisolone acetate, methylprednisolone sodium succinate, Duralone®, Medralone®, Medrol®, M-Prednisol®, Solu-Medrol®), prednisolone (Delta-Cortef®, ORAPRED@, Pediapred®, Prezone®), and prednisone (Deltasone®, Liquid Pred®, Meticorten®, Orasone®), and bisphosphonates (e.g., pamidronate (Aredia®), and zoledronic acid (Zometac®).

In some embodiments, the anti-cancer therapy comprises an anti-hormonal agent. In some embodiments, the methods provided herein comprise administering to the individual an anti-hormonal agent, e.g., in combination with another anti-cancer therapy. Anti-hormonal agents are agents that act to regulate or inhibit hormone action on tumors. Examples of anti-hormonal agents include anti-estrogens and selective estrogen receptor modulators (SERMs), including, for example, tamoxifen (including NOLVADEX® tamoxifen), raloxifene, droloxifene, 4-hydroxytamoxifen, trioxifene, keoxifene, LY117018, onapristone, and FARESTON® toremifene; aromatase inhibitors that inhibit the enzyme aromatase, which regulates estrogen production in the adrenal glands, such as, for example, 4(5)-imidazoles, aminoglutethimide, MEGACE® megestrol acetate, AROMASIN® exemestane, formestanie, fadrozole, RIVISOR® vorozole, FEMARA® letrozole, and ARIMIDEX® (anastrozole); anti-androgens such as flutamide, nilutamide, bicalutamide, leuprolide, and goserelin; troxacitabine (a 1,3-dioxolane nucleoside cytosine analog); antisense oligonucleotides, particularly those that inhibit expression of genes in signaling pathways implicated in aberrant cell proliferation, such as, for example, PKC-alpha, Raf, H-Ras, and epidermal growth factor receptor (EGF-R); vaccines such as gene therapy vaccines, for example, ALLOVECTIN® vaccine, LEUVECTIN® vaccine, and VAXID® vaccine; PROLEUKIN® rIL-2; LURTOTECAN® topoisomerase 1 inhibitor; ABARELIX® rmRH; and pharmaceutically acceptable salts, acids or derivatives of any of the above.

In some embodiments, the anti-cancer therapy comprises an antimetabolite chemotherapeutic agent. In some embodiments, the methods provided herein comprise administering to the individual an antimetabolite chemotherapeutic agent, e.g., in combination with another anti-cancer therapy. Antimetabolite chemotherapeutic agents are agents that are structurally similar to a metabolite, but cannot be used by the body in a productive manner. Many antimetabolite chemotherapeutic agents interfere with the production of RNA or DNA. Examples of antimetabolite chemotherapeutic agents include gemcitabine (GEMZAR®), 5-fluorouracil (5-FU), capecitabine (XELODA™), 6-mercaptopurine, methotrexate, 6-thioguanine, pemetrexed, raltitrexed, arabinosylcytosine ARA-C cytarabine (CYTOSAR-U®), dacarbazine (DTIC-DOMED), azocytosine, deoxycytosine, pyridmidene, fludarabine (FLUDARA®), cladrabine, and 2-deoxy-D-glucose. In some embodiments, an antimetabolite chemotherapeutic agent is gemcitabine. Gemcitabine HCl is sold by Eli Lilly under the trademark GEMZAR®.

In some embodiments, the anti-cancer therapy comprises a platinum-based chemotherapeutic agent. In some embodiments, the methods provided herein comprise administering to the individual a platinum-based chemotherapeutic agent, e.g., in combination with another anti-cancer therapy. Platinum-based chemotherapeutic agents are chemotherapeutic agents that comprise an organic compound containing platinum as an integral part of the molecule. In some embodiments, a chemotherapeutic agent is a platinum agent. In some such embodiments, the platinum agent is selected from cisplatin, carboplatin, oxaliplatin, nedaplatin, triplatin tetranitrate, phenanthriplatin, picoplatin, or satraplatin.

In some aspects, provided herein are therapeutic formulations comprising an anti-cancer therapy provided herein, and a pharmaceutically acceptable carrier, excipient, or stabilizer. A formulation provided herein may contain more than one active compound, e.g., an anti-cancer therapy provided herein and one or more additional agents (e.g., anti-cancer agents).

Acceptable carriers, excipients, or stabilizers are non-toxic to recipients at the dosages and concentrations employed, and include, for example, one or more of: buffers such as phosphate, citrate, and other organic acids; antioxidants, including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens such as methyl or propyl paraben, catechol, resorcinol, cyclohexanol, 3-pentanol, or m-cresol; low molecular weight polypeptides (e.g., less than about 10 residues); proteins such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); surfactants such as non-ionic surfactants; or polymers such as polyethylene glycol (PEG).

The active ingredients may be entrapped in microcapsules. Such microcapsules may be prepared, for example, by coacervation techniques or by interfacial polymerization, for example, hydroxymethylcellulose or gelatin-microcapsules and poly-(methylmethacylate) microcapsules, respectively; in colloidal drug delivery systems (for example, liposomes, albumin microspheres, microemulsions, nano-particles and nano-capsules); or in macroemulsions. Such techniques are known in the art.

Sustained-release compositions may be prepared. Suitable examples of sustained-release compositions include semi-permeable matrices of solid hydrophobic polymers containing an anti-cancer therapy of the disclosure. Such matrices may be in the form of shaped articles, e.g., films, or microcapsules. Examples of sustained-release matrices include polyesters, hydrogels (for example, poly(2-hydroxyethyl-methacrylate), or poly(vinylalcohol)), polylactides, copolymers of L-glutamic acid and γ ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), and poly-D-(−)-3-hydroxybutyric acid.

Formulations to be used for in vivo administration are sterile. This is readily accomplished by filtration through sterile filtration membranes or other methods known in the art.

In some embodiments, the anti-cancer therapy is administered as a monotherapy. In some embodiments, the anti-cancer therapy is administered in combination with one or more additional anti-cancer therapies or treatments. In some embodiments, the one or more additional anti-cancer therapies or treatments include one or more anti-cancer therapies described herein. In some embodiments, the additional anti-cancer therapy comprises one or more of surgery, radiotherapy, chemotherapy, anti-angiogenic therapy, anti-DNA repair therapy, and anti-inflammatory therapy. In some embodiments, the additional anti-cancer therapy comprises an anti-neoplastic agent, a chemotherapeutic agent, a growth inhibitory agent, an anti-angiogenic agent, a radiation therapy, a cytotoxic agent, or combinations thereof. In some embodiments, an anti-cancer therapy may be administered in conjunction with a chemotherapy or chemotherapeutic agent. In some embodiments, the chemotherapy or chemotherapeutic agent is a platinum-based agent (including, without limitation cisplatin, carboplatin, oxaliplatin, and staraplatin). In some embodiments, an anti-cancer therapy may be administered in conjunction with a radiation therapy.

In some embodiments, the anti-cancer therapy for use in any of the methods described herein (e.g., as monotherapy or in combination with another therapy or treatment) is an anti-cancer therapy or treatment described by Pietrantonio et al., J Natl Cancer Inst (2017) 109(12) and/or by Wang et al., Cancers (2020) 12(2):426, which are hereby incorporated by reference.

In some embodiments, an anti-cancer therapy of the disclosure comprises a kinase inhibitor (e.g., an ALK-targeted kinase inhibitor) and a chemotherapy. In some embodiments, an anti-cancer therapy of the disclosure comprises a kinase inhibitor (e.g., an ALK-targeted kinase inhibitor) and a PD-1 inhibitor. In some embodiments, an anti-cancer therapy of the disclosure comprises ceritinib and nivolumab. In some embodiments, an anti-cancer therapy of the disclosure comprises a kinase inhibitor (e.g., an ALK-targeted kinase inhibitor) and an anti-VEGF agent. In some embodiments, an anti-cancer therapy of the disclosure comprises alectinib and bevacizumab. In some embodiments, an anti-cancer therapy of the disclosure comprises a kinase inhibitor (e.g., an ALK-targeted kinase inhibitor) and an integrin β3 inhibitor. In some embodiments, an anti-cancer therapy of the disclosure comprises a kinase inhibitor (e.g., an ALK-targeted kinase inhibitor) and a statin or statin-based agent. In some embodiments, an anti-cancer therapy of the disclosure comprises a kinase inhibitor (e.g., an ALK-targeted kinase inhibitor) and an EGFR inhibitor. In some embodiments, an anti-cancer therapy of the disclosure comprises a kinase inhibitor (e.g., an ALK-targeted kinase inhibitor) and an mTOR inhibitor. In some embodiments, an anti-cancer therapy of the disclosure comprises a kinase inhibitor (e.g., an ALK-targeted kinase inhibitor) and a PI3K inhibitor. In some embodiments, an anti-cancer therapy of the disclosure comprises a kinase inhibitor (e.g., an ALK-targeted kinase inhibitor) and a MAPK inhibitor. In some embodiments, an anti-cancer therapy of the disclosure comprises a kinase inhibitor (e.g., an ALK-targeted kinase inhibitor) and a CK4/6 inhibitor.

Any of the anti-cancer therapies (optionally as monotherapies or in combination with another therapy or treatment) may find use in any of the methods described herein.

### Kits

Also provided herein are kits for detecting a COL5A2-ALK fusion nucleic acid molecule or a COL3A1-ALK fusion nucleic acid molecule of the disclosure. In some embodiments, a kit provided herein comprises a reagent (e.g., one or more oligonucleotides, primers, probes or baits of the present disclosure) for detecting a fusion nucleic acid molecule provided herein. In some embodiments, the kit comprises a reagent (e.g., one or more oligonucleotides, primers, probes or baits of the present disclosure) for detecting a wild-type counterpart of a fusion nucleic acid molecule provided herein. In some embodiments, the reagent comprises one or more oligonucleotides, primers, probes or baits of the present disclosure capable of hybridizing to a fusion nucleic acid molecule provided herein, or to a wild-type counterpart of a fusion nucleic acid molecule provided herein. In some embodiments, the reagent comprises one or more oligonucleotides, primers, probes or baits of the present disclosure capable of distinguishing a fusion nucleic acid molecule provided herein from a wild-type counterpart of the fusion nucleic acid molecule provided herein. In some embodiments, the kit is for use according to any method of detecting fusion nucleic acid molecules known in the art or described herein, such as sequencing, PCR, in situ hybridization methods, a nucleic acid hybridization assay, an amplification-based assay, a PCR-RFLP assay, real-time PCR, sequencing, next-generation sequencing, a screening analysis, FISH, spectral karyotyping, MFISH, comparative genomic hybridization, in situ hybridization, sequence-specific priming (SSP) PCR, HPLC, and mass-spectrometric genotyping. In some embodiments, a kit provided herein further comprises instructions for detecting a fusion nucleic acid molecule of the disclosure, e.g., using one or more oligonucleotides, primers, probes or baits of the present disclosure.

Also provided herein are kits for detecting a COL5A2-ALK fusion polypeptide or a COL3A1-ALK fusion polypeptide of the disclosure. In some embodiments, a kit provided herein comprises a reagent (e.g., one or more antibodies of the present disclosure) for detecting a fusion polypeptide described herein. In some embodiments, the kit comprises a reagent (e.g., one or more antibodies of the present disclosure) for detecting the wild-type counterparts of a fusion polypeptide provided herein. In some embodiments, the reagent comprises one or more antibodies of the present disclosure capable of binding to a fusion polypeptide provided herein, or to wild-type counterparts of the fusion polypeptide provided herein. In some embodiments, the reagent comprises one or more antibodies of the present disclosure capable of distinguishing a fusion polypeptide provided herein from wild-type counterparts of a fusion polypeptide provided herein. In some embodiments, the kit is for use according to any protein or polypeptide detection assay known in the art or described herein, such as mass spectrometry (e.g., tandem mass spectrometry), a reporter assay (e.g., a fluorescence-based assay), immunoblots such as a Western blot, immunoassays such as enzyme-linked immunosorbent assays (ELISA), immunohistochemistry, other immunological assays (e.g., fluid or gel precipitin reactions, immunodiffusion, immunoelectrophoresis, radioimmunoassay (RIA), immunofluorescent assays), and analytic biochemical methods (e.g., electrophoresis, capillary electrophoresis, high performance liquid chromatography (HPLC), thin layer chromatography (TLC), hyperdiffusion chromatography). In some embodiments, the kit further comprises instructions for detecting a fusion polypeptide of the disclosure, e.g., using one or more antibodies of the present disclosure.

### Expression Vectors, Host Cells and Recombinant Cells

Provided herein are vectors comprising a COL5A2-ALK fusion nucleic acid molecule or a COL3A1-ALK fusion nucleic acid molecule, a bait, a probe, or an oligonucleotide described herein, or fragments thereof.

In some embodiments, a vector provided herein comprises a COL5A2-ALK fusion nucleic acid molecule or a COL3A1-ALK fusion nucleic acid molecule described herein, or a nucleic acid molecule encoding a COL5A2-ALK fusion polypeptide or a COL3A1-ALK fusion polypeptide described herein.

In some embodiments, a vector provided herein is a nucleic acid molecule capable of transporting another nucleic acid to which it has been linked (e.g., fusion nucleic acid molecules, baits, probes, or oligonucleotides described herein, or fragments thereof). In some embodiments, a vector is a plasmid, a cosmid or a viral vector. The vector may be capable of autonomous replication, or it can integrate into a host DNA. Viral vectors (e.g., comprising fusion nucleic acid molecules, baits, probes, or oligonucleotides described herein, or fragments thereof) are also contemplated herein, including, e.g., replication defective retroviruses, adenoviruses and adeno-associated viruses.

In some embodiments, a vector provided herein comprises a COL5A2-ALK fusion nucleic acid molecule, a COL3A1-ALK fusion nucleic acid molecule, a bait, a probe, or an oligonucleotide of the disclosure in a form suitable for expression thereof in a host cell. In some embodiments, the vector includes one or more regulatory sequences operatively linked to the nucleotide sequence to be expressed. In some embodiments, the one or more regulatory sequences include promoters (e.g., promoters derived from polyoma, Adenovirus 2, cytomegalovirus and Simian Virus 40), enhancers, and other expression control elements (e.g., polyadenylation signals). In some embodiments, a regulatory sequence directs constitutive expression of a nucleotide sequence (e.g., fusion nucleic acid molecules, baits, probes, or oligonucleotides described herein, or fragments thereof). In some embodiments, a regulatory sequence directs tissue-specific expression of a nucleotide sequence (e.g., fusion nucleic acid molecules, baits, probes, or oligonucleotides described herein, or fragments thereof). In some embodiments, a regulatory sequence directs inducible expression of a nucleotide sequence (e.g., fusion nucleic acid molecules, baits, probes, or oligonucleotides described herein, or fragments thereof). Examples of inducible regulatory sequences include, without limitation, promoters regulated by a steroid hormone, by a polypeptide hormone, or by a heterologous polypeptide, such as a tetracycline-inducible promoter. Examples of tissue- or cell-type-specific regulatory sequences include, without limitation, the albumin promoter, lymphoid-specific promoters, promoters of T cell receptors or immunoglobulins, neuron-specific promoters, pancreas-specific promoters, mammary gland-specific promoters, and developmentally-regulated promoters. In some embodiments, a vector provided herein comprises a COL5A2-ALK fusion nucleic acid molecule, a COL3A1-ALK fusion nucleic acid molecule, a bait, a probe, or an oligonucleotide of the disclosure in the sense or the anti-sense orientation. In some embodiments, a vector (e.g., an expression vector) provided herein is introduced into host cells to thereby produce a fusion polypeptide, e.g., a COL5A2-ALK fusion polypeptide or a COL3A1-ALK fusion polypeptide described herein, or a fragment or mutant form thereof.

In some embodiments, the design of a vector provided herein depends on such factors as the choice of the host cell to be transformed, the level of expression desired, and the like. In some embodiments, expression vectors are designed for the expression of fusion nucleic acid molecules, baits, probes, or oligonucleotides described herein, or fragments thereof, in prokaryotic or eukaryotic cells, such as E. coli cells, insect cells (e.g., using baculovirus expression vectors), yeast cells, or mammalian cells. In some embodiments, a vector described herein is transcribed and translated in vitro, for example using T7 promoter regulatory sequences and T7 polymerase. In some embodiments, a vector (e.g., an expression vector) provided herein comprises a fusion nucleic acid molecule described herein, wherein the nucleotide sequence of the fusion nucleic acid molecule described herein has been altered (e.g., codon optimized) so that the individual codons for each encoded amino acid are those preferentially utilized in the host cell.

Also provided herein are host cells, e.g., comprising fusion nucleic acid molecules, fusion polypeptides, baits, probes, vectors, or oligonucleotides of the disclosure. In some embodiments, a host cell (e.g., a recombinant host cell or recombinant cell) comprises a vector described herein (e.g., an expression vector described herein). In some embodiments, a fusion nucleic acid molecule, bait, probe, vector, or oligonucleotide provided herein further includes sequences which allow it to integrate into the host cell's genome (e.g., through homologous recombination at a specific site). In some embodiments, a host cell provided herein is a prokaryotic or eukaryotic cell. Non limiting examples of host cells include, without limitation, bacterial cells (e.g., E. coli), insect cells, yeast cells, or mammalian cells (e.g., human cells, rodent cells, mouse cells, rabbit cells, pig cells, Chinese hamster ovary cells (CHO), or COS cells, e.g., COS-7 cells, CV-1 origin SV40 cells). A host cell described herein includes the particular host cell, as well as the progeny of such a cell. Because certain modifications may occur in succeeding generations due to either mutation or environmental influences, such progeny may not, in fact, be identical to the parent host cell.

Fusion nucleic acid molecules, baits, probes, vectors, or oligonucleotides of the disclosure may be introduced into host cells using any suitable method known in the art, such as conventional transformation or transfection techniques (e.g., using calcium phosphate or calcium chloride co-precipitation, DEAE-dextran-mediated transfection, lipofection, or electroporation).

Also provided herein are methods of producing a COL5A2-ALK fusion polypeptide or a COL3A1-ALK fusion polypeptide, e.g., by culturing a host cell described herein (e.g., into which a recombinant expression vector encoding a polypeptide has been introduced) in a suitable medium such that the fusion polypeptide is produced. In another embodiment, the method further includes isolating a fusion polypeptide from the medium or the host cell.

### EXEMPLARY EMBODIMENTS

The following exemplary embodiments are representative of some aspects of the invention:
Embodiment 1. A method of treating or delaying progression of cancer, comprising administering to an individual an effective amount of a treatment comprising an anti-cancer therapy, wherein the cancer comprises a collagen alpha-2(V) chain (COL5A2)-anaplastic lymphoma kinase (ALK) fusion nucleic acid molecule or polypeptide or a collagen alpha-1(III) chain (COL3A1)-ALK fusion nucleic acid molecule or polypeptide.
Embodiment 2. A method of treating or delaying progression of cancer, comprising, responsive to knowledge of a COL5A2-ALK fusion nucleic acid molecule or polypeptide or a COL3A1-ALK fusion nucleic acid molecule or polypeptide in a sample from an individual, administering to the individual an effective amount of a treatment comprising an anti-cancer therapy.
Embodiment 3. A method of identifying one or more treatment options for an individual having cancer, the method comprising:
(a) acquiring knowledge of a COL5A2-ALK fusion nucleic acid molecule or polypeptide or a COL3A1-ALK fusion nucleic acid molecule or polypeptide in a sample from the individual; and
(b) generating a report comprising one or more treatment options identified for the individual based at least in part on said knowledge, wherein the one or more treatment options comprise an anti-cancer therapy.
Embodiment 4. A method of selecting treatment for an individual having cancer, comprising acquiring knowledge of a COL5A2-ALK fusion nucleic acid molecule or polypeptide or a COL3A1-ALK fusion nucleic acid molecule or polypeptide in a sample from an individual having cancer, wherein responsive to the acquisition of said knowledge: (i) the individual is classified as a candidate to receive a treatment comprising an anti-cancer therapy; and/or (ii) the individual is identified as likely to respond to a treatment comprising an anti-cancer therapy.
Embodiment 5. A method of treating or delaying progression of cancer, comprising:
(a) acquiring knowledge of a COL5A2-ALK fusion nucleic acid molecule or polypeptide or a COL3A1-ALK fusion nucleic acid molecule or polypeptide in a sample from an individual; and
(b) responsive to said knowledge, administering to the individual an effective amount of a treatment comprising an anti-cancer therapy.
Embodiment 6. A method of predicting survival of an individual having cancer treated with an anti-cancer therapy, comprising acquiring knowledge of a COL5A2-ALK fusion nucleic acid molecule or polypeptide or a COL3A1-ALK fusion nucleic acid molecule or polypeptide in a sample from the individual, wherein responsive to the acquisition of said knowledge, the individual is predicted to have longer survival after treatment with the anti-cancer therapy, as compared to an individual whose cancer does not exhibit the COL5A2-ALK fusion nucleic acid molecule or polypeptide, or the COL3A1-ALK fusion nucleic acid molecule or polypeptide.
Embodiment 7. A method of screening an individual having cancer, suspected of having cancer, being tested for cancer, being treated for cancer, or being tested for a susceptibility to cancer, comprising acquiring knowledge of a COL5A2-ALK fusion nucleic acid molecule or polypeptide or a COL3A1-ALK fusion nucleic acid molecule or polypeptide in a sample from the individual, wherein responsive to the acquisition of said knowledge, the individual is predicted to have increased risk of cancer recurrence, aggressive cancer, anti-cancer therapy resistance, or poor prognosis, as compared to an individual whose cancer does not exhibit the COL5A2-ALK fusion nucleic acid molecule or polypeptide, or the COL3A1-ALK fusion nucleic acid molecule or polypeptide.
Embodiment 8. A method of monitoring an individual having cancer, comprising acquiring knowledge of a COL5A2-ALK fusion nucleic acid molecule or polypeptide or a COL3A1-ALK fusion nucleic acid molecule or polypeptide in a sample from the individual, wherein responsive to the acquisition of said knowledge, the individual is predicted to have increased risk of cancer recurrence, aggressive cancer, anti-cancer therapy resistance, or poor prognosis, as compared to an individual whose cancer does not exhibit the COL5A2-ALK fusion nucleic acid molecule or polypeptide, or the COL3A1-ALK fusion nucleic acid molecule or polypeptide, optionally wherein the individual is being treated for cancer.
Embodiment 9. The method of any one of embodiments 3-8, wherein the acquiring knowledge comprises detecting the COL5A2-ALK fusion nucleic acid molecule or polypeptide or the COL3A1-ALK fusion nucleic acid molecule or polypeptide in a sample from the individual.
Embodiment 10. A method of identifying an individual having cancer who may benefit from a treatment comprising an anti-cancer therapy, the method comprising detecting a COL5A2-ALK fusion nucleic acid molecule or polypeptide or a COL3A1-ALK fusion nucleic acid molecule or polypeptide in a sample from the individual, wherein the presence of the COL5A2-ALK fusion nucleic acid molecule or polypeptide or the COL3A1-ALK fusion nucleic acid molecule or polypeptide in the sample identifies the individual as one who may benefit from the treatment comprising an anti-cancer therapy.
Embodiment 11. A method of selecting a therapy for an individual having cancer, the method comprising detecting a COL5A2-ALK fusion nucleic acid molecule or polypeptide or a COL3A1-ALK fusion nucleic acid molecule or polypeptide in a sample from the individual, wherein the presence of the COL5A2-ALK fusion nucleic acid molecule or polypeptide, or the COL3A1-ALK fusion nucleic acid molecule or polypeptide in the sample identifies the individual as one who may benefit from a treatment comprising an anti-cancer therapy.
Embodiment 12. A method of identifying one or more treatment options for an individual having cancer, the method comprising:
(a) detecting a COL5A2-ALK fusion nucleic acid molecule or polypeptide or a COL3A1-ALK fusion nucleic acid molecule or polypeptide in a sample from the individual; and
(b) generating a report comprising one or more treatment options identified for the individual based at least in part on the presence of the COL5A2-ALK fusion nucleic acid molecule or polypeptide or the COL3A1-ALK fusion nucleic acid molecule or polypeptide in the sample, wherein the one or more treatment options comprise an anti-cancer therapy.
Embodiment 13. A method of treating or delaying progression of cancer, comprising:
(a) detecting a COL5A2-ALK fusion nucleic acid molecule or polypeptide or a COL3A1-ALK fusion nucleic acid molecule or polypeptide in a sample from an individual; and
(b) administering to the individual an effective amount of a treatment comprising an anti-cancer therapy.
Embodiment 14. A method of detecting a COL5A2-ALK fusion nucleic acid molecule or polypeptide or a COL3A1-ALK fusion nucleic acid molecule or polypeptide, the method comprising detecting the COL5A2-ALK fusion nucleic acid molecule or polypeptide, or the COL3A1-ALK fusion nucleic acid molecule or polypeptide in a sample from an individual.
Embodiment 15. A method of assessing a COL5A2-ALK fusion nucleic acid molecule or polypeptide or a COL3A1-ALK fusion nucleic acid molecule or polypeptide, the method comprising:
(a) detecting a COL5A2-ALK fusion nucleic acid molecule or polypeptide or a COL3A1-ALK fusion nucleic acid molecule or polypeptide in a sample from an individual; and
(b) providing an assessment of the COL5A2-ALK fusion nucleic acid molecule or polypeptide or the COL3A1-ALK fusion nucleic acid molecule or polypeptide.
Embodiment 16. The method of embodiment 14 or embodiment 15, wherein the individual has cancer, is suspected of having cancer, is being tested for cancer, is being treated for cancer, or is being tested for a susceptibility to cancer.
Embodiment 17. The method of any one of embodiments 9-16, further comprising selectively enriching for one or more nucleic acids comprising a COL5A2-ALK fusion nucleic acid molecule or a COL3A1-ALK fusion nucleic acid molecule nucleotide sequences to produce an enriched sample.
Embodiment 18. The method of any one of embodiments 1-13 and 16-17, wherein the cancer is a hematologic malignancy or a solid tumor malignancy.
Embodiment 19. The method of any one of embodiments 1-13 and 16-18, wherein the cancer is selected from the group consisting of anaplastic large cell lymphoma (ALCL), non-small cell lung cancer (NSCLC), colorectal cancer (CRC), sarcoma, sarcoma not otherwise specified (NOS), inflammatory myofibroblastic tumor (IMT), rhabdomyosarcoma, acute myeloid leukemia, histiocytosis, leiomyosarcoma, ALK-positive large B-cell lymphoma, epithelioid fibrous histiocytoma, a pulmonary carcinoma, a renal cell carcinoma, a thyroid carcinoma, a pancreatic carcinoma, carcinoma of unknown primary, ovarian carcinoma, glioma, mesothelioma, melanoma, and a Spitzoid tumor.
Embodiment 20. The method of any one of embodiments 1-13 and 16-18, wherein the cancer is a sarcoma, and wherein the sarcoma comprises COL3A1-ALK fusion nucleic acid molecule or polypeptide.
Embodiment 21. The method of embodiment 20, wherein the cancer is a uterus leiomyosarcoma, soft tissue inflammatory myofibroblastic tumor, or soft tissue sarcoma not otherwise specified (NOS).
Embodiment 22. The method of any one of embodiments 1-13 and 16-19, wherein the cancer is rhabdomyosarcoma, and wherein the rhabdomyosarcoma comprises a COL5A2-ALK fusion nucleic acid molecule or polypeptide.
Embodiment 23. The method of any one of embodiments 3-9 and 17-19, wherein the cancer is rhabdomyosarcoma, and wherein an anti-cancer therapy is administered to the individual responsive to acquiring knowledge of a COL5A2-ALK fusion nucleic acid molecule or polypeptide in the sample.
Embodiment 24. The method of any one of embodiments 3-9 and 17-19, wherein the cancer is rhabdomyosarcoma, and wherein the acquiring knowledge comprises acquiring knowledge of a COL5A2-ALK fusion nucleic acid molecule or polypeptide in the sample.
Embodiment 25. The method of any one of embodiments 9-19, wherein the cancer is rhabdomyosarcoma, and wherein the detecting comprises detecting a COL5A2-ALK fusion nucleic acid molecule or polypeptide in the sample.
Embodiment 26. The method of any one of embodiments 17-19, wherein the cancer is rhabdomyosarcoma, and wherein the selectively enriching comprises selectively enriching for one or more nucleic acids comprising COL5A2-ALK fusion nucleic acid molecule nucleotide sequences.
Embodiment 27. The method of any one of embodiments 1-13 and 16-19, wherein the cancer is leiomyosarcoma, inflammatory myofibroblastic tumor (IMT), or sarcoma not otherwise specified (NOS), and wherein the leiomyosarcoma, inflammatory myofibroblastic tumor (IMT), or sarcoma not otherwise specified (NOS) comprises a COL3A1-ALK fusion nucleic acid molecule or polypeptide.
Embodiment 28. The method of any one of embodiments 3-9 and 17-19, wherein the cancer is leiomyosarcoma, inflammatory myofibroblastic tumor (IMT), or sarcoma not otherwise specified (NOS), and wherein an anti-cancer therapy is administered to the individual responsive to acquiring knowledge of a COL3A1-ALK fusion nucleic acid molecule or polypeptide in the sample.
Embodiment 29. The method of any one of embodiments 3-9 and 17-19, wherein the cancer is leiomyosarcoma, inflammatory myofibroblastic tumor (IMT), or sarcoma not otherwise specified (NOS), and wherein the acquiring knowledge comprises acquiring knowledge of a COL3A1-ALK fusion nucleic acid molecule or polypeptide in the sample.
Embodiment 30. The method of any one of embodiments 9-19, wherein the cancer is leiomyosarcoma, inflammatory myofibroblastic tumor (IMT), or sarcoma not otherwise specified (NOS), and wherein the detecting comprises detecting a COL3A1-ALK fusion nucleic acid molecule or polypeptide in the sample.
Embodiment 31. The method of any one of embodiments 17-19, wherein the cancer is leiomyosarcoma, inflammatory myofibroblastic tumor (IMT), or sarcoma not otherwise specified (NOS), and wherein the selectively enriching comprises selectively enriching for one or more nucleic acids comprising COL3A1-ALK fusion nucleic acid molecule nucleotide sequences.
Embodiment 32. The method of any one of embodiments 1-13 and 17-31, wherein the anti-cancer therapy comprises a small molecule inhibitor, an antibody, a cellular therapy, or a nucleic acid.
Embodiment 33. The method of any one of embodiments 1-13 and 17-32, wherein the anti-cancer therapy comprises an ALK-targeted therapy.
Embodiment 34. The method of embodiment 33, wherein the ALK-targeted therapy is a kinase inhibitor.
Embodiment 35. The method of embodiment 34, wherein the kinase inhibitor is selected from the group consisting of crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, and TAE684 (NVP-TAE684).
Embodiment 36. The method of embodiment 32, wherein the cellular therapy is an adoptive therapy, a T cell-based therapy, a natural killer (NK) cell-based therapy, a chimeric antigen receptor (CAR)-T cell therapy, a recombinant T cell receptor (TCR) T cell therapy, or a dendritic cell (DC)-based therapy.
Embodiment 37. The method of embodiment 32, wherein the nucleic acid comprises a double-stranded RNA (dsRNA), a small interfering RNA (siRNA), or a small hairpin RNA (shRNA).
Embodiment 38. The method of any one of embodiments 1-13 and 17-32, wherein the anti-cancer therapy comprises a heat shock protein (HSP) inhibitor, a MYC inhibitor, an HDAC inhibitor, an immunotherapy, an ALK neoantigen a vaccine, or a cellular therapy.
Embodiment 39. The method of embodiment 38, wherein the HSP inhibitor is an HSP90 inhibitor.
Embodiment 40. The method of embodiment 39, wherein the HSP90 inhibitor is ganetespib.
Embodiment 41. The method of any one of embodiments 32-40, wherein the treatment or the one or more treatment options further comprise a second therapeutic agent.
Embodiment 42. The method of embodiment 41, wherein the second therapeutic agent is an immune checkpoint inhibitor, a VEGF inhibitor, an Integrin 03 inhibitor, a statin, an EGFR inhibitor, an mTOR inhibitor, a PI3K inhibitor, a MAPK inhibitor, or a CDK4/6 inhibitor.
Embodiment 43. The method of embodiment 42, wherein the immune checkpoint inhibitor is a PD-1 or a PD-L1 inhibitor.
Embodiment 44. The method of embodiment 43, wherein the PD-1 inhibitor is nivolumab.
Embodiment 45. The method of embodiment 42, wherein the VEGF inhibitor is bevacizumab.
Embodiment 46. The method of any one of embodiments 1-26 and 32-45, wherein the COL5A2-ALK fusion nucleic acid molecule comprises: exon 1 or a portion thereof of COL5A2 fused to intron 5 or a portion thereof of ALK; intron 1 or a portion thereof of COL5A2 fused to intron 5 or a portion thereof of ALK; exon 1 or a portion thereof of COL5A2 fused to exon 6 or a portion thereof of ALK; or intron 1 or a portion thereof of COL5A2 fused to exon 6 or a portion thereof of ALK.
Embodiment 47. The method of any one of embodiments 1-26 and 32-45, wherein the COL5A2-ALK fusion nucleic acid molecule comprises a nucleotide sequence comprising, in the 5′ to 3′ direction, exon 1 or a portion thereof of COL5A2, and exon 6 or a portion thereof and exons 7-29 of ALK.
Embodiment 48. The method of any one of embodiments 1-26 and 32-45, wherein the COL5A2-ALK fusion nucleic acid molecule results from a breakpoint in exon 1 or in intron 1 of COL5A2, and in intron 5 or in exon 6 of ALK.
Embodiment 49. The method of any one of embodiments 1-26 and 32-45, wherein the COL5A2-ALK fusion nucleic acid molecule comprises the nucleotide sequence of SEQ ID NO: 7, or a nucleotide sequence at least 70%, 75%, 80%, 85%, 90%, 91%, 92%, 93%, 94%, 95%, 96%, 97%, 98%, 99%, or 99.5% identical thereto.
Embodiment 50. The method of any one of embodiments 1-26 and 32-45, wherein the COL5A2-ALK fusion polypeptide comprises: an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK; or an amino acid sequence at least about 85% identical to an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK.
Embodiment 51. The method of any one of embodiments 1-26 and 32-45, wherein the COL5A2-ALK fusion polypeptide comprises the amino acid sequence of SEQ ID NO: 10, or an amino acid sequence at least 70%, 75%, 80%, 85%, 90%, 91%, 92%, 93%, 94%, 95%, 96%, 97%, 98%, 99%, or 99.5% identical thereto.
Embodiment 52. The method of any one of embodiments 1-19 and 27-45, wherein the COL3A1-ALK fusion nucleic acid molecule comprises:
(a) exon 48 or a portion thereof of COL3A1 fused to intron 18 or a portion thereof of ALK; exon 48 or a portion thereof of COL3A1 fused to exon 19 or a portion thereof of ALK; intron 48 or a portion thereof of COL3A1 fused to intron 18 or a portion thereof of ALK; or intron 48 or a portion thereof of COL3A1 fused to exon 19 or a portion thereof of ALK; or
(b) exon 2 or a portion thereof of COL3A1 fused to intron 18 or a portion thereof of ALK; exon 2 or a portion thereof of COL3A1 fused to exon 19 or a portion thereof of ALK; intron 2 or a portion thereof of COL3A1 fused to intron 18 or a portion thereof of ALK; or intron 2 or a portion thereof of COL3A1 fused to exon 19 or a portion thereof of ALK.
Embodiment 53. The method of any one of embodiments 1-19 and 27-45, wherein the COL3A1-ALK fusion nucleic acid molecule comprises a nucleotide sequence comprising, in the 5′ to 3′ direction:
(a) exons 1-47 and exon 48 or a portion thereof of COL3A1, and exon 19 or a portion thereof and exons 20-29 of ALK; or
(b) exon 1 and exon 2 or a portion thereof of COL3A1, and exon 19 or a portion thereof and exons 20-29 of ALK.
Embodiment 54. The method of any one of embodiments 1-19 and 27-45, wherein the COL3A1-ALK fusion nucleic acid molecule results from:
(a) a breakpoint in exon 2 or intron 2 of COL3A1, and in intron 18 or exon 19 of ALK; or a breakpoint joining Chr2:189849674 with Chr2:29448496; or
(b) a breakpoint in exon 48 or intron 48 of COL3A1, and in intron 18 or exon 19 of ALK; a breakpoint joining Chr2:189874528 with Chr2:29448490; or a breakpoint joining Chr2:189874814 with Chr2:29449440.
Embodiment 55. The method of any one of embodiments 1-19 and 27-45, wherein the COL3A1-ALK fusion nucleic acid molecule comprises the nucleotide sequence of SEQ ID NO: 8 or 9, or a nucleotide sequence at least 70%, 75%, 80%, 85%, 90%, 91%, 92%, 93%, 94%, 95%, 96%, 97%, 98%, 99%, or 99.5% identical thereto.
Embodiment 56. The method of any one of embodiments 1-19 and 27-45, wherein the COL3A1-ALK fusion polypeptide comprises:
(a) an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK; or an amino acid sequence at least about 85% identical to an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK; or
(b) an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK; or an amino acid sequence at least about 85% identical to an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK.
Embodiment 57. The method of any one of embodiments 1-19 and 27-45, wherein the COL3A1-ALK fusion polypeptide comprises the amino acid sequence of SEQ ID NO: 11 or 12, or an amino acid sequence at least 70%, 75%, 80%, 85%, 90%, 91%, 92%, 93%, 94%, 95%, 96%, 97%, 98%, 99%, or 99.5% identical thereto.
Embodiment 58. The method of any one of embodiments 1-45, 50-51, and 56-57, wherein the COL5A2-ALK fusion polypeptide, or the COL3A1-ALK fusion polypeptide has kinase activity.
Embodiment 59. The method of any one of embodiments 2-58, wherein the sample from the individual comprises fluid, cells, or tissue.
Embodiment 60. The method of embodiment 59, wherein the sample from the individual comprises a tumor biopsy or a circulating tumor cell.
Embodiment 61. The method of any one of embodiments 2-49, 52-55, and 59-60, wherein the sample from the individual is a nucleic acid sample.
Embodiment 62. The method of embodiment 61, wherein the nucleic acid sample comprises mRNA, genomic DNA, circulating tumor DNA, cell-free DNA, or cell-free RNA.
Embodiment 63. The method of any one of embodiments 9-49, 52-55, and 59-62, wherein the COL5A2-ALK fusion nucleic acid molecule, or the COL3A1-ALK fusion nucleic acid molecule is detected in the sample by one or more methods selected from the group consisting of a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), and mass-spectrometric genotyping.
Embodiment 64. The method of any one of embodiments 2-45, 50-51, and 56-60, wherein the sample from the individual is a protein sample.
Embodiment 65. The method of any one of embodiments 9-45, 50-51, 56-60, and 64, wherein the COL5A2-ALK fusion polypeptide, or the COL3A1-ALK fusion polypeptide is detected in the sample by one or more methods selected from the group consisting of immunoblotting, enzyme linked immunosorbent assay (ELISA), immunohistochemistry, and mass spectrometry.
Embodiment 66. An anti-cancer therapy for use in a method of treating or delaying progression of cancer, wherein the method comprises administering the anti-cancer therapy to an individual, wherein a COL5A2-ALK fusion nucleic acid molecule or polypeptide or a COL3A1-ALK fusion nucleic acid molecule or polypeptide has been detected in a sample obtained from the individual.
Embodiment 67. An anti-cancer therapy for use in the manufacture of a medicament for treating or delaying progression of cancer, wherein the medicament is to be administered to an individual, wherein a COL5A2-ALK fusion nucleic acid molecule or polypeptide or a COL3A1-ALK fusion nucleic acid molecule or polypeptide has been detected in a sample obtained from the individual.
Embodiment 68. In vitro use of one or more oligonucleotides for detecting a COL5A2-ALK fusion nucleic acid molecule or a COL3A1-ALK fusion nucleic acid molecule.
Embodiment 69. A kit comprising one or more oligonucleotides for detecting a COL5A2-ALK fusion nucleic acid molecule or a COL3A1-ALK fusion nucleic acid molecule.
Embodiment 70. In vitro use of a probe or bait for detecting a COL5A2-ALK fusion nucleic acid molecule or a COL3A1-ALK fusion nucleic acid molecule, wherein the probe or bait comprises a capture nucleic acid molecule configured to hybridize to a target nucleic acid molecule comprising a COL5A2-ALK fusion nucleic acid molecule or a COL3A1-ALK fusion nucleic acid molecule nucleotide sequences.
Embodiment 71. A kit comprising a probe or bait for detecting a COL5A2-ALK fusion nucleic acid molecule or a COL3A1-ALK fusion nucleic acid molecule.
Embodiment 72. An antibody or antibody fragment that specifically binds to a COL5A2-ALK fusion polypeptide or a COL3A1-ALK fusion polypeptide.
Embodiment 73. A kit comprising an antibody or antibody fragment that specifically binds to a COL5A2-ALK fusion polypeptide or a COL3A1-ALK fusion polypeptide for detecting the COL5A2-ALK fusion polypeptide or the COL3A1-ALK fusion polypeptide.
Embodiment 74. A vector comprising a COL5A2-ALK fusion nucleic acid molecule or a COL3A1-ALK fusion nucleic acid molecule, or a fragment thereof.
Embodiment 75. A host cell comprising a vector that comprises a COL5A2-ALK fusion nucleic acid molecule or a COL3A1-ALK fusion nucleic acid molecule, or a fragment thereof.

### EXAMPLES

The invention will be more fully understood by reference to the following examples. They should not, however, be construed as limiting the scope of the invention. It is understood that the examples and embodiments described herein are for illustrative purposes only and that various modifications or changes in light thereof will be suggested to persons skilled in the art and are to be included within the spirit and purview of this application and scope of the appended claims.

### Example 1: Fusion and Rearrangement Detection Using DNA and RNA-Based Comprehensive Genomic Profiling (CGP) of Sarcomas

Actionability of a growing subset of gene fusions and rearrangements (REs) is well established, with several alterations linked to approved targeted therapies. While there are FDA-approved assays for DNA-based detection of key recurring actionable RE, sarcomas can be enriched for rare REs that are not comprehensively covered on DNA panels.

In this Example, DNA and RNA CGP was performed on 9,969 sarcoma tissue specimens. DNA and RNA were co-extracted from 1.2 mm³ of FFPE tissue. Sequencing was performed for up to 406 cancer-related genes and introns from 28 genes commonly rearranged in cancer, as well as 265 RNA fusion genes.

Adaptor ligation-based hybrid capture-based sequencing was used. See, e.g., Frampton, G. M. et al. (2013) Nat. Biotech. 31:1023-1031. Mean coverage depth was >600×. Base substitutions, insertions, and deletions (short variants; SV) were detected. Actionable genes included NTRK1/2/3, BRAFMET, ALK, ERBB2, EGFR, FGFR1/2/3, ROS1, RET, and NRG1. Diagnostic genes/fusions included EWSR1, STAT6-NAB2, BCOR-ZC3H7B, BCOR-CCNB3, and FOXO1-PAX3/7. ALK fusions were breakpoints between intron 18 and 19 were considered canonical.

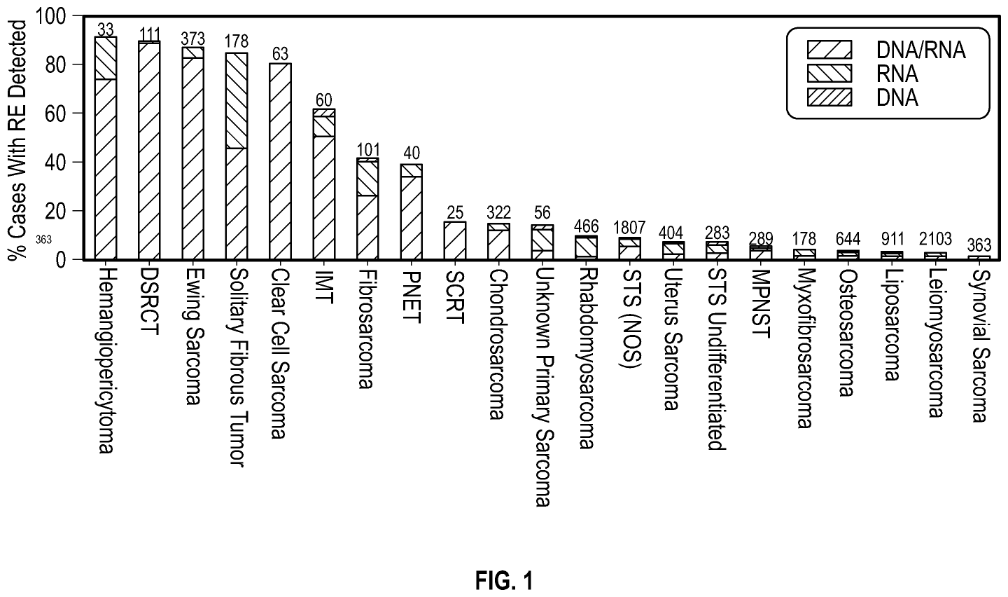

As shown in FIG. 1, detection of most REs in sarcoma occurred through DNA and RNA analysis. Exceptions were seen in gene fusions with rare breakpoints not covered by DNA baiting, particularly in hemangiopericytomas, solitary fibrous tumors, and rhabdomyosarcomas.

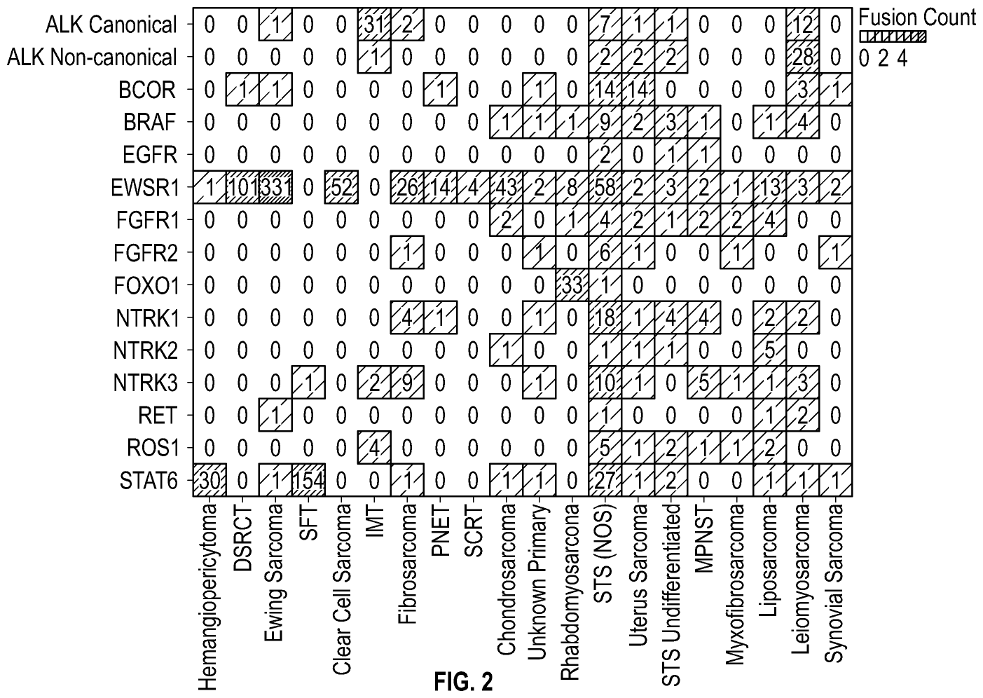

Diverse fusions were seen across a wide range of sarcomas. As shown in FIG. 2, 96% (892/927) of all fusions detected on DNA were confirmed in RNA. 25% (31/1271) of fusions were only detected in RNA, and 2% (30/1271) had complex DNA rearrangements resolved by analyzing RNA.

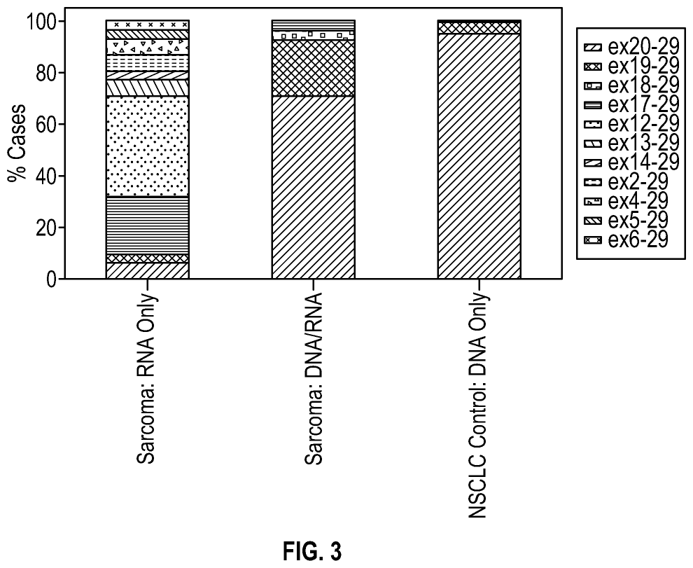

RNA analysis was able to detect ALK fusions with distinct breakpoints not covered by DNA baiting, which covers canonical NSCLC breakpoints (FIG. 3).

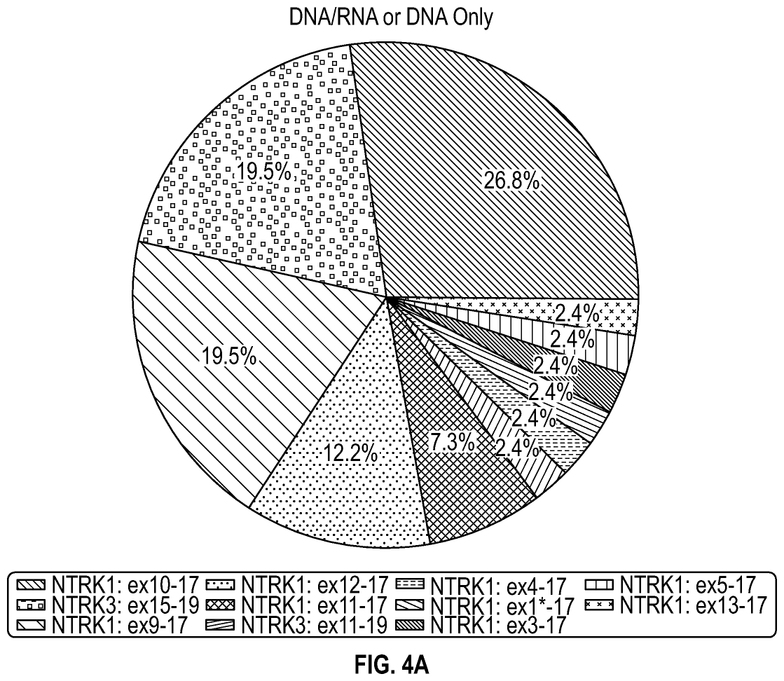

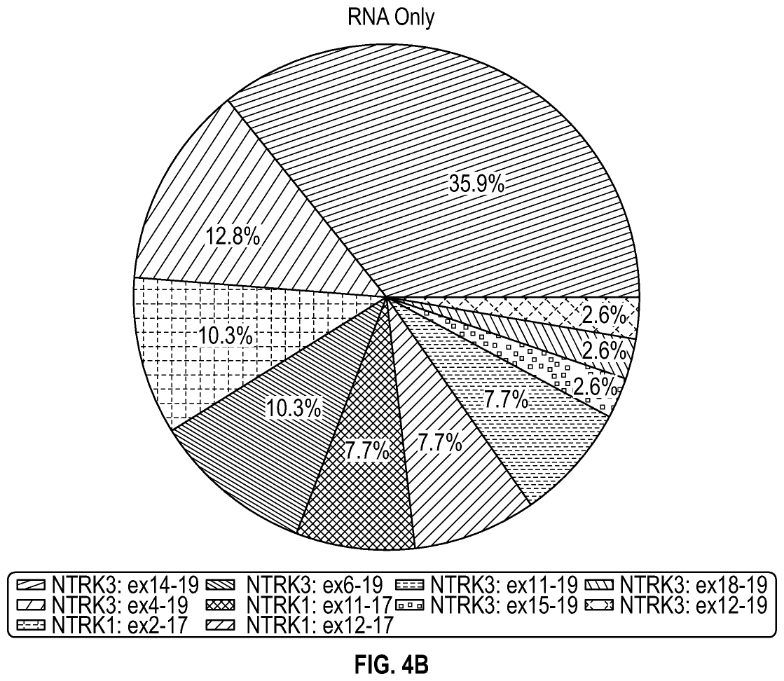

As shown in FIGS. 4A & 4B, of 41 NTRK1/3 gene fusions detected on DNA (5 DNA only; 36 DNA and RNA), 88% were confirmed in RNA. An additional 39 gene fusions were detected in RNA only; 100% were outside of the DNA baited region (NTRK1 intron 7, 8, and 9; NTRK3 no intron baiting).

These results demonstrated that, in most cases, rearrangements were detected in both DNA and RNA. However, RNA baiting increased the sensitivity for atypical fusions with non-canonical breakpoints. In ALK and NTRK1 non-fusion rearrangements, RNA further resolved the event as an actionable fusion or no RNA event was observed to support the actionability of the DNA finding.

1-7. (canceled)

8. A method of monitoring an individual having cancer, comprising:
(a) acquiring knowledge of a COL5A2-ALK fusion nucleic acid molecule or polypeptide or a COL3A1-ALK fusion nucleic acid molecule or polypeptide in a sample from the individual, and
(b) responsive to the acquisition of said knowledge, administering to the individual an effective amount of a treatment comprising an anti-cancer therapy, wherein the individual is predicted to have increased risk of cancer recurrence, aggressive cancer, anti-cancer therapy resistance, or poor prognosis, as compared to an individual whose cancer does not exhibit the COL5A2-ALK fusion nucleic acid molecule or polypeptide, or the COL3A1-ALK fusion nucleic acid molecule or polypeptide.

9-11. (canceled)

12. A method of identifying one or more treatment options for an individual having cancer, the method comprising:
(a) detecting a COL5A2-ALK fusion nucleic acid molecule or polypeptide or a COL3A1-ALK fusion nucleic acid molecule or polypeptide in a sample from the individual; and
(b) generating a report comprising one or more treatment options identified for the individual based at least in part on the presence of the COL5A2-ALK fusion nucleic acid molecule or polypeptide or the COL3A1-ALK fusion nucleic acid molecule or polypeptide in the sample, wherein the one or more treatment options comprise an anti-cancer therapy.

13. A method of treating or delaying progression of cancer, comprising:
(a) detecting a COL5A2-ALK fusion nucleic acid molecule or polypeptide or a COL3A1-ALK fusion nucleic acid molecule or polypeptide in a sample from an individual; and
(b) administering to the individual an effective amount of a treatment comprising an anti-cancer therapy.

14-16. (canceled)

17. The method of claim 13, further comprising selectively enriching for one or more nucleic acids comprising a COL5A2-ALK fusion nucleic acid molecule or a COL3A1-ALK fusion nucleic acid molecule nucleotide sequences to produce an enriched sample.

18. The method of claim 13, wherein the cancer is a hematologic malignancy or a solid tumor malignancy; or wherein the cancer is anaplastic large cell lymphoma (ALCL), non-small cell lung cancer (NSCLC), colorectal cancer (CRC), sarcoma, sarcoma not otherwise specified (NOS), inflammatory myofibroblastic tumor (IMT), rhabdomyosarcoma, acute myeloid leukemia, histiocytosis, leiomyosarcoma, ALK-positive large B-cell lymphoma, epithelioid fibrous histiocytoma, a pulmonary carcinoma, a renal cell carcinoma, a thyroid carcinoma, a pancreatic carcinoma, carcinoma of unknown primary, ovarian carcinoma, glioma, mesothelioma, melanoma, or a Spitzoid tumor.

19. (canceled)

20. The method of claim 13, wherein:
(a) the cancer is a sarcoma, and wherein the sarcoma comprises COL3A1-ALK fusion nucleic acid molecule or polypeptide;
(b) the cancer is rhabdomyosarcoma, and wherein the detecting comprises detecting a COL5A2-ALK fusion nucleic acid molecule or polypeptide in the sample; or
(c) the cancer is leiomyosarcoma, inflammatory myofibroblastic tumor (IMT), or sarcoma not otherwise specified (NOS), and wherein the detecting comprises detecting a COL3A1-ALK fusion nucleic acid molecule or polypeptide in the sample.

21-31. (canceled)

32. The method of claim 13, wherein the anti-cancer therapy comprises a small molecule inhibitor, an antibody, a cellular therapy, or a nucleic acid.

33. The method of claim 13, wherein the anti-cancer therapy comprises an ALK-targeted therapy.

34. The method of claim 33, wherein the ALK-targeted therapy is a kinase inhibitor comprising crizotinib, alectinib, ceritinib, lorlatinib, brigatinib, ensartinib (X-396), repotrectinib (TPX-005), entrectinib (RXDX-101), AZD3463, CEP-37440, belizatinib (TSR-011), ASP3026, KRCA-0008, TQ-B3139, TPX-0131, or TAE684 (NVP-TAE684).

35-37. (canceled)

38. The method of claim 13, wherein the anti-cancer therapy comprises a heat shock protein (HSP) inhibitor, a MYC inhibitor, an HDAC inhibitor, an immunotherapy, an ALK neoantigen a vaccine, or a cellular therapy.

39-40. (canceled)

41. The method of claim 13, wherein the treatment further comprises a second therapeutic agent comprising an immune checkpoint inhibitor, a VEGF inhibitor, an Integrin β3 inhibitor, a statin, an EGFR inhibitor, an mTOR inhibitor, a PI3K inhibitor, a MAPK inhibitor, or a CDK4/6 inhibitor.

42-45. (canceled)

46. The method of claim 13, wherein the COL5A2-ALK fusion nucleic acid molecule:
(a) comprises exon 1 or a portion thereof of COL5A2 fused to intron 5 or a portion thereof of ALK; intron 1 or a portion thereof of COL5A2 fused to intron 5 or a portion thereof of ALK; exon 1 or a portion thereof of COL5A2 fused to exon 6 or a portion thereof of ALK; or intron 1 or a portion thereof of COL5A2 fused to exon 6 or a portion thereof of ALK;
(b) comprises a nucleotide sequence comprising, in the 5′ to 3′ direction, exon 1 or a portion thereof of COL5A2, and exon 6 or a portion thereof and exons 7-29 of ALK;
(c) results from a breakpoint in exon 1 or in intron 1 of COL5A2, and in intron 5 or in exon 6 of ALK; or
(d) comprises the nucleotide sequence of SEQ ID NO: 7, or a nucleotide sequence at least 70%, 75%, 80%, 85%, 90%, 91%, 92%, 93%, 94%, 95%, 96%, 97%, 98%, 99%, or 99.5% identical thereto.

47-49. (canceled)

50. The method of claim 13, wherein:
(a) the COL5A2-ALK fusion polypeptide comprises: an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK; or an amino acid sequence at least about 85% identical to an amino acid sequence encoded by a nucleic acid molecule that comprises a nucleotide sequence comprising, in the 5′ to 3′ direction, exon 1 or a portion thereof of COL5A2, and exon 6 or a portion thereof and exons 7-29 of ALK; or
(b) the COL5A2-ALK fusion polypeptide comprises the amino acid sequence of SEQ ID NO: 10, or an amino acid sequence at least 70%, 75%, 80%, 85%, 90%, 91%, 92%, 93%, 94%, 95%, 96%, 97%, 98%, 99%, or 99.5% identical thereto.

51. (canceled)

52. The method of claim 13, wherein the COL3A1-ALK fusion nucleic acid molecule comprises:
(a) exon 48 or a portion thereof of COL3A1 fused to intron 18 or a portion thereof of ALK; exon 48 or a portion thereof of COL3A1 fused to exon 19 or a portion thereof of ALK; intron 48 or a portion thereof of COL3A1 fused to intron 18 or a portion thereof of ALK; or intron 48 or a portion thereof of COL3A1 fused to exon 19 or a portion thereof of ALK;
(b) exon 2 or a portion thereof of COL3A1 fused to intron 18 or a portion thereof of ALK; exon 2 or a portion thereof of COL3A1 fused to exon 19 or a portion thereof of ALK; intron 2 or a portion thereof of COL3A1 fused to intron 18 or a portion thereof of ALK; or intron 2 or a portion thereof of COL3A1 fused to exon 19 or a portion thereof of ALK;
(c) a nucleotide sequence comprising, in the 5′ to 3′ direction, exons 1-47 and exon 48 or a portion thereof of COL3A1, and exon 19 or a portion thereof and exons 20-29 of ALK; or
(d) a nucleotide sequence comprising, in the 5′ to 3′ direction, exon 1 and exon 2 or a portion thereof of COL3A1, and exon 19 or a portion thereof and exons 20-29 of ALK.

53. (canceled)

54. The method of claim 13, wherein:
(a) the COL3A1-ALK fusion nucleic acid molecule results from a breakpoint in exon 2 or intron 2 of COL3A1, and in intron 18 or exon 19 of ALK; or a breakpoint joining Chr2:189849674 with Chr2:29448496;
(b) the COL3A1-ALK fusion nucleic acid molecule results from a breakpoint in exon 48 or intron 48 of COL3A1, and in intron 18 or exon 19 of ALK; a breakpoint joining Chr2:189874528 with Chr2:29448490; or a breakpoint joining Chr2:189874814 with Chr2:29449440; or
(c) the COL3A1-ALK fusion nucleic acid molecule comprises the nucleotide sequence of SEQ ID NO: 8 or 9, or a nucleotide sequence at least 70%, 75%, 80%, 85%, 90%, 91%, 92%, 93%, 94%, 95%, 96%, 97%, 98%, 99%, or 99.5% identical thereto.

55. (canceled)

56. The method of claim 13, wherein the COL3A1-ALK fusion polypeptide comprises:
(a) an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK; or an amino acid sequence at least about 85% identical to an amino acid sequence encoded by a nucleic acid molecule that comprises a nucleotide sequence comprising, in the 5′ to 3′ direction, exons 1-47 and exon 48 or a portion thereof of COL3A1, and exon 19 or a portion thereof and exons 20-29 of ALK;
(b) an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK; or an amino acid sequence at least about 85% identical to an amino acid sequence encoded by a nucleic acid molecule that comprises a nucleotide sequence comprising, in the 5′ to 3′ direction, exon 1 and exon 2 or a portion thereof of COL3A1, and exon 19 or a portion thereof and exons 20-29 of ALK; or
(c) the amino acid sequence of SEQ ID NO: 11 or 12, or an amino acid sequence at least 70%, 75%, 80%, 85%, 90%, 91%, 92%, 93%, 94%, 95%, 96%, 97%, 98%, 99%, or 99.5% identical thereto.

57. (canceled)

58. The method of a claim 13, wherein the COL5A2-ALK fusion polypeptide, or the COL3A1-ALK fusion polypeptide has kinase activity.

59. The method of claim 13, wherein the sample from the individual:
(a) comprises fluid, cells, or tissue; or
(b) is a nucleic acid sample.

60. The method of claim 59, wherein the sample from the individual comprises a tumor biopsy or a circulating tumor cell; or wherein the nucleic acid sample comprises mRNA, genomic DNA, circulating tumor DNA, cell-free DNA, or cell-free RNA.

61-62. (canceled)

63. The method of claim 13, wherein the COL5A2-ALK fusion nucleic acid molecule, or the COL3A1-ALK fusion nucleic acid molecule is detected in the sample by a nucleic acid hybridization assay, an amplification-based assay, a polymerase chain reaction-restriction fragment length polymorphism (PCR-RFLP) assay, real-time PCR, sequencing, next-generation sequencing, a screening analysis, fluorescence in situ hybridization (FISH), spectral karyotyping, multicolor FISH (mFISH), comparative genomic hybridization, in situ hybridization, sequence-specific priming (SSP) PCR, high-performance liquid chromatography (HPLC), or mass-spectrometric genotyping.

64. The method of a claim 13, wherein the sample from the individual is a protein sample, and wherein the COL5A2-ALK fusion polypeptide, or the COL3A1-ALK fusion polypeptide is detected in the sample by immunoblotting, enzyme linked immunosorbent assay (ELISA), immunohistochemistry, and/or mass spectrometry.

65-75. (canceled)
